(19) United States
(12) Patent Application Publication
(10) Pub. No.: US 2024/0417703 A1
(43) Pub. Date: Dec. 19, 2024

(54) ERROR PRONE DNA POLYMERASE FOR ORGANELLE MUTATION

(71) Applicant: The University of Manchester, Manchester Greater Manchester (GB)

(72) Inventors: Junwei Ji, Manchester Greater Manchester (GB); Anil Day, Manchester Lancashire (GB)

(21) Appl. No.: 18/706,219

(22) PCT Filed: Oct. 31, 2022
(86) PCT No.: PCT/GB2022/052751

§ 371 Date: Apr. 30, 2024

(30) Foreign Application Priority Data
Nov. 1, 2021 (WO) PCT/GB2021/052823

(51) Int. Cl. C12N 9/12; C12N 15/10; C12N 15/82

(52) U.S. Cl. CPC C12N 9/1252; C12N 15/1024; C12N 15/8241; C12Y 207/07007

(57) ABSTRACT

The present invention relates to error prone DNA polymerases for organelle mutation, to methods of modifying a plant or pant thereof or organelle DNA of a plant, and plant products thereof. The invention further relates to a method of producing a plant having homoplastic modified organelle DNA using the error prone organellar DNA polymerase. The error prone organellar DNA polymerase allows plants with fixed homoplasmic modified organelle DNA to be produced.

### FIELD OF THE INVENTION

The present invention relates to error prone DNA polymerases for organelle mutation, and to nucleic acids, expression vectors, a plant cell, plant or part thereof, a seed and a method of modifying a plant or part thereof. The invention also relates to a method of modifying organelle DNA of a plant, a modified organelle and a plant comprising a modified organelle. The invention further relates to a method of producing a plant having homoplastic modified organelle DNA.

### BACKGROUND

Eukaryotic cells contain essential multi-copy organelle genomes in chloroplasts and mitochondria. Stable maintenance of these extra-nuclear genomes is essential for the proper functioning of mitochondria and chloroplasts. Mutants arising from mutations in organelle genomes have provided a valuable resource to study the roles of organelle genes. In animals and fungi, error-prone versions of gamma DNA polymerase have been used to elevate mutation rates in mitochondria to advance our understanding of mitochondrial genomes. Use of error-prone mutator DNA polymerases have led to new discoveries on the replication mechanisms and selective forces acting on animal mitochondrial genomes, and the impact of elevated mutation rates on organism biology including aging. By comparison, knowledge of these fundamental processes in the organelles of plants is limited.

In plant cells, plastids have their own set of genomes (Sakamoto and Takami, 2018). These genomes are in high copy number (up to 10,000 per cell) and highly conserved. Maintaining such genomes requires a stringent system for which the detailed mechanisms remain unknown. Plastid genomes are autonomously maintained but largely rely on the proteins encoded by the nucleus genome (Majeran et al., 2012).

Plant organelles contain a family of DNA polymerases, named Plant Organellar DNA Polymerases (POPs). The name POP now covers plant and protist organelle DNA polymerases to reflect the widespread distribution of POPs in a diverse range of algae and protozoans. POPs and gamma DNA polymerases are distantly related members of the DNA polymerase A family. In common with other DNA polymerases, POPs contain 5′-3′ DNA polymerisation and 3′-5′ exonuclease (proof-reading) domains in a single polypeptide. POPs are considered to be the sole enzymes responsible for replication of the mitochondrial and chloroplast genomes in plants. They are highly processive enzymes with a novel combination of activities including strand-displacement, translesion synthesis, microhomology-mediated-end-joining and 5′ deoxyribose phosphate removal. Plant POPs are expressed from nuclear genes and targeted to organelles.

There have been some efforts in the art to study mutations in plastid genomes of plants. Plastid DNA (ptDNA) and the DNA maintenance proteins are packed as DNA-protein complexes called nucleoids. Plant mutants with depleted nucleoid proteins have provided material to study the functions of some proteins, such as Whirly (Marechal et al., 2009), gyrase (Wall et al., 2004), MSH1 (Virdi et al., 2016) and plant organelle DNA polymerase (POP) (Parent et al., 2011). However, under natural conditions, spontaneous mutation is very rare in plastids, where the mutation rate is far lower than that in the nucleus (Smith, 2015). Several plastid/chloroplast mutator lines have also been created which have elevated mutation rate in ptDNA, such as Oenothera plastome mutator (pm) (Greiner, 2012) and barley chloroplast mutator (chm) (Prina, 1992, Landau et al., 2016) which are more frequently studied. However, the mutator alleles in these lines have not been isolated, limiting their use as tools for plastome mutagenesis to generate useful plant mutants.

There remains a need for a way to elevate mutagenesis in plastid DNA which produces plants having modified organelle DNA which is stable and which is retained in progeny. One or more aspects or embodiments of the present invention aim to provide novel error prone organelle DNA polymerases with elevated mutation rates in chloroplasts and mitochondria organelle DNA, and use thereof to produce and isolate plant mutants that carry advantageous traits such as herbicide resistance, male sterility, drought tolerance or higher yield.

### SUMMARY OF THE INVENTION

In a first aspect, the invention provides an organellar DNA Polymerase enzyme comprising an amino acid sequence according to SEQ ID NO: 1 or comprising an amino acid sequence having at least 35% identity thereto, or comprising a functional fragment thereof, wherein the amino acid sequence or functional fragment comprises a modification at or corresponding to position L903, and optionally one or more further modifications at the following positions: D390, E392, R862, E904, and N1065 of SEQ ID NO: 1, or positions corresponding thereto.

In one embodiment, the organellar DNA polymerase comprises an amino acid sequence which is a variant of SEQ ID NO: 1, or an amino acid sequence having at least 35% identity thereto, or a functional fragment thereof. By ‘variant’ it is meant that the reference sequence, such as SEQ ID NO: 1, contains one or more modifications. Suitably the one or more modifications listed above or corresponding thereto.

In one embodiment, the organellar DNA polymerase is an error prone organellar DNA polymerase. In one embodiment the organellar DNA polymerase is a modified organellar DNA polymerase. In one embodiment the organellar DNA polymerase is a mutated organellar DNA polymerase.

In one embodiment, the organellar DNA Polymerase enzyme comprises an amino acid sequence according to SEQ ID NO: 1 comprising a modification at position L903, and optionally one or more further modifications at the following positions: D390, E392, R862, E904, and N1065 of SEQ ID NO: 1.

In one embodiment, the organellar DNA Polymerase enzyme comprises an amino acid sequence according to SEQ ID NO: 1 comprising a modification at position L903, and further modifications at the following positions: D390 and E392 of SEQ ID NO: 1.

In one embodiment, the organellar DNA Polymerase enzyme comprises or consists of an amino acid sequence according to SEQ ID NO: 2.

For the sake of brevity, organellar DNA Polymerase enzymes in accordance with the various aspects and embodiments of the invention will be referred to herein as “the organellar DNA polymerase” or “polymerases of the invention”.

In a second aspect, the invention provides an isolated nucleic acid molecule comprising a sequence encoding the organellar DNA polymerase according to the first aspect of the invention.

In one embodiment, the isolated nucleic acid molecule comprises a sequence according to SEQ ID NO: 4.

It will be appreciated that nucleic acids in accordance with the second aspect of the invention may be expressed to yield an organellar DNA Polymerase enzyme in accordance with the first aspect of the invention.

In a third aspect, the invention provides an expression vector comprising the isolated nucleic acid molecule according to the second aspect of the invention.

In a fourth aspect, the invention provides an organelle comprising the organellar DNA polymerase according to the first aspect, the isolated nucleic acid molecule according to the second aspect, or the expression vector according to the third aspect of the invention.

In some embodiments, the organelle may be regarded as a host organelle. In some embodiments, the organelle is a plant organelle. In some embodiments the organelle is a plastid, suitably a chloroplast. In other embodiments the organelle is a mitochondria.

In a fifth aspect, the invention provides a cell comprising the organellar DNA polymerase according to the first aspect, the isolated nucleic acid molecule according to the second aspect, or the expression vector according to the third aspect, or the organelle according to the fourth aspect of the invention.

In some embodiments the cell may be regarded as a host cell. In some embodiments, the cell is a plant cell.

In a sixth aspect, the invention provides a plant or part thereof comprising the organellar DNA polymerase according to the first aspect, the isolated nucleic acid molecule according to the second aspect, or the expression vector according to the third aspect, or the organelle according to the fourth aspect or the cell according to the fifth aspect of the invention.

In a seventh aspect, the invention provides a seed capable of producing a plant or part thereof comprising the organellar DNA polymerase according to the first aspect, the isolated nucleic acid molecule according to the second aspect, the expression vector according to the third aspect, the organelle of according to the fourth aspect, or the cell according to the fifth aspect of the invention.

In an eighth aspect, the invention provides a plant produced from the seed according to the seventh aspect of the invention. Suitably the plant is directly produced from the seed, suitably it is directly grown from the seed.

In a ninth aspect, the invention provides a method of modifying a plant or part thereof, comprising:
a. Introducing the organellar DNA polymerase according to the first aspect, the isolated nucleic acid molecule according to the second aspect, or the expression vector according to the third aspect of the invention into the plant or part thereof;
b. Optionally inducing expression of the isolated nucleic acid molecule or expression vector in the plant or part thereof.

In one embodiment, introducing comprises transforming the organellar DNA polymerase according to the first aspect, the isolated nucleic acid molecule according to the second aspect, or the expression vector according to the third aspect of the invention into the plant or part thereof. In one embodiment, transforming into an organelle of the plant or part thereof. In one embodiment, transforming into a plastid of the plant or part thereof. In one embodiment, transforming into a chloroplast of the plant or part thereof. In other embodiments, transforming into a cell of the plant or part thereof, suitably wherein the isolated nucleic acid molecule or expression vector is expressed and subsequently targeted to the organelle.

In one embodiment, the method is a method of modifying the organelle DNA of a plant or part thereof.

In a tenth aspect, the invention provides a modified plant or part thereof produced by the method according to the ninth aspect of the invention.

In an eleventh aspect, the invention provides a method of modifying the organelle DNA of a plant or plant part, comprising, expressing in the plant or plant part, an organellar DNA polymerase according to the first aspect of the invention.

In one embodiment, the method of the eleventh aspect further comprises a step of introducing the organellar DNA polymerase according to the first aspect, the isolated nucleic acid molecule according to the second aspect, or the expression vector according to the third aspect of the invention into the plant or part thereof. In one embodiment, transforming into an organelle of the plant or part thereof. In one embodiment, transforming into a plastid of the plant or part thereof. In one embodiment, transforming into a chloroplast of the plant or part thereof. In other embodiments, transforming into a cell of the plant or part thereof, suitably wherein the organellar DNA polymerase is targeted to the organelle, or wherein the isolated nucleic acid molecule or expression vector is expressed and subsequently targeted to the organelle.

In an twelfth aspect, the invention provides a method of modifying organelle DNA in vitro or in vivo comprising:
a. Contacting the organellar DNA polymerase according the first aspect with organelle DNA under conditions suitable for the organellar DNA polymerase to modify the organelle DNA;
wherein optionally the contacting comprises introducing the organellar DNA polymerase according to the first aspect, the isolated nucleic acid molecule according to the second aspect, or the expression vector according to the third aspect into an organelle, and inducing expression thereof in the organelle.

In one embodiment, the method is a method of introducing transversion or transition mutations into organelle DNA. In one embodiment, the method is a method of introducing A-T transversion mutations, and A-G or C-T transition mutations into organelle DNA. In one embodiment, the method is a method of introducing A-T transversion mutations into organelle DNA.

In one embodiment, the method of modifying organelle DNA is in vivo. In one embodiment therefore the organelle is a plant organelle, and the method is method of modifying organelle DNA in a plant. In such embodiments, suitably the contacting comprises introducing the organellar DNA polymerase according to the first aspect, the isolated nucleic acid molecule according to the second aspect, or the expression vector according to the third aspect into the organelle, which is suitably a plant organelle, within a plant or plant part, and optionally inducing expression thereof in the organelle. In some embodiments the organelle is a plastid, suitably a chloroplast. In other embodiments the organelle is a mitochondria. In other embodiments, the contacting comprises introducing the organellar DNA polymerase according to the first aspect, the isolated nucleic acid molecule according to the second aspect, or the expression vector according to the third aspect into the plant cell, optionally within a plant or plant part, and optionally inducing expression thereof. Suitably wherein the organellar DNA polymerase is targeted to the organelle, or wherein the isolated nucleic acid molecule or expression vector is expressed and subsequently targeted to the organelle. Suitably therefore, introducing into an organelle may comprise targeting to an organelle.

In other embodiments, the method of modifying organelle DNA is in vitro. In one embodiment therefore the organelle is a plant organelle, and the method is method of modifying plant organelle DNA in vitro. Suitably by in vitro it is meant outside of a plant or plant part. Suitably in vitro may mean in a cell free system, or in a plant cell which is ex vivo. Therefore the method may be conducted by contacting the organellar DNA polymerase with organelle DNA in a cell free system, or contacting the organellar DNA polymerase with organelle DNA within an organelle, in a cell free system, or contacting the organellar DNA polymerase with organelle DNA in a plant cell, ex vivo.

In a thirteenth aspect, the invention provides a modified organelle comprising modified organelle DNA produced by the method according to the twelfth aspect of the invention.

In one embodiment, the modified organelle comprises a modified organelle genome. In one embodiment the organelle is a plant organelle. In some embodiments the organelle is a plastid, suitably a chloroplast. In other embodiments the organelle is a mitochondria.

In an fourteenth aspect, the invention provides a plant or plant part comprising the modified organelle according to the thirteenth aspect of the invention.

In a fifteenth aspect, the invention provides a method of producing a plant having homoplasmic modified organelle DNA comprising;
a. Introducing an error-prone organellar DNA polymerase, or a nucleic acid molecule encoding said polymerase, into one or more plants, and optionally inducing expression of the nucleic acid molecule in the or each plant, to modify the organelle DNA;
b. Taking an explant from said plant(s) and culturing one or more shoots therefrom, or generating one or more F1 seedling(s) from said plant(s);
c. Optionally exposing the or each shoot or seedling to a selection agent which selects for modified organelle DNA, and optionally a further a selection agent which selects for a trait of interest;
d. Selecting those shoots or seedlings having modified organelle DNA, optionally by selecting those shoots or seedlings having resistance to the selection agent which selects for modified organelle DNA;
e. Optionally selecting those shoots or seedlings having resistance to the further selection agent;
f. Regenerating the or each selected shoot or seedling into a plant; and g. Optionally repeating steps (b) to (f) one or more times;
   wherein the error prone-organellar DNA polymerase modifies the organelle DNA throughout the organelle genome, and is semi-dominant over endogenous organellar DNA polymerase present in the plant(s).

In one embodiment, the error prone organellar DNA polymerase is the organellar DNA polymerase according to the first aspect of the invention.

In one embodiment, the organelle DNA is endogenous organelle DNA. In one embodiment, the organelle DNA is an organelle genome. In one embodiment, therefore the plant has homoplasmic modified organelle genomes. In one embodiment, the organelle is a plastid, suitably a chloroplast. In other embodiments the organelle is a mitochondria.

In one embodiment the selection agent which selects for modified organelle DNA is spectinomycin. In one embodiment, the further a selection agent which selects for a trait of interest is a herbicide, suitable examples of which are described herein. Suitably in such embodiments, when the selection agent is spectinomycin, the modified organelle DNA is modified chloroplast DNA, and suitably step (c) is present in the method.

In other embodiments, no selection agent is required to select for modified organelle DNA. Suitably in such embodiments, step (c) may not be present in the method. Suitably in such embodiments, the modified organelle DNA is modified mitochondrial DNA.

In one embodiment, the error prone-organellar DNA polymerase makes modifications to the organelle DNA throughout the organelle genome.

In one embodiment, the error prone-organellar DNA polymerase is dominant over endogenous organellar DNA polymerase present in the plant(s).

In a sixteenth aspect, the invention provides a plant having homoplasmic modified organelle DNA produced by the method according to the fifteenth aspect of the invention.

In one embodiment, the organelle DNA is an organelle genome. In one embodiment, therefore the plant has homoplasmic modified organelle genomes.

In one embodiment the plant or part thereof referred to above is an agriculturally or economically significant species of plant or a part thereof. In one embodiment the plant or part thereof referred to above is a crop plant or part thereof. Suitably plant species are define hereinbelow.

In one embodiment of any of the aspects above, the organelle may be a chloroplast or a mitochondria. Suitably in some embodiments, the methods may be applied to both chloroplasts and mitochondria, optionally at the same time. Suitably therefore the methods may be methods of modifying both chloroplast and mitochondria, and may produce plants or parts thereof with modified chloroplasts and mitochondria, suitably in some cases plants or parts thereof having homoplasmic modified chloroplasts and mitochondria. Suitably in such embodiments, more than one organellar DNA polymerase is introduced into the plant or part thereof, comprising at least one organellar DNA polymerase having a chloroplast targeting peptide and at least one organellar DNA polymerase having a mitochondrial targeting peptide, or one or more isolated nucleic acid molecules or expression vectors encoding said polymerases are introduced into the plant or part thereof.

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps. These terms may equally be substituted with ‘having’ ‘has’ or ‘with’.

Suitably a reference organellar DNA polymerase as referred to herein is a non-modified organellar DNA polymerase. The reference organellar DNA polymerase may be a wild type organellar DNA polymerase. Suitably a reference plant, plant part, as referred to herein is a non-modified, non-transgenic, untransformed plant, plant part, of the same species as the modified plant, plant part of the invention. The reference plant, plant part, may be genetically equivalent to the modified plant, plant part, but unmodified. The reference plant, plant part, may be a wild type plant, plant part, cell or protoplast of the same species as the modified plant, plant part, cell.

### BRIEF DESCRIPTION OF THE DRAWINGS

FIG. 1. Shows neighbour-joining consensus tree of indicated plant organellar DNA polymerase (POP) sequences. Bootstrap values (1000 replicates) were 100% unless indicated otherwise. Sequences were retrieved from GenBank: Arabidopsis thaliana (PolA AEE32595; PolB AEE76393), Brassica rapa (PolA XP_009144938; PolB XP_009145617), Nicotiana tomentosiformis (XP_009610361), Oryza sativa (1. BAT04060; 2. XP_015636766), Physcomitrella patens (PNR49997), Solanum lycopersicon (XP_004244135), Zea mays (1. XP_020401293; w2 AQK46502). Petunia axillaris (Peaxi162Scf00450g00842.1) was from the SOL Genomics Network. P. patens was used as the outgroup. Indicated are taxa containing a single POP or two divergent POP paralogs. Asterisks (*) indicate duplication events responsible for POP paralogs. Scale bar: amino acid substitutions per site.

FIG. 2. Is a scheme showing the organisation of NtPOP^{tom} proteins. (A) NtPOP^{tom} 1152 amino acid native protein (top) aligned with recombinant proteins (bottom). Shown are N-terminal presequence (Pre), disordered region, exonuclease and polymerisation domains locating D390A, E392 and L903F substitutions, C-terminal Strep tag II, cleavage site preceding M283 (arrowed), and region deleted in Pol-enzyme. (B) Alignments of motifs A and Exo I in NtPOP^{tom} with E. coli DNA Pol I. E. coli DNA Pol I residues 1709, D355 and E357 align with the substituted L903, D390 and E392 amino acids in NtPOP^{tom}. Asterisks (*) indicate amino acids essential for function. (C) SWISS MODEL (Waterhouse et al., 2018) of NtPOP^{tom} based on homology to the Klenow fragment of E. coli DNA Pol I (57). Locations of substituted amino acids and antibody binding site are shown.

FIG. 3. Graphs showing DNA synthesis against time by the recombinant NtPOP^{tom} enzymes. (A) Comparison using 400 fmol of the indicated enzymes. (B) The influence of adding 300 fmol and 600 fmol of the Exo-L903F enzyme to 600 fmol of the WT enzyme on DNA synthesis rate. Results were from three independent replicates.

FIG. 4. Shows a map of pUN121 (Nilsson et al., 1983) showing the coding sequence for the lambda Cl repressor, and tet^{R} and amp^{R} genes conferring resistance to tetracycline and ampicillin. The Cl repressor prevents expression of tet^{R}. A single stranded 162 nucleotide gap (dashed line) on the non-coding strand of the c/gene was made using the nicking enzyme Nb. Bpu10I. The direction of DNA replication is shown and extended 353 nucleotides beyond the gap to the N-terminal coding region of cl.

FIG. 5. Part (A) shows cations of base substitution and indels in the c/gene resulting in loss of repressor function for the WT, Exo- and Exo-L903F NtPOP^{tom} enzymes. Domains for DNA binding, hinge region and dimerization are shown. Alpha helices 1-5 (α1-5) and beta sheets (ß1-2) are indicated. Part (B) presents the distance between mutations in mutant c/genes replicated by the Exo-L903F NtPOP^{tom} enzyme.

FIG. 6. Part (A) shows percentages of the different types of mutations associated with the WT, Exo- and Exo-L903F NtPOP^{tom} enzymes. Mispairing rates resulting in observed mutations for (B) WT and Exo-enzymes, (C) Exo- and Exo-L903F enzymes. Template base and mis-paired dNMP are indicated. Asterisks represent significant differences determined by chi squared tests (p<0.001).

FIG. 7. shows the relationship between mutant frequency and percentage of Exo-L903F enzyme in mixtures of Exo-L903F and WT NtPOP^{tom} enzymes in a gap replication assay. Exo-L903F and WT enzymes were combined in the proportions indicated to 3 pmol in total. Following replication of 30 fmol of gapped pUN121 with enzyme mixtures and transformation into bacteria, the mutant frequency was determined from the ratio of tetracycline to ampicillin resistant colonies. Results were from three independent replicates.

FIG. 8. Shows the constructs for expressing MuPOP and the protein structure of MuPOP. (A) Two DNA constructs for expressing MuPOP are presented. Two promoters, Native-P (1397 bp) and AtHSP70-P (260 bp) were used to regulate the expression of the MuPOP. Apart from the promoter region both constructs contained identical coding regions and 3′ UTR and termination regions. The substituted nucleotides for reducing the fidelity of NtPOP are labelled as A1178C, A1183C and G2718C, respectively. (B) The translational product from (A). The MuPOP (1169 aa) contains an N-terminal transit peptide (TP) for chloroplasts targeting and a C-terminal Streptag II linked with GS linker for expression analysis. The substituted amino acids D390A, E392A and L903F correspond to bases A1178C, A1183C and G2718C, respectively. (C) The transit peptide (64 aa) for MuPOP contains the N-terminal full length (58 aa) of the transit peptide from petunia Rubisco small subunit 8 (SSU8) and 6 aa from the original transit peptide of NtPOP at C-terminus.

FIG. 9. A. The structure of the chloroplast targeted GFP-GUS fusion protein in which the two proteins are joined by a LP4/2A self-cleaving peptide. B Confocal images showing the targeting properties of the transit peptide (TP) of Rubisco small subunit 8 using a scanning confocal laser microscope (Nikon SP8) The GFP protein is targeted to the chloroplasts of a mesophyll cell. GFP fluorescence is excited at 488 nm and detected with filter for 512 nm, and chlorophyll auto-fluorescence is detected with a filter for 650 nm. The GFP fluorescence and chlorophyll fluorescence co-localise to chloroplasts.

FIG. 10. Shows detection of MuPOP transcripts using RT-PCR. EF-1 alpha was used as the reference control (EF1 alpha-F, EF1 alpha-R). Primers (StrepTagRTPCR-F, AtUTRRTPCR-R) were specific to the MuPOP sequence, including the coding region of Streptag II (forward) and partial AtHSP18.2 3′ UTR (reverse). Therefore, no signal was detected for the wild type samples when these MuPOP specific primers were used. Two transgenic lines 1 and 6 were analysed for MuPOP regulated by the native promoter (Native-P). One transgenic line 4 regulated by the inducible promoter (AtHSP70-P) was analysed with/without heat shock induction at 40° C.

FIG. 11. Shows MuPOP protein accumulation in WT (wild type) NT-MuPOP plants. MuPOP expression was regulated by the Native-POP Promoter and 5′UTR (A) Western blot detection of MuPOP using a Streptactin alkali phosphatase conjugate, which binds to Strep tag II present in MuPOP. Three wild type tobacco plants and three plants from transgenic lines 1 and 6 were analysed. The expected molecular weight of MuPOP is 123 kDa. (B) Total proteins of the samples in (A) were visualised on 10% (W/V) polyacrylamide stain-free gel (Bio-Rad) suitable for SDS-PAGE. The large subunit of Rubisco (55 kDa) is labelled.

FIG. 12. is an image showing T1 seedlings of transgenic tobacco lines expressing MuPOP (A) 2 week old variegated seedlings growing on 100 μg/ml kanamycin MS medium. (B-C) Magnified images of variegated seedlings under a dissection microscope. (D) 6 weeks old seedling from (C) growing on MS medium containing 100 μg/ml kanamycin. The variegated phenotype was lost in the new leaves formed during growth and development.

FIG. 13. Shows growth of wild type and MuPOP transgenic tobacco with/without high light treatment. All plants have been grown for 2 months. WT, wild type. NT, transgenic tobacco with MuPOP regulated by the Native-Promoter and 5′UTR. SPR, spectinomycin resistant. (A-B) Leaf number and height of the plants grown under normal light (35 μmol photons/m²/s). (C) WT and NT1 have no distinguishable phenotype growing in under normal light. (D-F) Leaf number and height of the plants grown under high light (600 μmol photons/m²/s). (G) WT and NT1 growing under high light, their different plant heights are indicated by the arrow and dashed lines. NT1 has necrotic leaves near the base, whereas WT does not have any necrosis on the leaf. For each tested wild type and transgenic lines, six plants were tested. Significant differences are shown by asterisks.

FIG. 14. Shows (A) Regeneration of MuPOP transgenic tobacco (NT1) on shoot regeneration medium containing 200 μg/ml spectinomycin. (B) The frequency of spectinomycin resistant shoots per explant was tested for wild type (WT), NT1, NT6 and HS4. The data obtained from WT, NT1 and NT6 were the average of three biological replicates. HS4 was tested at 25° C. and 40° C., but only one experiment was performed at each temperature, respectively. (C) Spectinomycin resistant shoots regenerated from NTI leaf explants developed into green, variegated or pigment-deficient plants. The number of plants corresponding to each phenotype are shown. Data was the average of three biological replicates.

FIG. 15. Phenotypes of spectinomycin-resistant Mu-POP plants. (A) Green, (B) variegated, (C) pale-green, (D) white and (E) ivory. (F) Wild type tobacco. (G) Individual leaf isolated from (A) to (E) (left to right). The pigment-deficient phenotypes are indicative of mutations in plastid genes.

FIG. 16. Shows variegated MuPOP transgenic tobacco plants grown up in soil. (A) Top view of an 8 week old variegated MuPOP tobacco. (B) Top view of a three month old variegated MuPOP tobacco. (C) Side view of the plant in (B), which shows differences in the pattern of green-white variegation in different leaves. This plant is heteroplasmic and the random segregation, or sorting out, of plastids would give rise to this pattern of green/white sectoring in leaves.

FIG. 17. Tobacco plants used for sexual crosses, which confirmed maternal transmission of pigment-deficient sectors and spectinomycin-resistance. (A-B) Two variegated plants showing white or yellow sectors were isolated from NT1 and NT6 using spectinomycin selection on shoot regeneration medium. Both NT1(W) and NT6(Y) contained kanamycin resistant genes (nptII) in their nuclear genomes. Non-segregation of kanamycin resistance was consistent with these lines being homozygous for the nuclear located nptII genes. (A) NT1(W) had large white sectors on leaves, which showed maternal inheritance. (B) NT6(Y) had large yellow sectors on leaves, which were transmitted through eggs but not pollen. (C) 14C is a green transplastomic plant containing a plastic located bar gene (Iamtham and Day, 2000) conferring phosphinothricin (PTT) resistance.

FIG. 18. Shows and image of reciprocal crosses between 14C and NT1(W) and between 14C and NT6(Y). Wild type tobacco (a-d, 5) is used as control, which shows sensitivity to kanamycin, spectinomycin and PPT. The pigment phenotypes of the maternal line in the crosses are shown in (a, 1-4). All transgenic seedlings were resistant to kanamycin, as NT1(W) and NT6(Y) contain the kanamycin resistant gene and transmit this to the T1 seedlings (b, 1-4). The T1 hybrid seedlings were resistant to spectinomycin when NT1(W) or NT6(Y) was the mother ((c, 2) and (c, 4)), but the hybrids were sensitive to spectinomycin when 14C was the maternal parent (c, 1) and (c, 3)). White spectinomycin-resistant seedlings containing plastid mutations resulting in loss of chlorophyll cannot be distinguished by colour-phenotype from bleached wild type seedlings that are sensitive to the antibiotic. However, white resistant seedling developed true leaves on spectinomycin medium whereas bleached wild type plants were arrested at the cotyledon stage. The enlarged views of white seedlings are shown for column c. The white seedlings resistant to spectinomycin develop true leaves (c, 2&4), which are not observed in the seedlings sensitive to spectinomycin. The hybrid seedlings were resistant to PPT when 14C was the female parent (d, 1) and (d, 3)), but the hybrids were sensitive to PPT when NT1(W) or NT6(Y) was the mother ((d, 2) and (d, 4)). KANA200, kanamycin 200 μg/ml. SPEC200, spectinomycin 200 μg/ml. PTT, phosphinothricin ammonium 15 μg/ml.

FIG. 19. Shows Southern blot analysis on DNA from wild type (WT), ΔrbcL, and white NT1 plants. Genomic DNA samples extracted from all plants were digested with EcoRV and then loaded on an agarose gel. The control probe is specific to nuclear 26S rDNA, which enables detection of the bands with sizes of 10 kb and 5.6 kb. The probe specific for plastid DNA hybridizes with sequences containing the atpβ and rbcL genes, which allows detection of a 7.1 kb band. The rbcL gene has been removed from ΔrbcL plants resulting in the absence of the plastid 7.1 kb band in the ΔrbcL lane. The ratio of the band intensities between the plastid 7.1 kb band and nuclear 26 rDNA 5.6 kb band remained the same in DNA from WT and white NT1 plants. This indicated plastid DNA was not reduced in copy number in white NT1 plants.

FIG. 20. Map of the tobacco plastid genomes showing the locations of SNPs identified in green (G), white (W) and pale-green (PG) plants isolated following regeneration of MuPOP transgenic lines on spectinomycin medium. Homoplasmic and heteroplasmic SNPs were randomly distributed throughout the plastid genome. 16S rrn mutation conferring spectinomycin resistance was fixed in all samples, (indicated by arrow head in the inner circle. The map with was generated using Geneious Prime and based on data generated by nanopore sequencing.

FIG. 21. Confirmation of selected SNPs detected by nanopore sequencing by Sanger sequencing. (A) G to A substitution was confirmed in the coding sequence of rbcL of PG2. (B) G to A substitution was confirmed in an intron of ycf3 of W1.

FIG. 22. Heteroplasmic SNPs detected in W6 by Sanger sequencing. (A-B) The heteroplasmic status of the SNPs was visualised as overlapping peaks for two different bases. (A) Heteroplasmic T to A substitution is confirmed in the coding sequence of psaB. (B) Heteroplasmic T to A substitution is confirmed in the coding sequence of psbJ. (C) Heteroplasmic A to G substitution is confirmed in the coding sequence of ndhF.

FIG. 23. Shows base substitutions introduced by the wild type POP or MuPOP. (A) Numbers of different types of base substitutions by comparing chloroplast genomes between N. tabacum and N. tomentosiformis. (B) Number of different types of base substitutions in G1, PG2 and W6 plants. (C) Number of different types of base substitutions generated by wild type NtPOP or NtPOP^{Exo-}L903F in vitro

FIG. 24. Represents neighbour joining consensus tree of indicated POP sequences. Bootstrap values (1000 replicates) were 100% unless indicated at nodes. Physcomitrella patens was the outgroup. Sequences with indicated accession numbers were retrieved from GenBank. Brassicaceae, Muscaceae, Poaceae, Leguminosae, Solanaceae families are indicated. All families shown contain two POP paralogs apart from the Solanaceae, which contains a single POP. Scale bar: amino acid substitutions per site.

FIG. 25. Agarose gel showing gapped pUN121 after the replication step with the indicated DNA polymerases before (−) and after (+) treatment with Hind III. MW standards, linear (L) and open circular (OC) pUN121 bands are indicated.

FIG. 26. Region of the phage lambda c/gene encoding alpha helices 1 and 5. Error rate calculations require an estimate of detectable sites at which a base substitution gives rise to a detectable phenotype (Keith et al., 2013), which in this case is loss-of-repressor function giving rise to a tetracycline resistant phenotype. At each position the fraction of base substitutions giving rise to a loss of repressor function is indicated above the base shown. Amino acids amenable and not amenable to changes that retain repressor function have been documented in detail (Reidhaarolson and Sauer, 1990, Sauer, 2013). Positions at which all three potential base substitutions do not affect repressor function are scored as zero, whilst positions at which all three base substitutions result in loss of function are scored as one. Positions at which only one base substitution or two base substitutions results in loss of function are scored as ⅓ and ⅔, respectively. The sum of all the base substitutions giving rise to loss-of-function is the number of detectable sites within this coding region of 99 nucleotides. Indel mutations resulting in frameshift mutations would be detected at all 99 nucleotides.

FIG. 27. SDS-PAGE analyses of purified recombinant NtPOP^{tom} enzymes. (A) Total protein visualised with Bio-Rad (Watford, UK) tri-halo compound-based stain-free method. Protein blot analysis with (B) POP-specific polyclonal antibody, and (C) Strep tag specific monoclonal antibody. Shown are samples loaded, Pol-bands (arrow heads), bands lacking a strep-II tag (asterisks), MW size standards and sizes of bands in recombinant NtPOP^{tom} lanes. The purified WT, Exo- and Exo-L903F recombinant proteins fractionated by SDS-PAGE appeared as two bands (FIG. 27A, lanes 3-5). Both bands bound to a POP-specific antibody raised against a peptide in the palm region (see location in FIG. 2A) of the polymerisation domain. (FIG. 27, lanes 2-4). The larger band corresponded to the intact 1107 amino acid recombinant protein (123 kDa). Cleavage within the disordered region gives rise to the smaller 870 amino acid (99 kDa) band with methionine 283 at the N-terminus as determined by N-terminal sequencing. This 99 kDa protein contains the 3′-5′ exonuclease and polymerisation domains required for function (FIG. 2A). The 737 amino acid (82 kDa) Pol-protein accumulated to much lower amounts than the longer recombinant proteins hindering its purification. Multiple bands were visualised in Pol-preparations by SDS-PAGE (FIG. S7A, lane 2). These bands included the predicted 82 kDa Pol-protein and a 58 kDa protein resulting from cleavage within the disordered region, which contained the Strep II tag and bound to Strep-Tactin (arrowed in FIG. S7A and S7C, lane 2). These bands lack the polymerisation domain and did not bind the antibody recognising this region (FIG. S7B, lane 1). The other bands (FIG. S7A, lane 2, bands marked*) did not bind a monoclonal antibody recognising Strep tag-II (FIG. S7C, lane 2) and probably represent contaminating bacterial proteins. Pol-is an inactive recombinant protein and provided a negative control to verify the removal of bacterial DNA polymerases by our purification regime.

FIG. 28. Shows DNA Polymerase Specific Activity. Synthesis of double stranded DNA was from a 35 base oligonucleotide (M13-F) annealed to single-stranded M13mp18 DNA. The activity of the Exo-L903F enzyme was approximately 30% of the wild type enzyme (WT).

FIG. 29. Part A shows the percentage of mutant c/genes with single and multiple mutations for indicated NtPOP^{tom} enzymes. Significant differences were found for the Exo-L903F enzyme relative to the WT and Exo—enzymes (p<0.05). The differences between WT and Exo-enzymes were not significant (p<0.05) B. Number of mutant c/genes with the indicated number of mutations at separated sites after replication by the NtPOP^{tom} Exo-L903F enzyme FIG. 30. Shows the percentage of transition versus transversion base substitutions for the indicated recombinant NtPoI^{tom} enzymes. Chi-squared tests showed no significant differences between the enzymes for p<0.05.

FIG. 31. Shows seedlings from the indicated crosses grown on plant growth medium containing spectinomycin (200 μg/mL) for 6-8 weeks. A-D) Seedlings from self-fertilised plants expressing the chloroplast mutator POP (cmPOP). A) Photograph of complete plate. C-D) Photograph of individual seedlings showing green sectors (arrowed) containing chloroplast genome encoded spectinomycin resistance. E-F) Wild type seedlings from self-fertilised plants expressing the wild type (WT) POP. E) Complete plate with many bleached seedlings. F) Photograph of individual bleached WT seedlings. No green sectors were visible on WT seedlings.

FIG. 32. Scheme showing the organization of the DNA and protein sequences of the mitochondrial targeted POP DNA polymerase gene expressed in plants. (A) The DNA sequence shows the 5′ regulatory region including the promoter and 5′ UTR from the tobacco POP DNA polymerase gene, the DNA coding sequence and 3′ regulatory region including the 3′UTR and termination region from the Arabidopsis Heat Shock Protein 18.2. The substituted nucleotides for reducing the fidelity of NtPOP are labelled A1130C, A1136C and G2670C. (B) Protein sequence of the entire mitochondrial targeted MuPOP (1153 aa) shows the locations of the N-terminal mitochondrial targeting presequence (AOX1 MTpresequence), GS linker and C-terminal Streptag II tag. The substituted amino acids D390A, E392A and L903F correspond to bases A1130C, A1136C and G2670C, respectively. AOX1 MTpresequence is a recombinant presequence in the same fashion as the TP shown in FIG. 8C. AOX1 MTpre contains the N-terminal presequence from soybean mitochondrial Alternative Oxidase 1 followed by six amino acids from the C-terminal region of the predicted N-terminal presequence of the wildtype NtPOP. The mitochondrial presequence was from the Soybean mitochondrial targeted Alternative Oxidase 1.

FIG. 33. (A) Structure of the mitochondrial targeted mScarlet-I fluorescent protein. (B) Images of leaf mesophyll cells showing the locations of mitochondrial targeted mScarlet-I (top) and chloroplasts (middle). The bottom merged image shows the locations of mitochondria tagged with mScarlet-I (arrowed) and chloroplasts. Images captured by Zeiss Axio Imager D2 upright microscope with 40×/0.75 Plan Neofluar objective and Photometrics CoolSnap HQ camera allowing a pixel size of 6.5 μm. Cy3 and Texas red filters were used to detect mScarlet-I and chlorophyll fluorescence, respectively.

FIG. 34. Mutant phenotypes of plants expressing the mitochondrial mutator POP DNA polymerase. (A-B) MT-MuPOP transgenic line 1 and MT-MuPOP transgenic line 2 plants with distorted leaves (arrowed). (C-D) MT-MuPOP transgenic line 3 and MT-MuPOP transgenic line 4 plants showing variegated leaves containing pale-green/yellow/white pigment-deficient sectors (arrowed). (E-F) Examples of variegated leaves from MT-MuPOP transgenic lines 3 and 4 (G) Normal wild type plants do not contain these distorted and variegated leaves.

FIG. 35. Narrow leaf phenotype (NL) of a subclone of tobacco plant MT-MuPOP transgenic line 2, named Mt-MuPOP line 2_NL, expressing the mitochondrial-targeted mutator POP DNA polymerase following a cycle or regeneration in vitro. Mitochondrial mutations were identified in DNA extracted from the arrowed leaf using the Illumina Hi-seq platform.

FIG. 36. Confirmation of two heteroplasmic mutations in transgenic subclone Mt-MuPOP line 2_NL by Sanger sequencing. (A) A heteroplasmic T to A change in the mitochondrial 18S rDNA gene compared to the wild type sequence. (B) A heteroplasmic T to A change in an intergenic region of the mitochondrial genome compared to the wild type sequence. Reference genome NCBI accession No. NC006581.

### DETAILED DESCRIPTION OF THE INVENTION

Features and embodiments of the aspects of the invention will now be described under the following headed sections which apply to any aspect. Any feature under any section may be combined with any aspect in any workable combination.

### Organellar DNA Polymerase

The present invention primarily relates to a modified organellar DNA polymerase enzyme with a high error rate such that it introduces a plurality of mutations to organelle DNA during replication. This is useful for the generation of plants with modified organelle genomes which may have desirable traits.

DNA polymerase enzymes catalyse the replication of genomic DNA. An organellar DNA polymerase is a DNA polymerase enzyme which is nuclear encoded but is targeted to be expressed in the organelles of a cell. Organelles are defined herein below. Organellar DNA polymerase enzymes catalyse the replication of organelle DNA such as plastomers or mitogenomes.

Suitably the organellar DNA polymerase is a modified organellar DNA polymerase. Suitably the organellar DNA polymerase is an error-prone organellar DNA polymerase. Suitably the organellar DNA polymerase is modified to be an error-prone organellar DNA polymerase.

The term “modified organellar DNA polymerase” refers to an organellar DNA polymerase enzyme having a sequence that is mutated from a wild-type organellar DNA polymerase amino acid sequence and that confers an increased error rate to the polymerase.

Suitably the organellar DNA polymerase is a plant organellar DNA polymerase (POP). Suitably the plant organellar DNA polymerase may be derived from any species of plant, algae or protozoan. Suitably the organellar DNA polymerase may be derived from the following species of plant, for example: Arabidopsis thaliana, Brassica rapa, Nicotiana tomentosiformis, Oryza sativa, Physcomitrella patens, Solanum lycopersicum, Zea mays, Petunia axillaris, Nicotiana tabacum. In some embodiments, the organellar DNA polymerase may be derived from a species of moss, for example from Physcomitrella patens.

In one embodiment, the organellar DNA polymerase is derived from Nicotiana tabacum. Suitably the amino acid sequence of the wild type organellar DNA polymerase from Nicotiana tabacum is shown in SEQ ID NO: 1.

SEQ ID NO: 1 is a reference sequence in which the modifications to the organellar DNA polymerase are described herein, however the invention extends to other organelle DNA polymerase enzymes having the same corresponding mutations to those described herein. Other suitable organellar DNA polymerase sequences are described herein, for example the organellar DNA polymerase may comprise an amino acid sequence according to SEQ ID NO: 7, 8, 9, or 89. These sequences may equally be used as a reference sequence.

In one embodiment, the organellar DNA polymerase is derived from Zea Mays. Suitably the amino acid sequence of the wild type organellar DNA polymerase from Zea Mays is shown in SEQ ID NO: 7.

In one embodiment, the organellar DNA polymerase is derived from Arabidopsis thaliana. Suitably the amino acid sequence of the wild type organellar DNA polymerase A from Arabidopsis thaliana is shown in SEQ ID NO: 9. Suitably the amino acid sequence of the wild type organellar DNA polymerase B from Arabidopsis thaliana is shown in SEQ ID NO: 8.

In one embodiment, the organellar DNA polymerase is derived from Physcomitrella patens. Suitably the amino acid sequence of the wild type organellar DNA polymerase from Physcomitrella patens is shown in SEQ ID NO: 89.

Suitably, given the reference sequence such as SEQ ID NO: 1 contains one or more modifications as defined herein, it may be regarded as a variant of SEQ ID NO: 1 or another reference sequence defined herein. In one embodiment therefore, the organellar DNA polymerase comprises an amino acid sequence which is a variant of SEQ ID NO: 1, 7, 8, 9, or 89 or an amino acid sequence having at least 35% identity thereto, or a functional fragment thereof. By ‘variant’ it is meant that the reference sequence, such as SEQ ID NO: 1, contains one or more modifications. Suitably modification by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such modified sequences may also be termed ‘derivatives’ of a reference sequence. Suitably the variant or derivative comprises one or more modifications listed above or corresponding thereto in a different reference sequence.

Suitably the organellar DNA polymerase comprises an amino acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity to SEQ ID NO: 1, or a functional fragment thereof. In one embodiment, the organellar DNA polymerase comprises an amino acid sequence having at least 90%, at least 95%, at least 96%, at least 97%, at least 98%, at least 99% identity to SEQ ID NO: 1, or a functional fragment thereof. Suitably homologous organellar DNA polymerase enzymes derived from plants other than Nicotiana tabacum will comprise at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity to SEQ ID NO: 1. Suitably the organellar DNA polymerase comprises an amino acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity to SEQ ID NO: 7, 8, 9, or 89 or a functional fragment thereof. In one embodiment, the organellar DNA polymerase comprises an amino acid sequence having at least 90%, at least 95%, at least 96%, at least 97%, at least 98%, at least 99% identity to SEQ ID NO: 7, 8, 9, or 89 or a functional fragment thereof.

Suitably an organellar DNA polymerase from a different species may only have low sequence identity with SEQ ID NO: 1 but can be modified at the corresponding positions and still produce a desired error prone polymerase with the increased error rate required for the invention. For example, the organellar DNA polymerase from Physcomitrella patens has only 39.2% identity with the Nicotiana tabacum wild type POP (SEQ ID NO: 1), however it performs the same function of being an error prone polymerase.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of (Smith and Waterman, 1981), by the homology alignment algorithm of (Needleman and Wunsch, 1970), by the search for similarity method of (Pearson and Lipman, 1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in (Altschul et al., 1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., 1990) These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix ((Henikoff and Henikoff, 1992). In addition to calculating percent sequence identity, the BLAST algorithm also performs a statistical analysis of the similarity between two sequences (Karlin and Altschul, 1990). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Suitably the organellar DNA polymerase comprises an amino acid sequence according to SEQ ID NO: 1, 7, 8, 9 or 89 or a functional fragment thereof. Suitably the organellar DNA polymerase comprises an amino acid sequence which is a variant of SEQ ID NO: 1, 7, 8, 9 or 89 or a functional fragment thereof.

A “functional fragment” refers to a protein fragment that retains the function of the full length protein. As such, a functional fragment of an organellar DNA polymerase enzyme is a fragment, portion or part of such a protein that is capable of catalysing the replication of organellar DNA. In one embodiment, the organellar DNA polymerase may comprise a functional fragment of an amino acid sequence according to SEQ ID NO: 1, 7, 8, 9, or 89. In one embodiment, the organellar DNA polymerase may comprise a functional fragment of an amino acid sequence having at least 35% identity to SEQ ID NO: 1, 7, 8, 9 or 89.

In one embodiment, the organellar DNA polymerase comprises an amino acid sequence according to SEQ ID NO: 1. In one embodiment, the organellar DNA polymerase consists of an amino acid sequence according to SEQ ID NO: 1, 7, 8, 9 or 89. In one embodiment, the organellar DNA polymerase comprises an amino acid sequence which is a variant of SEQ ID NO: 1. In one embodiment, the organellar DNA polymerase consists of an amino acid sequence which is a variant of SEQ ID NO: 1, 7, 8, 9 or 89.

Suitably the organellar DNA polymerase further comprises one or more modifications as defined herein. Suitably the organellar DNA polymerase further comprises one or more amino acid modifications as defined herein.

Suitably the organellar DNA polymerase comprises a modification at position L903, and optionally one or more further modifications at the following positions: D390, E392, R862, E904, and N1065 of SEQ ID NO: 1, or positions corresponding thereto. Suitably any combination of modifications at these positions of SEQ ID NO: 1, or positions corresponding thereto, may be present.

Suitably the positions corresponding thereto in the organellar DNA polymerase from Zea mays (SEQ ID NO: 7) are position L784, and optionally one or more further modifications at the following positions: D285, E287, R743, E785, N946. In one embodiment, the invention provides an organellar DNA Polymerase enzyme comprising an amino acid sequence according to SEQ ID NO: 7 or comprising an amino acid sequence having at least 35% identity thereto, or a functional fragment thereof, wherein the amino acid sequence or functional fragment comprises a modification at position L784, and optionally one or more further modifications at the following positions: D285, E287, R743, E785, N946 of SEQ ID NO: 7.

Suitably the positions corresponding thereto in the organellar DNA polymerase A from Arabidopsis thaliana (SEQ ID NO: 9) are L803F, and optionally one or more further modifications at the following positions: D294A, E296A, R762, E804 and N963.

In one embodiment, the invention provides an organellar DNA Polymerase enzyme comprising an amino acid sequence according to SEQ ID NO: 9 or comprising an amino acid sequence having at least 35% identity thereto, or a functional fragment thereof, wherein the amino acid sequence or functional fragment comprises a modification at position L803, and optionally one or more further modifications at the following positions: D294, E296, R762, E804 and N963 of SEQ ID NO: 9.

Suitably the positions corresponding thereto in the organellar DNA polymerase B from Arabidopsis thaliana (SEQ ID NO: 8) are L802F, and optionally one or more further modifications at the following positions: D287A, E289A, R761A, E803A and N962A.

In one embodiment, the invention provides an organellar DNA Polymerase enzyme comprising an amino acid sequence according to SEQ ID NO: 8 or comprising an amino acid sequence having at least 35% identity thereto, or a functional fragment thereof, wherein the amino acid sequence or functional fragment comprises a modification at position L802, and optionally one or more further modifications at the following positions: D287, E289, R761, E803 and N962 of SEQ ID NO: 8.

Suitably the positions corresponding thereto in the organellar DNA polymerase from Physcomitrella patens (SEQ ID NO: 89) are L1209, and optionally one or more further modifications at the following positions: D691, E693, R1168, E1210 and N1368.

In one embodiment, the invention provides an organellar DNA Polymerase enzyme comprising an amino acid sequence according to SEQ ID NO: 89 or comprising an amino acid sequence having at least 35% identity thereto, or a functional fragment thereof, wherein the amino acid sequence or functional fragment comprises a modification at position L1209, and optionally one or more further modifications at the following positions: D691, E693, R1168, E1210 and N1368.

Suitably the modification at position L903, or a corresponding position thereto, is in the polymerase domain of the organellar DNA polymerase. Suitably the further optional modifications at positions R862, E904, and N1065, or positions corresponding thereto, are also in the polymerase domain. Suitably the optional further modifications D390 and E392, or corresponding positions thereto, are present in the exonuclease domain of the organellar DNA polymerase.

Suitably, the organellar DNA polymerase comprises a modification at position L903, or a corresponding position thereto, in the polymerase domain of the enzyme and at least one further modification in the exonuclease domain of the enzyme. Suitably the exonuclease domain spans from position 382 to 623 of SEQ ID NO: 1. Suitably the modification in the exonuclease domain of the enzyme may be selected from D390 and/or E392, or corresponding positions thereto.

Suitably therefore, the organellar DNA polymerase comprises a modification at position L903 and one or more further modifications selected from any of the following options:
(i) D390;
(ii) E392;
(iii) R862;
(iv) E904;
(v) N1065;
(vi) D390 and E392;
(vii) D390 and R862;
(viii) D390 and E904;
(ix) D390 and N1065;
(x) E392 and R862;
(xi) E392 and E904;
(xii) E392 and N1065;
(xiii) R862 and E904;
(xiv) R862 and N1065;
(xv) D390, E392 and R862;
(xvi) D390, E392 and E904;
(xvii) D390, E392, and N1065;
(xviii) E392, R862, and E904;
(xix) E392, R862 and N1065;
(xx) R862, E904 and N1065;
(xxi) D390, E392, R862 and N1065;
of SEQ ID NO: 1, or corresponding positions thereto.

In one embodiment, the organellar DNA polymerase comprises a modification at position L903 and further modifications at the following positions: D390 and E392 of SEQ ID NO: 1, or positions corresponding thereto.

Suitably ‘modification’ as used herein means a change in the amino acid sequence at the stated position with reference to SEQ ID NO: 1 or the corresponding position in a different organellar DNA polymerase amino acid sequence, suitably the modification may be an insertion, deletion or substitution of the amino acid at the recited position. Suitably the modification is a substitution of the amino acid at the recited position, suitably with a different amino acid. Suitably any amino acid may be used for the substitution. Suitably any proteinogenic amino acid may be used for the substitution. Suitably the substitution is a conservative substitution.

By ‘conservative’ it is meant that an amino acid with similar characteristics may be used for the substitution. Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with hydroxyl side chain may be substituted with another amino acid with a hydroxyl side chain, e.g., serine and threonine; an amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively.

Suitably the organellar DNA polymerase comprises a substitution at position L903, and optionally one or more further substitutions at the following positions: D390, E392, R862, E904, and N1065 of SEQ ID NO: 1, or positions corresponding thereto.

Suitably the organellar DNA polymerase comprises a conservative substitution at position L903, and optionally one or more further conservative substitutions at the following positions: D390, E392, R862, E904, and N1065 of SEQ ID NO: 1, or positions corresponding thereto.

Suitably position L903 or a position corresponding thereto is substituted with an amino acid selected from Methionine (M), Asparagine, Phenylalanine (F) and Alanine (A). In one embodiment, L903 or a position corresponding thereto is substituted with phenylalanine (F). Therefore the organellar DNA polymerase enzyme comprises the modification L903F, or the same modification at a corresponding position.

Suitably positions D390 and E392 or a position corresponding thereto are substituted with an amino acid selected from alanine (A), valine (V), Leucine (L), Isoleucine (I). In one embodiment, D390 or a position corresponding thereto is substituted with alanine (A). Therefore the organellar DNA polymerase enzyme comprises the modification D390A or the same modification at a corresponding position. In one embodiment, E392 or a position corresponding thereto is substituted with alanine (A). Therefore the organellar DNA polymerase enzyme comprises the modification E392A or the same modification at a corresponding position.

Suitably position R862 or a position corresponding thereto is substituted with alanine (A), serine (S) or leucine (L).

Suitably position E904 or a position corresponding thereto is substituted with alanine (A), serine (S) or leucine (L).

Suitably position N1065 or a position corresponding thereto is substituted with alanine (A), serine (S) or leucine (L).

Suitably ‘corresponding position’ as used herein means the same amino acid position in a different reference sequence, suitably in a different reference sequence to that of SEQ ID NO: 1, suitably in a different organellar polymerase sequence. Therefore whilst the statements herein refer to SEQ ID NO: 1, the invention is not restricted to the organellar DNA polymerase of SEQ ID NO: 1, each modification may be located at a position corresponding to an amino acid position denoted above in another organellar DNA polymerase enzyme sequence, such as SEQ ID NOs 7, 8, 9, 89. Therefore the invention equally refers to other organellar DNA polymerase enzymes having different amino acid sequences with the same modifications. It is possible to compare organellar DNA polymerase polypeptides by sequence comparison and locate conserved regions that correspond to the amino acid positions listed above. Sequence comparison to find corresponding positions may be carried out by aligning the amino acid sequences of two or more proteins, using an alignment program such as BLAST®. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7). In the present case, a corresponding position in a different organellar DNA polymerase sequence may be found by aligning the amino acid sequence of said other organellar DNA polymerase with SEQ ID NO: 1 and locating the same amino acid position as those listed. For example, L903 in SEQ ID NO: 1 corresponds to 1709 in the amino acid sequence of E. coli DNA polymerase I.

Suitably therefore the reference sequence may comprise an amino acid sequence according to SEQ ID NO: 7, 8, 9, or 89. Suitably these are the amino acid sequences of the wild type organellar DNA polymerase from Zea Mays, Arabidopsis thaliana POPB and POPA, and Physcomitrella patens respectively.

In one embodiment, the invention provides an organellar DNA Polymerase enzyme comprising an amino acid sequence according to SEQ ID NO: 1 or comprising an amino acid sequence having at least 35% identity thereto, or a functional fragment thereof, wherein the amino acid sequence or functional fragment comprises a modification at position L903, and optionally one or more further modifications at the following positions: D390, E392, R862, E904, and N1065 of SEQ ID NO: 1, or positions corresponding thereto in any one of the following amino acid sequences: SEQ ID NO: 7, 8, 9, or 89.

In one embodiment, the invention provides an organellar DNA Polymerase enzyme comprising an amino acid sequence according to SEQ ID NO: 1, 7, 8, 9 or 89 or comprising an amino acid sequence having at least 35% identity thereto, or a functional fragment thereof, wherein the amino acid sequence or functional fragment comprises a modification at position L903, and optionally one or more further modifications at the following positions: D390, E392, R862, E904, and N1065 of SEQ ID NO: 1, or positions corresponding thereto in SEQ ID NO: 7, 8, 9 or 89.

In one embodiment, the organellar DNA polymerase enzyme comprises an amino acid sequence according to SEQ ID NO: 1 wherein the amino acid sequence comprises the substitution L903F, and optionally one or more further substitutions selected from the following: D390A, E392A, R862A, E904A, and N1065A, or the same modifications at positions corresponding thereto. In one embodiment, the organellar DNA polymerase enzyme comprises an amino acid sequence according to SEQ ID NO: 1 wherein the amino acid sequence comprises one or more modifications, wherein the modifications consist of the substitution L903F, and optionally one or more substitutions selected from the following: D390A, E392A, R862A, E904A, and N1065A or the same modifications at positions corresponding thereto.

In one embodiment, the organellar DNA polymerase enzyme comprises an amino acid sequence according to SEQ ID NO: 1 wherein the amino acid sequence comprises the substitution L903F, and optionally one or more further substitutions selected from the following: D390A, E392A, R862A, E904A, and N1065A, or the same modifications at positions corresponding thereto in any one of the following amino acid sequences: SEQ ID NO: 7, 8, 9 or 89.

In one embodiment, the organellar DNA polymerase enzyme comprises an amino acid sequence according to SEQ ID NO: 1 wherein the amino acid sequence comprises one or more modifications, wherein the modifications consist of the substitution L903F, and optionally one or more substitutions selected from the following: D390A, E392A, R862A, E904A, and N1065A or the same modifications at positions corresponding thereto in any one of the following amino acid sequences: SEQ ID NO: 7, 8, 9 or 89.

In one embodiment, the organellar DNA polymerase enzyme comprises an amino acid sequence according to SEQ ID NO: 1 wherein the amino acid sequence comprises the substitution L903F or the same modification at a position corresponding thereto. In one embodiment, the organellar DNA polymerase enzyme comprises an amino acid sequence according to SEQ ID NO: 1 wherein the amino acid sequence comprises one or more modifications, wherein the modifications consist of the substitution L903F or the same modification at a position corresponding thereto.

In one embodiment, the organellar DNA polymerase enzyme comprises an amino acid sequence according to SEQ ID NO: 1 wherein the amino acid sequence comprises the substitution L903F, and the further substitutions D390A and E392A, or the same modifications at positions corresponding thereto. In one embodiment, the organellar DNA polymerase enzyme comprises an amino acid sequence according to SEQ ID NO: 1 wherein the amino acid sequence comprises one or more modifications, wherein the modifications consist of the substitution L903F, and the further substitutions D390A and E392A, or the same modifications at positions corresponding thereto.

Suitably the organellar DNA polymerase enzyme may comprise an amino acid sequence according to SEQ ID NO: 2, or an amino acid sequence having at least 35%, at least 40%, at least 45%, at least 50%, at least 55%, at least 60%, at least 65%, 70%, at least 75%, at least 80%, at least 85%, at least 90%, at least 95%, at least 96%, at least 97%, at least 98%, at least 99% identity to SEQ ID NO: 2, or a functional fragment thereof. Suitably the modification at position L903, or a position corresponding thereto, is retained. Suitably the modifications at positions D390A and E392A, or positions corresponding thereto, if present, are retained.

Suitably the organellar DNA polymerase enzyme comprises an amino acid sequence according to SEQ ID NO: 2 or a functional fragment thereof. Suitably the organellar DNA polymerase enzyme comprises an amino acid sequence according to SEQ ID NO: 2.

In one embodiment, the organellar DNA polymerase enzyme consists of an amino acid sequence according to SEQ ID NO: 2 or a functional fragment thereof. In one embodiment, the organellar DNA polymerase enzyme consists of an amino acid sequence according to SEQ ID NO: 2

Suitably the organellar DNA polymerase enzyme may comprise an amino acid sequence according to SEQ ID NO: 10 or 11, or an amino acid sequence having at least 35%, at least 40%, at least 45%, at least 50%, at least 55%, at least 60%, at least 65%, 70%, at least 75%, at least 80%, at least 85%, at least 90%, at least 95%, at least 96%, at least 97%, at least 98%, at least 99% identity to SEQ ID NO: 10 or 11, or a functional fragment thereof. Suitably the modification at position L903, or a position corresponding thereto, is retained. Suitably the modifications at positions D390A and E392A, or positions corresponding thereto, if present, are retained.

Suitably the organellar DNA polymerase enzyme comprises an amino acid sequence according to SEQ ID NO: 10 or 11 or a functional fragment thereof. Suitably the organellar DNA polymerase enzyme comprises an amino acid sequence according to SEQ ID NO: 10 or 11.

In one embodiment, the organellar DNA polymerase enzyme consists of an amino acid sequence according to SEQ ID NO: 10 or 11 or a functional fragment thereof. In one embodiment, the organellar DNA polymerase enzyme consists of an amino acid sequence according to SEQ ID NO: 10 or 11 The organellar DNA polymerase enzyme may be isolated or purified. That is to say it is substantially free of cellular material.

A protein or enzyme that is substantially free of cellular material includes preparations of protein or enzyme having less than about 30%, 20%, 10%, 5%, or 1% (by dry weight) of contaminating protein. When the protein or enzyme of the invention or functional fragment thereof is recombinantly produced, preferably culture medium represents less than about 30%, 20%, 10%, 5%, or 1% (by dry weight) of chemical precursors or non-protein-of-interest chemicals.

### Organellar DNA Polymerase Activity

As mentioned above, suitably the organellar DNA polymerase of the invention is error-prone which means that it introduces a plurality of mutations into organelle DNA during replication.

Suitably the organellar DNA polymerase of the invention has an increased error rate compared to a reference wild type organellar DNA polymerase. Suitably the increased error rate is caused by the modifications to the amino acid sequence of the organellar DNA polymerase. Suitably the modifications to the amino acid sequence of the organellar DNA polymerase described herein reduce the exonuclease activity of the enzyme, otherwise known as the proofreading activity of the enzyme. Suitably therefore errors made during replication by the polymerase are not corrected or are corrected to a lesser extent. Suitably therefore the organellar DNA polymerase has reduced exonuclease activity compared to a reference wild type organellar DNA polymerase. Suitably therefore the organellar DNA polymerase has reduced 3′-5′ exonuclease activity compared to a reference wild type organellar DNA polymerase. However suitably the polymerase activity of the organellar DNA polymerase enzyme is retained, suitably the polymerase activity of the organellar DNA polymerase is comparable to that of a reference wild type organellar DNA polymerase.

Suitably the organellar DNA polymerase has an error rate which is 5 to 140 times greater than a reference wild type organellar DNA polymerase. Suitably the organellar DNA polymerase has an error rate which is at least 5, at least 6, at least 7, at least 8, at least 10, at least 20, at least 30, at least 40, at least 50, at least 60, at least 70, at least 80, at least 90, at least 100, at least 110, at least 120, at least 130, up to 140 times greater than a reference wild type organellar DNA polymerase.

In some embodiments, the organellar DNA polymerase has an error rate which is about 140 times greater than a reference wild type organellar DNA polymerase. Suitably in such embodiments, the organellar DNA polymerase comprises the substitution L903F, and the further substitutions D390A and E392A, or the same modifications at corresponding positions.

Suitably the organellar DNA polymerase has an error rate of between 1×10⁻⁵ and 1×10⁻² mutations per base, suitably between 4×10⁻⁵ and 8×10⁻³ mutations per base.

Suitably the organellar DNA polymerase has an error rate of between 1×10⁻⁴ and 1×10⁻² mutations per base, suitably between 3×10⁻⁴ and 8×10⁻³ mutations per base.

Suitably the organellar DNA polymerase has an error rate of between 1×10⁻³ and 1×10⁻² mutations per base, suitably between 1×10⁻³ and 8×10⁻³ mutations per base.

In one embodiment the organellar DNA polymerase has an error rate of between 1.2×10⁻³ and 7.7×10⁻³ mutations per base. Suitably in such embodiments, the organellar DNA polymerase comprises the substitution L903F, and the further substitutions D390A and E392A, or the same modifications at corresponding positions.

Suitably the organellar DNA polymerase introduces mutations into the organelle DNA. Suitably the mutations are single base substitutions, or single base indels. Suitably the organellar DNA polymerase introduces single base substitutions into the organelle DNA. Suitably the organellar DNA polymerase introduces transition mutations or transversion mutations into the organelle DNA. In one embodiment, the organellar DNA polymerase introduces transversion mutations into the organelle DNA. Suitable transversion mutations include A-T, A-C, G-T, and G-C, or vice versa. In one embodiment, the organellar DNA polymerase introduces transition mutations into the organelle DNA. Suitable transition mutations include A-G, and C-T or vice versa. In one embodiment, the organellar DNA polymerase introduces A-T transversion mutations, and A-G or C-T transition mutations into organelle DNA. In one embodiment, the organellar DNA polymerase introduces A-T transversion mutations.

Suitably the organellar DNA polymerase introduces mutations into organelle DNA across the entire replication region. Suitably the replication region is the region of organelle DNA to be replicated by the enzyme. Suitably when the enzyme is expressed within an organelle, the replication region may be the entire organelle genome, suitably in the case of plastids, this may be known as the ‘plastome’ or in the case of mitochondria the ‘mitogenome’. Suitably therefore, in one embodiment the organellar DNA polymerase introduces mutations across the plastome. Suitably the mutations are introduced randomly. Suitably, the error prone organellar DNA polymerase introduces one or more mutations scattered across the organelle genome, suitably randomly across the organelle genome. Suitably these mutations may be spaced within a few hundred bases of each other or may be spaced as much as 75,000 bases apart. Suitably therefore, on average, the error prone organellar DNA polymerase introduces a mutation into the organelle genome every 100-500 bases, suitably every 100-400 bases, suitably every 100-300 bases, suitably every 100-200 bases.

Suitably the organellar DNA polymerase described herein will compete with a reference wild type organellar DNA polymerase when in the presence of organelle DNA. Suitably the organellar DNA polymerase described herein outcompetes reference wild type organellar DNA polymerases when in the presence of organelle DNA. Suitably the organellar DNA polymerase described herein is semi-dominant over reference wild type organellar DNA polymerases. Suitably the organellar DNA polymerase described herein is dominant over reference wild type organellar DNA polymerases. Suitably when both an organellar DNA polymerase as described herein, and a wild type organellar DNA polymerase are in the presence of organelle DNA, if the mutation rate of the organelle DNA is still elevated, this demonstrates that the organellar DNA polymerase described herein dominates replication. This may be determined by a gap-replication assay in which both the organellar DNA polymerase to be tested, and a reference wild type organellar DNA polymerase, as well as organelle DNA are present.

### Expression Vector

The organellar DNA polymerase of the invention may be encoded by a nucleic acid molecule, which nucleic acid molecule may be comprised upon an expression vector for expression in a cell.

Suitably therefore there is provided an isolated nucleic acid molecule comprising a nucleotide sequence which encodes an organellar DNA polymerase described herein.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

Suitably SEQ ID NO: 4 provides the nucleic acid sequence of a modified Nicotiana tabacum organellar DNA polymerase of the invention. Suitable SEQ ID NOs:12 and 13 provide the nucleic acid sequence of a modified Arabidopsis thaliana organellar DNA polymerase A and B respectively also of the invention.

Suitably the isolated nucleic acid molecule comprises a sequence according to SEQ ID NO: 4, or a nucleic acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity thereto. Suitably the isolated nucleic acid molecule retains its ability to encode an organellar DNA polymerase according to the invention.

Suitably the isolated nucleic acid molecule comprises a sequence according to SEQ ID NO: 4.

In one embodiment, the isolated nucleic acid molecule consists of a sequence according to SEQ ID NO: 4.

Suitably the isolated nucleic acid molecule comprises a sequence according to SEQ ID NO: 12 or 13, or a nucleic acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity thereto. Suitably the isolated nucleic acid molecule retains its ability to encode an organellar DNA polymerase according to the invention.

Suitably the isolated nucleic acid molecule comprises a sequence according to SEQ ID NO: 12 or 13.

In one embodiment, the isolated nucleic acid molecule consists of a sequence according to SEQ ID NO: 12 or 13.

Suitably SEQ ID NO: 3 provides the nucleic acid sequence of the wild type Nicotiana tabacum organellar DNA polymerase of the invention. In one embodiment, the isolated nucleic acid molecule comprises a sequence according to SEQ ID NO: 3 or a nucleic acid sequence having at least 35% identity thereto, wherein the sequence comprises one or more nucleotide modifications at positions which give rise to a modification at or corresponding to position L903 of SEQ ID NO: 1, and optionally one or more modifications at positions D390, E392, R862, E904, and N1065 of SEQ ID NO: 1, or positions corresponding thereto.

Suitably the isolated nucleic acid molecule comprises a sequence according to SEQ ID NO: 3, or a nucleic acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity thereto. Suitably the isolated nucleic acid molecule retains its ability to encode an organellar DNA polymerase according to the invention.

Suitably therefore any of the nucleic acid sequences encoding organellar polymerases described herein may be modified at nucleotide positions which in turn give rise to the amino acid modifications listed herein. Suitably, with reference to SEQ ID NO: 3, the sequence comprises one or more nucleotide modifications at positions 1178, 1183 and/or 2718, or corresponding positions thereof. Suitably said one or more nucleotide modifications comprise base substitutions. Suitably said one or more nucleotide modifications comprise A to C, or G to C substitutions. Suitably the nucleotide modification at position 1178 of SEQ ID NO: 3, or a position corresponding thereto, is A1178C. Suitably the nucleotide modification at position 1183 of SEQ ID NO: 3, or a position corresponding thereto, is A1183C. Suitably the nucleotide modification at position 2718 of SEQ ID NO: 3, or a position corresponding thereto, is G2718C.

Suitably nucleotide modification at position 1178, or a corresponding position thereof, gives rise to a modification at position D390 of SEQ ID NO: 1, or a corresponding position thereof. Suitably nucleotide modification at position 1183, or a corresponding position thereof, gives rise to a modification at position E392 of SEQ ID NO: 1, or a corresponding position thereof. Suitably nucleotide modification at position 2718, or a corresponding position thereof, gives rise to a modification at position L903 of SEQ ID NO: 1, or a corresponding position thereof.

In one embodiment, the isolated nucleic acid molecule comprises a sequence according to SEQ ID NO: 3, or a nucleic acid sequence having at least 35% identity thereto, wherein the sequence comprises one or more nucleotide modifications at positions which give rise to a modification at or corresponding to position L903 of SEQ ID NO: 1, and modifications at positions D390, and E392, of SEQ ID NO: 1, or positions corresponding thereto.

In one embodiment, the isolated nucleic acid molecule comprises a sequence according to SEQ ID NO: 3, or a nucleic acid sequence having at least 35% identity thereto, wherein the sequence comprises nucleotide modifications at positions 1178, 1183, and 2718 which give rise to a modification at or corresponding to position L903 of SEQ ID NO: 1, and modifications at positions D390, and E392, of SEQ ID NO: 1, or positions corresponding thereto.

Suitably the nucleotide modifications are base substitutions. Suitable base substitutions are shown in the nucleotide sequences provided herein.

In one embodiment, the isolated nucleic acid molecule comprises a sequence according to SEQ ID NO: 3, wherein the sequence comprises one or more nucleotide modifications at positions which give rise to a modification at position L903 of SEQ ID NO: 1, and modifications at positions D390, and E392, of SEQ ID NO: 1.

In one embodiment, the isolated nucleic acid molecule comprises a sequence according to SEQ ID NO: 3, wherein the sequence comprises one or more nucleotide modifications at positions which give rise to a modification at position L903F of SEQ ID NO: 1, and modifications at positions D390A, and E392A, of SEQ ID NO: 1.

An “isolated” nucleic acid molecule is substantially separated away from other nucleic acid sequences with which the nucleic acid is normally associated, such as, from the chromosomal or extrachromosomal DNA of a cell in which the nucleic acid naturally occurs. A nucleic acid molecule may be an isolated nucleic acid molecule when it comprises a transgene or part of a transgene present in the genome of another organism. The term also embraces nucleic acids that are biochemically purified so as to substantially remove contaminating nucleic acids and other cellular components. Isolated nucleic acids are substantially free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The isolated nucleic acid molecule may be flanked by its native genomic sequences that control its expression in the cell, for example, the native promoter, or native 3′ untranslated region.

Suitably the isolated nucleic acid molecule may be comprised upon a vector, suitably an expression vector.

Suitable expression vectors are those which are designed for expression in plant cells, suitably plant expression vectors. Suitably expression vectors which enable expression of the nucleic acid molecule, and therefore the encoded organellar DNA polymerase, in plant cells. Such vectors may contain, in addition to the nucleic acid molecule of the invention, other heterologous nucleic acid sequences, which are nucleic acid sequences that are not naturally found adjacent to a sequence encoding an organellar DNA polymerase, and that may be derived from a species other than the species from which the sequence encoding an organellar DNA polymerase is derived.

Suitably the vector can be either RNA or DNA, either prokaryotic or eukaryotic, and typically is a virus or a plasmid. In one embodiment the vector is a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. For example the vector may be pBIN 19 (Bevan, 1984) pART7 or pART27 (Gleave, 1992).

Suitably the expression vector may further comprise one or more regulatory elements to aid expression of the nucleic acid molecule. The term “regulatory element” or “regulatory sequence” as used herein refers to a nucleic acid that is capable of regulating the transcription and/or translation of an operably linked nucleic acid molecule. Regulatory elements include, but are not limited to, promoters, enhancers, introns, 5′ UTRs, and 3′ UTRs. For example, the expression vector may contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression cassette. The expression cassette may include one or more regulatory sequences that are functional in plants, thus allowing expression of the nucleic acid molecule encoding an organellar DNA polymerase enzyme in a plant.

“Expression cassette” as used herein means a nucleic acid sequence capable of directing expression of a particular nucleic acid sequence in an appropriate host cell, comprising a promoter operably linked to the nucleic acid sequence of interest, in this case a nucleic acid molecule comprising a sequence encoding an organellar DNA polymerase, which is operably linked to termination signal sequences. It also typically comprises sequences required for proper translation of the nucleic acid sequence. The expression cassette comprising the nucleic acid sequence of interest may be chimeric, meaning that at least one of its components is heterologous with respect to at least one of its other components, which is already defined above. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell. The expression of the nucleic acid molecule in the expression cassette may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

Expression cassettes may include in the 5′-3′ direction of transcription, a transcriptional and translational initiation region (e.g., a promoter), a nucleic acid molecule comprising a sequence encoding an organellar DNA polymerase of the invention, and a transcriptional and translational termination region (e.g., termination region) functional in plants.

In one embodiment, the expression vector or expression cassette may comprise in the 5′-3′ direction of transcription, a 5′UTR, a promoter, a nucleic acid molecule comprising a sequence encoding an organellar DNA polymerase of the invention, and a 3′UTR.

Suitably the 5′UTR, the promoter and the nucleic acid molecule comprising a sequence encoding an organellar DNA polymerase of the invention are operably linked.

Any promoter can be used in the production of the expression cassettes and vectors including such expression cassettes as described herein. The promoter may be native or analogous, or foreign or heterologous, to the plant host and/or to the organellar DNA polymerase nucleic acid sequence. Additionally, the promoter may be a natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the organellar DNA polymerase nucleic acid molecule, it is intended that the promoter is not the native or naturally occurring promoter for the operably linked organellar DNA polymerase nucleic acid molecule.

While it may be preferable to express the nucleic acid molecule of the invention using heterologous promoters, the native promoter sequences may be used in the preparation of the expression cassettes. Such expression cassettes may change expression levels of the organellar DNA polymerase enzyme in the plant or plant cell. Thus, the phenotype of the plant or plant cell is altered.

Any promoter can be used in the preparation of expression cassettes to control the expression of the nucleic acid molecule encoding the organellar DNA polymerase, such as promoters providing for constitutive, tissue-preferred, inducible, or other promoters for expression in plants. Constitutive promoters include, for example, the core promoter of the Rsyn7 promoter and other constitutive promoters disclosed in WO 99/43 838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to direct expression of the organellar DNA polymerase enzyme within a particular plant tissue. Such tissue-preferred promoters include, but are not limited to, leaf-preferred promoters, root-preferred promoters, seed-preferred promoters, and stem-preferred promoters. Tissue-preferred promoters include those described in Yamamoto et α/. (1997) Plant J. 12(2):255-265; Kawamata et α/. (1997) Plant Cell Physiol. 38(7):792-803; Hansen et al. (1997) Mol Gen Genet. 254(3):337-343; Russell et al. (1997) Transgenic Res. 6(2): 157-168; Rinehart et al. (1996) Plant Physiol. 1 12(3):1331-1341; Van Camp et al. (1996) Plant Physiol. 1 12(2):525-535; Canevascini et al. (1996) Plant Physiol. 112(2): 513-524; Yamamoto et al. (1994) Plant Cell Physiol. 35(5):773-778; Lam (1994) Results Probl. Cell Differ. 20:181-196; Orozco et al (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505.

In one embodiment, the promoter is the native promoter of the organellar DNA polymerase, suitably of the wild type organellar DNA polymerase from which the modified enzyme is derived. Suitably therefore, where the organellar DNA polymerase comprises an amino acid sequence according to SEQ ID NO: 1 with the modifications defined herein, suitably the promoter is the native Nicotiana tabacum organellar DNA polymerase promoter according to SEQ ID NO: 15. Advantageously use of the native promoter ensures that the organellar DNA polymerase of the invention will be expressed together with the other enzymes required for DNA replication.

The expression cassettes may also comprise transcription termination regions. Where transcription terminations regions are used, any termination region may be used in the preparation of the expression cassettes. For example, the termination region may be native to the transcriptional initiation region, may be native to the operably linked nucleic acid molecule comprising a sequence encoding the organellar DNA polymerase, may be native to the plant host, or may be derived from another source (i.e., foreign or heterologous to the promoter, the nucleic acid molecule of the invention, the plant host, or any combination thereof). Examples of termination regions that are available for use in the expression cassettes and vectors of the present invention include those from the Ti-plasmid of A. tumefaciens, such as the octopine synthase and nopaline synthase termination regions. See also Guerineau et al. (1991) Mol. Gen. Genet. 262: 141-144; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

The nucleic acid molecule may be optimized for increased expression in a transformed plant. That is, the nucleic acids encoding the organellar DNA polymerase enzyme can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498.

In addition, other sequence modifications can be made to the nucleic acid molecules of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

Other nucleic acid sequences may also be used in the preparation of the expression cassettes of the present invention, for example to enhance the expression of the nucleic acid molecule sequence. Such nucleic acid sequences include the introns of the maize Adhl, intronI gene (Callis et al. (1987) Genes and Development 1:1183-1200), and leader sequences, (W-sequence) from the Tobacco Mosaic virus (TMV), Maize Chlorotic Mottle Virus and Alfalfa Mosaic Virus (Gallie et al (1987) Nucleic Acid Res. 15:8693-8711, and Skuzeski et al. (1990) Plant Mol. Biol. 15:65-79, 1990). The first intron from the shrunken-1 locus of maize has been shown to increase expression of genes in chimeric gene constructs. U.S. Pat. Nos. 5,424,412 and 5,593,874 disclose the use of specific introns in gene expression constructs, and Gallie et al. ((1994) Plant Physiol. 106:929-939) also have shown that introns are useful for regulating gene expression on a tissue specific basis. Plant cells transformed with such modified expression cassettes or vectors, then, may exhibit overexpression or constitutive expression of a nucleic acid molecule of the invention.

Expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassettes and expression vectors described herein, the various nucleic acid molecules may be manipulated, so as to provide for the nucleic acid molecules in the proper orientation and, as appropriate, in the proper reading frame. Toward this end, adapters or linkers may be employed to join the nucleic acid molecules or other manipulations may be involved to provide for convenient restriction sites, removal of superfluous nucleic acid molecules, removal of restriction sites, or the like. For this purpose, in vitro mutagenesis, primer repair, restriction, annealing, resubstitutions, e.g., transitions and transversions, may be involved.

The expression cassettes of the present invention can also include nucleic acid sequences capable of directing the expression of the organellar DNA polymerase to the chloroplast. Such nucleic acid sequences include chloroplast targeting sequences that encode a chloroplast transit peptide which directs the organellar DNA polymerase to plant cell chloroplasts. Such transit peptides are known in the art. With respect to chloroplast-targeting sequences, “operably linked” means that the nucleic acid sequence encoding a transit peptide (i.e., the chloroplast-targeting sequence) is linked to the nucleic acid sequence encoding the organellar DNA polymerase such that the two sequences are contiguous and in the same reading frame. See, for example, Von Heijne et al. (1991) Plant Mol. Biol. Rep. 9:104-126; Clark et al (1989) J Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233 Al S-4SI.

Suitably the organellar DNA polymerase of the invention may already comprise a native chloroplast transit peptide. However, any chloroplast transit peptide known in the art can be fused to the amino acid sequence of a mature organellar DNA polymerase of the invention by operably linking a chloroplast-targeting sequence to the 5′-end of a nucleotide sequence encoding a mature organellar DNA polymerase enzyme of the invention.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J Biol. Chem. 270(1 I):6081-6087); plastocyanin (Lawrence et al. (1997) J Biol. Chem. 272(33):20357-20363); chorismate synthase (Schmidt et al. (1993) J Biol. Chem. 268(36):27447-27457); and the light harvesting chlorophyll a/b binding protein (LHBP) (Lamppa et al. (1988) J Biol. Chem. 263:14996-14999). See also Von Heijne et al. (1991) Plant Mol. Biol. Rep. 9:104-126; Clark et al. (1989) J Biol. Chem. 264:17544-17550; Della-Cioppa et al (1987) Plant Physiol. 84:965-968; Romer et al. (1993) Bio chem. Biophys. Res. Com. 196: 1414-1421; and Shah et al. (1986) Science 233:478-481.

Suitably the expression cassette comprises a sequence encoding a transit peptide, suitably a chloroplast transit peptide. Suitably the chloroplast transit peptide may be a rubisco small subunit transit peptide. Suitably the expression cassette may optionally comprise a sequence encoding a tag for isolation of the protein, for example a strep tag. Suitably the Strep Tag may comprise a sequence according to SEQ ID NO: 5. Suitably the tag may be attached to the organellar DNA polymerase of the invention by a linker. Suitably the expression cassette may optionally comprise a sequence encoding the linker, wherein the linker may comprise a sequence according to SEQ ID NO: 6.

In one embodiment, the expression cassette comprises a sequence encoding a rubisco small subunit transit peptide operably linked to a sequence encoding an organellar DNA polymerase of the invention. In one embodiment, the expression cassette comprises a promoter according to SEQ ID NO: 15 operably linked to a sequence encoding a rubisco small subunit transit peptide operably linked to a sequence encoding an organellar DNA polymerase of the invention. In such an embodiment, suitably the organellar DNA polymerase is a N. tabacum organellar DNA polymerase. Optionally the expression cassette may further optionally be operably linked to a sequence encoding a strep tag according to SEQ ID NO: 6 by a linker according to SEQ ID NO: 5.

In one embodiment, the expression cassette may comprise a sequence encoding an amino acid sequence according to SEQ ID NO: 14. In one embodiment, the expression vector may comprise the expression cassette, therefore the expression vector may comprise a sequence encoding an amino acid sequence according to SEQ ID NO: 14.

The expression cassettes of the present invention can also include nucleic acid sequences capable of directing the expression of the organellar DNA polymerase to the mitochondria. Such nucleic acid sequences include mitochondrial targeting sequences that encode a mitochondrial targeting peptide, otherwise known as a mitochondrial presequence, which directs the organellar DNA polymerase to plant cell mitochondria. Such mitochondrial targeting peptides or presequences are known in the art. With respect to mitochondrial-targeting sequences, “operably linked” means that the nucleic acid sequence encoding a mitochondrial targeting peptide (i.e., the mitochondrial-targeting presequence) is linked to the nucleic acid sequence encoding the organellar DNA polymerase such that the two sequences are contiguous and in the same reading frame. See, for example, Von Heijne et al. (1991) Plant Mol. Biol. Rep. 9:104-126; Clark et al (1989) J Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233 Al S-4SI.

Suitably the organellar DNA polymerase of the invention may already comprise a native mitochondrial targeting peptide or presequence. However, any mitochondria targeting peptide or presequence known in the art can be fused to the amino acid sequence of a mature organellar DNA polymerase of the invention by operably linking a mitochondrial-targeting presequence to the 5′-end of a nucleotide sequence encoding a mature organellar DNA polymerase enzyme of the invention.

Mitochondrial targeting sequences are known in the art and include the soybean (Glycine max) Alternative Oxidase 1 (AOX1) presequence (Lee and Whelan (2004) Plant Mol Biol 54: 193-203, or any presequences from other mitochondrial targeting proteins, such as the Arabidopsis mitochondrial Isovaleryl-coenzyme A dehydrogenase (Däschner et al (2001) Plant Physiol 126: 601-612, the Arabidopsis mitochondrial ATPase delta prime subunit (Arimura et al (2002) Proc Natl Acad Sc. USA 99: 5727-5731), and the Nicotiana plumbaginifolia mitochondrial F1-ATPase beta subunit (Chaumont et al (1990) J Biol Chem 265: 16856-16862) Suitably the expression cassette comprises a sequence encoding a targeting peptide, suitably a mitochondrial targeting peptide or presequence. Suitably the mitochondrial N-terminal presequence may be a mitochondrial Alternative Oxidase 1 (AOX1) presequence. Suitably the expression cassette may optionally comprise a sequence encoding a tag for isolation of the protein, for example a strep tag. Suitably the Strep Tag may comprise a sequence according to SEQ ID NO: 5. Suitably the tag may be attached to the organellar DNA polymerase of the invention by a linker. Suitably the expression cassette may optionally comprise a sequence encoding the linker, wherein the linker may comprise a sequence according to SEQ ID NO: 6.

In one embodiment, the expression cassette comprises a sequence encoding an Alternative Oxidase 1 (AOX1) presequence operably linked to a sequence encoding an organellar DNA polymerase of the invention. In one embodiment, the expression cassette comprises a promoter according to SEQ ID NO: 15 operably linked to a sequence encoding an Alternative Oxidase 1 (AOX1) presequence operably linked to a sequence encoding an organellar DNA polymerase of the invention. In such an embodiment, suitably the organellar DNA polymerase is a N. tabacum organellar DNA polymerase. Optionally the expression cassette may further optionally be operably linked to a sequence encoding a strep tag according to SEQ ID NO: 6 by a linker according to SEQ ID NO: 5.

In one embodiment, the expression cassette may comprise a sequence encoding an amino acid sequence according to SEQ ID NO: 91. In one embodiment, the expression vector may comprise the expression cassette, therefore the expression vector may comprise a sequence encoding an amino acid sequence according to SEQ ID NO: 91.

In one embodiment, the expression cassette or the expression vector may comprise a sequence according to SEQ ID NO: 90.

The expression cassettes and vectors of the invention may be prepared to direct the expression of the nucleic acid molecule from the plant cell chloroplast. Alternatively, the expression cassettes and vectors of the invention may direct expression of the nucleic acid molecule from the nucleus of the plant cell, and the resulting polymerase is targeted to the chloroplast, suitably by the chloroplast targeting peptide.

The nucleic acid molecule to be targeted to the chloroplast may be optimized for expression in the chloroplast to account for differences in codon usage between the plant nucleus and this organelle. In this manner, the nucleic acid molecule may be synthesized using chloroplast-preferred codons. See, for example, U.S. Pat. No. 5,380,831.

The expression cassettes and vectors of the invention may be prepared to direct the expression of the nucleic acid molecule from the plant cell mitochondria. Alternatively, the expression cassettes and vectors of the invention may direct expression of the nucleic acid molecule from the nucleus of the plant cell, and the resulting polymerase is targeted to the mitochondria, suitably by the mitochondrial targeting peptide.

The nucleic acid molecule to be targeted to the mitochondria may be optimized for expression in the mitochondria to account for differences in codon usage between the plant nucleus and this organelle. In this manner, the nucleic acid molecule may be synthesized using mitochondria-preferred codons.

Expression vectors may include additional features. For example, they may include additional features such as selectable markers, e.g. Phosphomannose Isomerase (PMI), and antibiotic resistance genes that can be used to aid recovery of stably transformed plants. In one embodiment, the expression vector comprises a kanamycin resistance gene for selection of stably transformed plants or plant parts.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence or nucleic acid molecule that is operably linked to a second nucleotide sequence or nucleic acid molecule, means a situation when the first nucleotide sequence or nucleic acid molecule is placed in a functional relationship with the second nucleotide sequence or nucleic acid molecule. For instance, a promoter is operably associated with a nucleotide sequence or nucleic acid molecule if the promoter effects the transcription or expression of said nucleotide sequence or nucleic acid molecule. Those skilled in the art will appreciate that the control sequences (e.g., promoter) need not be contiguous with the nucleotide sequence or nucleic acid molecule to which it is operably associated, as long as the control sequences function to direct the expression thereof. Thus, for example, intervening untranslated, yet transcribed, sequences can be present between a promoter and a nucleotide sequence or nucleic acid molecule, and the promoter can still be considered “operably linked” to or “operatively associated” with the nucleotide sequence or nucleic acid molecule.

### Organelle

The organellar DNA polymerase of the invention may be present within an organelle, in order to modify the organelle genome. Therefore an organelle comprising and optionally expressing the organellar DNA polymerase of the invention is envisaged, as are plants or plant cells comprising said organelles.

Suitably the organelle may be a plastid or a mitochondria. Suitable plastids are chloroplasts, proplastids, etioplasts, chromoplasts, leucoplast, amyloplasts, gerontoplasts, elaioplasts, proteinoplasts, muroplasts, cyanoplasts, rhodoplasts, and apicoplasts. In one embodiment the organelle is a chloroplast. In another embodiment the organelle is a mitochondria.

Suitably the entire organelle DNA within a plastid is a plastome. Suitably each plastid comprises multiple copies of the plastome. Suitably each plastid comprises between 5-100 copies of the plastome.

Suitably the entire organelle DNA within a mitochondrion is a mitogenome. Suitably each mitochondrion comprises multiple copies of the mitogenome. Suitably each mitochondrion comprises between 2-10 copies of the mitogenome.

Suitably, the organellar DNA polymerase modifies the plastome of a plastid, or the mitogenome or a mitochondrion. Suitably, the organellar DNA polymerase may modify one or more copies of the plastome within a plastid, or one or more copies of the mitogenome in a mitochondrion. Suitable modifications that may be made to the organelle DNA by the organellar DNA polymerase are described elsewhere herein.

### Plant or Part Thereof

Further provided herein is a plant or a part thereof comprising and suitably expressing the organellar DNA polymerase of the invention. Suitably, this is achieved by the plant or part thereof comprising an organelle which in turn comprises the organellar DNA polymerase of the invention. Suitably the plant or part thereof is modified to comprise and express the organellar DNA polymerase. Therefore, other aspects of the invention further define a method of modifying a plant or part thereof, by introducing into the plant or part thereof, the organellar DNA polymerase of the invention or a nucleic acid molecule or expression vector of the invention which comprise a sequence encoding the organellar DNA polymerase.

As used herein unless clearly indicated otherwise, the term “plant” is intended to mean a plant at any developmental stage, as well as any part or parts of a plant that may be attached to or separate from a whole intact plant. The term “plant” is used in its broadest sense as it pertains to organic material and is intended to encompass eukaryotic organisms that are members of the Kingdom Plantae, examples of which include but are not limited to vascular plants, vegetables, grains, flowers, trees, herbs, bushes, grasses, vines, ferns, mosses, fungi and algae, etc, as well as clones, offsets, and parts of plants used for asexual propagation.

Such parts of a plant include, but are not limited to, organs, tissues, and cells of a plant including, plant calli, plant clumps, plant protoplasts and plant cell tissue cultures from which plants can be regenerated. Examples of particular plant parts include a stem, a leaf, a root, an inflorescence, a flower, a floret, a fruit, a pedicle, a peduncle, a stamen, an anther, a stigma, a style, an ovary, a petal, a sepal, a carpel, a root tip, a root cap, a root hair, a leaf hair, a seed hair, a pollen grain, a microspore, an embryos, an ovule, a cotyledon, a hypocotyl, an epicotyl, xylem, phloem, parenchyma, endosperm, a companion cell, a guard cell, and any other known organs, tissues, and cells of a plant. Furthermore, it is recognized that a seed is a plant part.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. A “plant organ” is a distinct and visibly structured and differentiated part of a plant such as a root, stem, leaf, flower bud, or embryo.

Suitable plants for use in the present invention may comprise any species of plant, suitably any agriculturally or economically significant plant species. Suitable agriculturally significant plant species may comprise crop plants.

Suitable economically significant plant species may comprise species of plant which produce or which can be used to produce valuable products for purposes other than food.

In one embodiment, the plant is selected from the following species: corn or maize (Zea mays), Brassica sp. (e.g., B. napus, B. rapa, B. juncea), including those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Primus amygdalus), sugar beets (Beta vulgaris), sugarcane (Saccharum spp.), oats (Avena sativa), barley (Hordeum vulgare), vegetables, ornamentals, and conifers. Preferably, plants of the present invention are crop plants (for example, sunflower, Brassica sp., cotton, sugar, beet, soybean, peanut, alfalfa, safflower, tobacco, corn, rice, wheat, rye, barley triticale, sorghum, millet, etc.).

In one embodiment, the plant is tobacco (Nicotiana tabacum).

### Seeds

The invention further relates to a seed capable of producing a plant or part thereof comprising the organellar DNA polymerase of the invention, or a nucleic acid molecule or expression vector of the invention which comprises a sequence encoding the organellar DNA polymerase.

The term “seed” embraces seeds and plant propagules of all kinds including but not limited to true seeds, seed pieces, suckers, corms, bulbs, fruit, tubers, grains, cuttings, cut shoots and the like.

Seeds may be treated or untreated seeds. For example, the seeds can be treated to improve germination, for example, by priming the seeds, or by disinfection to protect against seed-born pathogens. In another example, seeds can be coated with any available coating to improve, for example, plantability, seed emergence, and protection against seed-born pathogens. Seed coating can be any form of seed coating including, but not limited to pelleting, film coating, and encrustments.

The seed may be germinated and used to produce or grow a plant or part thereof of the invention. That is a plant including a nucleic acid molecule, organellar DNA polymerase enzyme or expression vector of the invention.

Also provided herein is a container including seeds of the invention. A container of seeds may contain any number, weight or volume of seeds. For example, a container can contain at least, or greater than, about 10, 25, 50, 75, 100, 200, 300, 400, 500, 600, 700, 800, 900, 1000, 1500, 2000, 2500, 3000, 3500, 4000, 4500, 5000 or more seeds. Alternatively, the container can contain at least, or greater than, about 1 ounce, 5 ounces, 10, ounces, 1 pound, 2 pounds, 3 pounds, 4 pounds, 5 pounds or more seeds.

Containers of plant seeds may be any container available in the art. By way of non-limiting example, a container may be a box, a bag, a packet, a pouch, a tape roll, a pail, a foil, or a tube.

Seeds contained in a containers may be treated or untreated seeds.

At least 10% of seeds within a container may be seeds of the invention. For example, at least 10%, 20%, 30%, 40%, 50%, 60%, 70%, 80%, 90%, 95%, 99% or 100% of the seeds in the container may be seeds of the invention.

### Method of Modifying a Plant or Organelle

The invention also includes methods for modifying plants or parts thereof to express an organellar DNA polymerase enzyme of the invention.

Suitably the methods described herein are not essentially biological processes for the production of plants. Suitably the methods described herein are not processes for modifying the germ line genetic identity of human beings.

Methods of modifying plants may include introducing a nucleic acid molecule according of the invention, or an expression vector according to the invention into a plant or part thereof and expressing the nucleic acid molecule to produce an organellar DNA polymerase enzyme of the invention in the plant or part thereof.

In one embodiment, a plant, or a plant part, is transformed with a nucleic acid molecule or an expression vector of the invention. Suitably in such an embodiment, the method comprises step (b) of inducing expression of the nucleic acid molecule or expression vector in the plant or part thereof.

Suitably expression may occur constitutively, suitable therefore no induction of expression is required. Alternatively, the methods as described herein may further comprise a step of inducing expression of the nucleic acid molecule or expression vector in the plant or part thereof. Inducing expression in a plant may be achieved by exposing the plant to an inducer. Suitable inducers include alcohol, tetracycline, dexamethasone, heat, cold, metals, pathogenesis related proteins. Suitably in such embodiments, the nucleic acid molecule encoding organellar DNA polymerase enzyme of the invention is under the control of an inducible promoter. Suitably therefore this step may comprise contacting the plant, plant part, cell or protoplast with an effective concentration of an inducer. Suitably an effective concentration is a concentration sufficient to induce expression of the organellar DNA polymerase. Suitably the inducer is capable of stimulating transcription from the inducible promoter, for example if the inducible promoter is an ethanol-inducible promoter, then the inducer used is ethanol.

“Transformation” refers to a process of introducing an exogenous nucleic acid molecule (for example, a recombinant polynucleotide) into a cell or protoplast and that exogenous nucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign nucleic acid, such as an expression vector or nucleic acid molecule has been introduced. The nucleic acid molecule can be stably integrated into the genome of the host or the nucleic acid molecule can also be present as an extrachromosomal molecule. Such an extrachromosomal molecule can be auto-replicating. The nucleic acid molecule can also be introduced into the genome of the chloroplast or the mitochondria of a plant cell.

Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al, Nature 303:209 (1983); Bevan, Nucleic Acids Res. 12:8711-8721 (1984); Klee et ak, Bio-Technology 3(7):637-642 (1985). In addition to plant transformation vectors derived from the Ti or root-inducing (Ri) plasmids of Agrobacterium, alternative methods can be used to insert the nucleic acid molecules of this invention into plant cells. Such methods may involve, but are not limited to, for example, the use of liposomes, electroporation, chemicals that increase free DNA uptake, free DNA delivery via microprojectile bombardment, and transformation using viruses or pollen.

Methods for transformation of chloroplasts and mitochondria (in algae and yeasts) are known in the art. See, for example, Boynton et al. (1988) Science 240:1534-1538; Johnston et al (1988) Science 240:1538-41; Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606; Remacle et al (2006)Proc Natl Acad Sci USA 103:4771-4776. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Suitably in the present methods, plants or parts thereof, suitably plant cells, are transformed with the error prone organellar DNA polymerase of the invention or nucleic acid or vector encoding said polymerase, and expression of the polymerase is induced, and the polymerase is subsequently targeted to the organelle, suitably by use of a targeting peptide. Suitably therefore the host cell genome is transformed, suitably the host nuclear genome is transformed.

Whole plants, plant material or plant parts may be stably or transiently transformed as desired, wherein stable transformation refers to polynucleotides which become incorporated into the plant host chromosomes such that the host genetic material may be permanently and heritably altered and the transformed cell may continue to express traits caused by this genetic material, even after several generations of cell divisions. In such embodiments, the modified plant, plant part, cell or protoplast may be referred to as a transgenic plant, plant part, cell or protoplast. Transiently transformed plant cells refer to cells which contain heterologous DNA or RNA, and are capable of expressing the trait conferred by the heterologous genetic material, without having fully incorporated that genetic material into the cell's DNA. Heterologous genetic material may be incorporated into nuclear or plastid (chloroplastic or mitochondrial) genomes as required to suit the application of the invention. In such embodiments, the modified plant, plant part, cell or protoplast may be referred to as a non-transgenic plant, plant part, cell or protoplast. Where plants are transformed with more than one polynucleotide it is envisaged that combinations of stable and transient transformations are possible.

Generally after transformation, plant cells or cell groupings are selected for the presence of one or more markers which are encoded by plant-expressible genes co-transferred with the gene of interest, following which the transformed material is regenerated into a whole plant.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as an antibiotic resistance marker, for example kanamycin resistance.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Suitably the method of modifying a plant or part thereof produces a modified plant or part thereof. Suitably said modified plant or plant part may be a transgenic or transformed plant or plant part.

A “transgenic” or “transformed” plant also includes progeny of the plant and progeny produced from a breeding program employing such a “transgenic” plant as a parent in a cross and exhibiting an altered phenotype resulting from the presence of the nucleic acid molecule encoding the organellar DNA polymerase.

The transgenic plants may be homozygous for the nucleic acid molecule encoding an organellar DNA polymerase enzyme described herein (i.e. those that contain two added genes encoding an organellar DNA polymerase enzyme at the same position on each chromosome of the chromosome pair). Homozygous transgenic plants may be obtained by crossing (self-pollinating) independent transgenic plant isolates containing a single added gene, germinating some of the resulting seeds, and transforming the resulting plant with the nucleic acid molecule or expression vector of the invention.

The modified plants of the present invention include both non-transgenic plants and transgenic plants. By “non-transgenic plant” is intended to mean a plant lacking recombinant DNA in its genome, but containing the mutant nucleic acid molecule in the plant cell genome which has been mutated using mutagenic techniques, such as chemical mutagenesis or by those methods provided herein. Non-transgenic plants may encompass those plants having mutant sequences as a result of natural processes, such as plants including spontaneous organellar DNA polymerase enzymes that correspond to the organellar DNA polymerase enzymes of the invention. By “transgenic plant” is intended to mean a plant comprising recombinant DNA in its genome. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

In one embodiment, any of the plants produced herein are transgenic.

### Method of Producing a Plant Having Homoplasmic Modified Organelle DNA

The invention further relates to producing plants having homoplasmic modified organelle DNA by using an error prone DNA polymerase, such as that described herein, and a series of specific selection steps. A plant having homoplasmic modified organelle DNA is also part of the invention, suitably which is produced from the method.

By ‘homoplasmic’ it is meant that the organelle DNA within the plant is the same in each organelle of the same type. Suitably this means that the modifications introduced by the error prone DNA polymerase by the method of the invention into an organelle genome are present in every organelle genome, in every organelle of the same type, in every cell of the plant. Suitably, for a chloroplast, this means that the modifications introduced by the error prone DNA polymerase into a chloroplast plastome are present in every chloroplast plastome, in every chloroplast, in every cell of the plant. Suitably, for a mitochondrion, this means that the modifications introduced by the error prone DNA polymerase into a mitogenome are present in every mitogenome, in every mitochondrion, in every cell of the plant.

Suitably the method comprises a first step of introducing an error prone organellar DNA polymerase or a nucleic acid molecule encoding said polymerase into a plant and optionally inducing expression thereof, so that the polymerase is expressed in the plant and modifies the organelle DNA. Suitably the step of introducing comprises transforming the plant with the error prone organellar DNA polymerase or a nucleic acid molecule encoding said polymerase, suitable methods of transformation are explained elsewhere herein. Suitably, the polymerase replicates the organelle DNA in the plant and thereby introduces errors into the organelle DNA. Suitably therefore this step may comprise introducing the polymerase, or nucleic acid molecule encoding said polymerase, into the plant to replicate the organelle DNA which thereby modifies the organelle DNA. Suitably by error prone replication of the organelle DNA. Suitable modifications introduced by the error prone polymerase are discussed elsewhere herein.

Suitably the error prone organellar DNA polymerase may be any error prone organellar DNA polymerase. By ‘error prone’ it is meant that it introduces a plurality of mutations into organelle DNA during replication. Suitably the organellar DNA polymerase of the invention has an increased error rate compared to a reference organellar DNA polymerase. Suitably the organellar DNA polymerase has an increased error rate of mutations per base than a reference organellar DNA polymerase. Suitably which may be a wild type organellar DNA polymerase, suitably a wild type endogenous organellar DNA polymerase from the plant to be modified. Suitable error rates for an error prone organellar DNA polymerase are discussed above.

Suitably the error prone organellar DNA polymerase is a modified enzyme. Suitably the enzyme has been modified to increase its error rate. Suitably such modifications are discussed elsewhere herein, but other modifications may be envisaged which may also produce an error prone organellar DNA polymerase with an increased error rate. Suitably the modified error prone organellar DNA polymerase has an increased error rate compared to a reference organellar DNA polymerase. Suitably a reference organellar DNA polymerase which is not modified, suitably which is a wild type organellar DNA polymerase from the same plant.

Suitably the error prone organellar DNA polymerase has characteristics which contribute towards a generating a homoplasmic modified organelle DNA. Suitably the error prone-organellar DNA polymerase modifies organelle DNA throughout the organellar genome, and is semi-dominant to the endogenous organellar DNA polymerases present in the plant(s).

Suitably the error prone-organellar DNA polymerase modifies organelle DNA throughout the organellar genome, suitable organelles and their corresponding genomes are defined elsewhere herein. Suitably the error prone organellar DNA polymerase introduces mutations into organelle DNA across the entire replication region. Suitably the replication region is the region of organelle DNA to be replicated by the enzyme. Suitably when the enzyme is expressed within an organelle, the replication region may be the entire organelle genome, suitably in the case of plastids, this may be known as the ‘plastome’. Suitably, the error prone organellar DNA polymerase introduces one or more mutations scattered across the organelle genome, suitably randomly across the organelle genome. Suitably these mutations may be spaced within a few hundred bases of each other or may be spaced as much as 75,000 bases apart. Suitably therefore, on average, the error prone organellar DNA polymerase introduces a mutation into the organelle genome every 100-500 bases, suitably every 100-400 bases, suitably every 100-300 bases, suitably every 100-200 bases.

Suitably the error prone organellar DNA polymerase is semi-dominant to the endogenous organellar DNA polymerases present in the plant(s). This means that the error prone organellar DNA polymerase competes with reference wild type organellar DNA polymerases. Suitably the error prone organellar DNA polymerase outcompetes reference wild type organellar DNA polymerases. Suitably the error prone organellar DNA polymerase is semi-dominant to reference wild type organellar DNA polymerases. Suitably the error prone organellar DNA polymerase is dominant to reference wild type organellar DNA polymerases. Suitably when both the error prone organellar DNA polymerase is present in the plant to be modified, together with the wild type endogenous organellar DNA polymerases, the mutation rate of DNA is still elevated, thereby demonstrating that the error prone organellar DNA polymerase dominates replication. This may be determined by a gap-replication assay in which both the error prone organellar DNA polymerase to be tested, and a reference wild type organellar DNA polymerase, suitably endogenous to the plant to be modified, are present. A suitable gap replication assay is conducted in the examples herein. The error rate in the subsequently replicated strand can be determined and attributed to either polymerase. If the error rate is the same as the error rate of the error prone organellar DNA polymerase then the error pone organellar DNA polymerase is dominant. If the error rate is higher than expected when using a reference wild type organellar DNA polymerase, but not the same as the error rate of the error prone organellar DNA polymerase then the error-prone DNA polymerase is semi-dominant. If the error rate is the same as the error rate of a reference wild type organellar DNA polymerase then the error prone organellar DNA polymerase is not dominant but is recessive to the wild type organellar DNA polymerase.

In preferred embodiments the error prone organellar DNA polymerase is the error prone organellar DNA polymerase of the first aspect of the invention, as further described in detail herein. Suitably the error prone organellar DNA polymerase of the invention has the characteristics identified above.

Suitably step (b) of the method comprises (i) taking an explant from the modified plant and culturing one or more shoots therefrom, or (ii) generating F1 seedlings from the plant.

Suitably an explant is a cutting taken from the modified plant. Suitably the explant is a cutting taken from the leaf of the modified plant. Suitably the explant comprises a small number of cells, suitably between 1-10 cells of the modified plant. Suitably the explant comprises only 1 cell of the modified plant. Suitably therefore each explant comprises a single cell from the leaf of a modified plant.

Suitably the explant is cultured, suitably on growth media. Suitably this stimulates the growth of one or more shoots from the explant. Suitably the explant is cultured for 21 to 42 days. Suitably under aseptic condition on agar (0.6 to 0.8% W/V) solidified shoot regeneration medium which may be comprised of MS medium (pH 5.8) (Murashige and Skoog, 1962) containing 2-(N-morpholino)ethanesulfonic acid, 3% (W/V sucrose and supplemented with 1 μg/mL 6-benzylaminopurine and 0.1 μg/ml naphthaleneacetic acid. Suitably using shoot regeneration media. Suitably such culture conditions are generally applicable to any plant species, and are well known in the art. Suitable culture conditions for a variety of plants including dicots and monocots may be found in: Dodds and Roberts (1982) Experiments in Plant Tissue Culture, Cambridge University Press, Cambridge; Vasil and Thorpe T A Eds (1994) Plant Cell Tissue Culture, Springer Netherlands, Jackson and Linskens Eds (2003) Mol Meth Plant Analysis vol 23: Genetic Transformation of plants, Springer-Verlag, Berlin Dordrech; Pena Ed (2005) Transgenic plants: methods and protocols, Humana Press, New Jersey; Loyola-Vargas and Vezquez-Flota Eds (2005) Plant Cell Culture Protocols, Humana Press, New Jersey; Suitably each shoot is a modified shoot in that it comprises modified organelle DNA. Suitably comprising the same modified organelle DNA as the plant of step (a) from which the shoot was derived.

Alternatively, F1 seedlings may be generated from the modified plant. Suitably F1 seedlings are generated by crossing a modified plant produced from step (a) with a non-modified wild type plant, suitably of the same species. Suitably the female stigma of the modified plant from step (a) is contacted with male pollen from the non-modified plant. Suitably since organelle DNA is typically maternally inherited, this ensures that the F1 progeny inherit the modified organelle DNA. Suitably after crossing, F1 seeds are produced. Suitably the seeds may be grown into seedlings.

Suitably the seedlings are grown under suitable conditions for the species of plant which will be known to the skilled person. For example, N. tabacum seedlings may be grown in soil at a temperature of 25-28° C., for 12 to 16 hour days using a light intensity of 100 to 300 microEinsteins m⁻² s⁻¹ Suitably each seedling comprises modified organelle DNA. Suitably comprising the same modified organelle DNA as the maternal plant of step (a) from which the seed was derived.

Suitably step (c) of the method comprises exposing the shoots or seedlings to a selection agent which selects for modified organelle DNA. Suitably step (c) is optional. Suitably in methods relating to homoplasmic modified chloroplast DNA, and modified chloroplast organelles, step (c) is present.

Suitably a selection agent may be selected from one of the following: spectinomycin, atrazine, terbuthylazine, streptomycin, chloramphenicol, paromomycin, oligomycin, tentoxin and lincomycin, or any other herbicide which targets organelle functions for example.

In one embodiment step (c) comprises exposing shoots or seedlings to spectinomycin. Suitably in embodiments where the method relates to generating homoplasmic modified chloroplasts.

Suitably exposing the shoots or seedlings comprises contacted the shoots or seedlings with the selection agent. Suitably by adding the selection agent to the growth media or soil in which the shoots or seedlings are growing. Suitably the selection agent is added at an effective concentration to select the resistant shoots or seedlings. A suitable effective concentration of the selection agent may be between 50 ug/ml up to 500 ug/ml, suitably between 100 ug/ml up to 300 ug/ml, suitably 200 ug/ml.

In one embodiment thereof, the method of producing a plant having homoplasmic modified organelle DNA comprises;
(a) Introducing an error-prone organellar DNA polymerase, or a nucleic acid molecule encoding said polymerase, into one or more plants, and optionally inducing expression of the nucleic acid molecule in the or each plant, to modify the organelle DNA;
(b) Taking an explant from said plant(s) and culturing one or more shoots therefrom, or generating one or more F1 seedling(s) from said plant(s);
(c) Exposing the or each shoot or seedling to a selection agent which selects for modified organelle DNA, and optionally a further a selection agent which selects for a trait of interest;
(d) Selecting those shoots or seedlings having resistance to the selection agent which selects for modified organelle DNA;
(e) Optionally selecting those shoots or seedlings having resistance to the further selection agent;
(f) Regenerating the or each selected shoot or seedling into a plant; and
(g) Optionally repeating steps (b) to (f) one or more times;
   wherein the error prone-organellar DNA polymerase modifies the organelle DNA throughout the organelle genome, and is semi-dominant over endogenous organellar DNA polymerase present in the plant(s).

Suitably such an embodiment is a method of producing a plant having homoplasmic modified chloroplast DNA, or in some cases mitochondrial DNA.

Suitably, however, in some methods relating to homoplasmic modified mitochondria, step (c) may not be present. Suitably in such methods, after step (b), shoots or seedlings may be selected that have modified organelle DNA by virtue of their phenotypic appearance. Suitably in such methods selecting shoots or seedlings may comprise selecting those shoots or seedlings having atypical leaf colours or leaf appearances as explained below. In some embodiments, the shoots or seedlings having a narrow leaf phenotype may be selected. Suitably in such methods, no selection agent is needed to fix the organelle mutations.

In an alternative embodiment, step (c) may simply comprise selecting the shoots or seedlings with modified organelle DNA. Suitably by physical assessment of the shoots or seedlings. Suitably physical assessment may comprise selecting the shoots or seedlings on the basis of pigment. Suitably step (c) may comprise selecting shoots or seedlings having one or more bleached areas, suitably one or more bleached areas on one or more leaves. Suitably physical assessment may comprise selecting the shoots or seedlings on the basis of fluorescence. Suitably fluorescence changes in the shoots or seedlings may be observed by conducting fluorescence microscopy on one or more leaves. Suitably step (c) may comprise selecting shoots or seedlings having a change in leaf fluorescence relative to a non-modified reference plant of the same species.

In one embodiment therefore the method of producing a plant having homoplasmic modified organelle DNA comprises;
(a) Introducing an error-prone organellar DNA polymerase, or a nucleic acid molecule encoding said polymerase, into one or more plants, and optionally inducing expression of the nucleic acid molecule in the or each plant, to modify the organelle DNA;
(b) Taking an explant from said plant(s) and culturing one or more shoots therefrom, or generating one or more F1 seedling(s) from said plant(s);
(c) Selecting the or each shoot or seedling with modified organelle DNA;
(d) Optionally exposing the or each shoot or seedling to a further a selection agent which selects for a trait of interest;
(e) Optionally selecting those shoots or seedlings having resistance to the further selection agent;
(f) Regenerating the or each selected shoot or seedling into a plant; and
(g) Optionally repeating steps (b) to (f) one or more times;
   wherein the error prone-organellar DNA polymerase modifies the organelle DNA throughout the organelle genome, and is semi-dominant over endogenous organellar DNA polymerase present in the plant(s).

Suitably such an embodiment is a method of producing a plant having homoplasmic modified mitochondrial DNA.

Optionally step (d) may take place before step (c).

Suitably steps (b) and (c) of the method may be combined, for example culturing the shoots or growing the seedlings may occur at the same time as exposing the shoots or seedlings to a selection agent which selects for modified organelle DNA and optionally a further selection agent which selects for a trait of interest. Suitably this may be achieved by directly culturing the shoots or growing the seedlings in media or soil containing an effective concentration of the selection agent as discussed above.

Suitably step (d) comprises selecting those shoots or seedlings having resistance to the selection agent. Suitably the selection agent which selects for modified organelle DNA is an agent which would normally kill the shoot or seedling, unless it has a mutation in the organelle DNA which confers resistance to the agent. For example, several point mutations in chloroplast 16S rDNA can confer resistance to spectinomycin.

Suitably this step allows the fixing of mutations within the organelle DNA of the shoots or seedlings. Suitably whilst resistance to the selection agent is selected for, a plurality of other mutations in the organelle DNA are also present in these shoots and seedlings, which are selected for in the same step.

Optionally steps (c) and (e) may comprise exposing the shoots or seedlings to a further selection agent which selects for a trait of interest. Suitably in addition to the selection agent which selects for modified organelle DNA. Suitably the further selection agent may be any selection agent which would normally kill the shoot or seedling, unless it has a mutation which prevents this. Suitably use of the further selection agent selects for shoots or seedlings having advantageous mutations in their organelle DNA.

A suitable trait of interest may be herbicide resistance. Suitably, step (c) may therefore comprise exposing the shoots or seedlings to a herbicide and step (e) may therefore comprise selecting those shoots or seedlings which have resistance to the herbicide. Suitable herbicides may be selected from those herbicides that target plastid gene products. One example of suitable herbicides are the Triazine herbicides such as terbuthylazine. Advantageously herbicide resistant plants may be used in combination with a herbicide for the removal of unwanted plants such as weeds, whilst the plant of interest remains unaffected. This is of most advantage in crops, where herbicides are commonly used to control weed populations and invasive species.

Suitably exposing the shoots or seedlings comprises contacted the shoots or seedlings with the further selection agent. Suitably by adding the further selection agent to the growth media or soil in which the shoots or seedlings are growing, or by spraying with the further selection agent. Suitably the further selection agent is added or sprayed at an effective concentration to select the resistant shoots or seedlings. A suitable effective concentration of the further selection agent may be between 50 ug/ml up to 500 ug/ml, suitably between 100 ug/ml up to 300 ug/ml, suitably 200 ug/ml.

Suitably the steps of selection described may also be applied to the methods of the ninth, eleventh and twelfth aspects of the invention.

Step (f) of the method comprises regenerating the shoots or seedlings into a mature plant. By regenerating it may simply mean growing the shoots or seedlings on appropriate growth media as discussed above.

Optionally in step (g) of the method, the steps of taking an explant from the plant and culturing one or more shoots therefrom and then exposing the shoots to selection agents may be repeated one or more times, equally the steps of generating F1 seedlings from the plant and exposing the seedlings to selection agents may be repeated one or more times. Suitably the plants from step (f) are then used for taking explants or generating seedlings as described above. Suitably steps (b) to (e) of the method may be repeated between 1-10 times, suitably between 1-5 times, suitably between 1-3 times. Suitably each round of selection may increase the homoplasmy of the plant. Suitably steps (b) to (e) are repeated until the plant is homoplasmic. Advantageously however the present method achieves homoplasmy with one round of regeneration, such that step (g) is not required.

The invention will now be described by way of reference to several non-limiting examples.

### EXAMPLES

The polymerases and methods of the invention were investigated by the inventors and are discussed further below. References to ‘mutator POP’ or ‘mutPOP’ or ‘MuPOP’ indicate the organellar DNA polymerase of the invention.

### Example 1

### 1.1 Introduction

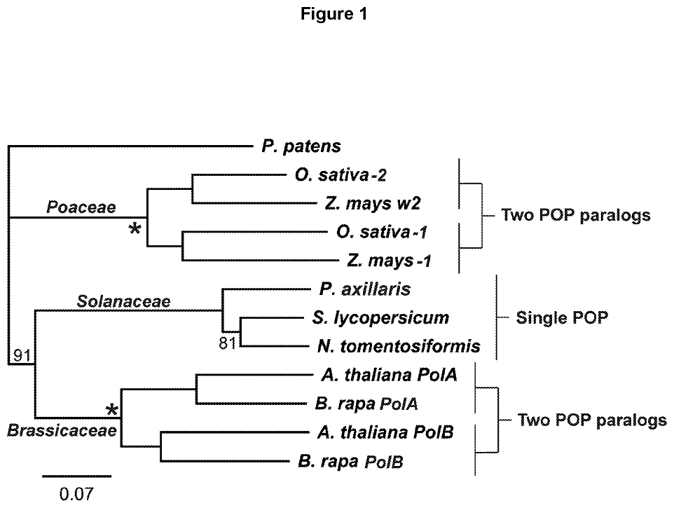

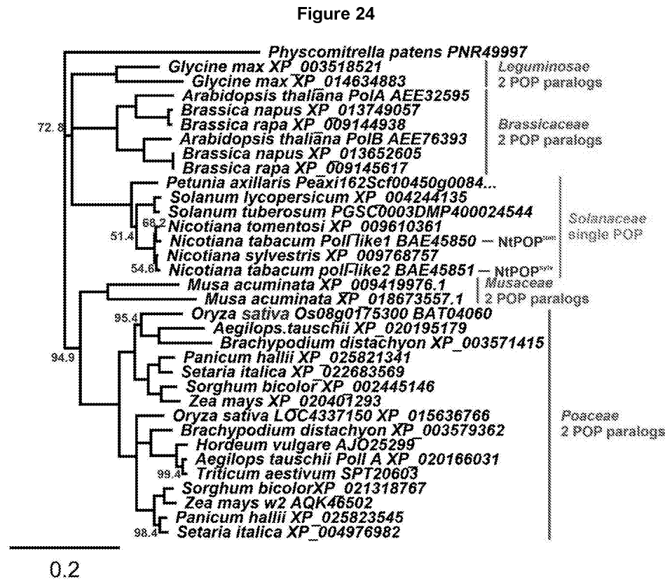

Phylogenetic analysis of POPs (FIG. 1, FIG. 24) revealed two patterns of POP distribution in angiosperms. Dicot families such as the Solanaceae contain a single POP gene in diploid (2n) species such as Solanum lycopersicum, Nicotiana tomentosiformis and Petunia hybrida. The second group of plants contain two divergent POP genes whose products share 70-76% amino acid identity in taxonomically distant dicot and monocot families exemplified by the Brassicaceae and Poaceae families (FIG. 1, FIG. 24). The gene duplications giving rise to these POP paralogs in the Brassicaceae and Poaceae took place after their divergence from a common ancestor. Plant POPs from Nicotiana tabacum (Solanaceae) and Arabidopsis thaliana (Brassicaceae) were shown to be dual targeted to both organelles (Ono et al., 2007, Christensen et al., 2005, Carrie et al., 2009). Single gene knockouts of POP genes are viable in A. thaliana (Parent et al., 2011) but not in Zea mays (Poaceae), where chloroplast DNA but not mitochondrial DNA was reduced to low lethal amounts (Han et al., 1993, Udy et al., 2012). This difference indicates redundancy of POP genes in A. thaliana but not in Zea mays. Despite this redundancy, differences have been found between the two A. thaliana POPs (AtPolA and AtPolB), with respect to their enzymatic properties (Ayala-Garcia et al., 2018), roles in DNA replication versus repair (Parent et al., 2011, Ayala-Garcia et al., 2018, Mori et al., 2005), interactions with other proteins (Morley et al., 2019) and relative importance in mitochondria versus chloroplasts (Cupp and Nielsen, 2013).

To develop an error-prone mutator POP we chose to engineer an enzyme from the Solanaceae. Use of a Solanaceous POP has the advantage of engineering the sole enzyme responsible for the DNA polymerase-related replication/repair activities in plant organelles. We chose a POP from Nicotiana tabacum (tobacco), which is the leading model for transgenic research on organelle genomes (Day, 2012). N. tabacum is allotetraploid (4n) resulting from a relatively recent fusion between diploid (2n) N. tomentosiformis and N. sylvestris parents (Sierro et al., 2014).

To evaluate the impact of amino acid substitutions on the replication fidelity of DNA polymerases we developed a novel mutation screening assay based on the positive selection scheme described by Nilsson et al (1983) (Nilsson et al., 1983). In the assay a single stranded stretch of the bacteriophage lambda cI gene encoding the Cl repressor protein is replicated by a DNA polymerase in vitro before transformation of the plasmid into E. coli. Replication errors resulting in loss-of-function prevent Cl repressor binding to its target sequence upstream of the tetracycline resistance gene. This approach gives rise to tetracycline-resistant colonies containing plasmids with mutations in the cI gene that can be sequenced and compared to the large data set of previously mapped loss-of-function mutations in the cI gene (Reidhaarolson and Sauer, 1988, Reidhaarolson and Sauer, 1990, Bell et al., 2000, Sauer, 2013). At high plating densities, positive selection has the advantage of ease of identifying resistant mutant colonies compared to colony screening methods based on colour (Maor-Shoshani et al., 2000, Bebenek and Kunkel, 1995, Jozwiakowski and Connolly, 2009). Here we used the assay to construct and characterise a highly error prone N. tabacum POP suitable for elevating mutation rates in organelles.

### MATERIALS AND METHODS

### 1.2 Sequence Analysis Software

In silico vector assembly and sequence analyses were carried out using SnapGene (San Diego), Vector NTI Advance (Thermo Fisher Scientific, Paisley) and Geneious Prime (Biomatters, Auckland). Protein alignments from Geneious Aligner were used in GeneiousTree Builder to assemble neighbour-joining trees (43).

### 1.3 Cloning, Overexpression and Purification of Recombinant NtPOP^{tom} Enzymes

General methods for recombinant DNA work and molecular biology procedures including media composition and buffers were from Sambrook et al. (1989) (Sambrook et al., 1989). The NtPOP^{tom} WT cDNA was isolated from N. tabacum var Petit Havana. The amino acid substitutions in the exonuclease and polymerisation domains were introduced into the coding region using the Q5 site directed mutagenesis kit (New England Biolabs). The polymerisation domain was excised by replacing the internal Nde I and Pst I fragment in the NtPOP^{tom} cDNA with annealed oligos delNdelPstI-F and delNdelPstI-R (Table 4). Coding sequences were cloned into pET30b (Invitrogen) and expressed in Rosetta 2(DE3) cells (Novogen, Cambridge, UK). Recombinant protein expression was induced with 1 mM IPTG for 3 hours in cells grown in Terrific Broth (Sigma-Aldrich, Southampton, UK) containing 50 μg/ml kanamycin and 37 μg/ml chloramphenicol. All next steps were done on ice. Sedimented cells were resuspended in chilled buffer P (50 mM Bis-tris pH 8.0, 150 mM NaCl and 1 mM EDTA) supplemented with 0.1% Triton X100 w/v, 1 mg/ml lysozyme, protease inhibitor cocktail (Roche UK, Welwyn Garden City, UK) and lysed by sonication. RNase A (10 μg/ml) and DNase I (5 μg/ml) were added to the lysate and incubated for 15 min. The mixture was spun 21,000×g for 15 min. The protein was purified using a Strep-Tactin®-XT purification column (IBA Life Sciences, Goettingen, Germany) and stored in buffer P containing 50% (V/V) glycerol and 1 mM dithiothreitol at −20° C. The five N-terminal amino acids of the purified 99 kDa NtPOP^{tom} WT enzyme were determined by Edman degradation (AltaBioscience, Redditch, UK).

### 1.4 DNA Replication Assays

We followed the protocol of Tveit and Kristensen (2001) substituting PicoGreen (Tveit and Kristensen, 2001) with Quantifluor One dsDNA fluorescence dye (Promega, Southampton). Synthesis of double-stranded DNA was from a 35 base oligonucleotide (M13-F, Table 4) annealed to single-stranded M13mp18 DNA in buffer R (10 mM Tris-HCl pH 8.0, 100 mM NaCl, 2.5 mM MgCl₂, 1 mM DTT, 333 μM dNTPs and 100 μg/ml bovine serum albumin). Reactions at 30° C. were initiated by the addition of enzyme and terminated by adding EDTA to 8 mM and placing in ice. Each reaction in 30 μl contained 12 to 400 fmol of purified recombinant DNA polymerase with the primed M13mp18 template in excess apart from competition experiments using 600 fmol of WT enzyme when the template was saturated. Double stranded DNA was quantified using the Quantifluor One dsDNA fluorescence dye and a Synergy HI Multi-Mode Microplate Reader (BioTek Instruments) set at 504 nm_{Ex}/531 nm_{Em}.

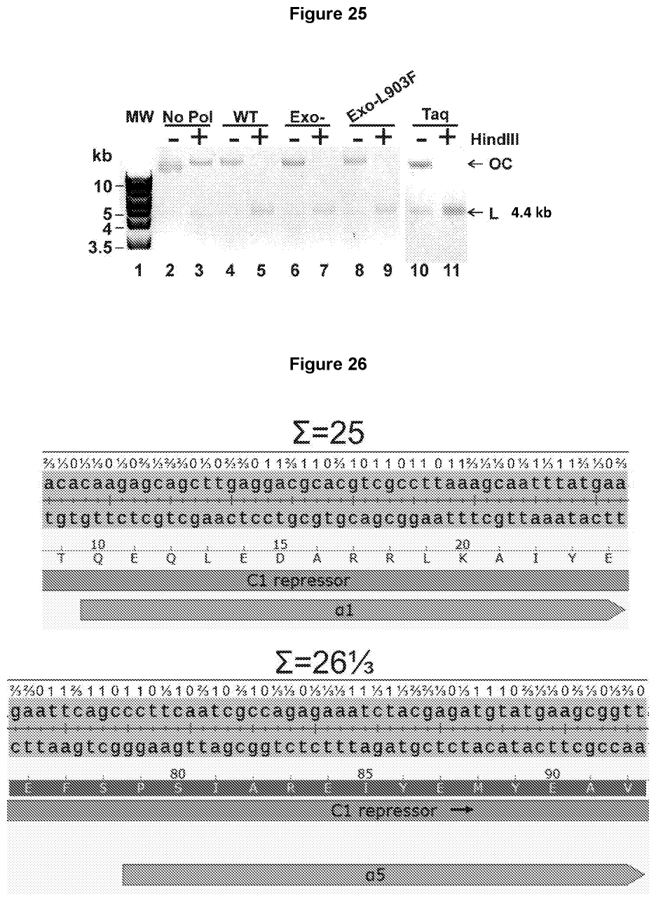

Gapped DNA was prepared using the competing oligonucleotide-method (Jozwiakowski and Connolly, 2009)). pUN121 (Nilsson et al., 1983) was nicked with Nb.bpu101 (New England Biolabs) and mixed with three competing oligonucleotides (Table 4) corresponding to the nicked non-coding strand in 50-fold molar excess. The mixture in 10 mM Tris-HCl pH 8.5, 10 mM MgCl₂, 100 mM KCl and BSA 100 μg/ml was heated to 95° C. and cooled gradually to 75° C. over 30 minutes and then left to cool to room temperature. Competitor oligonucleotides were removed using QIAquick purification columns (QIAGEN, Manchester). Gapped plasmids were purified using benzoylated naphthoylated DEAE cellulose (Sigma-Aldrich, Poole) as described by Wang and Hays (2001)(Wang and Hays, 2001). Purified gapped plasmid was digested with Hind Ill before use in replication assays to linearize any double-stranded DNA contaminating the gapped plasmids. This step effectively removes contaminating double-stranded DNA from the bacterial colony screen because linear DNA is an ineffective transformation substrate in E. coli. The gapped plasmid was ready for use after removal of Hind Ill using a QIAquick purification column. Replication of gapped plasmid was for 15 minutes in 30 μL of buffer R at 30° C. for recombinant POP enzymes and 72° C. for Taq DNA Pol. Replication was verified using Hind Ill digestion FIG. 25). The replicated plasmids were transformed into DH5α competent cells (New England Biolabs). Transformed cells were plated on LB agar medium containing either 100 μg/ml ampicillin or 15 μg/ml tetracycline and incubated at 37° C. to visualise colonies.

### 1.5 Mutant Frequency and Error Rate

Mutant frequency was calculated by dividing the number of tetracycline-resistant colonies by the number of ampicillin resistant colonies after accounting for the difference in plating efficiency. Using a pUN121 plasmid with a loss-of-function mutation in the cI gene, the number of colonies on tetracycline medium were 61% of the number obtained on ampicillin medium. The error rate (ER) was calculated by scoring mutations in the coding region containing the well-studied alpha 1 and 5 helices (Reidhaarolson and Sauer, 1990, Sauer, 2013) in the cI gene. ER was determined from the equation ER=MF/(D×P) (Bebenek and Kunkel 1995, Keith et al. 2013) where MF is the mutation frequency of tetracycline resistant colonies resulting from mutations in the alpha 1 and 5 coding regions, D the number of detectable sites in this sequence stretch and P the probability that a mutation in the newly synthesized strand will be expressed. P was determined experimentally. A 5′ phosphorylated oligonucleotide (pUN121_mut) with a 2-base deletion in the Hind Ill site was annealed and ligated to gapped pUN121. This heteroduplex region was then extended with Taq DNA polymerase in buffer W. A temperature of 30° C. was used to prevent strand displacement activity. The replicated plasmid was purified using a QIAquick purification column and treated with Hind Ill to linearize any pUN121 lacking the heteroduplex at the Hind Ill site. Following transformation of E. coli the ratio of tetracycline to ampicillin colonies provided an estimate of the probability of expression, which was 2.5%. Estimation of detectable sites required identification of base changes at every position in the alpha 1 and 5 coding region that inactivate the Cl repressor (FIG. 26) using published data ((Reidhaarolson and Sauer, 1990, Sauer, 2013). These include 51.3 base substitutions and 99 indels providing a total of 150.3 detectable sites in coding sequences for alpha helices 1 and 5.

### 1.6 DNA Sequencing

Plasmids were purified using the Isolate II kit (Bioline, London) and sequenced (Eurofins Genomics Germany, Ebersberg)) with primers pUN121-F and pUN121-R (Table 4). Sequences were analysed using Geneious Prime software (Biomatters, Auckland).

### 1.7 Protein Blot Analyses

Bacterial cells were lysed in sample buffer (50 mM Tris-HCl, pH 6.8, 12.5 mM EDTA, 10% (v/v) glycerol, 2% (w/v) SDS, 2% (v/v) ß-mercaptoethanol, 0.1% (w/v) bromophenol blue) and placed in a boiling water bath for 5 minutes. Following centrifugation for 5 minutes at 14,000 rpm (Eppendorf 5415c, Stevanage) supernatants were fractionated on 10% (w/v) polyacrylamide gels prepared using TGX FastCast acrylamide solutions (Bio-Rad, Hemel Hempstead) in a mini-Protean 3 electrophoresis tank (BioRad) in running buffer (25 mM Tris, 192 mM glycine, 0.1% w/v SDS). Following electrophoresis gels were viewed with the molecular imager gel doc XR system (BioRad) after UV activation of tri-halo compounds. Proteins from SDS-PAGE gels were transferred using Turbo-Blot Turbo Mini 0.2 μm nitrocellulose transfer packs and the Trans-blot Turbo transfer system (Bio-Rad). Proteins were detected as previously described (Madesis et al., 2010). Primary antibodies used were a monoclonal antibody against Strep-tag II (IBA Lifesciences, Göttingen) and a rabbit polyclonal antibody raised against the peptide NTETGRLSARRPNLQ in the POP polymerisation domain, which was affinity-purified using the same peptide (Eurogentec, Liege). Secondary antibodies linked to alkaline phosphatase (Sigma-Aldrich, Poole, UK) were stained with 5-bromo-4-chloro3-indolyl phosphate/nitro blue tetrazolium (BCIP/NBT) liquid substrate (Sigma-Aldrich, Southampton).

### 1.8 Statistical Analyses

We followed the method of Stone et al (2009)(Stone et al., 2009) involving two tailed chi squared analyses to identify significant differences between base substitution error rates for the POP enzymes.

### Results

### 1.9 Structure of WT and Mutant N. tabacum POPs

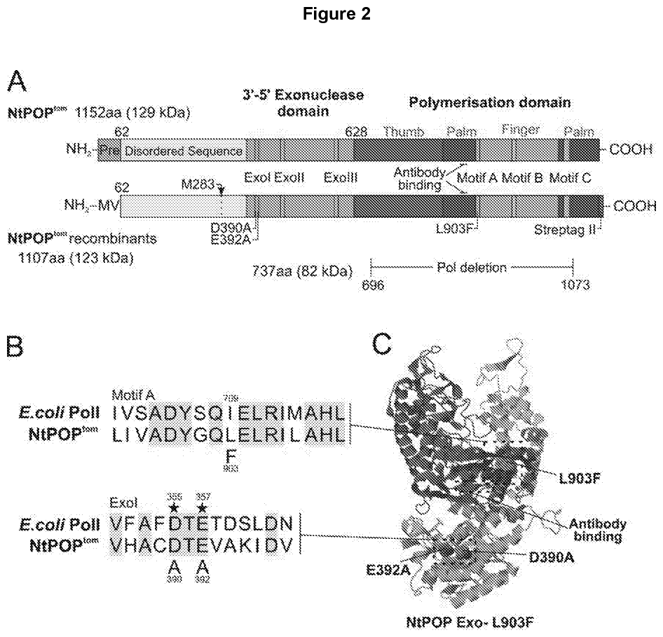

N. tomentosiformis and N. sylvestris, the diploid parents of N. tabacum tabacum (Sierro et al., 2014), contain a single POP enzyme. Whilst N. tabacum does not contain POP paralogs, it has inherited the POP orthologs present in its parents. We identify these orthologs as NtPOP^{tom} and NtPOPsylv to indicate their parental origins. NtPOP^{tom} and NtPOPsylv correspond to the NtPol1-like 1 and NtPol1-like 2 proteins in Ono et al (2007), respectively. NtPOP^{tom} (NtPol1-like 1) studied here shares 98% amino acid identity with its parental POP in N. tomentosiformis. The domain organisation of the 1152 amino acid NtPOP^{tom} enzyme is shown schematically in FIG. 2A. The protein contains a predicted 61 amino acid N-terminal organelle targeting sequence (Emanuelsson et al., 2007) followed by a disordered region of unknown function with low sequence conservation. The disordered regions from NtPOP^{tom} and A. thaliana (AtPolB) POPs only share 18% amino acid identity whereas the regions containing the 3′-5′ exonuclease and polymerisation domains share 71% amino acid identity (not shown). The disordered region is not found in other members of the DNA polymerase A family, which includes the first characterised member of the group: Escherichia coli DNA Polymerase I (Pol I). NtPOP^{tom} amino acids 352 to 1152 aligned with amino acids 340 to 925 of the Klenow fragment of E. coli DNA Pol I share 24% amino acid identity. Within this region are highly conserved sequence motifs located in the 3′-5′ exonuclease (proof-reading) and 5′-3′ polymerisation domains (Delarue et al., 1990). FIG. 2A locates exonuclease motifs Exo I-III, and polymerisation domain motifs A-C, on a schematic diagram of the NtPOP^{tom} primary sequence. In FIG. 2B a highly conserved eight amino acid sequence DYSQIELR (Astatke et al., 1998) in motif A of the polymerisation domain in E. coli DNA Pol I is aligned with the corresponding region of NtPOP^{tom}. Within this DYSQIELR motif in E. coli DNA Pol I, substitutions at isoleucine 709 gave rise to an efficient mutator DNA polymerase (Shinkai and Loeb, 2001). The equivalent L979F mutation in Saccharomyces cerevisiae DNA polymerase zeta also gave rise to a functional and highly error prone enzyme (Stone et al., 2009). Other amino acids that reduce E. coli DNA Pol I replication fidelity include R668, E710 and N845 (Minnick et al., 1999). These residues are conserved in NtPOP^{tom} and represent additional residues that could be targeted to develop an error prone enzyme. Replacement of aspartic acid with alanine in the DYSQIELR motif in a rice POP destroyed DNA synthesis activity (Takeuchi et al., 2007).

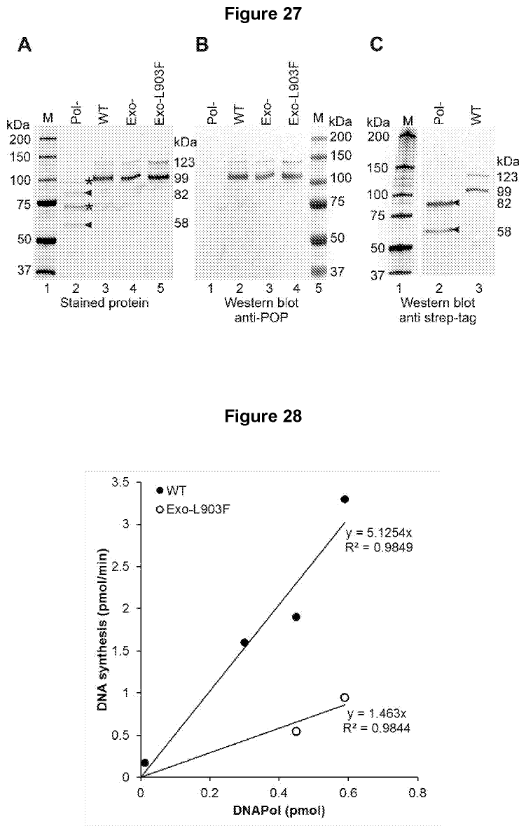

Four recombinant NtPOP^{tom} proteins were expressed in E. coli. All lacked the first N-terminal 61 amino acids corresponding to the predicted organelle targeting sequence (Emanuelsson et al., 2007). The changes to the WT protein are summarised in the diagrammatic scheme of the 1107 amino acid recombinant protein in FIG. 2A. The N-terminal 61 amino acids were replaced by an initiator methionine followed by a valine for expression in E. coli. We refer to the recombinant protein containing the wild type (WT) exonuclease and polymerisation domains as WT. The exonuclease deficient (Exo-) recombinant protein contained D390A and E392A substitutions in the Exo I motif (FIGS. 2A and 2B). The corresponding D355A and E357A substitutions in E. coli Pol I (FIG. 2B) destroy exonuclease activity (Bebenek et al., 1990). The Exo-L903F recombinant protein contained a L903F substitution in the polymerisation domain in addition to the D390A and E392A substitutions. The locations of changed amino acids on the 3D-model (56,57) are shown in FIG. 2C. Pol—was a defective recombinant enzyme lacking amino acids 696-1073 of the polymerisation domain (FIG. 2A). A C-terminal strep-II tag (58) preceded by a GSGSGS linker facilitated purification. The purified recombinant NtPOP^{tom} enzymes were fractionated by SDS-PAGE on stain-free gels (Bio-Rad) and studied by protein blot analyses using antibodies recognising the POP polymerisation domain and strep-tag-II (FIG. 27).

### 1.10 DNA Synthesis Activity of Recombinant NtPOP^{tom} Enzymes

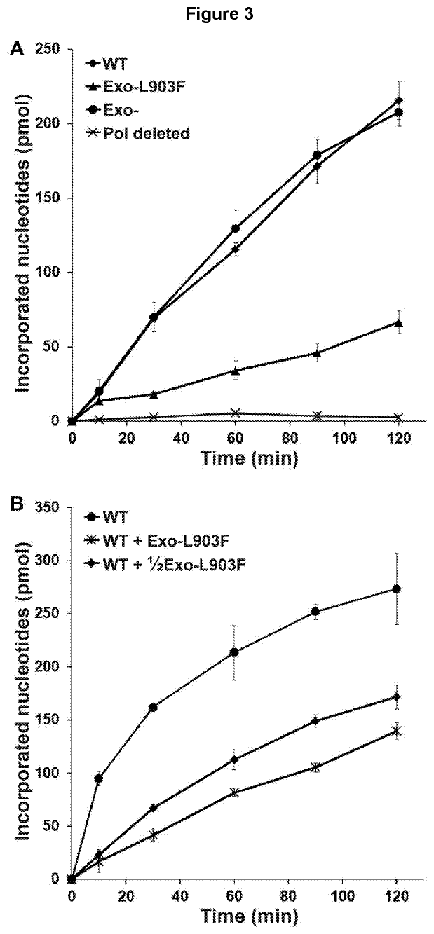

DNA synthesis by the four recombinant NtPOP^{tom} enzymes (WT, Exo-, Exo-L903F and Pol-) was measured by replication of M13 single stranded DNA from an annealed 35-mer oligonucleotide. FIG. 3A shows the synthesis of double-stranded DNA against time catalysed by the recombinant NtPOP^{tom} enzymes. The replication activities of the WT and Exo-enzymes were indistinguishable. This confirmed that the amino acid substitutions introduced into the exonuclease domain (FIG. 2B) did not affect polymerase activity, consistent with the E. coli DNA Pol I data (Derbyshire et al., 1991). DNA synthesis by the Exo-L903F enzyme was reduced by about 70% (FIG. 3A, FIG. 28) reflecting a detrimental effect of the polymerisation domain L903F amino substitution on DNA synthesis. Removal of bacterial DNA polymerases by our purification regime was demonstrated by the negligible rate of DNA synthesis observed using the Pol-protein, which lacks the DNA polymerisation domain responsible for DNA synthesis (FIG. 3A).

To assess the potential of the recombinant Exo-L903F enzyme to compete with the WT enzyme during replication of templates a competition experiment was conducted. Different amounts of Exo-L903F were added to a fixed amount of the WT enzyme under conditions where the enzymes were in excess relative to the DNA template. Increasing amounts of Exo-L903F reduced the overall rate of DNA synthesis (FIG. 3B) consistent with effective competition between the Exo-L903F and WT enzyme for template replication.

TABLE 1

| | | | | 4 | | | | 6 | |
|---|---|---|---|---|---|---|---|---|---|
| | | 2 | 3 | Average | 5 | | | Relative | |
| 1 | | Mutant | Relative | Mutation | Error Rates | | | Error | |
| DNA | | Colony | Mutant | No. per | (Mutations/Base) | | | Rates | |
| Polymerase | | Frequency | Frequency | Gene | A | B | C³ | D | E |
| NtPOP^{tom} | WT | 8.43 × 10⁻⁵ | 1.0 | 1.1 | 5.6 × 10⁻⁵ | 8.5 × 10⁻⁶ | 5 × 10⁻⁶ | 1 | 1 |
| | Exo- | 4.50 × 10⁻⁴ | 5.3 | 1.1 | 3.0 × 10⁻⁴ | 4.5 × 10⁻⁵ | 4 × 10⁻⁵ | 5 | 8 |
| | Exo- | 5.30 × 10⁻³ | 63 | 2.4 | 7.7 × 10⁻³ | 1.2 × 10⁻³ | 7 × 10⁻⁴ | 140 | 140 |
| | L903F | | | | | | | | |

| | | | | | | | | |
|---|---|---|---|---|---|---|---|---|
| Taq | 4.70 × 10⁻⁴ | 5.6 | 1.0 | (2.85 × 10⁻⁴)¹ | (4.3 × 10⁻⁵)² | nd | 5 | nd |

Table 1 shows mutant frequencies and DNA polymerase error rates.

Error rates in columns 5A and 5B were calculated from the data in columns 3 and 4 and Taq DNA error rates shown in brackets from: ¹the supplier (New England Biolabs) and ²McInerney et al. 2014 (McInerney et al., 2014). Column 5C error rates were from scoring mutations in the alpha 1 and 5 coding regions in the ci gene (this work). Columns 6D and 6E show relative error rates based on columns 5A and C respectively. nd—not determined

### 1.11 Genetic Screen to Estimate DNA Polymerase Replication Fidelity

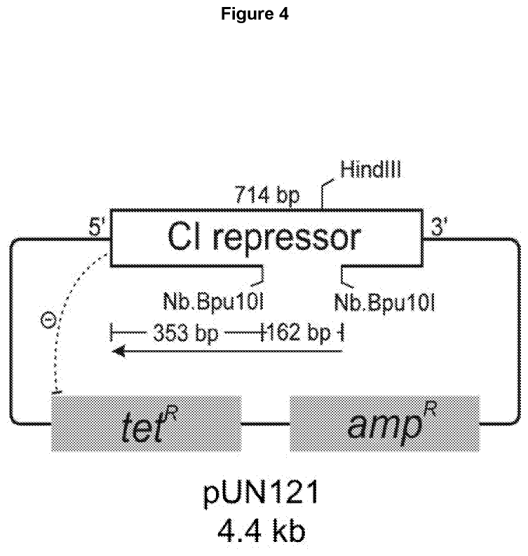

The assay involved replication across the coding sequence of the lambda Cl repressor in the positive selection vector pUN121 (Nilsson et al., 1983), which contains ampicillin (ampR) and tetracycline (tetR) resistance genes (FIG. 4). The Cl repressor binds upstream of the tetR gene preventing its expression. Replication errors that inactivate the Cl repressor gene in pUN121 allow tetR expression and survival of bacterial colonies on tetracycline medium. The presence of the ampR gene enables the total number of plasmid-containing colonies to be estimated on ampicillin plates. A single-stranded gap in the cI gene was prepared by removing 162 nucleotides of the non-coding strand using the nicking enzyme (Nb.Bpu10I) and the competitor oligonucleotide method (Jozwiakowski and Connolly, 2009, Wang and Hays, 2001). This single-stranded gap is complementary to bases 354 to 515 of the 714 nucleotide cI gene and encodes amino acids 119 to 172, which includes the hinge region and residues in the C-terminal domain of the repressor important for dimer formation and cooperative binding of two repressor molecules to two operator sites (Bell et al., 2000). Replication of the single-stranded gap was towards the N-terminal coding region of the cI gene (FIG. 4). Continuation of replication beyond the 162 base gap requires strand displacement of the 353 bases to the ATG initiating codon and increases the region of the Cl repressor gene replicated to 515 nucleotides. The complementary template strand encodes amino acids 1-118 of the N-terminal DNA binding domain of the Cl repressor protein (Reidhaarolson and Sauer, 1990, Bell et al., 2000).

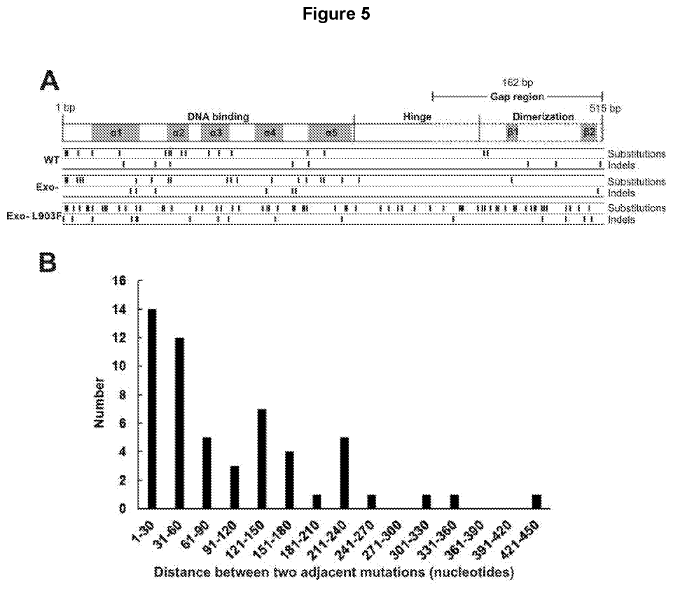

We compared the recombinant NtPOP^{tom} enzymes to the well-studied Taq DNA polymerase, which lacks 3′-5 exonuclease activity (McInerney et al., 2014, Potapov and Ong, 2017). Following replication of the single-strand gap with the recombinant DNA polymerases, the replicated plasmids were transformed into E. coli cells and transformants selected on media supplemented with tetracycline or ampicillin. Samples of the replicated plasmids were treated with Hind Ill to monitor conversion of the single-stranded gap to newly replicated double stranded DNA (FIG. 25). The frequency of colonies containing plasmids with loss-of-function mutations in the cl gene was calculated by dividing the number of tetracycline resistant colonies by the number of ampicillin-resistant colonies (Table 1). The WT NtPOP^{tom} enzyme gave rise to the lowest frequency of mutant tetracycline colonies, which was about five-fold lower than those obtained with the Exo-enzyme and Taq DNA polymerase. The Exo-L903F enzyme gave rise to the highest frequency of tetracycline resistant colonies, which was 63-fold higher than that obtained with the WT NtPOP^{tom} enzyme. All plasmids sequenced from tetracycline-resistant colonies contained mutations in the cl gene verifying the absence of false positive colonies. The locations of the mutations in the cI gene (FIG. 5A) showed that the recombinant NtPOP^{tom} enzymes were efficient strand displacement enzymes capable of displacing hundreds of base-paired nucleotides ahead of the replication fork. Mutations included changes to the ATG start codon located in a double-stranded region 353 base pairs beyond the single-stranded gapped region.

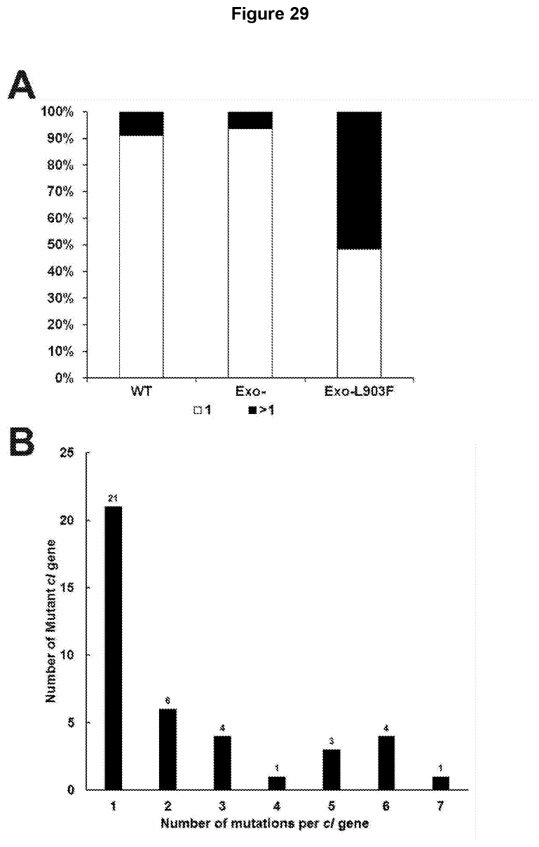

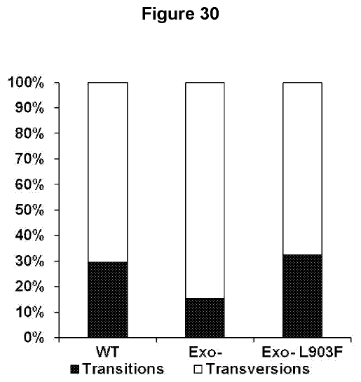

Mutant cI genes resulting from replication errors by the WT and Exo-NtPOP^{tom} enzymes contained an average of 1.1 mutations. This was raised to an average of 2.4 mutations in cI genes replicated by the Exo-L903F enzyme. Over 90% of mutant cl genes replicated using the WT and Exo-enzymes contained a single mutation (FIG. 29A). These were more common in the region encoding the N-terminal DNA binding region indicating the influence of sequence context on error frequencies and the location of codons essential for repressor function (FIG. 5A). Replication by the Exo-L903F NtPOP^{tom} enzyme gave rise to multiple single base substitutions and/or single base indels at two to seven sites in about 50% of the cI genes sequenced (FIG. 29A-B). Exo-L903F mutations were distributed throughout the region replicated (FIG. 5A). In cI genes with multiple mutations, 48% of mutations were located within 60 bases of each other and the frequency decreased with distance (FIG. 5B). Ninety percent of these mutations were separated by over ten nucleotides and as much as 445 nucleotides. These multiple mutations in a single cI gene cannot be explained by replication errors from single events. The uneven pattern of decrease in frequency of adjacent mutations with distance observed is likely to reflect the influence of specific DNA sequences on Exo-L903F error rate. Uneven distribution of replication errors due to sequence context is well established (Kunkel and Bebenek, 2000).

### 1.12 Estimation of Recombinant DNA Polymerase Error Rates

Estimates of recombinant NtPOP^{tom} error rates were based on comparisons with Taq DNA polymerase. The Taq DNA polymerase error rate in the pH 8.8 buffer provided by the supplier (New England Biolabs) was 2.85×10−4 mutations per base, which is consistent with other reports (Potapov and Ong, 2017, Ling et al., 1991). Variation in buffer composition and methods to measure error rates including different DNA replication templates have led to lower estimates, for example 4.3×10−5 (60). We used a pH 8.0 buffer, which was reported to reduce the Taq DNA polymerase error rate by around three-fold from 2.0×10−4 at pH 8.8 to 7.2×10−5 at pH 8.0 (Ling et al., 1991). Using Taq DNA polymerase high and low error rates as comparators provided an estimated error rate for the WT POPtom enzyme that lies within a 7-fold range between 5.6×10−5 and 8.5×10−6 mutations per base (Table 1, columns 5A and 5B). Error rate determinations require identifying all the detectable sites within a sequence whose mutation would result in a defective protein (Keith et al., 2013). To provide an estimate of mutation rate based on the frequency of mutations in the c1 gene we identified the detectable sites present in the region coding for 33 amino acids that include the alpha 1 and 5 helices (FIG. 26). Systematic studies on this region have identified the impact of changes to all 33 amino acids on repressor function (Reidhaarolson and Sauer, 1990, Sauer, 2013). Analysis of cl mutations in the alpha 1 and 5 coding regions provided an estimated error rate for the WT NtPOP^{tom} enzyme of 5×10−6 mutations per base (Table 1 column 5C). Higher error rates of 4×10−5 and 7×10−4 were estimated for the Exo- and Exo-L903F enzymes, respectively (Table 1, column 5C). Error rate values for the recombinant NtPOP^{tom} enzymes based on mutations at detectable sites were in closer agreement with relative values calculated using the lower error rate of 4.3×10−5 reported for Taq DNA polymerase (Table 1, column 5B) (Keith et al., 2013, McInerney et al., 2014). The relative error rates for the NtPOP^{tom} enzymes based on Taq DNA polymerase (Table 1, column 6D) and detectable sites in the cI gene (Table 1, column 6E) were in close agreement. The error rate was increased by five to eight-fold in the exonuclease deficient enzyme and by 140 fold in the Exo-L903F enzyme relative to the WT enzyme.

### 1.13 Mutation Spectrum of Recombinant NtPOP^{tom} Enzymes

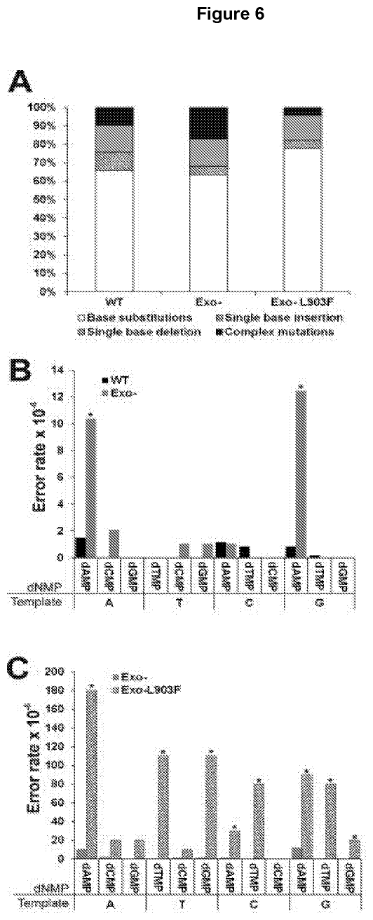

Base substitutions were the most common type of mutation and represented 66%, 63% and 78% of the cl mutations associated with WT, Exo- and Exo-L903F NtPOP^{tom} enzymes, respectively (FIG. 6A, Table 2). The percentage of transversion mutations were 70%, 85% and 68% for the WT, Exo- and Exo-L903F enzymes, respectively (FIG. 30). A common mismatch (A:A) shared by the WT and error-prone NtPOP^{tom} polymerases involved a template adenine mis-pairing with an incoming dATP (FIG. 6B-C, Table 3). The Exo-enzyme also gave rise to a high proportion of G:A mispairings (FIG. 6B). Other frequent mutations associated with the Exo-L903F enzyme arose from T:T, T:G, C:T, G:A and G:T mispairings (FIG. 6C). Single base deletions were markedly more frequent than single base insertions for the Exo- and Exo-L903F enzymes (FIG. 6A). Here we define complex mutations as deletions/insertions of more than one base or substitutions of two adjacent bases, multiple base substitutions at closely spaced sites and a mixture of these changes. Complex mutations accounted for 10%, 17% and 4% of the total number of mutations for the WT, Exo- and Exo-L903F enzymes, respectively (Table 2). For the WT enzyme, a complex mutation can be explained by deletion of 6-base direct repeat by a slippage event. The majority of different types of mutations (base substitutions, INDELs and complex mutations) did not co-localise to the same position on the cI gene.

### 1.14 Influence of WT Enzyme on Exo-L903F Mutation Rate

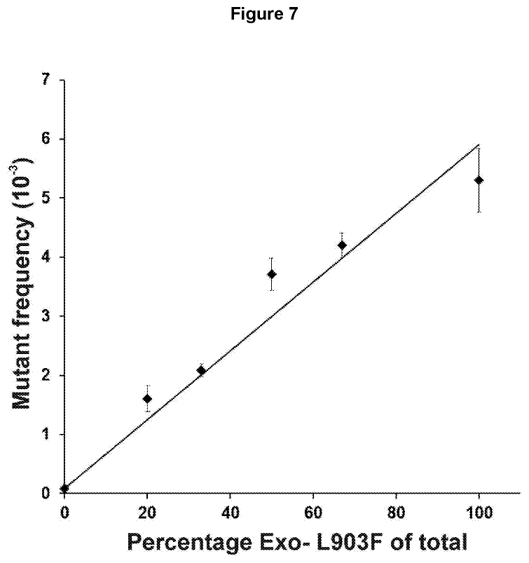

To evaluate potential interactions between the WT enzyme and the error-prone NtPOP Exo-L903F DNA polymerase that might influence mutation rate, we tested mixtures of the two enzymes in the gap-filling replication assay (FIG. 7). The results showed that mutant frequency increased in proportion to the amount of error-prone NtPOP Exo-L903F present. The mutation rate was elevated even when the WT enzyme was in 4-fold excess. The data suggests that the error-prone enzyme is semi-dominant to the WT enzyme.

### DISCUSSION

Introducing amino acid substitutions into the exonuclease and polymerisation (L903F) domains of a tobacco POP produced a functional and highly error-prone enzyme. The WT NtPOP^{tom} enzyme had an estimated error rate of between 6×10−5 to 5×10−6 mutations per base. This was raised by 140-fold in the Exo-L903F enzyme. Removal of exonuclease activity alone increased the error-rate by 5-8 fold. In vitro competition experiments indicated the Exo-L903F enzyme was semi-dominant to the WT enzyme. High error rate and effective mutator activity in the presence of the WT enzyme makes the Exo—L903F enzyme a strong candidate for developing an organelle mutator system in plants. Mutation frequency was determined using a new genetic screen involving positive selection in E. coli, based on gain of tetracycline resistance (Nilsson et al., 1983).

Positive selection has the advantage of ease of isolation of mutant colonies due to the absence of surrounding bacterial colonies associated with mutant screens involving colour identification such as those based on the lacZ (Bebenek and Kunkel, 1995) or cro (Maor-Shoshani et al., 2000) genes. It also overcomes potential technical issues linked to poor development of colour resulting from uneven distribution of substrates such as 5-bromo-4-chloro-3-indolyl-β-D-galactopyranoside (X-gal) on solid media plates. Furthermore, the development of new genetic screens increases the number of template DNA sequences available for testing the fidelities of DNA polymerases. The assay involved in vitro replication of the coding sequence for the well-characterised bacteriophage lambda Cl repressor protein (Reidhaarolson and Sauer, 1990, Sauer, 2013). The assay showed the NtPOP^{tom} enzymes were efficient at displacing double stranded regions over 300 bp ahead of the replication fork. Previous work had shown that POPs were capable of displacing small 30 to 35 base oligonucleotides (Takeuchi et al., 2007, Garcia-Medel et al., 2019). Most single nucleotide mutations leading to loss-of-repressor function were found in the N-terminal DNA binding region of the repressor (Reidhaarolson and Sauer, 1990, Bell et al., 2000, Sauer, 2013). This may reflect the influence of sequence context on POP error rates as well as the location of mutation sites resulting in loss of repressor function. Error rate estimates were determined from detectable sites in 99 nucleotides encoding the alpha 1 and 5 helices of the DNA binding domain. The estimated error rates for the recombinant NtPOP^{tom} enzymes based on mutations in the coding regions for alpha 1 and 5 helices were in reasonable agreement with the values calculated using relative mutation frequency and error rate for Taq DNA polymerase. Closer agreement was found with calculations based on the lower range of estimated error rates reported for Taq DNA polymerase, which vary from ˜3×10−4 to 4×10−5 (McInerney et al., 2014, Potapov and Ong, 2017). Here, we used a pH 8.0 buffer which has been shown to reduce Taq DNA polymerase error rate compared to the standard conditions of pH 8.8 (Ling et al., 1991). Error rates vary from 10-3 for low fidelity enzymes to 10-6 for high fidelity enzymes (Kunkel and Bebenek, 2000). The WT NtPOP^{tom} with an error rate of 6×10−5 to 5×10−6 would appear to be a medium to high fidelity enzyme similar to the Klenow fragment of E. coli Pol I with an error rate of 6×10−6 (Bebenek et al., 1990). The error rate of the WT NtPOP^{tom} enzyme was not too dissimilar from the error rate of 7.3×10−5 reported for the A. thaliana POP AtPolA, which is proposed to be the main replicative enzyme in A. thaliana organelles (Ayala-Garcia et al., 2018). The AtPolB paralog with a higher reported error rate of 5.45×10−4 is considered to have a predominant role in repair (Ayala-Garcia et al., 2018).

Loss of 3′-5′ exonuclease activity increased the error rate of the NtPOP^{tom} Exo-enzyme by 5-8 fold which was comparable to the 4 to 7 fold increase in error rates reported for 3′-5′ exonuclease-deficient derivatives of the Klenow fragment (Shinkai and Loeb, 2001, Bebenek et al., 1990). This was higher than the 1.3 to 1.7-fold increase in error rates reported for the 3′-5′ exonuclease deficient A. thaliana organellar DNA polymerases using lacZ as the template (Ayala-Garcia et al., 2018). Loss of 3′-5′ exonuclease activity increased the error rate of the NtPOP^{tom} Exo-enzyme by 5-8 fold which was comparable to the 4 to 7 fold increase in error rates reported for 3′-5′ exonuclease-deficient derivatives of the Klenow fragment (Shinkai and Loeb, 2001, Bebenek et al., 1990). This was higher than the 1.3 to 1.7-fold increase in error rates reported for the 3′-5′ exonuclease deficient A. thaliana organellar DNA polymerases using lacZ as the template (Ayala-Garcia et al., 2018). The data may indicate variation in the importance of the exonuclease domain of POPs in different plant taxa. The limited impact of removing exonuclease activity on POP error rates contrasts with the much larger error rate increases observed for exonuclease deficient gamma DNA polymerases used as mitochondrial mutators (Foury and Vanderstraeten, 1992, Trifunovic et al., 2004, Longley et al., 2001). This reflects a fundamental difference between the DNA polymerases present in animal and fungal mitochondria versus those present in the organelles of other taxa. A 20-fold increase in error rate was reported for the 3′-5′ exonuclease-deficient human mitochondrial gamma DNA polymerase (Longley et al., 2001). To reduce the fidelity of the NtPOPtom enzyme beyond the 5 to 8 fold decrease achieved by ablating exonuclease activity we introduced the L903F substitution into the polymerisation domain. Discrimination of the correct nucleotide during polymerisation is the major determinant of replication fidelity (Kunkel and Bebenek, 2000). Combining a defective exonuclease domain with a L903F substitution in the polymerisation domain of the NtPOP^{tom} enzyme raised the mutant frequency by 63-fold and error rate by about 140 fold. By comparison, combining mutations in the exonuclease and polymerisation domains of E. coli Pol I raised the mutation rate by around 400-fold (Shinkai and Loeb, 2001). The highly error prone NtPOP^{tom} Exo-L903F enzyme exhibited reduced DNA synthesis activity compared to the WT and Exo-enzymes. This is in contrast to the results obtained with the Klenow fragment of E. coli DNA Pol I in which the equivalent 1709F substitution did not impact on DNA synthesis activity (Shinkai and Loeb, 2001) but is consistent with a reduction in DNA synthesis reported for the equivalent L979F substitution in Pol ζ, which is a family B polymerase (Stone et al., 2009)(. The native NtPOP^{tom} enzyme contains a C-terminal lysine residue. All recombinant NtPOP^{tom} enzymes contained this C-terminal lysine followed by a linker peptide (GSGSGS SEQ ID NO: 5) and C-terminal strep-II tag (WSHPQFEK SEQ ID NO: 6). The potential influence of the tag on activity was not investigated. In the distantly related bacteriophage T7 DNA polymerase, replacement of the C-terminal histidine with alanine reduces the activity of the enzyme (Kumar et al., 2001).

About half of the mutant cI genes replicated by the Exo-L903F enzyme contained a single mutation whereas the remainder contained multiple mutations varying from two to seven (FIG. 29A-B). The frequency of cI genes with multiple mutations was much higher than the product of single mutation frequencies. This rules out their origin from independent events and can be explained by replication models in which the first error increases the probability of a DNA polymerase introducing further replication errors (Stone et al., 2009). Multiple mutations per replicated template is a feature associated with low fidelity enzymes such as translesion DNA polymerases (Maor-Shoshani et al., 2000, Stone et al., 2009). POPs have been identified as translesion DNA polymerases (Baruch-Torres and Brieba, 2017) and the Exo-L903F enzyme is a highly error prone POP derivative. Whilst the frequency of two adjacent mutations made by the NtPOP^{tom} Exo-L903F enzyme reduced with the length of the intervening sequence, 35% of the mutations were separated by over 100 nucleotides. The propensity of Exo-L903F to make multiple mutations in vitro may be a useful characteristic to monitor the action of mutagenesis by the enzyme in plant organelles.

Sequencing mutant cI genes showed that seventy-eight percent of the mutations associated with the NtPOP^{tom} Exo-L903F enzyme were base substitutions of which 68% were transversion mutations. Frequent A:A mispairings of template to dNMP were common to WT and error prone NtPOP^{tom} enzymes (Table 3). This gave rise to T→A transversions in the synthesized strand. For the NtPOP^{tom} Exo-L903F enzyme, A:A and T:T mispairings accounted for 58% of the total transversion mutations. T:T mispairings were also a feature of a mutant E. coli DNA Pol I lacking exonuclease activity and containing a 1709F substitution in the polymerisation domain (Shinkai and Loeb, 2001). NtPOP^{tom} Exo-L903F gave rise to single base deletions at a 3-fold higher frequency than single base insertions, which was similar to the properties of many other DNA polymerases (Shinkai and Loeb, 2001, Kunkel and Bebenek, 2000). In the assay Taq DNA polymerase showed a preference for A to G substitutions resulting from a template thymine mispairing with a guanine in the cI gene (Table 3). This was consistent with previous results showing that base substitutions involving T:G mispairings are the most frequent for Taq DNA polymerase (McInerney et al., 2014, Potapov and Ong, 2017).

Genetic screens using E. coli to identify errors introduced during in vitro replication of DNA templates by DNA polymerases have provided a robust method to assay the fidelities and mutation spectra of DNA polymerases. The results from these genetic screens obtained over several decades support errors introduced during in vitro replication by DNA polymerases as the primary causes of the mutation patterns observed (Maor-Shoshani et al., 2000, Jozwiakowski and Connolly, 2009, Keith et al., 2013, Minnick et al., 1999, Bebenek et al., 1990, Kunkel, 1985). We used a recA mutant in common with other studies (Maor-Shoshani et al., 2000, Jozwiakowski and Connolly, 2009, Keith et al., 2013). Complex mutations involving more than one nucleotide have been previously documented using genetic screens (Maor-Shoshani et al., 2000, Stone et al., 2009, Bebenek et al., 1990). These mutations were associated with the NtPOP^{tom} enzymes but not Taq DNA polymerase. As far as we are aware the potential contribution of bacterial repair pathways to complex mutations, which was not the main focus of this work, has not been investigated in previous studies. The use of alternative E. coli strains such as the low mutation rate MDS42pdu strain (Csorgo et al., 2012) could be used to study this theoretical possibility. The influence of plant organelle repair pathways on the mutation spectrum of the NtPOP^{tom} Exo-L903F enzyme requires the transformation of this enzyme into plants. Comparison of the mutation spectra from the in vitro data obtained from replication of the cI gene (this work) with in vivo data obtained by expressing NtPOP^{tom} Exo-L903F enzyme in plant organelles, will improve our understanding of organelle genome maintenance pathways in plants.

TABLE 2

| Enzyme | WT | Exo- | Exo-Pol903F |
|---|---|---|---|
| Sequenced mutants | 37 | 38 | 40 |
| Total number of mutations | 41 | 41 | 95 |
| Base substitutions | 27 | 26 | 74 |
| Single base insertions | 4 | 2 | 4 |
| Single base deletions | 6 | 6 | 13 |
| Complex mutations | 4 | 7 | 4 |

Table 2 shows the number (percentage) of types of mutations found in mutant ci genes replicated by the recombinant WT, Exo- and Exo-L903F NtPOP^{tom} enzymes.

TABLE 3

| DNA Polymerase | | | Mismatch |
|---|---|---|---|
| (No. cl genes | Synthesised strand | Number | formed |
| sequenced) | mutation | found | (Template:dNMP) |
| Taq DNA polymerase | A→G | 7 | T:G |
| (11) | C→A | 1 | G:A |
| | C→T | 1 | G:T |
| | G→A | 1 | C:A |
| | G→T | 1 | C:T |
| NtPOP | T→A | 9 | A:A |
| (37) | C→A | 5 | G:A |
| | C→T | 1 | G:T |
| | G→A | 7 | C:A |
| | G→T | 5 | C:T |
| | Single indels | 10 | |
| | Complex mutations | 4 | |
| Exo- (38) | A→C | 1 | T:C |
| | A→G | 1 | T:G |
| | T→A | 10 | A:A |
| | T→C | 2 | A:C |
| | C→A | 11 | G:A |
| | G→A | 1 | C:A |
| | Single indels | 8 | |
| | Complex mutations | 7 | |
| Exo- L903F (40) | A→T | 11 | T:T |
| | A→G | 11 | T:G |
| | T→A | 18 | A:A |
| | T→C | 2 | A:C |
| | T→G | 2 | A:G |
| | C→A | 9 | G:A |
| | C→T | 8 | G:T |
| | C→G | 2 | G:G |
| | G→A | 3 | C:A |
| | G→T | 8 | C:T |
| | Single indels | 17 | |
| | Complex mutations | 4 | |

Table 3 shows the number of different mutation types found in mutant c1 genes replicated by the recombinant WT, Exo- and Exo-L903F NtPOP^{tom} enzymes. Details of single base indels are shown in Table 2.

TABLE 4

| | | SEQ |
|---|---|---|
| | | ID |
| Name | Sequence 5′ → 3′ | NO |
| delNdeIPstI- | TACGAGAAAGAATTCAAAGTTGCA | 19 |
| F | | |
| delNdeIPstI- | ACTTTGAATTCTTTCTCG | 20 |
| R | | |
| M13-F | TTCCCAGTCACGACGTTGTAAAACGACGG | 21 |
| | CCAGTG | |
| pUN121-F | CCCCCAAGTCTGGCTATGCA | 22 |
| pUN121-R | CAACCATTATCACCGCCAGAG | 23 |
| CompetpUN- | GCTTAGAACCTTTACCAAAGGTGATGCGG | 24 |
| R1 | AGAGATGGGTAAGCACAACCAAAAAAGC | |
| CompetPUN- | CAGTGATTCTGCATTCTGGCTTGAGGTTG | 25 |
| R2 | AAGGTAATTCCATGACCGCACCAA | |
| CompetPUN- | CAGGCTCCAAGCCAAGCTTTCCTGACGGA | 26 |
| R3 | ATGTTAATTCTCGTTGACCCTGA | |
| pUN121_mut | TCAGGGTCAACGAGAATTAACATTCCGTC | 27 |
| | AGGAGCTTGGCTTGGAGCCTG | |

Table 4 shows oligonucleotides used (Sigma-Aldrich, Southampton).

### Example 2

### Introduction

The inventors have proposed the use of a recombinant POP targeted to plastids to use as a tool to mutagenize plastomes in plants. The POP has been shown to be the sole DNA polymerase essential for DNA replication in both plastids and mitochondria (Parent et al. 2011; Udy et al. 2012). Since this enzyme has also been found in plants and protists, it is named Plant and Protists Organelle DNA Polymerase—POP (Moriyama et al. 2011). The inventors have shown that in vitro that a mutator POP (NtPOP^{Exo-}L903F) with decreased replication fidelity but retaining replicative function can be made by modifying amino acids in key motifs in the proofreading and polymerisation domains. This would make the mutator POP a strong candidate to mutagenize plastomes in plants. Synthetic biology would allow assembly of a construct expressing the plastid mutator POP (MuPOP) which is controllable and detectable in vivo.

Applications of the error-prone DNA polymerases using 3′-5′ exonuclease deficient DNA polymerase gamma (Pol γ) involve elevating the mutation rate in mitochondrial DNA (mtDNA). The mutations produced by the proof-reading deficient Pol γ are mainly point mutations in addition to occasional deletions (Szczepanowska and Trifunovic 2015). In budding yeast, mutation rate in mtDNA elevated by proof-deficient Pol γ resulting in increased production of petite colonies by 10-15-fold (Foury and Vanderstraeten 1992; Chan and Copeland 2009). These petite mutants lack functional mitochondrial DNA and cannot respire. A mouse harbouring homozygous proof-reading deficient Pol γ exhibited a ˜2500× higher mutation frequency (1×10⁻³ per bp) in mitochondria than that in the wild type (6×10⁻⁷ per bp) (Vermulst et al. 2007), showed premature aging (Trifunovic et al. 2004; Kujoth et al. 2005). Fruit flies with a similar error-prone Pol γ exhibited less sensitivity to mtDNA mutations than mitochondria mutator mice (Kauppila et al. 2018). Progenies of the former has inherited 9.8×10⁻⁴ per bp mutations in mtDNA but they did not show early ageing phenotype. These cases have provided useful models for studying mitochondrial mutations linked to aging and diseases such as Parkinson and diabetes (Park and Larsson 2011).

Unlike Pol γ for yeasts and animals, POP is dual-targeted to both mitochondria and plastids in plants (Christensen 2005). A plastid-targeting peptide is required to deliver the MuPOP exclusively into plastids. The pair of paralogous POPs in Arabidopsis (AtPolA and AtPolB) have been frequently studied in recent years (Parent et al. 2011; Baruch-Torres and Brieba 2017), of which divergent roles were suggested for replication (AtPolA) and repair (AtPolB) (Ayala-Garcia et al. 2018). However, the interaction between two AtPOPs has not been clarified. To ensure the simplicity of the mutator system, a N. tabacum (common tobacco) POP was used to establish the mutator plastome. N. tabacum is not only the model species for studying plastids by reverse genetics, but it contains only one type of POPrather than two paralogous POPs. Following transformation, the mutator NtPOP is expected to compete for DNA substrates with wild type NtPOPs. As a result, the plastome mutator tobacco would be expected to have dysfunctional chloroplasts due to an elevated mutation rate in ptDNA, which might result in variegation or albinism. On the other hand, the mutated ptDNA might not be phenotypically detectable due to the efficient repair pathways in plastids. Furthermore, the phenotype in plastome mutator tobacco might also be influenced by the dosage of the mutator POP. In the mitochondrial mutator mouse, the early ageing phenotype was only seen in homozygous Pol γ deficient mice but not in heterozygous ones (Vermulst et al. 2008).

The expression of a phenotype due to dysfunctional mitochondria depends on the ‘threshold effect’ (Stewart et al. 2008). In animals, this term is explained as the bearable mutation frequency or heteroplasmy level of the mutant mitochondrial genome before causing respiratory chain dysfunction in a tissue or organ (Poulton et al. 2010). The phenotypic threshold varies depending on the mutation type (Trifunovic and Larsson 2008). Usually, the phenotypic threshold is presented as percentage, indicating the chance for a gene containing at least one mutation in mitochondria. The threshold for point mutations (90%) is higher than indels (60%) (Edgar and Trifunovic 2009). The phenotypic threshold has not been tested for chloroplasts.

In animals, a certain type of the mutated mtDNA can be enriched in a tissue or organ through random segregation of mtDNA into the daughter cells (Fayzulin et al. 2015; Kauppila et al. 2018). These mutant mitochondrial genomes can be isolated by fusing cells with rho zero cells lacking mtDNA (Wilkins et al. 2014). They can be studied in vivo if they are transmitted into the germline and segregated to homoplasmy. Back-crossing with wild type would remove the mutator Pol γ. This scheme is difficult because maternally inherited heteroplasmic mitochondrial genome require generations to sort out (Stewart et al. 2008). Strong purifying selection on mitochondrial protein coding sequences has been shown in mouse and human oocytes (Stewart et al. 2008; Burr et al. 2018), which could be more efficient when facilitated by the genetic bottleneck, which reduces mitochondria and mitochondrial genomes during oocyte division (Floros et al. 2018). These selective forces for functional wild type mtDNA would decrease the chances of obtaining a mitochondrial genetic mutant. Nonetheless, purifying selection may be disrupted by positive selection (Klucnika and Ma 2019). For the mutator mitochondrial gamma DNA polymerase expressed in the fruit fly, a method was developed using a nuclear expressed restriction enzyme (XhoI) targeted to a unique site in mtDNA, enabling targeted selection on the gene which resulted in mutations in the Xho I site that prevented cleavage and removal of mitochondrial genomes (Xu et al. 2008). More recently, isolation of a mouse cell line harbouring homoplasmic mutant mtDNA has been possible, using an inducible mutator Pol γ combined with an artificially introduced bottleneck (mtDNA copy number decreased by ethidium bromide) (Fayzulin et al. 2015).

Purifying selection has been suggested for ptDNA, especially photosynthesis related genes from phylogenetic studies (Zheng et al. 2017). Elevated mutation rate in plastids provides a pool of mutant ptDNA, which could produce homoplasmic mutants through segregation. For this purpose, tobacco is more advantageous than the mouse and fruit fly in at least two aspects: 1) Spectinomycin resistance resulting from point mutations in 16S rDNA is easily scored by screening antibiotic-resistant shoots, derived from cells containing resistant mutations in the plastid 16S rDNA gene, placed on regeneration medium (Fluhr et al. 1985; Svab and Maliga 1991). 2) Tobacco ptDNA in somatic leaf cells experience a bottleneck during shoot regeneration from cells present in plant explants placed on regeneration medium (Lutz and Maliga 2008). These features could enable isolation of homoplasmic plastome mutants resistant to spectinomycin. Furthermore, spectinomycin selection can be replaced or used in combination with other positive selection agents, allowing selections for other gain-of-function mutations, such as 1) atrazine resistance conferred by a point mutation in psbA, 2) enhanced photosynthesis conferred by alleles developed from photosynthetic related genes (rbcL pigment genes and PSI & II genes).

This example will aim to elevate mutations rate in plastids using the following objectives: 1) Introduce mutator NtPOP (NtPOP^{Exo-}L903F) into N. tabacum. 2) Isolate transgenic lines expressing mutator NtPOP and studying their phenotypes. 3) Investigating mutation rate in mutator plants. 4) Analysing the mutator plastome using both next- and 3^{rd} generation sequencing. 5) Isolating homoplasmic plastome mutants.

### Materials and Methods

### DNA Vector Construction

The expression cassettes containing the plastid mutator POP (MuPOP) were assembled using Golden Gate cloning (Engler et al. 2008). The native promoter and coding sequence of the wild type NtPoII-like 1 (Ono et al. 2007) (AB174898.1) were PCR cloned from Nicotiana tabacum cv. Petit Havana DNA or RNA (following reverse transcription), respectively. The plastid targeting sequence from the rbcS8 gene (X03820.1) was PCR cloned from Petunia hybrida DNA. The Heat Shock Protein 18.2 3′ UTR and transcription termination region was PCR cloned from Arabidopsis thaliana DNA (Nagaya et al. 2010). The complete expression cassettes of MuPOP comprised of the promoter, coding sequence and 3′ regulatory elements were assembled and cloned into the binary vector pART27 (Gleave 1992). All PCR primers are listed in Table 6 (see below).

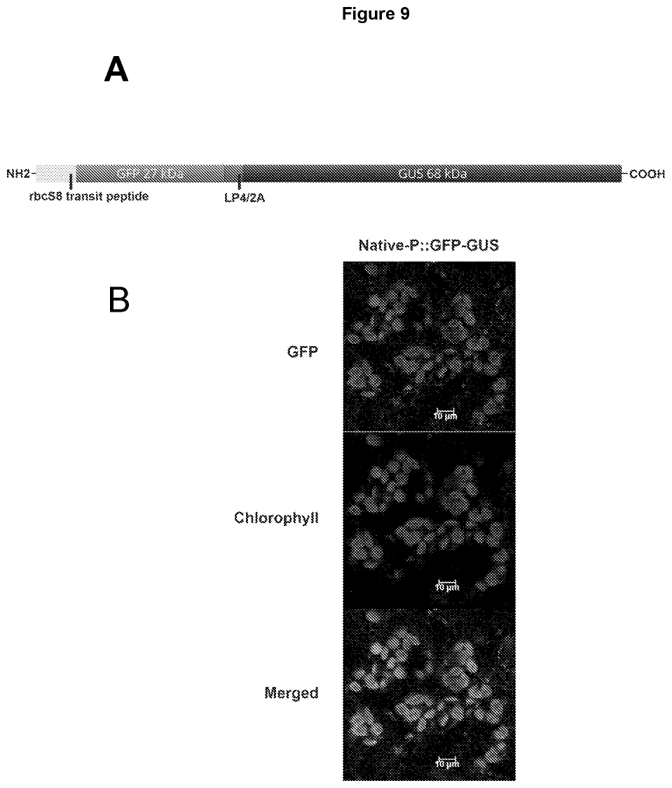

The coding region for the transit peptide of the petunia rbcS8 gene was fused to the N-terminus of a modified green fluorescent protein, GFP (Primavesi et al, 2008). The C-terminus of the GFP was linked to the reporter protein beta glucuronidase (GUS) using a LP4/2A peptide (François et al. 2004). The plastid targeted GFP-GUS fusion protein is shown in FIG. 9A. This allowed the screening of lines for GUS expression before visualising the subcellular location of GFP using confocal microscopy.

### Plant Propagation

Seeds from the wild type Nicotiana tabacum cv. Petit havana were sterilised with 100% ethanol for 1 min then 30% (w/v) bleach for 10 min. The sterilised seeds were germinated on ½ Murashige and Skoog (MS) medium (Murashige and Skoog, 1962). Seedlings were transferred to MS medium (Table 5) and grown ascetically in Magenta™ GA-7 vessels. Plants were incubated at 25° C. with 12-hour day/night cycle and were ready for transformation after 3-4 weeks.

### Agrobacterium Mediated Plant Transformation

Agrobacterium tumefaciens GV3101 (Holsters et al. 1980) was transformed with the binary vector pART27 (Gleave 1992) containing expression cassettes containing the plastid mutator POP (MuPOP) or the GFP-GUS fusion protein. Transgenic antibiotic-resistant shoots were selected on medium containing 50 mg/L kanamycin.

For stable expression of MuPOP, Nicotiana tabacum was transformed with Agrobacteria containing pART27:MuPOP, the procedures followed (Dandekar and Fisk 2005). Tobacco transformants were selected on regeneration medium containing 200 mg/L kanamycin. Stable transformants were isolated and grown on MS medium containing 200 mg/L kanamycin in Magenta™ GA-7 vessels to allow development of roots. The isolated shoots were grown to 4-week old before used for spectinomycin assay.

Stable transgenic lines expressing the plastid targeted GFP under the regulation of the plastid organellar DNA polymerase promoter and 5′ UTR were examined using a Leica SP8 inverted confocal florescence microscope.

TABLE 5
Composition of plant media

| Name | Function | Components |
|---|---|---|
| MS or ½ MS | seeds germination, | 1x or ½x Murashige and Skoog (MS) basal salts and |
| | rooting and routine | vitamins with 2.5 mM 2-(4-morpholino) |
| | culture | ethanesulfonic acid (MES) and 3% w/v sucrose. |
| | | Solidified with 0.7% agar, pH 5.8 |
| Regeneration | shoot regeneration | MS medium supplemented with 4.5 μM |
| | | benzylaminopurine (BA), 0.5 μM naphthalene acetic |
| | | acid (NAA) and solidified with 0.7% agar, pH 5.8 |

### Spectinomycin Assay

Four week old tobacco plants (T1 generation) expressing MuPOP were used for the spectinomycin resistance assay. Wild type tobacco was used as control. The 2^{nd} to 4^{th} expanded leaves from the top of the plants were excised into approximately 3 mm×3 mm explants and transferred to shoot regeneration medium (Table 5) containing 200 mg/L spectinomycin. Explants were transferred to fresh shoot regeneration medium after three weeks. Explants were cultured for 6 weeks before recording the number of spectinomycin resistant shoots present. The resistant shoots were isolated and transferred onto MS medium containing 200 mg/L spectinomycin and grown in Magenta™ GA-7 vessels to allow the development of roots. Photoautotrophic plants were transferred to soil and grown to maturity, whereas heterotrophic plants (e.g. white mutants) were maintained on MS medium containing 2% (w/v) sucrose and 200 mg/L spectinomycin.

The phenotypes of spectinomycin resistant tobacco plants were determined following the formation of roots and leaves in young plantlets growing on MS medium containing 200 mg/L spectinomycin.

### Plant Reciprocal Crosses

Spectinomycin-resistant MuPOP plants (variegated) and phosphinothricin (PPT) resistant transplastomic plants 14C (Iamtham and Day, 200) were grown to the flowering stage in temperature and light controlled walk-in growth rooms (25° C., 12 h day/night cycle). The 14C lines contains a plastid-localised bar gene conferring PPT resistance. The 14C line is resistant to PPT but sensitive to spectinomycin (Iamtham and Day, 2000). Spectinomycin-resistant MuPOP and 14C lines were reciprocally crossed to each other. Anthers of the recipient flower were removed before pollen development. Pollen was collected from the donor flower and applied onto pistils of recipient flowers. Successful pollination was confirmed by the formation of seed pods.

To test for maternal inheritance of spectinomycin resistance, seeds from the crosses were germinated on half strength MS medium alone or containing 200 mg/L kanamycin, 200 mg/L spectinomycin or 15 mg/L PPT, respectively.

### Plant DNA and RNA Extraction

Total DNA was extracted from plant young leaves using DNeasy® Plant Mini Kit (Qiagen, UK). Purified DNA samples were stored at −20° C. Plant RNA was extracted from young leaves using the TRIzol™ Reagent according to the manufacturer's instructions (Invitrogen, UK). Purified RNA samples were stored at −80° C.

### Polymerase Chain Reaction (PCR)

All primers used for PCR are listed in the table 6 below. For DNA fragments (promoter, presequence, coding sequence and 3′UTR) used for cloning, the target DNA fragments were amplified by standard PCR using MyTaq™ Red Mix (Bioline, UK) DNA polymerase in a BioRad T100 thermal cycler (BioRad, UK). For the amplification of DNA fragments from MuPOP plants, Mytaq polymerase was replaced with the high fidelity Q5 DNA polymerase (NEB, UK). Sequences of all PCR products were determined by Sanger sequencing (Eurofins Genomics Germany, Ebersberg). Oligonucleotides were ordered from Sigma-Aldrich, Poole.

TABLE 6
Oligonucleotide primers for Example 2.

| Name | Sequence 5′→3′ | SEQ ID NO |
|---|---|---|
| Tobacco | | |
| Native-POP | | |
| promoter | | |
| and 5′UTR | | |
| cloning | | |
| N.tomNative | GATTCGGTAAGGGGTGAGACGGGT | 28 |
| DPProm-F | | |
| N.tomNative | GGAGAAGGAATGAGGGGAAAACCA | 29 |
| DPProm-R | | |
| N.tomNative | GAGCTCGTTTAAGGAAGCATATCAAGG | 30 |
| DPProm- | | |
| SacI-F | | |
| N.tomNative | ACTGAACTGAGAACCCCAA | 31 |
| DPProm- | | |
| NcoI-R | | |
| AtHSP70- | | |
| Promoter | | |
| cloning | | |
| AtHSP70-F | GACAATTTAAAAAAGACCCATCTCGAAG | 32 |
| AtHSP70-R | TTCATGGCGACTTGATTCTTGG | 33 |
| AtHSP70- | GGAGCTCGAAGGAGCTAGAAGCGA | 34 |
| Sacl-F1 | | |
| AtHSP70- | CCGTCTCCCCATTATTAGAGATCAGAATTGTTCGCC | 35 |
| BsmB1-R1 | | |
| PetrbcS-JJ-F | ACCACATAATCCAATGGTTAGCTTTACT | 36 |
| cloning | | |
| PetrbcS-JJ-R | CGAGTTTCAAGTTCCGAGTAATTACATA | 37 |
| PetrbcS-JJ- | CCGTCTCCATGGCTTCCTCTGTGATTTCCTCTG | 38 |
| F_BsmBI | | |
| PetrbcS-JJ- | CCGTCTCCTGGACATGCATTGGACTCTTCCACCA | 39 |
| R_BsmBI | | |
| GFP cloning | | |
| GFP_BsmB1- | GACGTCTCTGCATGGTGAGCAAGGGCGAGG | 40 |
| F | | |
| GFP_XhoI-R | GAGACTCGAGTTACTTGTACAGCTCGTCCAT | 41 |
| cpTargetingG | CGTCTCTGCATGGTGAGCAAGGGCGAGGAGCTGTT | 42 |
| FP_BsmB1-F | | |
| cpTargetingG | CGTCTCGCTTGTACAGCTCGTCCATGCCGTGAGTG | 43 |
| FP_BsmB1-R | | |
| LP4/2A | | |
| LP4-2A | CGTCTCCCAAGTCTAATGCTGCTGATGAAGTTGCTACTCAA | 44 |
| linker-F | CTTCTTAATTTTGATCTTCT | |
| LP4-2A | CGTCTCCACATTGGTCCTGGATTAGATTCAACATCTCCAGC | 45 |
| linker-R | AAGCTTAAGAAGATCAAAATTAAGAAGTTGAGTAGCA | |
| GUS cloning | | |
| GUStoLinker_ | CGTCTCCATGTTACGTCCTGTAGAAACCCCAA | 46 |
| BsmB1-F | | |
| GUStoLinker_ | CTCGAGCTATTGTTTGCCTCCCTGCTGCG | 47 |
| BsmB1-R | | |
| 3′UTR | | |
| cloning | | |
| AtHSP18.2-F | AGAACGAAGAGAAGAACGATAAGTGG | 48 |
| AtHSP18.2-R | TGTTACATGAGGAGCCTTAACCAAAACA | 49 |
| AtHSP18.2XhoI- | CCTCGAGATATGAAGATGAAGATG | 50 |
| F | | |
| AtHSP18.2NotI- | GGCGGCCGCTCGACACGATAGGAGGGTCGGT | 51 |
| R | | |
| 16S rrn | | |
| PCR/ | | |
| sequencing | | |
| Ntcp16S-F | CGAACTCCGGGCGAATATGAA | 52 |
| Ntcp16S-R | GGAAAGAAGACTTCCATCTCCAAGT | 53 |
| SeqNtcp16S-R | CCGCTTTGTCTACGAACAAGG | 54 |
| RT-PCR to | | |
| detect | | |
| MuPOP | | |
| StrepTagRTPCR- | TCTGGATCTGGATCTTGGTCTCAT | 55 |
| F (MuPOP) | | |
| AtUTRRTPCR- | GCCACAAATTCATAACACAACAAGC | 56 |
| R (MuPOP) | | |
| EF1alpha-F | TGAGATGCACCACGAAGCTC | 57 |
| (control) | | |
| EF1alpha-R | CCAACATTGTCACCAGGAAGTG | 58 |
| (control) | | |
| Plastid Gene | | |
| primers to | | |
| confirm | | |
| mutations | | |
| PG2rbcl-F | TCCCCTGTTGGGATGTACTATTAAAC | 59 |
| PG2rbcl-R | CTTATCCAAAACGTCCACTGCTG | 60 |
| W1ycf3-F | TCATACGGCTCAGCAGTCAATTCT | 61 |
| W1ycf3-R | CGATTTTTATCAGCTGTAGCAAAGA | 62 |
| psaB_TA-F | CAGTAGCCCAAACAAGATGTCCA | 63 |
| psaB_TA-R | CGGGTTGGTTAAATGCTGTTAATG | 64 |
| psbJ_TA-F | TTACGCTTTCATGTTGACGACTCA | 65 |
| psbJ_TA-R | TCATTCGGAAGGATCTCATAATTT | 66 |
| ndhF_AG-F | AAACTCCCTGTTATCCAATAAAAACCT | 67 |
| ndhF_AG-R | AGTCATGGTTTTTTCAATCTACCTGTC | 68 |

### Reverse Transcription PCR (RT-PCR)

RNA samples were reverse transcribed using GoScript™ Reverse Transcription System (Promega, UK) in a BioRad T100 thermal cycler. Semi-quantification of MuPOP transcripts was by RT-PCR using primers specific for the Streptag II and 3′UTR region. Transcripts from the housekeeping gene EF-1α were used as the reference control. RNA samples without reverse transcription did not give rise to PCR bands verifying the absence of DNA contamination in the RNA samples tested. PCR products were fractionated on 2% W/V agarose gels in Tris-Borate-EDTA buffer (Sambrook et al., 1989)

### Enrichment and Amplification of Plant Organelle DNA

As plant organelle genomes are not methylated whereas nuclear DNA is highly methylated (Feng et al. 2010), nuclear DNA can be captured by MBD2-Fc-bound magnetic beads (NEBNext® Microbiome DNA Enrichment Kit, NEB, UK). Removal of methylated DNA (nuclear DNA) results in the preparation of highly purified organelle DNA (Yigit et al., 2014). Organelle DNA purified using the NEBNext® Microbiome DNA Enrichment Kit followed the Manufacturer's Instructions. Twenty to fifty nanograms of organelle DNA was purified from 1 microgram of total plant DNA. Ten to twenty nanograms of purified organelle DNA was amplified by Multiple strand Displacement Amplification (MDA) using the RPLI-g UltraFast Mini Kit (Qiagen, UK). Each amplifying reaction was carried out at 30° C. for 6 hours, then 65° C. for 3 min to inactive the Phi29 enzyme. The amplified DNA product was purified using 3× volumes of SPRI JetSeq™ Clean beads (Bioline, UK). The purified amplified DNA was quantified using the Quantifour® ONE dsDNA fluorescent dye (Promega, UK) and a Synergy HI Multi-Mode Microplate Reader (BioTek Instruments) set at 504 nm_{Ex}/531 nm_{Em}.

### Extraction of Total Plant Protein

Young leaf samples taken from plants grown in soil or in vitro were frozen in liquid nitrogen and then ground into a fine powder. 100 mg powder was resuspended in four volumes of freshly prepared RIPA buffer (10 mM Tris-HCl pH 8.0, 150 mM NaCl, 1% NP40 (v/v) and 1% SDS (w/v). The protein suspension was placed for 10 min a boiling water bath before removing insoluble material by sedimentation by centrifugation at 14,000 rpm of 10 minutes in an Eppendorf Microfuge 5415c with a 18-place rotor for 1.5 ml microfuge tubes.

### Protein Gel Electrophoresis and Protein Blot Analysis

Total plant protein extracts were fractionated using a 10% (W/V) polyacrylamide stain-free gel (Bio-Rad, UK) by SDS-PAGE and then transferred to nitrocellulose membranes using a Trans-Blot® Turbo™ (Bio-Rad, UK) transfer system. Successful transfer was confirmed by staining with Ponceau S solution (0.02% w/v). Strep-Tactin® alkaline phosphatase conjugate (IBA, Germany) was used with SuperSignal™ western blot enhancer (Thermo Scientific, UK) to detect the Streptag II fused to C-terminal MuPOP. The detailed procedures provided by the manufacturers were followed.

### Sanger Sequencing

Selected plastid genes were amplified using the PCR primers listed in Table 6 and sequenced by Sanger sequencing (Eurofins Genomics Germany, Ebersberg). Sequencing data were analysed using the Geneious Prime DNA analysis program (Biomatters, Auckland).

### Next Generation Sequencing Library Construction

The amplified organelle DNA from the MDA reaction has a hyper-branched structure, which was resolved into linear DNA using T7 endonuclease (NEB, UK) at 37° C. for 20 min. DNA clean-up and size-selection was performed using SPRI JetSeq® Clean beads (Bioline, UK) to select DNA with a size >1 kbp for preparing the library. Organelle DNA from plant lines G1, PG2 and W6 were sequenced using the Illumina Hi-Seq platform and 150 base pair end reads by Novogene (Hongkong). Over 90% of the reads ≥Q30. W1 and W4 were sequenced in-house using Oxford Nanopore Technology (ONT, Oxford). Library preparation and sequencing procedures followed the protocol SQK-LSK109 (ONT) in combination with NEBNext® Ultra DNA Library Prep Kit (New England Biolabs, Hitchin). Long read sequencing was performed on the MinION with Flowcell R9.4 (ONT, Oxford).

### Quality Processing of Sequencing Data

Next-generation Hi-Seq data (Novogene, Hongkong) was processed to remove read lengths of less than 50 nucleotides and select a quality ≥Q35. Filtered reads were used for genome assembly and SNP analysis. Long read nanopore data was base-called using Guppy software (ONT). Adapter sequences were trimmed with Porechop (https://github.com/rrwick/Porechop). The trimmed reads were passed through quality control (size >1 kb and >Q9) using NanoFilt (De Coster et al. 2018).

### Extraction of Plastid Reads

Plastid reads from Illumina HiSeq and Nanopore sequencing technologies were extracted by mapping to the linearised reference plastid genome, Nicotiana tabacum cv. BY4 (NCBI Z00044.2) using Geneious Prime 2020 (Biomatters, Auckland). Geneious Aligner (Geneious Prime 2020) was used on the Illumina HiSeq data with iterative mapping (5×). Minimum sequencing coverage was ˜2000×. ONT reads were mapped to the reference genome using Minimap2 (Li 2018) using the default parameters. Minimum sequence coverage was 100×.

### Identification of Single Nucleotide Polymorphisms (SNPs)

The plastid genome of wild type Nicotiana tabacum cv Petit Havana was assembled through reference (Z00044.2) based assembly. Inverted repeat B (IRB) was removed from the alignment consensus, and the resulting sequence was used as the reference plastid genome sequence to call SNPs on plastid reads.

For the G1, PG2 and W6 lines, the extracted plastid short reads (Illumina Hi-seq, 150 base PE) from each MuPOP sample were re-mapped to the reference genome (wild type plastid genome without IRB), using Geneious Aligner (up to 5 times iterative mapping, minimum mapping quality (MP) 90 ‘Trim paired read overhangs’ turned on and ‘accurately map reads with error to repeat regions’ turned on). SNPs were called using the Geneious program ‘find SNPs/variants’ function. SNPs were called if they represented over 25% of total reads for any given location on the plastid genome.

For W1 and W4, the extracted plastid long reads from the MINion with Flowcell R9.4, were processed and aligner Minimap2 (Li, 2018) was used for re-mapping the long reads to the reference genome using the default parameters.

### Results

### 2.1 Design of the Constructs for Different Expression of NtPOP^{Exo-}L903F

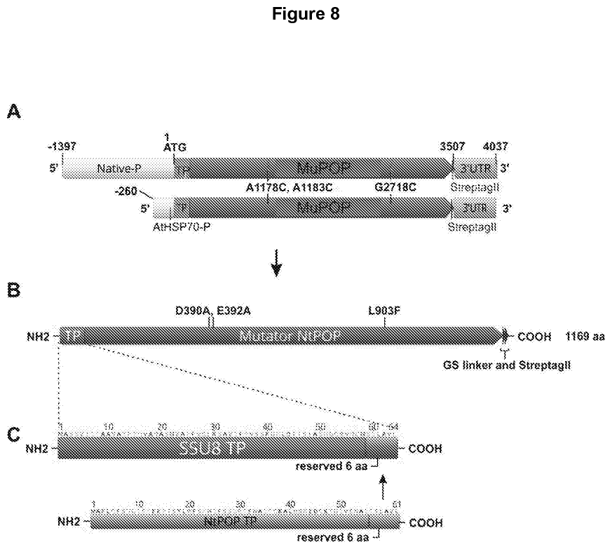

Two expression cassettes were designed for expressing of a chloroplast targeting mutator POP-NtPOP^{Exo-}L903F with either its native promoter (Native-P) or a promoter derived from Arabidopsis heat shock protein 70 (AtHSP70-P) (FIG. 8A). The expression of the Native-P driven MuPOP is expected to be under the same regulation as that of the wild type NtPOP. AtHSP70-P was used as a heat shock inducible promoter to provide control over the expression of the MuPOP. The N-terminal transit peptide (TP) for targeting MuPOP to chloroplasts was the full-length transit peptide of petunia SSU8 plus six amino acids from the POP upstream of the predicted cleavage site (FIG. 8B&C). The addition of 6 aa from the POP transit peptide was to ensure proper cleavage of the transit peptide. FIG. 9B shows that the GFP fused to the SSU8 transit peptide is targeted to chloroplasts.

The two expression constructs were cloned into a binary vector pART27 and transformed into wild type N. tabacum by Agrobacterium mediated transformation. The nptII conferred kanamycin resistance to the TO generation of transgenic plants. For the ease of description, the transformants with the Native-P or AtHSP70-P promoters were named NT or HS, respectively. More than 50 kanamycin resistant TO plants were isolated for each type of transformant (NT or HS), from which the seeds were collected and stored. No obvious phenotype was observed in the TO plants. Seeds from −10 TO plants were sown on kanamycin media. Most lines showed a proportion of sensitive seedlings, indicating a segregating nptII gene (Table 7). Seedlings from four NT lines and three HS lines were studied in more detail. Three NT lines (NT1, 4 and 6) contained a few T1 variegated seedlings, whereas this phenotype was not observed in the HS T1 seedlings. Other seedlings were green and indistinguishable from wild type. The variegated seedlings provided an early indication that had a MuPOP phenotype. Two NT lines (NT1 and NT6) and one HS line (HS4) were selected for further studies.

TABLE 7

| | | | Rate of Kan^{R} | Variegated |
|---|---|---|---|---|
| Construct | Lines | Total | seedlings¹ | seedlings |
| Native::MuPOP (NT) | 1 | 118 | 81% | 4 |
| | 3 | 66 | 71% | 0 |
| | 4 | 89 | 88% | 3 |
| | 6 | 128 | 95% | 10 |
| AtHSP70::MuPOP | 1 | 213 | 98% | 0 |
| (HS) | | | | |
| | N1 | 161 | 76% | 0 |
| | 4 | 84 | 65% | 0 |

Table 7 shows isolated transgenic lines (T1 generation). Seedlings grown on 200 μg/ml kanamycin MS medium.

### 2.2 Detection of the MuPOP in the Transgenic Lines

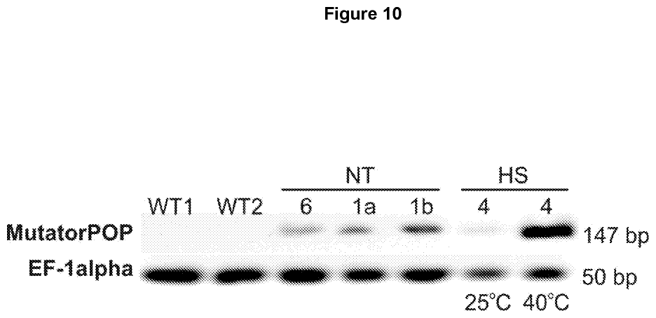

A mixture of two sets of primers were used in RT-PCR to investigate transcript accumulation of MuPOP and elongation factor 1 alpha (EF-1 alpha) mRNA (a housekeeping gene). FIG. 10 shows that MuPOP mRNA was not detectable in the wild type tobacco plants. In NT1a, NT1b and NT6, the transcript of MuPOP was detected but at different levels. Two samples were taken from the same HS4 plant before (25° C.) and after heat shock treatment (40° C.). MuPOP RNA was barely detectable at 25° C. but increased after heat shock (FIG. 10).

The expression of the MuPOP protein was investigated by Western blot analysis. Streptactin was used to detect the strep tag II at the C-terminus of MuPOP. A 100-150 kDa band was consistently detected in NT1a, NT1b and NT6, suggesting the translation of the full-length (123 kDa) MuPOP enzyme (FIG. 11). Such a signal could not be detected in HS4 samples with/without heat shock.

### 2.3 Phenotypes of the Transgenic Lines Expressing MuPOP

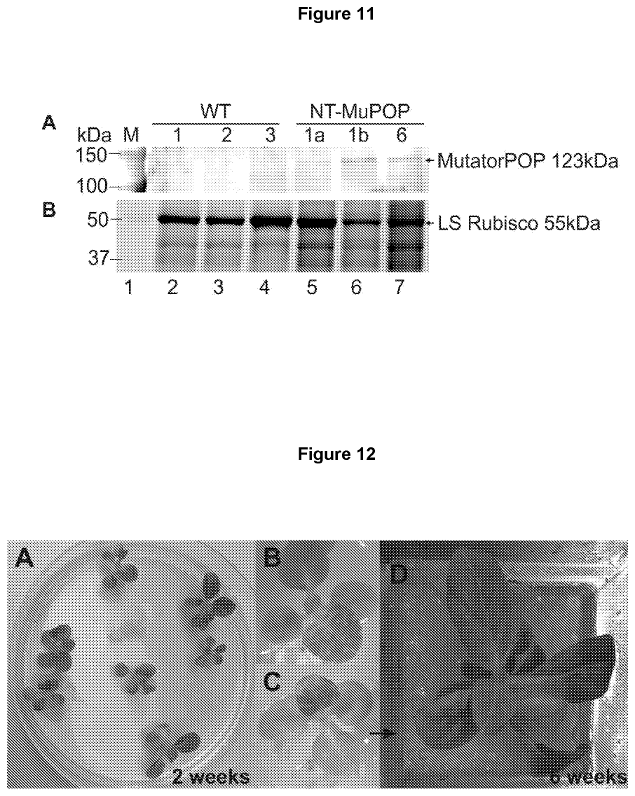

Given the ability of MuPOP to elevate the mutation rate in vitro, we predicted mutated plastid genomes in the transgenic plants expressing MuPOP. Mutations in chloroplast genes give rise to albino and pale-green phenotypes. Sorting-out of heteroplasmic mutant plastomes may explain the presence of variegated seedlings in the T1 generation of NT lines (FIG. 12A-C). The variegated phenotype was lost in new leaves in plants grown to 6 weeks old (FIG. 12D). This observation applied to variegated seedlings from both NT1 and NT6 lines. As the expression of the wild type POP relates to the proliferation of cells in rice and tobacco, expression of the MuPOP driven by the native promoter and 5′ UTR of NtPOP should show the same pattern of expression. The loss of the white sectors during development of MuPOP plants indicates a mechanism, such as purifying selection, that removes plastid mutations.

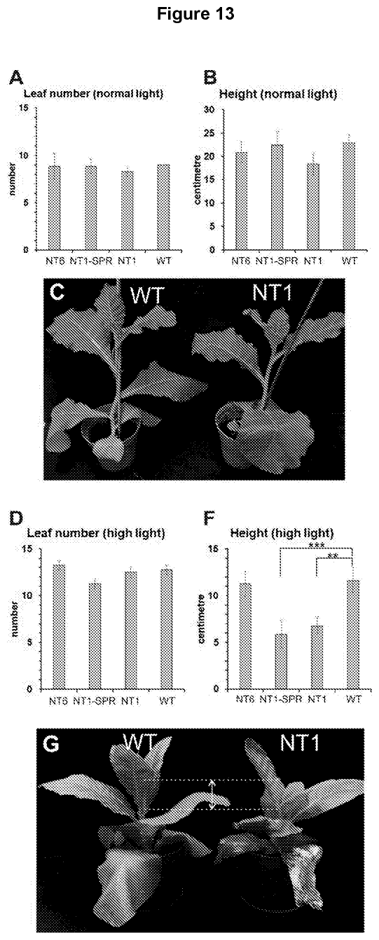

Removing mutations introduced by the mutator polymerase may have a genetic cost, which might have physiological consequences on MuPOP plants. To investigate this, the NT lines (1 and 6) were grown under high light stress conditions (600 μmol photons/m²/s) (FIG. 13). In addition, a green plant from NT1 with spectinomycin resistance (NT1-SPR) was also tested in this experiment. The generation of NT1-SPR plants will be discussed later (Result 2.4). The number of leaves and height of plants were recorded. Under normal light conditions (35 μmol photons/m²/s), all tested MuPOP plants were indistinguishable from the wild type plants in terms of their leaf numbers, heights and visual appearances (FIG. 13A-C). Plants flowered and set seed more rapidly under high light. The plants were also shorter in high-light as previously observed (Feng et al. 2019). Statistical analysis shows that the dwarf effect was more severe in NT1 (P<0.01) and NT1-SPR plants (P<0.001) than that in NT6 and wild type plants (FIG. 13D&F). A further difference was the presence of necrotic leaves in NT1 but not NT6 (FIG. 13G). No necrotic leaves were observed in wild type under high light. Plants stressed by high light may be less able to cope with the genetic load conferred by elevated mutation rate in plastids.

### 2.4 Effect of Positive Selection on Chloroplast Genomes in MuPOP Plants

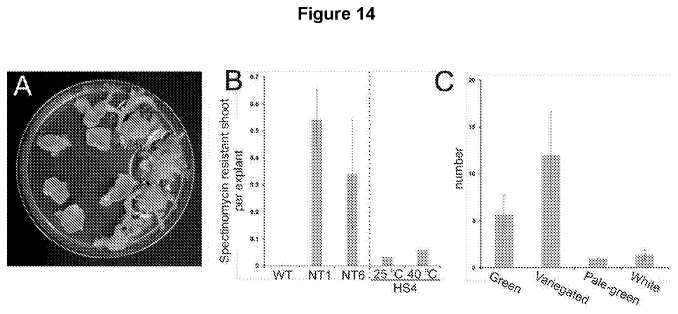

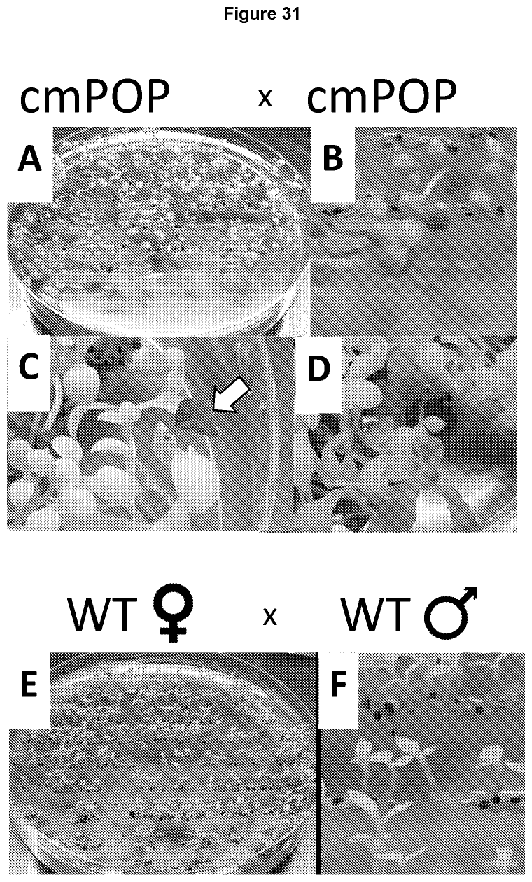

Similar to the mutator Pol γ in the mouse mitochondria, one hypothesis that the MuPOP would elevate mutation rate in the plastid is proposed here. The MuPOP most likely mutates the whole plastome randomly and generate both gain-of-function and loss-of-function mutations. Given that loss-of-functions such as white sectors could not be identified phenotypically in MuPOP plants, another assay was designed for screening gain-of-function mutations. Several point mutations in the chloroplast 16S rrn gene can confer spectinomycin resistance (Svab and Maliga 1991). Here the mutation rate is presented as shoots per explant to estimate relative differences in acquisition of spectinomycin resistance. These point mutations in the 16S rrn gene can occur in wild type plants, at a rate of about 1/500-1/1000 shoots per explant on regeneration medium containing spectinomycin (Wang et al. 2014). Here, one green spectinomycin resistant shoot was isolated from 600 wild type explants, giving a rate of 1/600 shoots per explant for the wild type (cv. Petit havana) we use. This number is increased by 331- and 209-fold when explants from NT1 and NT6 were used for the assay, respectively (FIG. 14B). HS4 plants also give rise to a higher mutation rate than wild type leaves but only by 21- and 37-fold for the samples with and without heat shock treatment, respectively. The distribution of regenerated spectinomycin resistant shoots was not even on each explant but exhibited as either none or multiple shoots on one explant (FIG. 14A). The more regenerative explants might be sourced from younger leaves where contains more developing cells and hence higher expression of MuPOP. FIG. 31 shows that plastid mutation rate was elevated in the female germ lines of MuPOP plants. This resulted in the maternal transmission of spectinomycin-resistant plastids to seedling, which were visualized as green sectors that were resistant to the bleaching by spectinomycin. Because of a presumed bottle neck that reduces the copy number of plastid genomes in the female germ-line this method of screening seedlings provides an alternative method for fixing plastid mutations. Use of the native POP promoter ensures the mutator POP is active in amplifying mutant plastid genomes following the bottle neck during the development of the egg and zygote following fertilisation.

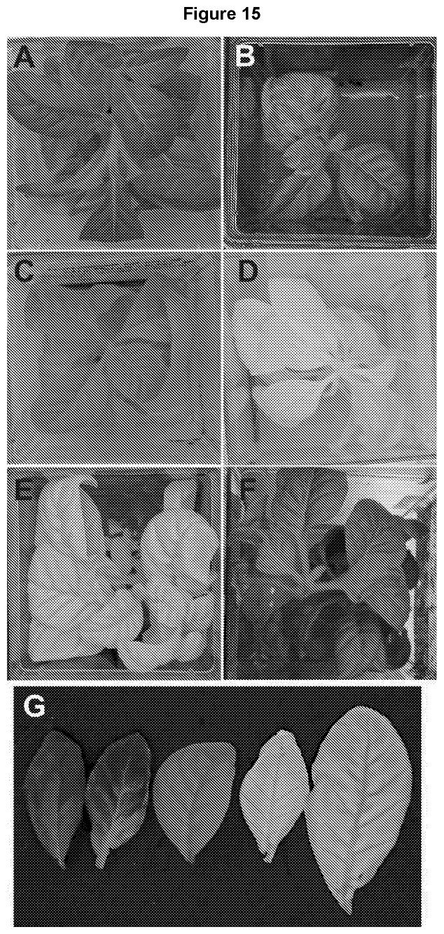

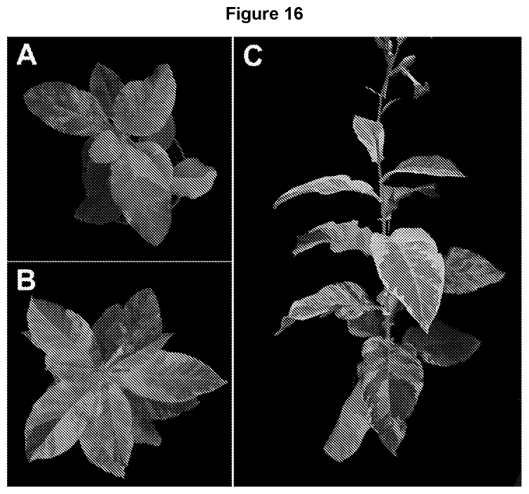

The phenotypes of spectinomycin resistant shoots could be categorized into green, variegated, pale-green and white leaves. The number of shoots corresponding to each type of phenotype varied. The spectinomycin selection assay has been repeated three times on NT1 explants to investigate the distribution of the population for each phenotype. From NT1 35 explants (averaged from three repeats), the number of each phenotype was 12 variegated>6 green>1 pale-green=1 white (FIG. 14C). This pattern was consistent in the repeated assays. All the regenerated shoots could be isolated and grown up in vitro (FIG. 15A-E). Additionally, a plant with an ‘ivory’ coloured appearance (FIG. 15E) was identified at this stage as it was not distinguishable from white shoots at the earlier regeneration stage. Green plants could be transferred and grown in soil, showed no phenotypic difference from wild type plants (FIG. 15A&F). The variegated plant could also survive in soil and give rise to sectorial, peripheral chimeric and mosaic leaves within one plant (FIG. 16A-C). Such dynamic variegation within an individual plant indicates highly heteroplasmic plastid genomes in the shoot apical meristem (SAM). The remaining plants (pale-green, white and ivory) were photosynthesis deficient, requiring sucrose supplemented medium to grow heterotrophically.

The isolation of spectinomycin resistant shoots with different phenotypes from a single plant, suggests a heteroplasmy of chloroplast genomes in the green MuPOP plants even before positive selection. The spectinomycin selection on these heteroplasmic genomes resulted in fixation of the gain-of-function mutation in the 16S rrn gene throughout all regenerated shoots regardless their different phenotypes. This result also shows that multiple mutations were present, even though only resistance to spectinomycin was selected. The emergence of photosynthesis deficient shoots indicates that detrimental mutations co-exist with the mutations responsible for spectinomycin resistance.

### 2.5 Maternal Inheritance of Mutated Plastid Genomes

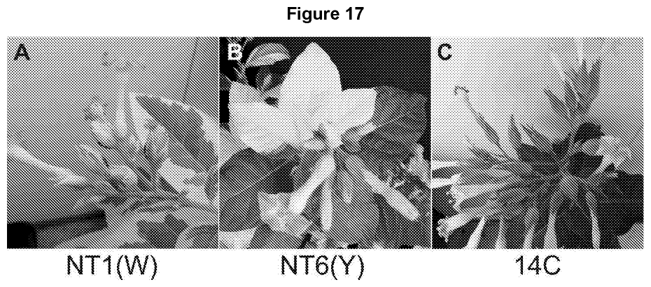

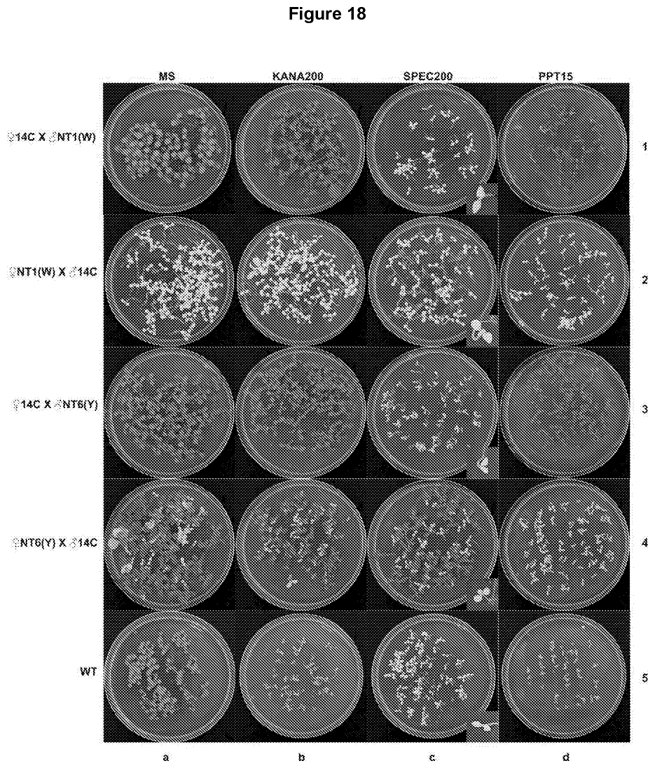

The inheritance of pale green and white sectors were studied to determine maternal versus Mendelian inheritance. Mutant plastids would show maternal inheritance whereas the mutator POP linked to kanamycin would show Mendelian inheritance (FIG. 17). One NT1-SPR plant was named NT1(W) due to its large white (W) sectors (FIG. 17A). The other NT1-SPR plant was named NT6(Y) because of its large yellow (Y) sectors (FIG. 17B). 14C contained a phosphinothricin (PTT) resistant gene (bar) in the plastome, while its nuclear genome is wild type (Iamtham and Day 2000). In tobacco, the plastid genome is uniparentally inherited from the mother. Therefore, the plastid genotype in the hybrid can only inherited from the mother. FIG. 18 shows the result of the reciprocal crosses. The hybrid F1 seedlings are spectinomycin resistant only when their mother is one of the NT1-SPR plants (FIG. 18, column c), whereas the F1 hybrids are PTT resistant if only their mother was 14C (FIG. 18, column d). The pigmentation trait is also maternally inherited. The chlorophyll deficient chloroplasts in the NT1(W) plant was uniformly and maternally transmitted to its progeny (FIG. 18 2a and 2b). The mixed population of yellow, variegated and green progenies resulted from unfinished sorting-out of the chloroplast genomes in NT6(Y) maternally transmitted to its progeny (FIG. 18 4a and 4b). Either phenotype from NT1(W) or NT6(Y) was not seen when they were the pollen donor to 14C plants (FIG. 18 1a-b and 3a-b). In contrast to transmission of spectinomycin resistance and pale-green or white sectors which were inherited maternally, kanamycin resistance linked to the mutator was transmitted through pollen (FIG. 18b). This shows that the mutations do not result from the activity of MuPOP in seedlings, which would inherit the nuclear localised copy of the gene from the paternal parent.

The reciprocal cross experiments have ruled out the nuclear mutation but only the cytoplasmic mutation as the cause for the phenotypes in the NT-SPR plants. Given MuPOP has been shown exclusively targeting to the plastid, those phenotypes are attributed to plastome mutations.

### 2.6 Genetic Analysis of the Mutated ptDNA in MuPOP Plants

The maintenance system of ptDNA remains unclear. Mutated ptDNA may be repaired or degraded, based on purifying selection observed in the MuPOP seedlings. In this case, white NT-SPR plant may result from 1) the maintenance system failing to repair the highly mutated ptDNA, or 2) the mutation induced photosynthesis deficiency.

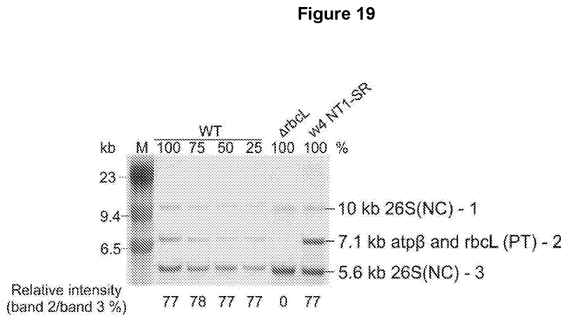

Degradation of mutated ptDNA could lead to a reduction in ptDNA copy number, resulting in an albino phenotype in seedlings. The white seedlings in maize w2 POP mutants have been shown to be related to a severe reduction in ptDNA copy number. To investigate if that is the case in the white NT-SPR plants, a Southern blot was performed to compare the ptDNA copy number between the wild type tobacco and a white NT1-SPR plant (W4). A ΔrbcL tobacco was used to identify bands due to nuclear DNA copies only (FIG. 19). The probe mixture binds to both atpβ and rbcL genes in ptDNA and the 26S rDNA in nuclear DNA. The negative control (ΔrbcL) does not contain the region binding to probe for ptDNA, resulting in no signal of ptDNA on the blot. W4 shows the intensity of the ptDNA signal is similar to that in the wild type, when they have the same level of nuclear DNA. This result indicates that, at least in W4, the white phenotype is not caused by reduced ptDNA copy number.

To summarize, the white NT1-SPR plant has been confirmed for its resistance to spectinomycin which is maternally inherited pigment-deficient mutations. Green NT1-SPR plants resistant to spectinomycin may also contain mutations unlinked to the mutations in the 16S rDNA genes (16S rrn gene).

### 2.7 MuPOP can Mutagenize Chloroplast Genome at Random Position

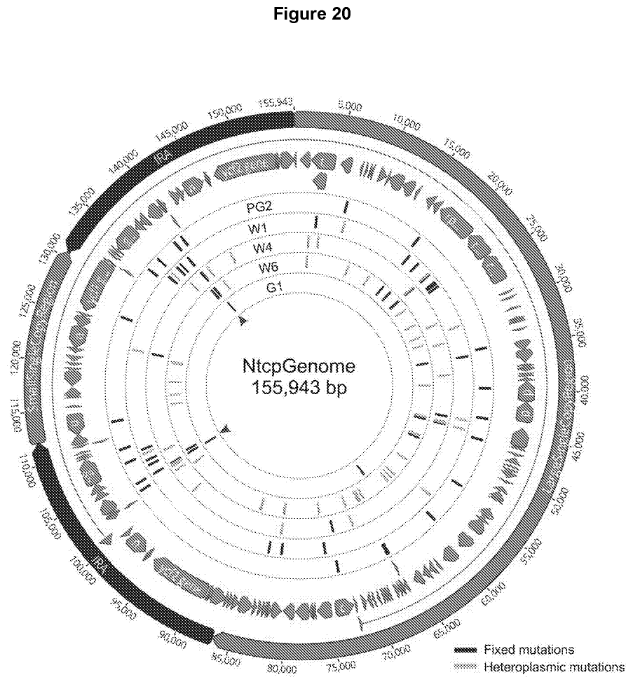

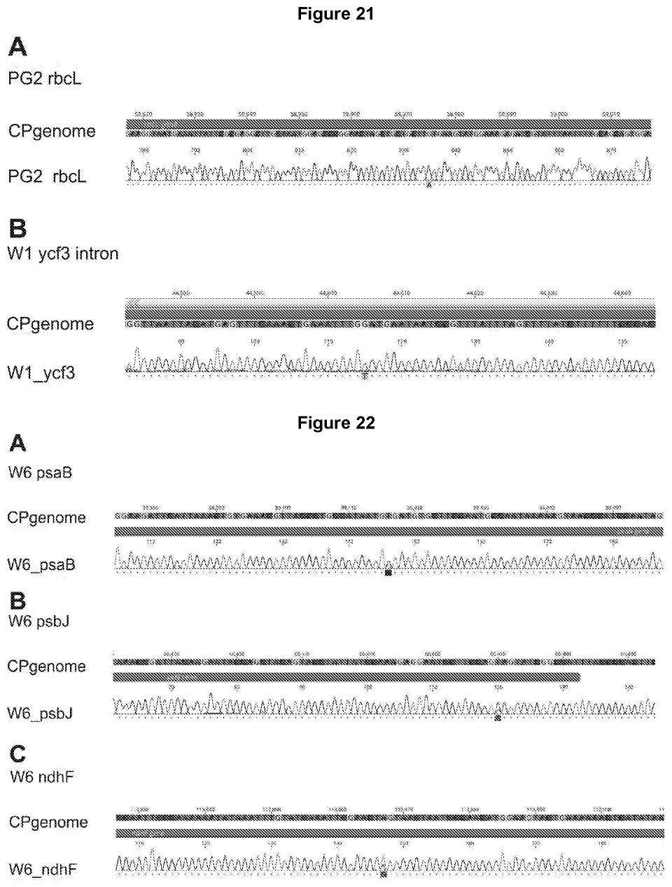

MuPOP has been shown to mutagenize a 500 bp long sequence randomly at multiple bases in vitro (Example 1). In plastids, the MuPOP may act in a similar way as it does in vitro. To investigate mutations in these NT-SPR plants, I initially used Oxford Nanopore Technology (ONT) 3^{rd} generation sequencing technology on three white (W1, W4 and W6), one pale-green (PG2) and one green (G1) NT-SPR samples. Illumina next-generation sequencing technology was then used to investigate W6, PG2 and G1 lines. Data from both technologies were aligned to the reference chloroplast genome (NCBI Z00044.2). On average, 200-300× and 2000-4000× coverage were achieved using ONT and Illumina data, respectively. Illumina reads were 150 bp paired end reads. ONT reads were >1 kb long. Single nucleotide polymorphism (SNP) mutations for each line were identified and mapped to the chloroplast genome using ONT data (FIG. 20). These SNPs were called using Nanopolish program with the ‘fix homopolymers’ function turned on and ‘variant frequency’>40%. Setting 40% as the minimum variant frequency, 29, 26, 42, 21 and three SNPs were called in W1, W4, W6, PG2 and G1 samples, respectively. The mutations within individual lines were randomly distributed throughout the genome, but no mutations were found in ycf1 and ycf2 genes. One possible reason might be strong purifying selection on these two genes, as they are essential for tobacco survival (Drescher et al. 2000). The mutations in 16S rDNA responsible for spectinomycin resistance were found in all sequenced samples at >99% variant frequency (FIG. 20). This result validates positive selection using spectinomycin. In this study, I defined SNPs with >70% frequency as fixed mutations, while the other SNPs (40-70%) were defined as heteroplasmic mutations. To validate our method for determining fixed or heteroplasmic SNPs using ONT data, several SNPs were randomly selected for Sanger sequencing (FIG. 21 and FIG. 22). Sanger sequencing was definitive for identifying the SNPs, which gave a single clear peak for fixed SNPs (FIG. 21) but double peaks for heteroplasmic SNPs (FIG. 22). The result shows that all genomes contained fixed SNPs (not including those for spectinomycin resistance in 16S) but co-existed with varying numbers of heteroplasmic SNPs. The ratio of fixed SNPs out of the total in each sample were 20/29 (W1), 6/26 (W4), 10/42 (W6), 21/21 (PG2) and 3/3 (G1). Although it has been reported that some SNPs might be missed when using ONT reads for SNP analysis, the data above was sufficient to show MuPOP was a functional and effective mutator DNA polymerase in vivo. The heteroplasmic chloroplast genomes in W1, W4 and W6 implied that the NT-SPR plants are regenerated from explants containing heteroplasmic chloroplast genomes. In contrast, the fixed chloroplast genomes in PG2 and G1 showed that a number of mutations could be fixed within a round of positive selection.

Illumina next-generation sequencing was used for more comprehensive analysis on the SNPs in W6, PG2, and G1 samples. Given the reads had high accuracy (>99.9%), the variant frequency for calling SNPs was reduced to 30%. To avoid the possibility of false positives, SNPs were not called below 30%. The number of called SNPs in each tested sample increased to 72 (W6), 25 (PG2) and five (G1). These additional SNPs included those located in homopolymeric tracts. Three single base deletions were identified in W6, which were not identified using ONT data.

All SNPs identified in W6, PG2 and G1 were located on the reference genome and listed in Table 8. Despite the two SNPs within 16S rDNA which were responsible for spectinomycin resistance, G1 only contained a SNP in the coding sequence (CDS) of ycf4 gene. The SNP resulted in amino acid substitution K112I in ycf4. PG2 also contained a nearly fixed chloroplast genome. One of the SNPs resulted in an early stop codon in the rpoC2 gene near the end of its translational product, which may not affect enzyme function. Subtracting those SNPs in the intergenic region and introns, the SNPs within the CDSs of photosynthetic genes (psaB, psbD) might be the cause for the photosynthesis deficiency in PG2. W6 contains a highly heteroplasmic genome with a ratio of 10/72 (fixed/heteroplasmic SNPs). But its albino phenotypes might result from the dominant mutations. If the heteroplasmic SNPs and those located in non-coding regions are subtracted from the list, the fixed SNPs in rpoC1 and ropC2 are likely to be the reason for the albino phenotype in W6. The rpoC1 and rpoC2 mutants have been shown to have an albino phenotype due to diminution of transcription in plastids (Serino and Maliga 1998).

TABLE 8

| Affected | CDS/intron/ | Nucleotide/ | | |
|---|---|---|---|---|
| genes¹ | intergenic | Codon changes | AA changes | FX/HT² |
| intergenic | | C1742T | | HT |
| matK | CDS | ACA > ACT | Silent | HT |
| intergenic | | G7183A | | HT |
| intergenic | | A7830T | | HT |
| intergenic | | A9211T | | HT |
| trnK | intron | C1916T | | HT |
| trnG | intron | G10123A | | HT |
| intergenic | | A14558T | | HT |
| rpoC2* | CDS | GAA > AAA | E1158K | FX |
| | CDS | ATC > TTC | I603F | FX |
| rpoC1* | CDS | AGC > AGT | Silent | FX |
| | CDS | AAG > ATG | K401M | HT |
| | CDS | AGG > TGG | R108M | HT |
| | CDS | CGC > CCC | R619P | HT |
| | CDS | CCC > CCT | Siilent | HT |
| rpoB | CDS | TTT > TTC | Silent | HT |
| intergenic | | C29252A | | HT |
| intergenic | | C30038T | | HT |
| intergenic | | G31663A | | HT |
| psbC | CDS | GAA > AAA | E71K | HT |
| psbD | CDS | CCA > CAA | P238Q | FX |
| psaA | CDS | TTA > TAA | L360 > stop | HT |
| psaB | CDS | ACA > TCA | T600S | HT |
| ycf3 | Intron | G44605T | | HT |
| | Intron | G45419A | | HT |
| | intron | C45736T | | HT |
| | intron | A45531T | | HT |
| | intron | C45914A | | FX |
| rps4 | CDS | CAT > CAA | H57Q | HT |
| intergenic | | A49192G | | HT |
| Intergenic | | A49252G | | HT |
| trnL | intron | T49737A | | FX |
| intergenic | | C54497T | | HT |
| Intergenic | | G57540A | | HT |
| rbcL | CDS | CGC > CGT | Silent | HT |
| intergenic | | T59148A | | HT |
| accD | CDS | ACC > ATC | T51I | HT |
| | CDS | AAA > ATA | K40I | HT |
| intergenic | | C65754T | | HT |
| rpl33 | CDS | del A | K49 > frame | |
| | | | shift | |
| psbJ | CDS | ACT > TCT | T5S | HT |
| psbE | CDS | TAG > TGG | Stop > W | HT |
| intergenic | | T69415A | | HT |
| clpP | intron | G72862A | | HT |
| | intron | del T | | HT |
| rps 12 | intron | C74739T, | | HTx2 |
| | | A140953G | | |
| rpoA* | CDS | CGC > CAC | R26H | HT |
| | CDS | CTA > CTT | Silent | HT |
| | CDS | AAT > TAT | N132Y | HT |
| rps8 | CDS | CGA > CAA | R14Q | HT |
| rps 12 | intron | G105380A | | HT X2 |
| rpl22 | CDS | GGT > GGA | Silent | HT |
| | CDS | ACG > ATG | T131M | HT |
| intergenic | | T101677C | | FX x2 |
| 16S ** | CDS | C861A | | FX X2 |
| trnl | intron | G104597A | | HT X2 |
| trnA | intron | T106083A | | HT X2 |
| ndhF | CDS | ACT > ACC | Silent | HT |
| intergenic | | A115674T | | HT |
| ndhD | CDS | ATA > TTA | I283L | HT |
| | CDS | AAT > ATT | N496 | HT |
| ndhA | CDS | TAT > AAT | Y315N | HT |
| | CDS | CCT > TCT | P230S | HT |
| ndhH | CDS | del A | Y239 > | HT |
| | | | frame shift | |

Table 8 shows W6 SNPs analysis using Illumina next-generation sequencing. Mutations linked to albino phenotype are indicated with a single Asterix (*). Mutations linked to spectinomycin resistance are indicated with a double Asterix (**). The genes are arranged in ascending order by the position of identified mutations on the reference genome (NCBI Z00044.2). FX, fixed mutation, variant reads coverage >70% total coverage. HT, heteroplasmic mutation, variant reads coverage between 40-70% total coverage.

### 2.8 MuPOP Introduces a Distinct Spectrum of Mutations in Tobacco Chloroplast Genomes

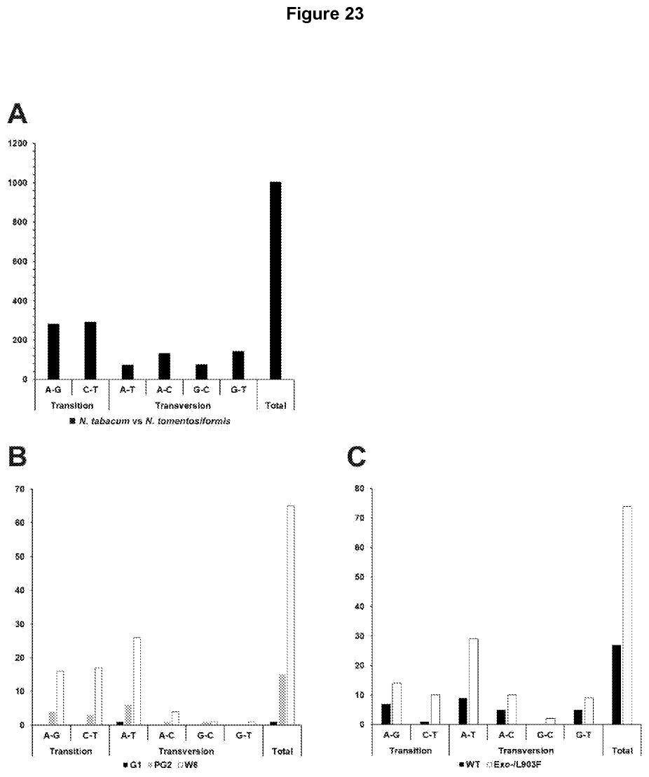

To investigate the spectrum of polymorphisms between tobacco species, chloroplast genomes from N. tabacum and N. tomentosiformis were compared (FIG. 23A). In a total of 1005 identified polymorphisms, there are 576 transitions and 429 transversions. Within the transitions, two types of polymorphisms (A-G and C-T) had similar frequencies: 283 for A-G and 293 for C-T. For transversions, A-C substitutions were double that of A-T transversions (133 to 74, respectively), and the ratio of G-T to G-C follows the same pattern (145 to 77, respectively). A-G and C-T are the most frequent types of polymorphisms, followed by A-C and G-T then A-T and G-C. This data provides information of SNPs found in chloroplast genomes over evolutionary time.

The spectrum of substitutions made by the MuPOP in vivo (FIG. 23C) showed a preference for A-T transversions due to drastically increased frequencies of A:A and T:T mispairings. The same mutation preferences were shown in the sequenced PG2 and W6 plastid genomes (FIG. 23B). These preferences corresponded to those observed in vitro (FIG. 6C). The same mutation preferences were shown in the sequenced PG2 and W6 plants (FIG. 23B). These preferences corresponded to those observed in vitro (FIG. 6C). This was not expected as plastids have a more complex environment where the DNA maintenance system involving DNA repair enzymes may be expected to influence the consequences of mispairings introduced by the MuPOP. This observation shows the importance of DNA replication fidelity in controlling the pattern of base substitutions.

### DISCUSSION

An active error-prone version of POP namely NtPOP^{Exo-}L903F was introduced into plastids in N. tabacum. The transgenic tobacco harbouring mutator plastomes showed a proportion of variegated seedlings in its progeny, which was lost during development. Positive selection using spectinomycin allowed enrichment of mutated plastomes in spectinomycin resistant shoots. These shoots could be isolated and propagated in vitro, and presented with a range of pigmentation phenotypes, including green, pale-green, ivory, white and variegated leaves. Applying both Illumina and ONT sequencing technologies on the green, pale-green and white plants revealed relatively large numbers of mutations in ptDNA. These mutations were mainly single base substitutions with occasional single base indels. No large sequence rearrangement was identified in the sequenced samples, confirmed with the ONT long reads data with a size of >1 kb. The plastome mutator system revealed the importance of purifying selection and positive selection.

Sequencing of plastome mutants revealed that mutagenesis of MuPOP appears random throughout the plastome. The mutated genes include those under strong purifying selection during evolution, such as matK (Young and DePamphilis 2000). SNP analysis also showed a wide spectrum of base substitutions, which was characterized by preferential A-T transversions compared to the naturally occurred polymorphisms between two tobacco species (N. tabacum and N. tomentosiformis) (FIG. 23). The preference for A-T transversion could result from altered nucleotide discrimination in the polymerisation domain by L903F, as it was also found in vitro

### Example 1

The variegated phenotype observed at the seedling stage in transgenic plants expressing MuPOP driven by the native promoter was transient. The relative impact of the mutator is likely to depend on its relative abundance with respect to wild type POP. Tobacco is tetraploid with four wild type POP genes compared to a single mutator POP gene. The ratio is reduced in T1 plants with two copies of the mutator POP genes. In mitochondria mutator animals, the phenotype correlates with the dosage of the mutator Pol γ (Vermulst et al. 2007; Samstag et al. 2018). Loss of variegation suggested the existence of purifying selection during plant development that removes mutant plastids. This result is in contrast with the findings in mouse and human, where strong purifying selection was suggested during oocyte division, but the leaked mutant mitochondrial genomes can accumulate to phenotypically detectable levels in tissues and organs (Poulton et al. 2010; Floros et al. 2018).

The high light treatment of MuPOP plants results in reduced height and necrosis in old leaves in NT1 plants, but these phenotypes were not observed in the wild type and NT6 plants. Taking the ‘threshold effect’ into account, NT1 and NT6 should contain a certain level of heteroplasmic mutations in plastids but below the phenotypic threshold. The high light can increase oxidative stress in chloroplasts (Dorrell and Howe 2012). Combining the increased oxidative stress with the existing level of mutations, NT1 might cross the phenotypic threshold. The absence of a necrosis phenotype in NT6 might result from its lower mutation frequency, which was shown in the spectinomycin selection assay.

It has been shown that a plastome with the minor population in the mixture with the major one could be enriched by the endogenous bottleneck, and hence the minor plastome could develop to an individual plant with homoplasmic plastome (Lutz and Maliga 2008). The bottleneck during explant regeneration is able to decrease the plastid number by 10-fold, from ˜100 per somatic cell to ˜10 per stematic cell (Shaver et al. 2006). But this size of bottleneck might be not efficient enough. As no shoots showing distinct phenotypes were achieved in a regeneration experiment of 25 MuPOP explants on the normal regenerative medium, each explant giving rise to 20-25 wild-type-like shoots. A very tight bottleneck has been shown necessary for isolating mouse mitochondrial mutant, which decreased the mtDNA copy number to one or even null per cell using ethium bromide (Fayzulin et al. 2015). The regenerative plant cell may still contain ˜10 copies of ptDNA after bottleneck, in which the plastome mutant could be outcompeted by the wild type ones.

Therefore, the positive selection has played an important role in the isolation of plastome mutants from plastome mutator plants. Spectinomycin allows positive selection on the point mutations in 16S rDNA gene in tobacco (Svab and Maliga 1991). These point mutations do not interfere with the function of 16S rRNA. Therefore, spectinomycin selection allows detection of the phenotypes caused by other mutations outside of 16S rDNA gene. Taking PG2 and W6 as examples, their chlorophyll deficient phenotypes were due to hitchhiker mutations unrelated to spectinomycin selection.

The spectinomycin selection facilitated the uniform fixation of mutations in 16S rDNA in each mutator plant line (FIG. 20). There was no positive selection on hitchhiker mutations, but the fixed hitchhikers (>70% heteroplasmy) were also detected (Table 8 and Table 10 and 11). SNP calling at different heteroplasmy level showed a highly heteroplasmic plastome in W6, whereas PG2 and G1 contained mainly fixed plastomes. The fixed hitchhikers might have originated from a dominant mutant plastome, or be escapes from the repair system. To clarify this question, an experiment is required to track certain mutations within a germline lineage of a plastome mutants. Regardless of the limited number of samples, our data shows that the number of SNPs correlates with the severity of chlorophyll deficiency (Table 9, SNPs called at 30% heteroplasmy). This correlation is lost for the number of fixed SNPs, as W6 has less fixed SNPs than that in PG2. As a result, the chlorophyll deficiency phenotype in the pale-green or white samples might be induced by a small number of fixed mutations rather than a complication effect of overall mutations, such as the threshold effect in mitochondria-mutator mice (Edgar and Trifunovic 2009). This hypothesis can be tested by recovering fixed mutations to wild type by plastid transformation.

TABLE 9

| Sample | 30% | 40%¹ | 50% | 70% |
|---|---|---|---|---|
| W6 | 72 | 54 (42) | 56 | 13 |
| PG2 | 25 | 21 (21) | 21 | 21 |
| G1 | 3 | 3 (3) | 3 | 3 |

Table 9 shows The number of SNPs called with different heteroplasmy level using Illumina data. The number of SNPs called using ONT data is in the brackets.

Combining the mutator POP with appropriate selection schemes would allow isolation of additional traits beyond spectinomycin resistance. Such a system could potentially be applied to other species. Instead of spectinomycin, it would be worthwhile to test some herbicidal compounds targeting chloroplasts, such as atrazine (psbA), Tentoxin (atpE) and Sorgoleone (PSII subunits) (Dayan and Duke 2014). Furthermore, plant mitochondria can be the alternative target for MuPOP and used for screening mitochondria related traits such as cytoplasmic male sterility.

TABLE 10

| Affected | | Nucleotide/Codon | AA | FX or |
|---|---|---|---|---|
| genes | CDS/intron/intergenic | changes | changes | HT |
| intergenic | | G6772A | | FX |
| rpoC2 | CDS | AAA > TAA | K1340 | FX |
| | | | stop | |
| psaB | CDS | TCA > TTA | S471L | FX |
| | CDS | | | |
| | CDS | | | |
| intergenic | | A43827C | | FX |
| intergenic | | A46729T | | FX |
| intergenic | | G53330A | | FX |
| rbcL | CDS | TGT > TAT | C459Y | FX |
| CDS4 | | G66363A | | FX |
| intergenic | | A66539T | | FX |
| clpP | intron | A73318T | | FX |
| 16S | CDS | G62A | | FXx2 |
| 16S | CDS | C1440T | | FXx2 |
| 23S | CDS | A633T | | FXx2 |
| 23S | CDS | G2520A | | FXx2 |
| ndhF | CDS | CCT > CTT | P519L | FX |
| ycf1 | CDS | TAC > TTC | Y1644F | FXx2 |

Table 10 shows PG2 SNPs analysis using Illumina next-generation sequencing.

TABLE 11

| Affected | CDS/intron/non- | Nucleotide/Codon | AA | FX or |
|---|---|---|---|---|
| genes | CDS | changes | changes | HT |
| ycf4 | CDS | AAA > ATA | K112I | FX |
| 16S | CDS | C1015A | | FXx2 |

Table 11 shows 1 NPs analysis using Illumina next-generation sequencing.

### Example 3

The chloroplast mutator POP of the present invention (cmPOP) makes mutations in the female germ line providing a method to introduce chloroplast mutations into seedlings.

Chloroplasts are inherited through the female germ line in many crops including tobacco, Brassicas and cereals such as maize, wheat and rice (Corriveau and Coleman, 1988). As a result plastid mutations made by the mutator plastid POP in the female germ line will be transmitted to the progeny. The number of chloroplast genomes undergoes a reduction in copy number during the development of egg cells (Christie and Beekman, 2017). This reduction in chloroplast number, the so called bottle neck, means that chloroplast mutations are more easily fixed when they are introduced in the female germ-line. This results in homoplasmy. The chloroplast DNA copy number then increases following fertilisation of the egg cell and growth and development of the zygote into seedlings. Chloroplast DNA replication is controlled by the native POP promoter for these processes. Expression of the chloroplast mutator DNA polymerase driven by the native POP promoter provides a powerful means to introduce mutations into the female germ line and zygote. Use of the native POP promoter ensures expression of the chloroplast mutator POP at the key time points when chloroplast DNA replication would normally take place resulting in its amplification following the bottle neck drop in the number of chloroplast genomes per cell.

Growing seedlings on spectinomycin enables visualisation of plastid mutations formed during the development of egg cells and growth of the zygote. Cells with wild-type chloroplast genomes bleach white, whereas cells with chloroplast mutations conferring spectinomycin-resistance are green. In some cases the majority of the seedling was green indicating fixation and homoplasmy of mutations conferring spectinomycin-resistance (green seedling indicated with arrow in FIG. 31C). In FIGS. 31A-D, seedlings with green spectinomycin-resistant sectors were obtained following self-fertilisation of chloroplast mutator POP plants. On average, one seedling with green sectors was obtained for every 200 bleached seedlings. In contrast, no green sectors were present in wild-type seedlings germinated on spectinomycin medium (FIGS. 31E-F), which was previously determined to be less than one green sector per 15,000 wild type seedlings (Ruf et al., 2007).

### Example 4

### Introduction

The inventors proposed the use of the active error-prone version of POP namely NtPOP^{Exo-}L903F targeted to mitochondria to use as a tool to mutagenize mitochondrial genomes in plants in a similar manner to that demonstrated for chloroplasts in example 2.

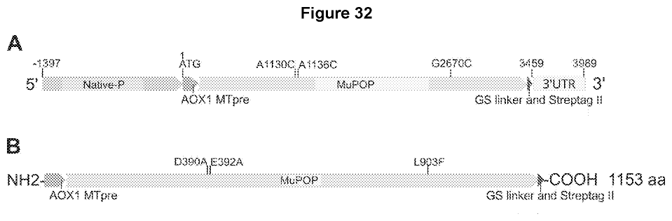

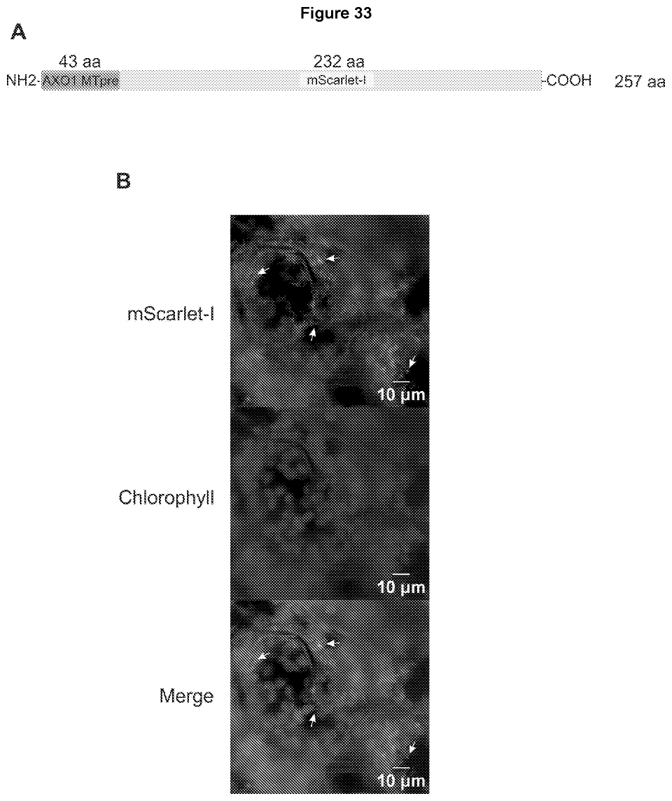

The methods used for mitochondrial application are the same as that of example 2, except modifications as below.
1. The construct for expressing the recombinant POP targeted to plant mitochondria was the same as the construct for expressing MuPOP in Example 2, except the chloroplast transit peptide (petunia Rubisco Small Subunit 8 transit peptide, SSU8 TP, FIG. 8) was replaced with a mitochondrial targeting presequence from the mitochondrial Alternative Oxidase 1 from soybean (AOX1 MTpre, FIG. 32A). The AOX1 Mtpre presequence is predicted to be cleaved between amino acids S41 and E42 by the TargetP 2.0 program (Armenteros et al. (2019) Life Science Alliance 2: e201900429). The presequence is cleaved naturally following import of the protein into the mitochondria by mitochondrial proteases. The native tobacco POP promoter and 5′ UTR (Native-P) was used to regulate the expression of the mitochondrial-targeted MuPOP (MT-MuPOP).
2. Mitochondrial targeting conferred by the AOX1 N-terminal peptide (43 aa) was confirmed by localization of the fluorescent protein mScarlet-I (Bindels et al. 2016) to mitochondria. The expression construct (FIG. 33A) was cloned into the vector pART27.
3. Illumina sequencing data from the transgenic tobacco plant harbouring MT-MuPOP (FIG. 36) was mapped to a reference mitochondrial genome (NCBI accession No. NC_006581). The mapped reads were used to reveal the mutations using the Geneious Prime program as described in Example 2.

### Results

### Phenotypes of the Transgenic Lines Expressing MT-MuPOP

Given the successful application of MuPOP in the increasing the mutation rate in plant chloroplasts, MuPOP was also expected to elevate the mutation rate in plant mitochondria.

The presequence from the soybean mitochondrial Alternative Oxidase 1 (AOX1) enzyme was used to target MuPOP to mitochondria in tobacco. To confirm the mitochondrial targeting properties of the mitochondrial AOX1 presequence it was fused at the N-terminus to the fluorescent mScarlet-I protein (FIG. 33A). mScarlet-I fluorescence was localised to mitochondria confirming mitochondrial targeting of the mitochondrial AOX1 presequence (FIG. 33B, Top image). No mScarlet-I fluorescence was found in chloroplasts, visualised by chlorophyll fluorescence (FIG. 33B, middle image). The merged image shows both chloroplasts and mScarlet-I tagged mitochondria (FIG. 33B, bottom image).

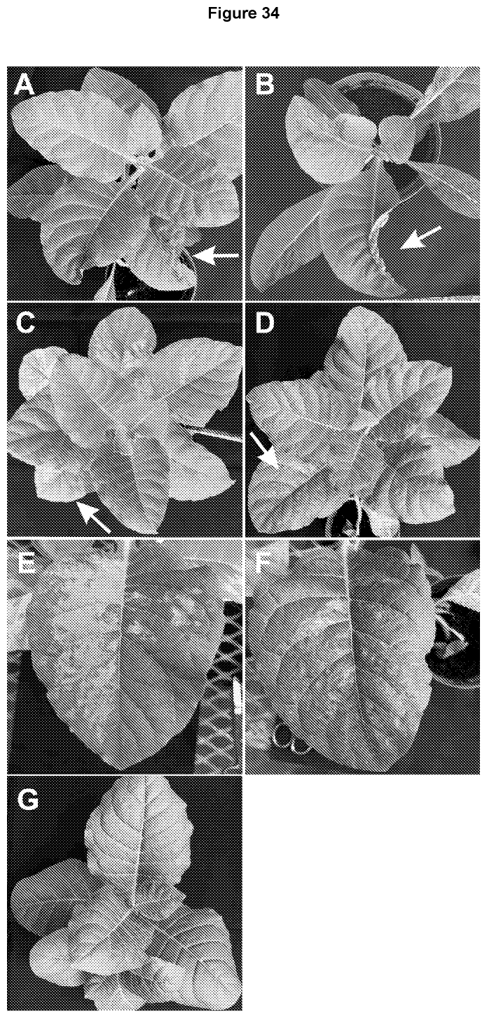

The expression construct of the mitochondrial-targeting MuPOP (MT-MuPOP) (FIG. 32) was then cloned into the binary vector pART27 and transformed into wild type N. tabacum by Agrobacterium-mediated transformation. The TO transgenic plants were selected in vitro with kanamycin. Twenty-five kanamycin resistant plants from independent transgenic events growing in vitro were screened for new phenotypes. Four transgenic plants corresponding to MT-MuPOP lines 1-4 showed visible phenotypes that were not found in wild type plants. The remaining 21 transgenic plant lines were indistinguishable from wild type plants in their pigmentation and morphology. The phenotypes were also visible following transfer of in vitro-propagated transgenic plants to soil resulting in photosynthetic growth to larger plants (FIG. 34).

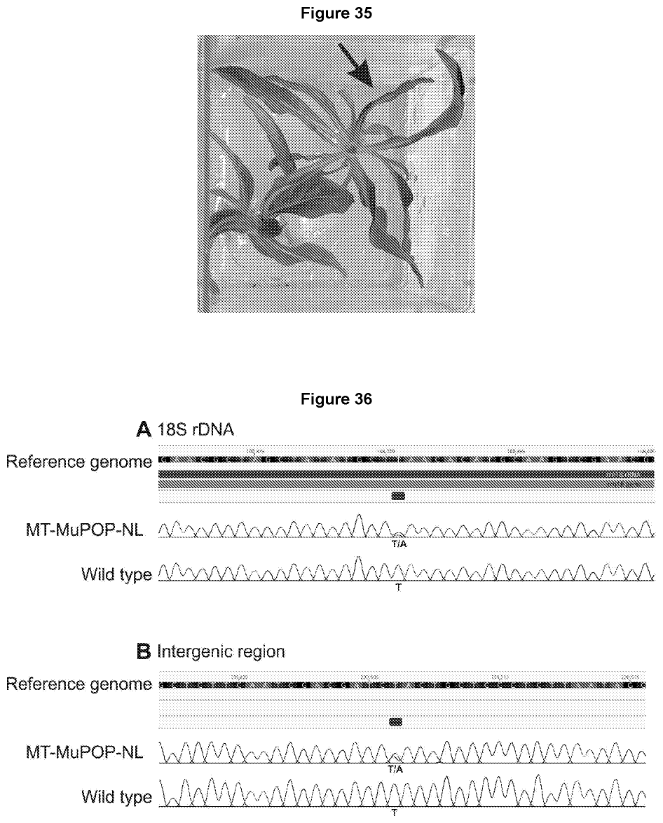

The phenotypes in MT-MuPOP1-4 could be categorised into distorted leaves (MT-MuPOP lines 1 and 2, FIG. 34A-B) and variegation caused by pigmentation deficiency (MU-MuPOP lines 3 and 4, FIG. 34C-F). Additionally, a subclone of MT-MuPOP line 2 (named MT-MuPOP2-NL) regenerated in vitro from the parent MT-MuPOPline 2 showed a narrow-leaf phenotype (FIG. 35). As no selective agent was used, the presence of the pigmentation deficiency or distorted leaf phenotypes in the MT-MuPOP transgenic plants is linked to the elevated mutation rate in mitochondria by MT-MuPOP. This result suggests a different organelle genome maintenance system in plant mitochondria compared to chloroplasts. In chloroplasts, the pigment-deficient and leaf shape phenotypes observed in seedlings and young transgenic tobacco plants expressing the chloroplast-targeting MuPOP (FIG. 12) were lost during further growth and development in soil.

### MuPOP can Mutagenize Mitochondrial Genome

The MT-MuPOP2-NL plant grown in vitro retained its narrow leaf phenotype and was used for Next-generation sequencing analysis. Four single nucleotide polymorphism (SNP) mutations were detected in the mitochondrial genome at various positions across the genome including intergenic and coding regions, including the 18S rDNA and nad9 genes (Table 12). The mutations in 18S rDNA and an intergenic position T230570A were further confirmed by Sanger sequencing (FIG. 36) following PCR amplification using the oligonucleotide primers listed in Table 13. It is noted that the intergenic mutation was fixed and therefore homoplasmic within the plant.

TABLE 12

| | | SNP | | |
|---|---|---|---|---|
| Affected | CDS/intron/ | locations | AA | |
| gene | intergenic | (codon changes) | changes | FX/HT |
| 18S rDNA | CDS | T18390A | N/A | HT |
| Intergenic | | T230570A | N/A | HT |
| nad9 | CDS | T336568C | V153A | HT |
| | | (GTG > GCG) | | |

| | | | |
|---|---|---|---|
| Intergenic | A355848G | N/A | FX |

Table 12 shows SNPs in the MT-MuPOP-NL plant, revealed by Illumina sequencing. The mutations are arranged in ascending order according to the locations of identified SNPs on the reference genome (NCBI NC_006581). FX, fixed mutation, variant reads were >90% of all the reads at the indicated locations. HT, heteroplasmic mutation, variant reads were between 40-90% of all the reads at the indicated locations

TABLE 13

| | | SEQ |
|---|---|---|
| Name | Sequence 5′ > 3′ | ID NO |
| B18SHetero-F | ACTACTATACCAAACGCACTCCAC | 94 |
| B18SHetero-R | CAACTACCTAATACTACGCAGGCT | 95 |
| Inter1 Hetro-F | ATTAGTCGAATGAGCCCATCTCTC | 96 |
| Inter1Hetro-R | AGAAGTTTGACTAGTTGCTCCTCC | 97 |

Oligonucleotide primers used to amplify the 18S rDNA and intergenic regions containing heteroplasmic mutations identified by Ilumina sequencing. Sanger sequencing results of the amplified PCR products is shown in FIG. 36

### Example 5

The error-prone version of POP i.e. MutPOP produced herein is a better tool than traditional chemical mutagens such as ethidium bromide (EtBr), ethyl methane sulfonate (EMS), N-nitroso-N-methylurea (NMU) and N-nitroso-N-ethylurea (NEU) for introducing mutations into organelle genomes. The reasons include a) chemical mutagens affect all three genomes (nucleus, chloroplast and mitochondria) in plant cells, while the error-prone POP can be used to selectively mutate chloroplast or mitochondrial genomes using specific organelle targeting N-terminal presequences (as exemplified in Example 2 and Example 4). Alternatively, both chloroplast targeted POP and mitochondrial targeted POP can be combined to mutate both organelle genomes simultaneously without mutating nuclear genes; b) the error-prone POP is a more effective mutator of organelle genomes than chemical mutagens, which can have pleiotropic effects such as impeding growth and regeneration; c) chemical mutagens are extremely hazardous with harmful effects on human users whereas the error-prone POP is not hazardous to humans.

To compare mutagenesis by the error-prone POP with a chemical mutagen, chloroplast mutations in the 16S rDNA gene conferring spectinomycin resistance were scored. Leaf disks from a transgenic line expressing the plastid mutator POP (Nt1) were placed on regeneration media containing 200 mg/L spectinomycin. Leaf disks from wild type (WT) plants were placed on the same regeneration media containing 200 mg/L spectinomycin supplemented with 0.001% (w/v) ethidium bromide (EtBr), which is a typical concentration used to elevate mutation rate. Spectinomycin resistant shoots were scored eight weeks after the start of the assay. The result showed that the leaves expressing the error-prone POP gave rise to spectinomycin resistant shoots at a 51-fold higher frequency than the WT leaves exposed to EtBr (Table 14).

TABLE 14

| | | No. of | | | |
|---|---|---|---|---|---|
| | | spectinomycin | | Shoots | |
| Leaf | Chemical | resistant | No. of leaf | per leaf | Relative |
| Explant | mutagen | shoots | disks | disk | frequency |
| WT | EtBr | 1 | 76 | 0.013 | 1 |
| Plastid | None | 40 | 60 | 0.58 | 51 |
| MuPOP | | | | | |

We also compared the plastid mutation rate for plastid MuPOP with the chemical mutagen N-nitroso-N-methyl urea (NMU) reported in the literature (Fluhr et al. 1985). In both cases, spectinomycin resistance rates were scored by identifying green sectors on otherwise bleached seedlings placed on MS medium containing spectinomycin. Seeds from two plastid mutator POP lines NT1 and NT6 previously described in Example 2 and FIG. 14 were germinated on MS medium containing 200 mg/L spectinomycin. The seedlings showing green sectors on first true leaves from NT1 and NT6 seeds were scored as shown in Table 15. Compared to 5 mM NMU, plastid MuPOP gave rise to spectinomycin resistant seedlings at comparable or higher (4 fold) frequencies than frequencies of NMU as reported in the literature (Fluhr et al. 1985).

TABLE 15

| | | | | No. of | |
|---|---|---|---|---|---|
| | | | | seedlings | |
| | | | | showing | |
| | | | | green | |
| | | | | sectors on | |
| | | [Spectin- | No. of | the | |
| Plant | Mutagenesis | omycin] | germinated | first true | Relative |
| type | method | mg/L | seeds | leaves (%) | Frequency |
| Wild | 5 mM NMU¹ | 50 | 1000 | 12 | 1 |
| type | | | | | |
| NT1 | MuPOP | 200 | 196 | 2 | 1 |
| NT6 | MuPOP | 200 | 134 | 5 | 4 |

1. Fluhr et al. 1985

### REFERENCES

ALTSCHUL, S. F., GISH, W., MILLER, W., MYERS, E. W. & LIPMAN, D. J. 1990. Basic local alignment search tool. J Mol Biol, 215, 403-410.
ASTATKE, M., GRINDLEY, N. D. F. & JOYCE, C. M. 1998. How E. coli DNA polymerase I (Klenow fragment) distinguishes between deoxy- and dideoxynucleotides. J Mol Biol, 278, 147-165.
AYALA-GARCIA, V. M., BARUCH-TORRES, N., GARCIA-MEDEL, P. L. & BRIEBA, L. G. 2018. Plant organellar DNA polymerases paralogs exhibit dissimilar nucleotide incorporation fidelity. FEBS J, 285, 4005-4018.
BARUCH-TORRES, N. & BRIEBA, L. G. 2017. Plant organellar DNA polymerases are replicative and translesion DNA synthesis polymerases. Nucl Acids Res, 45, 10751-10763.
BEBENEK, K., JOYCE, C. M., FITZGERALD, M. P. & KUNKEL, T. A. 1990. The fidelity of DNA synthesis catalyzed by derivatives of Escherichia coli DNA polymerase 1. J Biol Chem, 265, 13878-13887.
BEBENEK, K. & KUNKEL, T. 1995. Analyzing fidelity of DNA polymerases. Meth Enzymol, 262, 217-232.
BELL, C. E., FRESCURA, P., HOCHSCHILD, A. & LEWIS, M. 2000. Crystal structure of the lambda repressor C-terminal domain provides a model for cooperative operator binding, Cell, 101, 801-811.
BEVAN, M. 1984. Binary Agrobacterium vectors for plant transformation. Nucl Acids Res, 12, 8711-8721.
BURR, S. P., PEZET, M. & CHINNERY, P. F. 2018. Mitochondrial DNA heteroplasmy and purifying selection in the mammalian female germ line. Develop Growth Differen, 60, 21-32.
CARRIE, C., KUHN, K., MURCHA, M. W., DUNCAN, O., SMALL, I. D., O'TOOLE, N. & WHELAN, J. 2009. Approaches to defining dual-targeted proteins in Arabidopsis. Plant J, 57, 1128-1139.
CHAN, S. S. L., NAVIAUX, R. K., BASINGER, A. A., CASAS, K. A. & COPELAND, W. C. 2009. De novo mutation in POLG leads to haplotype insufficiency and Alpers syndrome. Mitochondrion, 9, 340-345.
CHRISTENSEN, A. C., LYZNIK, A., MOHAMMED, S., ELOWSKY, C. G., ELO, A., YULE, R. & MACKENZIE, S. A. 2005. Dual-domain, dual-targeting organellar protein presequences in Arabidopsis can use non-AUG start codons. Plant Cell, 17, 2805-2816.
CHRISTIE, J. R. & BEEKMAN, M. 2017. Uniparental inheritance promotes adaptive evolution in cytoplasmic genomes. Ml Bio Evol, 34, 677-691.
CORRIVEAU, J. L. & COLEMAN, A. W 1988. Rapid screening method to detect potential biparental inheritance of plastid DNA and results for over 200 angiosperm species. Amer J Bot, 75, 1443-1458.
CSORGO, B., FEHER, T., TIMAR, E., BLATTNER, F. R. & POSFAI, G. 2012. Low-mutation-rate, reduced-genome Escherichia coli: an improved host for faithful maintenance of engineered genetic constructs. Microb Cell Factories, 11, e11
CUPP, J. D. & NIELSEN, B. L. 2013. Arabidopsis thaliana organellar DNA polymerase IB mutants exhibit reduced mtDNA levels with a decrease in mitochondrial area density. Physiol Plant, 149, 91-103.
DANDEKAR, A. M. and FISK, H. J. (2005). Plant Transformation: Agrobacterium-mediated gene transfer. In Transgenic Plants. New Jersey: Humana Press, pp. 035-046.
DAY, A. (ed.) 2012. Reverse Genetics in Flowering Plant Plastids. Adv. Photosynth. Resp., 35, 415-441. [symbol]
DAYAN, F. E. & DUKE, S. O. 2014. Natural compounds as next-generation herbicides. Plant Physiol, 166, 1090-1105.
DE COSTER, W., D'HERT, S., SCHULTZ, D. T., CRUTS, M. & VAN BROECKHOVEN, C. 2018. NanoPack: visualizing and processing long-read sequencing data. Bioinformatics, 34, 2666-2669.
DELARUE, M., POCH, O., TORDO, N., MORAS, D. & ARGOS, P. 1990. An attempt to unify the structure of polymerases. Protein Engineer, 3, 461-467.
DERBYSHIRE, V., GRINDLEY, N. D. F. & JOYCE, C. M. 1991. The 3-5 exonuclease of DNA polymerase I of Escherichia coli: contribution of each amino-acid at the active-site to the reaction. EMBO J, 10, 17-24.
DORRELL, R. G. & HOWE, C. J. 2012. What makes a chloroplast? Reconstructing the establishment of photosynthetic symbioses. J Cell Sci, 125, 1865-1875.
DRESCHER, A., RUF, S., CALSA, T., CARRER, H. & BOCK, R. 2000. The two largest chloroplast genome-encoded open reading frames of higher plants are essential genes. Plant J, 22, 97-104.
EDGAR, D. & TRIFUNOVIC, A. 2009. The mtDNA mutator mouse: Dissecting mitochondrial involvement in aging. Aging, 1, 1028-1032.
EMANUELSSON, O., BRUNAK, S., VON HELJNE, G. & NIELSEN, H, 2007. Locating proteins in the cell using TargetP, SignalP and related tools. Nature Protocols, 2, 953-971.
ENGLER, C., KANDZIA, R. & MARILLONNET, S. 2008. A one pot, one step, precision cloning method with high throughput capability. Plos One, 3. e3647
FAYZULIN, R. Z., PEREZ, M, KOZHUKHAR, N., SPADAFORA, D., WILSON, G. L. & ALEXEYEV, M. F. 2015. A method for mutagenesis of mouse rntDNA and a resource of mouse mtDNA mutations for modeling human pathological conditions. Nucl Acids Res, 43. e62-e62
FENG, L. Y., RAZA, M. A., L I, Z. C., CHEN, Y. K., BIN KHALID, M. H., DU, J. B., LIU, W G., WU, X. L., SONG, C., YU, L, ZHANG, Z. W., YUAN, S. YANG, W Y. & YANG, F. 2019. The Influence of light Intensity and leaf movement on photosynthesis characteristics and carbon balance of soybean. Front Plant Sci, 9. e1952
FENG, S. H., COKUS, S. J., ZHANG, X. Y., CHEN, P. Y., BOSTICK, M., GOLL, M. G., HETZEL, J., JAIN. J., STRAUSS, S. H., HALPERN, M. E., UKOMADU, C., SADLER, K. C., PRADHAN, S., PELLEGRINI, M. & JACOBSEN, S. E. 2010. Conservation and divergence of methylation patterning in plants and animals. Proc Natl Acad Sci USA, 107, 8689-8694.
FLOROS, V. I., PYLE, A., DIETMANN, S., WEI, W., TANG, W. W. C., IRIE, N., PAYNE, B., CAPALBO, A., NOLI, L., COXHEAD, J., HUDSON, G., CROSIER, M., STRAHL, H., KHALAF, Y, SAITOU, M., ILIC, D., SURANI, M. A. & CHINNERY, P. F. 2018. Segregation of mitochondrial DNA heteroplasmy through a developmental genetic bottleneck in human embryos. Nature Cell Biol, 20, 144-151.
FLUHR, R., AVIV, D., GALUN, E. & EDELMAN, M. 1985. Efficient induction and selection of chloroplast-encoded antibiotic-resistant mutants in Nicotiana. Proc Natl Acad Sci USA, 82, 1485-1489.
FOURY, F. & VANDERSTRAETEN, S. 1992. Yeast mitochondrial DNA mutators with deficient proofreading exonucleolytic activity. EMBO J, 11, 2717-2726.
FRANCOIS, I., VAN HEMELRIJCK, W., AERTS, A. M., WOUTERS, P. F. J., PROOST, P., BROEKAERT, W. F. & CAMMUE, B. P. A. 2004. Processing in Arabidopsis thaliana of a heterologous polyprotein resulting in differential targeting of the individual plant defensins. Plant Sci, 166, 113-121.
GARCIA-MEDEL, P. L., BARUCH-TORRES, N., PERALTA-CASTRO, A., TRASVINA-ARENAS, C. H., TORRES-LARIOS, A. & BRIEBA, L. G. 2019. Plant organellar DNA polymerases repair double-stranded breaks by microhomology-mediated end-joining. Nucl Acids Res, 47, 3028-3044.
GLEAVE, A. P 1992. A versatile binary vector system with a T-DNA organizational-structure conducive to efficient integration of cloned DNA into the plant genome. Plant Mol Biol, 20, 1203-1207.
GREINER, S. 2012. Plastome mutants of higher plants. Adv Photosyn Resp, 35, 237-266.
HAN, C. D., PATRIE, W., POLACCO, M. & COE, E. H. 1993. Aberrations in plastid transcripts and deficiency of plastid DNA in striped and albino mutants in maize. Planta, 191, 552-563.
HENIKOFF, S. & HENIKOFF, J. G. 1992. Amino acid substitution matrices from protein blocks. Proc Natl Acad Sci USA, 89, 10915-10919.
HOLSTERS, M., SILVA, B., VANVLIET, F., GENETELLO, C., DEBLOCK, M., DHAESE, P., DEPICKER, A., INZE, D., ENGLER, G., VILLARROEL, R. VANMONTAGU, M. & SCHELL, J. 1980. The functional-organization of the nopaline A. tumefaciens plasmid pTic58. Plasmid, 3, 212-230.
IAMTHAM, S. & DAY, A. 2000. Removal of antibiotic resistance genes from transgenic tobacco plastids. Nature Biotechnol, 18, 1172-1176.
JOZWIAKOWSKI, S. K. & CONNOLLY, B. A. 2009. Plasmid-based lacZα assay for DNA polymerase fidelity: application to archaeal family-B DNA polymerase. Nucl Acids Res, 37, e102.
KARLIN, S. & ALTSCHUL, S. F. 1990. Methods for assessing the statistical significance of molecular sequence features by using general scoring schemes. Proc Natl Acad Sci USA, 87, 2264-2268.
KAUPPILA, T. E. S., BRATIC, A., JENSEN, M. B., BAGGIO, F., PARTRIDGE, L., JASPER, H., GRONKE, S. & LARSSON, N. G. 2018. Mutations of mitochondrial DNA are not major contributors to aging of fruit flies. Proc Natl Acad Sci USA, 115, E9620-E9629.
KEITH, B. J., JOZWIAKOWSKI, S. K. & CONNOLLY, B. A. 2013. A plasmid-based lacZα gene assay for DNA polymerase fidelity measurement. Anal Biochem, 433, 153-161.
KLUCNIKA, A. & MA, H. 2019. A battle for transmission: the cooperative and selfish animal mitochondrial genomes. Open Biology, 9, 180267.
KUJOTH, G. C., HIONA, A., PUGH, T. D., SOMEYA, S., PANZER, K., WOHLGEMUTH, S. E., HOFER, T., SEO, A. Y., SULLIVAN, R., JOBLING, W. A., MORROW, J. D, VAN REMMEN, H., SEDIVY, J. M., YAMASOBA, T., TANOKURA, M., WEINDRUCH, R., LEEUWENBURGH, C. & PROLLA, T. A. 2005. Mitochondrial DNA mutations, oxidative stress, and apoptosis in mammalian aging. Science, 309, 481-484.
KUMAR, J. K., TABOR, S. & RICHARDSON, C. C. 2001. Role of the C-terminal residue of the DNA polymerase of bacteriophage T7. J Biol Chem, 276, 34905-34912.
KUNKEL, T. A. 1985. The mutational specificity of DNA polymerase beta during in vitro DNA synthesis: production of frameshift, base substitution, and deletion mutations. J Biol Chem, 260, 5787-5796.
KUNKEL, T. A. & ALEXANDER, P. S. 1986. The base substitution fidelity of eucaryotic DNA polymerases: Mispairing frequencies, site preferences, insertion preferences, and base substitution by dislocation. J Biol Chem, 261, 160-166.
KUNKEL, T. A. & BEBENEK, K. 2000. DNA replication fidelity. Ann Rev Biochem, 69, 497-529.
LANDAU, A., LENCINA, F., PACHECO, M. G. & PRINA, A. R. 2016. Plastome Mutations and recombination events in barley chloroplast mutator seedlings. J Hered, 107, 266-273.
LI, H. 2018. Minimap2: pairwise alignment for nucleotide sequences. Bioinformatics, 34, 3094-3100.
LI, X. (2011). Infiltration of Nicotiana benthamiana Protocol for Transient Expression via Agrobacterium. Bio-Protocol, 1(14) Bio-101:e95.
LING, L. L., KEOHAVONG, P., DIAS, C. & THILLY, W. G. 1991. Optimization of the polymerase chain reaction with regard to fidelity: modified T7, Taq, and vent DNA polymerases. Genome Res, 1, 63-69.
LONGLEY, M. J., NGUYEN, D., KUNKEL, T. A. & COPELAND, W. C. 2001. The fidelity of human DNA polymerase gamma with and without exonucleolytic proofreading and the p55 accessory subunit. J Biol Chem, 276, 38555-38562.
LUTZ, K. A. & MALIGA, P. 2008. Plastid genomes in a regenerating tobacco shoot derive from a small number of copies selected through a stochastic process. Plant J, 56, 975-983.
MADESIS, P., OSATHANUNKUL, M., GEORGOPOULOU, U., GISBY, M. F., MUDD, E. A., NIANIOU, I., TSITOURA, P., MAVROMARA, P., TSAFTARIS, A. & DAY, A. 2010. A hepatitis C virus core polypeptide expressed in chloroplasts detects anti-core antibodies in infected human sera. J Biotechnol, 145, 377-386.
MAJERAN, W., FRISO, G., ASAKURA, Y., QU, X., HUANG, M. S., PONNALA, L., WATKINS, K. P., BARKAN, A. & VAN WIJK, K. J. 2012. Nucleoid-Enriched Proteomes in developing plastids and chloroplasts from maize leaves: a new conceptual framework for nucleoid functions. Plant Physiology, 158, 156-189.
MAOR-SHOSHANI, A., REUVEN, N. B., TOMER, G. & LIVNEH, Z. 2000. Highly mutagenic replication by DNA polymerase V (UmuC) provides a mechanistic basis for SOS untargeted mutagenesis. Proc Natl Acad Sci USA, 97, 565-570.
MARECHAL, A., PARENT, J. S., VERONNEAU-LAFORTUNE, F., JOYEUX, A., LANG, B. F & BRISSON, N. 2009. Whirly proteins maintain plastid genome stability in Arabidopsis. Proc Natl Acad Sci USA, 106, 14693-14698.
McINERNEY, P., ADAMS, P. & HADI, M. Z. 2014. Error rate comparison during polymerase chain reaction by DNA polymerase. Mol Biol Int, 2014, 1-8.
MINNICK, D. T., BEBENEK, K., OSHEROFF, W. P., TURNER, R. M., ASTATKE, M., LIU, L. X., KUNKEL, T. A & JOYCE, C. M. 1999. Side chains that influence fidelity at the polymerase active site of Escherichia coli DNA polymerase I (Klenow fragment). J Biol Chem, 274, 3067-3075.
MORI, Y., KIMURA, S., SAOTOME, A, KASAI, N., SAKAGUCHI, N., UCHIYAMA, Y., ISHIBASHI, T., YAMAMOTO, T., CHIKU, H. & SAKAGUCHI, K. 2005. Plastid DNA polymerases from higher plants: Arabidopsis thaliana. Biochem Biophys Res Commun, 334, 43-50.
MORIYAMA, T., TERASAWA, K. & SATO, N. 2011. Conservation of POPs, the plant organellar DNA polymerases, in eukaryotes. Protist, 162, 177-187.
MORLEY, S. A., AHMAD, N. & NIELSEN, B. L 2019. Plant organelle genome replication. Plants-Basel, 8. e358
MURASHIGE, T. & SKOOG, F. 1962. A revised medium for rapid growth and bioassays with tobacco tissue cultures. Physiol Plant., 15, 473-497.
NAGAYA, S., KAWAMURA, K., SHINMYO, A. & KATO, K. 2010. The HSP Terminator of Arabidopsis thaliana increases gene expression in plant cells. Plant Cell Physiol, 51, 328-332.
NEEDLEMAN, S. B. & WUNSCH, C. D. 1970. A general method applicable to search for similarities in amino acid sequence of 2 proteins. J Mol Biol, 48, 443-453.
NILSSON, B., UHLEN, M., JOSEPHSON, S., GATENBECK, S. & PHILIPSON, L. 1983. An improved positive selection plasmid vector constructed by oligonucleotide mediated mutagenesis. Nucl Acids Res, 11, 8019-8030.
ONO, Y., SAKAI, A., TAKECHI, K., TAKIO, S., TAKUSAGAWA, M. & TAKANO, H. 2007. NtPoII-like1 and NtPoII-like2, bacterial DNA polymerase I homologs isolated from BY-2 cultured tobacco cells, encode DNA polymerases engaged in DNA replication in both plastids and mitochondria. Plant Cell Physiol, 48, 1679-1692.
PARENT, J. S., LEPAGE, E. & BRISSON, N. 2011. Divergent roles for the two Pol I-like organelle DNA polymerases of Arabidopsis. Plant Physiol, 156, 254-262.
PARK, C. B. & LARSSON, N. G. 2011. Mitochondrial DNA mutations in disease and aging. J Cell Biol, 193, 809-818.
PEARSON, W. R. & LIPMAN, D. J. 1988. Improved tools for biological sequence comparison. Proc Natl Acad Sci USA, 85, 2444-2448.
POTAPOV, V. & ONG, J. L. 2017. Examining sources of error in PCR by single molecule sequencing. Plos ONE, 12, e0169774-e0169774.
POULTON, J., CHIARATTI, M. R., MEIRELLES, F. V., KENNEDY, S., WELLS, D. & HOLT, I. J. 2010. Transmission of Mitochondrial DNA Diseases and Ways to Prevent Them. Plos Genet, 6, e1001066
PRIMAVESI, L. F., WU, H. X., MUDD, E. A., DAY, A. & JONES, H. D. 2008. Visualisation of plastids in endosperm, pollen and roots of transgenic wheat expressing modified GFP fused to transit peptides from wheat SSU RubisCO, rice FtsZ and maize ferredoxin III proteins. Transgenic Res, 17, 529-543
PRINA, A. R. 1992. A mutator nuclear gene inducing a wide spectrum of cytoplasmically inherited chlorophyll deficiencies in barley. Theor Appl Genet
REIDHAAROLSON, J. F. & SAUER, R. T. 1988. Combinatorial cassette mutagenesis as a probe of the informational content of protein sequences Science, 241, 53-57.
REIDHAAROLSON, J. F. & SAUER, R. T. 1990. Functionally acceptable substitutions in 2 alpha-helical regions of lambda repressor. Proteins, 7, 306-316.
RUF, S., KARCHER, D. & BOCK, R. 2007. Determining the transgene containment level provided by chloroplast transformation. Proc Natl Acad Sci USA, 104, 6998-7002.
SAKAMOTO, W. & TAKAMI, T. 2018. Chloroplast DNA Dynamics: Copy Number, Quality Control and Degradation. Plant Cell Physiol, 59, 1120-1127.
SAMBROOK, J., FRITSCH, E. F. & MANIATIS, T. 1989. Molecular Cloning: a laboratory manual. Cold Spring Harbor, 2nd Edn.
SAMSTAG, C. L., HOEKSTRA, J. G., HUANG, C. H., CHAISSON, M. J., YOULE, R. J., KENNEDY, S. R. & PALLANCK, L. J. 2018. Deleterious mitochondrial DNA point mutations are overrepresented in Drosophila expressing a proofreading-defective DNA polymerase gamma. Plos Genetics, 14, e1007805
SAUER, R. T. 2013. Mutagenic dissection of the sequence determinants of protein folding, recognition, and machine function. Protein Sci, 22, 1675-1687.
SERINO, G. & MALIGA, P. 1997. A negative selection scheme based on the expression of cytosine deaminase in plastids. Plant Journal, 12, 697-701.
SHAVER, J. M., OLDENBURG, D. J. & BENDICH, A. J. 2006. Changes in chloroplast DNA during development in tobacco, Medicago truncatula, pea, and maize. Planta, 224, 72-82.
SHINKAI, A. & LOEB, L. A. 2001. In vivo mutagenesis by Escherichia coli DNA polymerase I: IIe(709) in motif A functions in base selection. J Biol Chem, 276, 46759-46764.
SIERRO, N., BATTEY, J. N. D., OUADI, S., BAKAHER, N., BOVET, L., WILLIG, A., GOEPFERT, S., PEITSCH, M. C. & IVANOV, N. V. 2014. The tobacco genome sequence and its comparison with those of tomato and potato. Nature Comm, 5, e3833.
SMITH, D. R. 2015. Mutation rates in plastid genomes: they are lower than you might think. Genome biology and evolution, 7, 1227-34.
SMITH, T. F. & WATERMAN, M. S. 1981. Identification of common molecular subsequences. Journal of Molecular Biology, 147, 195-197.
STEWART, J. B., FREYER, C., ELSON, J. L., WREDENBERG, A., CANSU, Z., TRIFUNOVIC, A. & LARSSON, N. G. 2008. Strong purifying selection in transmission of mammalian mitochondrial DNA. Plos Biology, 6, 63-71.
STONE, J. E., KISSLING, G. E., LUJAN, S. A., ROGOZIN, I. B., STITH, C. M., BURGERS, P. M. J. & KUNKEL, T. A. 2009. Low-fidelity DNA synthesis by the L979F mutator derivative of Saccharomyces cerevisiae DNA polymerase ζ. Nucl Acids Res, 37, 3774-3787.
SVAB, Z. & MALIGA, P. 1991. Mutation proximal to the transfer RNA binding region of the Nicotiana plastid 16s ribosomal-RNA confers resistance to spectinomycin. Mol Gen Genet, 228, 316-319.
SZCZEPANOWSKA, K. & TRIFUNOVIC, A. 2015. Different faces of mitochondrial DNA mutators. Biochim Biophys Acta-Bioenergetics, 1847, 1362-1372.
TAKEUCHI, R., KIMURA, S., SAOTOME, A. & SAKAGUCHI, K. 2007. Biochemical properties of a plastidial DNA polymerase of rice. Plant Mol Biol, 64, 601-611.
TRIFUNOVIC, A. & LARSSON, N. G. 2008. Mitochondrial dysfunction as a cause of ageing, Journal of Internal Medicine, 263, 167-178.
TRIFUNOVIC, A., WREDENBERG, A., FALKENBERG, M., SPELBRINK, J. N., ROVIO, A. T., BRUDER, C. E., BOHLOOLY Y, M., GIDLOF, S., OLDFORS, A. WIBOM, R., TORNELL, J., JACOBS, H. T. & LARSSON, N. G. 2004. Premature ageing in mice expressing defective mitochondrial DNA polymerase. Nature, 429, 417-423.
TVEIT, H. & KRISTENSEN, T. 2001. Fluorescence-based DNA polymerase assay. Anal Biochem, 289, 96-98.
UDY, D. B. BELCHER, S., WILLIAMS-CARRIER, R., GUALBERTO, J. M. & BARKAN, A. 2012. Effects of reduced chloroplast gene copy number on chloroplast gene expression in maize. Plant Physiol, 160, 1420-1431.
VERMULST, M., BIELAS, J. H., KUJOTH, G. C., LADIGES, W. C., RABINOVITCH, P. S., PROLLA, T. A, & LOEB, L. A. 2007. Mitochondrial point mutations do not limit the natural lifespan of mice. Nature Genetics, 39, 540-543.
VERMULST, M., WANAGAT, J., KUJOTH, G. C., BIELAS, J. H., RABINOVITCH, P. S., PROLLA, T. A. & LOEB, L. A. 2008. DNA deletions and clonal mutations drive premature aging in mitochondrial mutator mice. Nature Genet, 40, 392-394.
VIRDI, K. S., WAMBOLDT, Y., KUNDARIYA, H., LAURIE, J. D., KEREN, I., KUMAR, K. R. S., BLOCK, A., BASSET, G., LUEBKER, S., ELOWSKY, C., DAY, P. M. ROOSE, J. L., BRICKER, T. M., ELTHON, T. & MACKENZIE, S. A. 2016. MSH1 Is a Plant Organellar DNA Binding and Thylakoid Protein under Precise Spatial Regulation to Alter Development. Mol Plant, 9, 245-260.
WALL, M. K., MITCHENALL, L. A. & MAXWELL, A. 2004. Arabidopsis thaliana DNA gyrase is targeted to chloroplasts and mitochondria. Proc Natl Acad Sci USA, 101, 7821-7826.
WANG, H. & HAYS, J. B. 2001. Simple and rapid preparation of gapped plasmid DNA for incorporation of oligomers containing specific DNA lesions. Appl Biochem Biotechnol, 19, 133-140.
WANG S. H, ZHANG, S. M, LIU, H. Y. SUN, N., GAO, S. C., WANG, X. Q. & LIU, Z. (2014). Evolution of chloroplast 16S ribosome RNA dependent spectinomycin resistance and implications for chloroplast transformation. J Plant Studies, 3, 50-57.
WATERHOUSE, A., BERTONI, M., BIENERT, S., STUDER, G., TAURIELLO, G., GUMIENNY, R., HEER, F. T., DE BEER, T. A. P., REMPFER, C., BORDOLI, L., LEPORE, R. & SCHWEDE, T. 2018. SWISS-MODEL: homology modelling of protein structures and complexes. Nucl Acids Res, 46, W296-W303.
WILKINS, H. M., CARL, S. M. & SWERDLOW, R. H. 2014. Cytoplasmic hybrid (cybrid) cell lines as a practical model for mitochondriopathies. Redox Biol, 2, 619-631.
XU, H., DELUCA, S. Z. & O'FARRELL, P. H. 2008. Manipulating the metazoan mitochondrial genome with targeted restriction enzymesu. Science, 321, 575-577.
YIGIT, E., HERNANDEZ, D. I., TRUJILLO, J. T., DIMALANTA, E. & BAILEY, C. D. 2014. Genome and metagenome sequencing: using the human methyl-binding domain to partition genomic DNA derived from plant tissues. Appl Plant Sci, 2, e1400064
YOUNG, N. D. & DEPAMPHILIS, C. W. 2000. Purifying selection detected in the plastid gene matK and flanking ribozyme regions within a group II intron of nonphotosynthetic plants. Mol Biol Evol, 17, 1933-1941.
ZHENG, X. M., WANG, J. R., FENG, L., PANG, H. B., QI, L., LI, J., SUN, Y., QIAO, W. H., ZHANG, L. F., CHENG, Y. L. & YANG, Q. W. 2017. Inferring the evolutionary mechanism of the chloroplast genome size by comparing whole chloroplast genome sequences in seed plants. Scientific Rep., 7, e1555

SEQUENCE INFORMATION

SEQ ID NO: 1 Nicotiana tabacum wild type POP amino acid sequence
MAFLGFSVQS SPFKPTSYLW FSPHSFSSSR SFWASSGKAL HRREDCKTQS
VENASSSLAV LGDSIKQISS HERKLFSSGL QHKIEEDSTY GWIAETNALK
ASKAKSSYNS YKKISAANCN VSASTNRRVK DEFFDVPTEV NTRMMRERIT
SSYSATTCIS GGNLSSKSKP PYNPAGGEKK VVGNWREYEN HLPQVSVGLT
HSRVNGARSV NKVDGSNVSH YKPLSKGSHL NGQLSSKIME PKLEKVNKLR
EGHASDQLRH SVNGTETKVV TVKAKGVIQE RAMNKMEKNV IQAVTADVMN
GAEANAKGVI LERATNKMEK NAIESMATDV VNGTKTRIVN DEGTGVSQVS
LRERLGAMYD KVHIVDNLSA AKEVVRKLTS QYRHLVHACD TEVAKIDVKQ
QTPVDHGEII CFSIYSGPEA DFGDGKSCIW VDVLDGDGKN LLVEFAPFFQ
DPSIRKVWHN YSFDNHVIEN YGFKVSGFHA DTMHMARLWD SSRRTSGGYS
LEALTGDSTV MRDARPVHAE RLFHGEGLFG KISMKTIFGR KKLKKDGTEG
KVTVIPSVEE LQKTERELWI CYSALDSIST LMLYESLKNK LAKRIWTFDG
VRKGSMYEFY EKYWRPFGEL LVQMETEGVL VDRAYLAEIE KVAKAEQQVA
ANRFRNWAAK YCHDAKYMNV GSDTQLRQLF FGGIQNRKNS DESLPYEKEF
KVPNIDKVTE EGKKAPTKFR KIRLHRICDL IDTEMYTASG WPSVSGDALK
ALSGKVSADF DILDEADDNA EEDPETSIDE ALATNNEVPS QEPEVSIYGS
AYNAFGGGQK GIEACHAIAA LCEMCSIGSL ISNFILPLQG QDVSGENGRI
HCSLNINTET GRLSARRPNL QNQPALEKDR YKIRQAFVAA QGNSLIVADY
GQLELRILAH LANCKSMLDA FKAGGDFHSR TAMNMYTHIR EAVENGQVLL
EWHPQPGEEK PPVPLLKDAF GSERRKAKML NFSIAYGKTT IGLARDWKVS
VKEAKETVDR WYRDRKEVSD WQEQRKFEAR EFRRVHTLLG RARWFPSVKN
ATGSVKGHIE RAAINTPVQG SAADVAMCAM LEISKNARLE ELGWKLLLQV
HDEVILEGPE ESENEAMAIV VDCMSKPFGG KNILRVDLSV DSKCAKNWYS
AK
Note that positions in bold are those which may be modified as described herein.
SEQ ID NO: 2 Nicotiana tabacum modified POP amino acid sequence
MAFLGFSVQS SPFKPTSYLW FSPHSFSSSR SFWASSGKAL HRREDCKTQS
VENASSSLAV LGDSIKQISS HERKLFSSGL QHKIEEDSTY GWIAETNALK
ASKAKSSYNS YKKISAANCN VSASTNRRVK DEFFDVPTEV NTRMMRERIT
SSYSATTCIS GGNLSSKSKP PYNPAGGEKK VVGNWREYEN HLPQVSVGLT
HSRVNGARSV NKVDGSNVSH YKPLSKGSHL NGQLSSKIME PKLEKVNKLR
EGHASDQLRH SVNGTETKVV TVKAKGVIQE RAMNKMEKNV IQAVTADVMN
GAEANAKGVI LERATNKMEK NAIESMATDV VNGTKTRIVN DEGTGVSQVS
LRERLGAMYD KVHIVDNLSA AKEVVRKLTS QYRHLVHACA TAVAKIDVKQ
QTPVDHGEII CFSIYSGPEA DFGDGKSCIW VDVLDGDGKN LLVEFAPFFQ
DPSIRKVWHN YSFDNHVIEN YGFKVSGFHA DTMHMARLWD SSRRTSGGYS
LEALTGDSTV MRDARPVHAE RLFHGEGLFG KISMKTIFGR KKLKKDGTEG
KVTVIPSVEE LQKTERELWI CYSALDSIST LMLYESLKNK LAKRIWTFDG
VRKGSMYEFY EKYWRPFGEL LVQMETEGVL VDRAYLAEIE KVAKAEQQVA
ANRFRNWAAK YCHDAKYMNV GSDTQLRQLF FGGIQNRKNS DESLPYEKEF
KVPNIDKVTE EGKKAPTKFR KIRLHRICDL IDTEMYTASG WPSVSGDALK
ALSGKVSADF DILDEADDNA EEDPETSIDE ALATNNEVPS QEPEVSIYGS
AYNAFGGGQK GIEACHAIAA LCEMCSIGSL ISNFILPLQG QDVSGENGRI
HCSLNINTET GRLSARRPNL QNQPALEKDR YKIRQAFVAA QGNSLIVADY
GQFELRILAH LANCKSMLDA FKAGGDFHSR TAMNMYTHIR EAVENGQVLL
EWHPQPGEEK PPVPLLKDAF GSERRKAKML NFSIAYGKTT IGLARDWKVS
VKEAKETVDR WYRDRKEVSD WQEQRKFEAR EFRRVHTLLG RARWFPSVKN
ATGSVKGHIE RAAINTPVQG SAADVAMCAM LEISKNARLE ELGWKLLLQV
HDEVILEGPE ESENEAMAIV VDCMSKPFGG KNILRVDLSV DSKCAKNWYS
AK
Note modified positions D390A, E392A and L903F are in bold and underlined.
SEQ ID NO: 3 Nicotiana tabacum wild type POP nucleotide sequence
ATGGCATTCTTGGGGTTCTCAGTTCAGTCTTCCCCTTTCAAACCCACCTCTTA
TCTTTGGTTTTCCCCTCATTCCTTCTCCTCTTCTCGCTCCTTTTGGGCTTCTTC
TGGTAAAGCCCTCCACAGGAGGGAAGATTGCAAAACTCAGAGTGTAGAAAAT
GCATCGTCCAGCTTAGCAGTGCTTGGCGATTCCATTAAACAAATATCAAGTCA
CGAAAGGAAGTTATTTTCTAGTGGACTACAACATAAAATTGAGGAAGACAGTA
CTTACGGATGGATCGCTGAAACTAATGCTCTTAAAGCTTCTAAAGCTAAAAGC
TCTTATAATAGTTACAAAAAGATTTCAGCAGCAAATTGCAATGTTAGTGCCTCT
ACAAATCGGAGGGTGAAAGATGAATTTTTTGATGTTCCAACTGAAGTCAATAC
GAGAATGATGCGTGAACGCATCACCTCTAGCTATTCTGCCACAACCTGTATTT
CAGGAGGCAACTTATCTTCAAAAAGCAAACCACCTTATAACCCTGCTGGAGG
AGAAAAGAAGGTAGTTGGAAACTGGAGAGAGTATGAAAACCATTTGCCACAA
GTATCTGTAGGTCTTACCCATTCAAGAGTCAATGGAGCTAGGTCAGTAAATAA
GGTTGATGGTTCTAATGTATCTCATTACAAACCCTTATCAAAAGGTTCCCATC
TTAATGGGCAATTATCAAGCAAAATTATGGAACCAAAGTTGGAGAAGGTAAAT
AAACTAAGGGAGGGTCATGCATCTGATCAACTAAGACACAGTGTGAATGGGA
CCGAGACCAAGGTAGTTACTGTCAAAGCAAAAGGTGTTATCCAGGAGCGTGC
TATGAATAAAATGGAGAAGAATGTAATTCAAGCCGTGACAGCTGATGTCATGA
ATGGAGCTGAGGCCAATGCAAAAGGGGTTATCCTGGAGCGTGCAACTAATAA
AATGGAAAAGAATGCAATTGAATCCATGGCAACTGATGTCGTGAATGGAACC
AAGACTAGGATTGTCAATGATGAAGGAACAGGTGTAAGTCAGGTTTCCCTCC
GTGAGAGGCTGGGTGCAATGTATGACAAAGTTCATATAGTTGACAATTTATCA
GCAGCTAAGGAAGTTGTCAGAAAGCTTACAAGTCAGTACAGGCATCTCGTCC
ATGCGTGTGACTACAGACGGTAGCAAAGATTGATGTTAAGCAGCAAACACCT
GTTGATCATGGAGAAATTATATGCTTCAGTATTTATTCTGGACCAGAGGCTGA
TTTTGGTGATGGGAAATCTTGTATCTGGGTAGATGTTCTTGATGGAGATGGC
AAGAACCTCTTAGTTGAATTTGCTCCATTTTTCCAAGACCCATCCATTAGAAA
GGTCTGGCACAATTATAGCTTTGACAACCATGTCATCGAGAACTATGGGTTTA
AAGTATCTGGCTTTCACGCTGACACAATGCACATGGCACGACTTTGGGATTC
CTCTAGGCGAACTTCAGGTGGTTATTCACTAGAGGCACTAACAGGTGATTCC
ACTGTCATGCGTGATGCTAGACCGGTCCATGCTGAAAGGCTGTTCCATGGTG
AAGGTCTGTTTGGTAAAATATCTATGAAAACGATCTTTGGTCGGAAAAAGCTG
AAGAAAGACGGAACTGAGGGTAAAGTTACTGTCATTCCTTCTGTTGAAGAGT
TGCAAAAAACTGAAAGAGAATTATGGATTTGTTATTCTGCATTAGACTCCATA
AGCACATTGATGCTTTACGAGAGTTTGAAGAATAAACTGGCTAAACGGATCT
GGACATTCGATGGAGTTCGTAAAGGATCGATGTATGAGTTTTATGAGAAATAC
TGGCGTCCATTTGGTGAGCTTCTGGTTCAAATGGAAACTGAGGGTGTGCTGG
TTGACCGTGCCTATCTTGCTGAGATTGAGAAAGTGGCTAAAGCTGAGCAGCA
GGTTGCTGCTAATAGATTTCGTAACTGGGCAGCTAAGTACTGTCATGATGCA
AAGTACATGAATGTTGGAAGTGATACACAGTTGCGTCAGCTGTTTTTTGGTG
GCATCCAGAATAGAAAGAATTCTGATGAGAGTCTACCATATGAGAAAGAATTC
AAAGTTCCAAATATCGATAAAGTAACTGAAGAAGGAAAGAAGGCTCCCACCA
AATTTCGTAAAATCCGACTACATAGAATTTGTGATCTTATCGACACTGAGATG
TACACTGCCAGTGGCTGGCCTTCTGTTAGTGGGGATGCTTTGAAGGCTCTCT
CTGGCAAAGTTTCTGCGGACTTTGATATTCTTGATGAAGCGGATGACAATGC
TGAGGAAGATCCTGAAACAAGCATTGATGAAGCTTTAGCTACAAATAATGAAG
TTCCCAGTCAAGAACCAGAAGTTTCTATTTATGGATCAGCGTACAACGCCTTT
GGAGGTGGGCAGAAAGGAATTGAGGCCTGCCATGCCATTGCAGCATTATGT
GAAATGTGCTCCATAGGCTCTTTAATATCCAACTTTATACTCCCATTGCAAGG
CCAAGATGTATCGGGTGAGAATGGACGAATTCATTGTTCCCTGAATATTAACA
CTGAAACCGGGCGCCTGTCTGCGAGGAGACCAAATTTACAGAACCAGCCTG
CTCTGGAGAAAGATAGGTACAAAATTCGTCAAGCTTTTGTAGCTGCACAAGG
GAATTCCTTGATTGTGGCCGACTATGGGCAGTTGGAACTTAGGATTCTTGCA
CATCTTGCCAACTGTAAGAGCATGTTGGATGCTTTCAAAGCTGGTGGAGACT
TTCATTCAAGGACTGCTATGAACATGTATACTCACATCCGTGAAGCCGTTGAA
AATGGTCAGGTACTTCTTGAGTGGCATCCTCAACCAGGTGAAGAAAAACCTC
CGGTTCCTCTTCTAAAGGATGCTTTTGGCTCTGAAAGAAGGAAGGCAAAGAT
GCTCAATTTTTCAATTGCATACGGGAAAACTACAATTGGACTTGCCCGCGATT
GGAAGGTATCCGTAAAGGAAGCTAAGGAAACAGTTGATCGGTGGTATAGAGA
TAGAAAAGAAGTGTCAGATTGGCAGGAACAACGCAAATTTGAAGCACGCGAG
TTCAGACGTGTTCACACACTTCTAGGACGGGCACGCTGGTTCCCATCAGTAA
AAAATGCAACTGGATCTGTAAAAGGTCACATAGAACGAGCTGCTATAAATACT
CCAGTGCAGGGAAGTGCTGCAGATGTTGCTATGTGCGCCATGTTAGAGATAT
CAAAGAATGCACGACTAGAGGAGCTTGGATGGAAATTGCTTTTACAGGTTCA
TGATGAAGTTATTTTGGAAGGGCCGGAAGAATCTGAAAACGAAGCCATGGCA
ATAGTGGTTGACTGCATGTCCAAGCCTTTTGGTGGAAAGAATATTCTCAGAGT
TGACCTATCTGTTGATTCTAAATGTGCCAAAAACTGGTATTCTGCCAAGTAG
SEQ ID NO: 4 Nicotiana tabacum modified POP nucleotide sequence
ATGGCTTCCTCTGTGATTTCCTCTGCAGCTGTTGCTACTCGCACTAATGTGGCTCAAGC
TAGCATGGTTGCACCTTTTAATGGTCTTAAGTCTGCTGTCTCCTTCCCAGTTTCAAGCAA
GCAAAACCTTGACATCACTTCCATTGCTAGCAATGGTGGAAGAGTCCAATGCATGTCCA
GCTTAGCAGTGCTTGGCGATTCCATTAAACAAATATCAAGTCACGAAAGGAAGTTATTTT
CTAGTGGACTACAACATAAAATTGAGGAAGACAGTACTTACGGATGGATCGCTGAAACT
AATGCTCTTAAAGCTTCTAAAGCTAAAAGCTCTTATAATAGTTACAAAAAGATTTCAGCA
GCAAATTGCAATGTTAGTGCCTCTACAAATCGGAGGGTGAAAGATGAATTTTTTGATGTT
CCAACTGAAGTCAATACGAGAATGATGCGTGAACGCATCACCTCTAGCTATTCTGCCAC
AACCTGTATTTCAGGAGGCAACTTATCTTCAAAAAGCAAACCACCTTATAACCCTGCTG
GAGGAGAAAAGAAGGTAGTTGGAAACTGGAGAGAGTATGAAAACCATTTGCCACAAGT
ATCTGTAGGTCTTACCCATTCAAGAGTCAATGGAGCTAGGTCAGTAAATAAGGTTGATG
GTTCTAATGTATCTCATTACAAACCCTTATCAAAAGGTTCCCATCTTAATGGGCAATTAT
CAAGCAAAATTATGGAACCAAAGTTGGAGAAGGTAAATAAACTAAGGGAGGGTCATGCA
TCTGATCAACTAAGACACAGTGTGAATGGGACCGAGACCAAGGTAGTTACTGTCAAAGC
AAAAGGTGTTATCCAGGAGCGTGCTATGAATAAAATGGAGAAGAATGTAATTCAAGCCG
TGACAGCTGATGTCATGAATGGAGCTGAGGCCAATGCAAAAGGGGTTATCCTGGAGCG
TGCAACTAATAAAATGGAAAAGAATGCAATTGAATCCATGGCAACTGATGTCGTGAATG
GAACCAAGACTAGGATTGTCAATGATGAAGGAACAGGTGTAAGTCAGGTTTCCCTCCGT
GAGAGGCTGGGTGCAATGTATGACAAAGTTCATATAGTTGACAATTTATCAGCAGCTAA
GGAAGTTGTCAGAAAGCTTACAAGTCAGTACAGGCATCTCGTCCATGCGTGTGCTACA
GCGGTAGCAAAGATTGATGTTAAGCAGCAAACACCTGTTGATCATGGAGAAATTATATG
CTTCAGTATTTATTCTGGACCAGAGGCTGATTTTGGTGATGGGAAATCTTGTATCTGGG
TAGATGTTCTTGATGGAGATGGCAAGAACCTCTTAGTTGAATTTGCTCCATTTTTCCAAG
ACCCATCCATTAGAAAGGTCTGGCACAATTATAGCTTTGACAACCATGTCATCGAGAAC
TATGGGTTTAAAGTATCTGGCTTTCACGCTGACACAATGCACATGGCACGACTTTGGGA
TTCCTCTAGGCGAACTTCAGGTGGTTATTCACTAGAGGCACTAACAGGTGATTCCACTG
TCATGCGTGATGCTAGACCGGTCCATGCTGAAAGGCTGTTCCATGGTGAAGGTCTGTTT
GGTAAAATATCTATGAAAACGATCTTTGGTCGGAAAAAGCTGAAGAAAGACGGAACTGA
GGGTAAAGTTACTGTCATTCCTTCTGTTGAAGAGTTGCAAAAAACTGAAAGAGAATTATG
GATTTGTTATTCTGCATTAGACTCCATAAGCACATTGATGCTTTACGAGAGTTTGAAGAA
TAAACTGGCTAAACGGATCTGGACATTCGATGGAGTTCGTAAAGGATCGATGTATGAGT
TTTATGAGAAATACTGGCGTCCATTTGGTGAGCTTCTGGTTCAAATGGAAACTGAGGGT
GTGCTGGTTGACCGTGCCTATCTTGCTGAGATTGAGAAAGTGGCTAAAGCTGAGCAGC
AGGTTGCTGCTAATAGATTTCGTAACTGGGCAGCTAAGTACTGTCATGATGCAAAGTAC
ATGAATGTTGGAAGTGATACACAGTTGCGTCAGCTGTTTTTTGGTGGCATCCAGAATAG
AAAGAATTCTGATGAGAGTCTACCATATGAGAAAGAATTCAAAGTTCCAAATATCGATAA
AGTAACTGAAGAAGGAAAGAAGGCTCCCACCAAATTTCGTAAAATCCGACTACATAGAA
TTTGTGATCTTATCGACACTGAGATGTACACTGCCAGTGGCTGGCCTTCTGTTAGTGGG
GATGCTTTGAAGGCTCTCTCTGGCAAAGTTTCTGCGGACTTTGATATTCTTGATGAAGC
GGATGACAATGCTGAGGAAGATCCTGAAACAAGCATTGATGAAGCTTTAGCTACAAATA
ATGAAGTTCCCAGTCAAGAACCAGAAGTTTCTATTTATGGATCAGCGTACAACGCCTTT
GGAGGTGGGCAGAAAGGAATTGAGGCCTGCCATGCCATTGCAGCATTATGTGAAATGT
GCTCCATAGGCTCTTTAATATCCAACTTTATACTCCCATTGCAAGGCCAAGATGTATCGG
GTGAGAATGGACGAATTCATTGTTCCCTGAATATTAACACTGAAACCGGGCGCCTGTCT
GCGAGGAGACCAAATTTACAGAACCAGCCTGCTCTGGAGAAAGATAGGTACAAAATTC
GTCAAGCTTTTGTAGCTGCACAAGGGAATTCCTTGATTGTGGCCGACTATGGGCAGTTC
GAACTTAGGATTCTTGCACATCTTGCCAACTGTAAGAGCATGTTGGATGCTTTCAAAGC
TGGTGGAGACTTTCATTCAAGGACTGCTATGAACATGTATACTCACATCCGTGAAGCCG
TTGAAAATGGTCAGGTACTTCTTGAGTGGCATCCTCAACCAGGTGAAGAAAAACCTCCG
GTTCCTCTTCTAAAGGATGCTTTTGGCTCTGAAAGAAGGAAGGCAAAGATGCTCAATTT
TTCAATTGCATACGGGAAAACTACAATTGGACTTGCCCGCGATTGGAAGGTATCCGTAA
AGGAAGCTAAGGAAACAGTTGATCGGTGGTATAGAGATAGAAAAGAAGTGTCAGATTG
GCAGGAACAACGCAAATTTGAAGCACGCGAGTTCAGACGTGTTCACACACTTCTAGGA
CGGGCACGCTGGTTCCCATCAGTAAAAAATGCAACTGGATCTGTAAAAGGTCACATAGA
ACGAGCTGCTATAAATACTCCAGTGCAGGGAAGTGCTGCAGATGTTGCTATGTGCGCC
ATGTTAGAGATATCAAAGAATGCACGACTAGAGGAGCTTGGATGGAAATTGCTTTTACA
GGTTCATGATGAAGTTATTTTGGAAGGGCCGGAAGAATCTGAAAACGAAGCCATGGCA
ATAGTGGTTGACTGCATGTCCAAGCCTTTTGGTGGAAAGAATATTCTCAGAGTTGACCT
ATCTGTTGATTCTAAATGTGCCAAAAACTGGTATTCTGCCAAGGGATCTGGATCTGGA
TCTTGGTCTCATCCTCAGTTCGAGAAGTGA
Nucleotide substitutions which give rise to amino acid modifications D390A, E392A
and L930F are in bold and underlined.
SEQ ID NO: 5 Linker peptide
GSGSGS
SEQ ID NO: 6 Strep-tag
WSHPQFEK
SEQ ID NO: 7 Maize wild type POP amino acid sequence
MAVAGLAPSP APALLRRFPC SAPPWAPSPF RPRRPRRGRS MMSPFTGARR
QEYSHSSVLG IQDNRALKLP VCVNENLQNG GAQEWADESR RLSLSKAGNS
NGSTHLGSGI FHHEPLEDFK SSNQSLLHSV RQRMAPNSLA NRHVNTELAK
HHAIKHAAVA VSALTSVVND DLKSVKRPKE SEVEVHWSNG PKFHSHIPKI
SEVETSLRFD ENATDGYAKD VNECSPEETV QPSRTRAPSS QESLDARKAL
STIYDKVLVV DNVMSARSVV QLLTTKYRNY IHACDTEVAN INVKQETPVG
HGKVTCFSIY SGTKGAEADF GNGKTCIWVD VLDGGPDVLM EFVPFFEDSS
IRKVWHNYSF DSHVIENYGI KVAGFHADTM HLARLWDSSR RTDGGYSLEG
LTNDHRVMGV VPKELQKIGK RSMKTIFGRK KIKKDGSEGK ITAIEPVEIL
QREDRELWIC YSSLDSMSTL RLYESLKSKL ERKPWTEDGY PRPGGSLYDF
YEEYWCPFGA ILVKMETAGM LVDRAYLSEI EKVAVAQRES AADKERKWAS
KYCPDAKYMN VNSDTQIRQL FFGGIENIYK SGDFLPKSKA IKVPNNETAV
SEGKKVPKYR TIDLFSIVED LKTDIFTASG WPSVSGAALR NLAGKVPSDL
VYSTDDVNDD ECGIHSEISH CDLEDTSSYG TAYDAFGGGK EGKEACHAIA
ALCEICSINS LISNFILPLQ GNHISCKEGR IHCSLNINTE TGRLSARAPN
LQNQPALEKD RYKIRQAFVA APGNTLIVAD YGQLELRILA HLADCKSMLD
AFKAGGDFHS RTAMNMYQHI REAVEEEKVI LEWHPQPGQE KPPVPLLKDA
FGAERRKAKM LNFSIAYGKT AHGLARDWKV SVKEAKDTLK LWYSDRKEVL
AWQMKQKQLA HEKREVYTLL GRSRRFPNMA HATSGQRGHI ERAAINAPVQ
GSAADVAMCA MLEIDRNTRL KELGWTLLLQ VHDEVILEGP SESAELAKSI
VVECMSKPFY GTNILKVDLA VDAKCAQNWY AAK
Note positions which may be modified as described herein are in bold and underlined.
SEQ ID NO: 8 Arabidopsis wild type POPB amino acid sequence
MGVSLRHLSP SSFWVSRRPR VSSSILSELV PRRRILCTRS RLKMCVCSIA
LFRKKVAIIK GNAGYSTATD CGGSHGFHHS GHQRSSSVEF SGEWKLNLGS
KTARMVPPTV KQAGAVSAWR EEVNNKLRGR NREYANNQDD AFGNGSYILK
GFVPKIDDVH SYGNGQNFDY NLKPGTDITT LGRELNGEMQ TNSIRGSVVA
LPSKDIEVGE TTDVTLKPLN SDTTLDNASY KKTATISKVE KCTNLSQVRA
NLKKIYNRVR VVDNVSSAKE TVALLMNQYR NLVHACDTEV SRIDVKTETP
VDHGEMICFS IYCGSEADFG DGKSCIWVDV LGENGRDILA EFKPFFEDSS
IKKVWHNYSF DNHIIRNYGI KLSGFHGDTM HMARLWDSSR RISGGYSLEA
LTSDPKVLGG TETKEEAELF GKISMKKIFG KGKLKKDGSE GKLVIIPPVK
ELQMEDREAW ISYSALDSIS TLKLYESMKK QLQAKKWFLD GKLISKKNMF
DFYQEYWQPF GELLAKMESE GMLVDRDYLA QIEIVAKAEQ EIAVSRERNW
ASKHCPDAKH MNVGSDTQLR QLFFGGISNS CNDEDLPYEK LFKVPNVDKV
IEEGKKRATK FRNIKLHRIS DRPLPTEKFT ASGWPSVSGD TLKALAGKVS
AEYDYMEGVL DTCLEENIGD DDCISLPDEV VETQHVNTSV ESDTSAYGTA
FDAFGGGESG KEACHAIAAL CEVCSIDSLI SNFILPLQGS NVSGKDGRVH
CSLNINTETG RLSARRPNLQ NQPALEKDRY KIRQAFIASP GNSLIVADYG
QLELRILAHL ASCESMKEAF IAGGDFHSRT AMNMYPHIRE AVENGEVLLE
WHPQPGQEKP PVPLLKDAFA SERRKAKMLN FSIAYGKTAI GLSRDWKVSR
EEAQDTVNLW YNDRQEVRKW QELRKKEAIQ KGYVLTLLGR ARKFPEYRSR
AQKNHIERAA INTPVQGSAA DVAMCAMLEI SNNQRLKELG WKLLLQVHDE
VILEGPSESA ENAKDIVVNC MSEPENGKNI LSVDLSVDAK CAQNWYAGK
Note positions D287, E289 , R761, L802 , E803 and N962, which may be modified as
described herein are in bold and underlined.
SEQ ID NO: 9 Arabidopsis wild type POPA amino acid sequence
MAMGVSLTSH NNPLLRHLSP SSSWVSRSSS RLSSSPLPSF LFPCRRTLLQ
RKLASTDGNV GYCTTTVCQG FQHSVHQRSS SVVENGEWEL RSESNKVRMV
PKIIKVGNQT EVAETHQVPG TVSAWREEAN KLRERNGQIA RNLDDNGYEN
GSVPIISSAP SYETSQKIDY EFKPRGTTRS TTATLNKELI GITQSEPVVS
LPRKGLDVGD NMDVNPKGEG IQRPLISDKS SGTANGNKNT VAISKVERST
EPSNVRENLG KIYDKVLIVD NVQAAKDTVA KLVNQFRNHV HSCDTEVSGI
EVKEETPVDH GELICFSIYC GPEADFGNGK SCIWVDVLGE NGREVLAEFK
PYFEDSFIRK VWHNYSFDSH IIRNHGIEIS GFHADTMHMA RLWDSARRIK
GGYSLEALTS DPKVLGGTQT KEEAEFLGKI SMKTIFGKRK LKKDGSEGKI
VVIPPVEELQ REDREAWISY SALDAISTLK LYESMTKKLQ LMDWHLDGKP
VLGRTMLDFY HEFWRPFGEL LVKMEAEGIL VDREYLAEIE KVAKAEQQVA
GSRFRNWASK YCPDAKYMNI GSDTQLRQLF FGGISNSHDE VLPVEKLFKV
PNIDKVIEEG KKTPTKERNI KLHRISDSPL STENFTASGW PSVGGDVLKE
LAGKVSAEYD FMDDVSDISL EEVVEDDDVE TSETQKSKTD DETDTSAYGT
AYVAFGGGER GKEACHAIAS LCEVCSIDSL ISNFILPLQG SNVSGKDGRV
HCSLNINTET GRLSARRPNL QNQPALEKDR YKIRKAFVAS PGNTLVVADY
GQLELRILAH LTGCKSMMEA FKAGGDFHSR TAMNMYPHVR EAVENGQVIL
EWHPEPGEDK PPVPLLKDAF GSERRKAKML NFSIAYGKTA VGLSRDWKVS
TKEAQETVDL WYNDRQEVRK WQEMRKKEAI EDGYVLTLLG RSRRFPASKS
RAQRNHIQRA AINTPVQGSA ADVAMCAMLE ISINQQLKKL GWRLLLQIHD
EVILEGPIES AEIAKDIVVD CMSKPENGRN ILSVDLSVDA KCAQNWYAAK
Note positions D294, E296, R762, L803, E804 and N963 which may be modified as
described herein are in bold and underlined.
SEQ ID NO: 10 Arabidopsis modified POPA amino acid sequence
MAMGVSLTSH NNPLLRHLSP SSSWVSRSSS RLSSSPLPSF LFPCRRTLLQ
RKLASTDGNV GYCTTTVCQG FQHSVHQRSS SVVENGEWEL RSESNKVRMV
PKIIKVGNQT EVAETHQVPG TVSAWREEAN KLRERNGQIA RNLDDNGYEN
GSVPIISSAP SYETSQKIDY EFKPRGTTRS TTATLNKELI GITQSEPVVS
LPRKGLDVGD NMDVNPKGEG IQRPLISDKS SGTANGNKNT VAISKVERST
EPSNVRENLG KIYDKVLIVD NVQAAKDTVA KLVNQFRNHV HSCATAVSGI
EVKEETPVDH GELICFSIYC GPEADFGNGK SCIWVDVLGE NGREVLAEFK
PYFEDSFIRK VWHNYSFDSH IIRNHGIEIS GFHADTMHMA RLWDSARRIK
GGYSLEALTS DPKVLGGTQT KEEAEFLGKI SMKTIFGKRK LKKDGSEGKI
VVIPPVEELQ REDREAWISY SALDAISTLK LYESMTKKLQ LMDWHLDGKP
VLGRTMLDFY HEFWRPFGEL LVKMEAEGIL VDREYLAEIE KVAKAEQQVA
GSRFRNWASK YCPDAKYMNI GSDTQLRQLF FGGISNSHDE VLPVEKLFKV
PNIDKVIEEG KKTPTKERNI KLHRISDSPL STENFTASGW PSVGGDVLKE
LAGKVSAEYD FMDDVSDISL EEVVEDDDVE TSETQKSKTD DETDTSAYGT
AYVAFGGGER GKEACHAIAS LCEVCSIDSL ISNFILPLQG SNVSGKDGRV
HCSLNINTET GRLSARRPNL QNQPALEKDR YKIRKAFVAS PGNTLVVADY
GQFELRILAH LTGCKSMMEA FKAGGDFHSR TAMNMYPHVR EAVENGQVIL
EWHPEPGEDK PPVPLLKDAF GSERRKAKML NFSIAYGKTA VGLSRDWKVS
TKEAQETVDL WYNDRQEVRK WQEMRKKEAI EDGYVLTLLG RSRRFPASKS
RAQRNHIQRA AINTPVQGSA ADVAMCAMLE ISINQQLKKL GWRLLLQIHD
EVILEGPIES AEIAKDIVVD CMSKPENGRN ILSVDLSVDA KCAQNWYAAK
Note modified positions D294A, E296A and L803F are shown in bold and underlined.
SEQ ID NO: 11 Arabidopsis modified POPB amino acid sequence
MGVSLRHLSP SSFWVSRRPR VSSSILSFLV PRRRILCTRS RLKMCVCSIA
LERKKVAIIK GNAGYSTATD CGGSHGFHHS GHQRSSSVEF SGEWKLNLGS
KTARMVPPTV KQAGAVSAWR EEVNNKLRGR NREYANNQDD AFGNGSYILK
GFVPKIDDVH SYGNGQNEDY NLKPGTDITT LGRELNGEMQ TNSIRGSVVA
LPSKDIEVGE TTDVTLKPLN SDTTLDNASY KKTATISKVE KCTNLSQVRA
NLKKIYNRVR VVDNVSSAKE TVALLMNQYR NLVHACATAV SRIDVKTETP
VDHGEMICFS IYCGSEADFG DGKSCIWVDV LGENGRDILA EFKPFFEDSS
IKKVWHNYSF DNHIIRNYGI KLSGFHGDTM HMARLWDSSR RISGGYSLEA
LTSDPKVLGG TETKEEAELF GKISMKKIFG KGKLKKDGSE GKLVIIPPVK
ELQMEDREAW ISYSALDSIS TLKLYESMKK QLQAKKWFLD GKLISKKNMF
DFYQEYWQPF GELLAKMESE GMLVDRDYLA QIEIVAKAEQ EIAVSRERNW
ASKHCPDAKH MNVGSDTQLR QLFFGGISNS CNDEDLPYEK LFKVPNVDKV
IEEGKKRATK FRNIKLHRIS DRPLPTEKFT ASGWPSVSGD TLKALAGKVS
AEYDYMEGVL DTCLEENIGD DDCISLPDEV VETQHVNTSV ESDTSAYGTA
FDAFGGGESG KEACHAIAAL CEVCSIDSLI SNFILPLQGS NVSGKDGRVH
CSLNINTETG RLSARRPNLQ NQPALEKDRY KIRQAFIASP GNSLIVADYG
QFELRILAHL ASCESMKEAF IAGGDFHSRT AMNMYPHIRE AVENGEVLLE
WHPQPGQEKP PVPLLKDAFA SERRKAKMLN FSIAYGKTAI GLSRDWKVSR
EEAQDTVNLW YNDRQEVRKW QELRKKEAIQ KGYVLTLLGR ARKFPEYRSR
AQKNHIERAA INTPVQGSAA DVAMCAMLEI SNNQRLKELG WKLLLQVHDE
VILEGPSESA ENAKDIVVNC MSEPENGKNI LSVDLSVDAK CAQNWYAGK
Note modified positions D287A, E289A and L802F shown in bold and underlined.
SEQ ID NO: 12 Arabidopsis modified POPA nucleotide sequence
ATGGCCATGGGGGTTTCTCTTACTTCCCACAATAACCCTCTTCTTCGCCACTTATCTCCT
TCCTCTTCCTGGGTTTCTCGTTCCTCCTCTCGCCTCTCTTCTTCTCCACTCCCTTCCTTC
CTCTTTCCTTGCCGTCGAACCCTTCTTCAAAGGTTTGCACTTTTTTTCACCTTTCGTGGG
TTTTAGTAAATTTTGAGACTACACTTGATTATCGATTACTATGCTCTGTTCTATGCTAAA
TTTTACTTGATTGTCGTTACTATGCTCTGTTGTATGCTCAATTTTACCTTATTTTTGACT
AATTTGCTCTGCTCAAGCCTCAGTTTTACTTGATTATCGATTAAGCTACTCTGTTATAGG
CTCTGTTTTACTTGATTGTAGATTAATTTGCTCTGTTCGAAGCTCAATTTGTTCTTGAGT
ATGGGATAAAGCTGCTTCGTTCTAGGCTCATTTTGTTCTTGTGGGGTTTGTTTTGTTTTG
CTAGCTATGTTTGTGACTGCTTTTGAATCTTTTCAGGAAACTAGCTTCCACTGATGGAAA
TGTCGGTTATTGTACTACTACAGTTTGTCAGGGATTTCAGCATTCGGTTCATCAACGGTC
GTCTTCTGTTGTGTTTAACGGAGAGTGGGAACTTCGATCGGAATCTAACAAGGTAAGGA
TGGTTCCTAAGATTATCAAGGTTGGAAATCAGACGGAGGTAGCCGAAACTCATCAAGTT
CCTGGAACTGTAAGTGCTTGGAGGGAAGAAGCTAATAAGCTTAGAGAAAGAAATGGTC
AAATTGCTAGAAACCTTGATGACAATGGTTATTTCAATGGCTCTGTCCCAATTATATCTA
GTGCTCCGTCTTATGAAACCTCTCAGAAGATTGATTATGAGTTCAAGCCCAGAGGGACC
ACTAGGTCCACTACTGCTACACTCAATAAGGAATTGATTGGCATTACCCAATCTGAACCT
GTTGTGTCATTGCCAAGGAAGGGTTTAGATGTTGGGGATAATATGGATGTAAATCCTAA
AGGAGAGGGAATACAGAGACCTCTTATAAGCGATAAGTCTTCTGGTACCGCAAATGGAA
ACAAGAATACTGTTGCCATTTCCAAAGTGGAGAGAAGCACTGAACCTTCGAATGTCCGT
GAAAACCTTGGGAAAATATATGACAAGGTTCTCATTGTTGATAATGTGCAAGCTGCGAA
GGATACCGTGGCTAAGCTCGTGAATCAGTTTAGGAATCATGTCCATTCCTGTGCTACAG
CGGTGTGCTACATTTTTCTATTTAAGTTGTCATCCCTGCAGCTCAGAAATATTTATACAAT
TGACAAACTGATTACTCTATGCTTATAGGTGTCCGGGATTGAGGTTAAGGAAGAAACGC
CTGTCGACCACGGTGAATTAATATGTTTCAGTATTTACTGTGGGCCAGAAGCAGATTTT
GGGAATGGAAAATCGTGCATCTGGGTTGATGTTCTTGGTGAAAATGGCAGGGAGGTGT
TGGCCGAGTTTAAACCATATTTTGAAGACTCGTTCATCAGAAAAGTAAGTTATAAACTAC
ACCTTGATTGTTTCTACATATTCTGTGATATTTTCATTTTCTGAGTCCTTCTAAAACTTATT
CCACTTTCTTGTTGTCTTTTTTACTGTGAACATTTTTAGCTATCCTTAAACCCTTACAGGT
TGGCTTTCTTCGTTTCAGGTCTGGCACAACTACAGTTTCGATAGCCACATCATTAGGAA
CCATGGAATTGAGATTTCTGGTTTTCATGCCGACACTATGCACATGGCACGATTATGGG
ATTCTGCACGGCGGATAAAAGGTGGTTATTCGCTTGAAGCGCTTACAAGTGACCCAAAA
GTTCTTGGGGGCACACAGACAAAGGAGGAAGCTGAGTTCCTTGGTAAAATTTCGATGA
AGACTATTTTTGGCAAACGGAAACTGAAAAAAGATGGATCTGAAGGGAAAATCGTTGTC
ATTCCCCCTGTTGAAGAGCTTCAGCGAGAAGATCGAGAGGCTTGGATTTCATACTCTGC
CTTGGATGCGATAAGCACACTAAAGCTTTACGAGAGCATGACAAAGAAACTGCAATTGA
TGGATTGGCATCTTGATGGAAAGCCAGTTTTAGGAAGGACAATGTTGGATTTTTACCAT
GAGTTCTGGCGACCCTTTGGTGAACTTCTTGTAAAAATGGAAGCAGAAGGGATACTTGT
AGATAGAGAGTATCTTGCTGAGATAGAGAAAGTAGCCAAAGCGGAACAACAAGTTGCT
GGTAGTAGGTTTCGTAATTGGGCATCTAAGTATTGCCCCGATGCGAAGTATATGAATAT
TGGCAGTGACACACAATTGCGTCAGCTCTTTTTTGGTGGCATTTCCAACAGGTATCTTT
CTCCTACGTCATCGTTAAATTTCTGCTAAGCATAGGTTTAAATTGAAGTTATTATAGGGA
AAATAGTCATATCTTGGTATCTTAGAGGTTCTTTCTTTATTATAGAGATTTGAGCTTATAA
TCCTCTTATCTCAAAAGTTGTTGAAGACTGTTTTTCTTTCTCTTGGTTCAACCAGAACTG
CATACATAGAAATCTGAGAAGAAATGCCTTTCTTATCAGGTCATTAACAAGAATTACATG
ATTAGTCTCTCAGTGTTACCTCTAATAATTAACTTCTGTTTATTTCTTCCTCAGTAGTCAT
GACGAGGTGCTTCCAGTTGAAAAGCTCTTCAAAGTCCCCAATATTGATAAGGTTATTGA
AGAAGGCAAAAAAACACCGACGAAGTTTCGAAACATCAAACTGCATAGGATAAGTGATA
GCCCACTGTCAACGGAAAATTTTACTGCCAGTGGTTGGCCCTCTGTAGGTGGGGATGT
TTTGAAAGAGTTAGCTGGGAAAGTTTCTGCTGAATACGACTTTATGGATGATGTCTCAG
ATATTTCTCTGGAAGAAGTTGTTGAGGATGATGATGTAGAAACATCAGAAACACAAAAGT
CCAAGACAGATGATGAAACCGACACATCTGCTTATGGAACAGCATATGTTGCATTTGGA
GGGGGTGAAAGGGGAAAGGAGGCATGTCATGCTATTGCCTCATTATGTGAAGTTTGCT
CTATAGACTCTTTGATCTCAAATTTTATTCTTCCGTTACAGGTAATTCTGTTTTCTTAACA
TCACCTTCTGAATTTTGAGCTGCTTTATCTGGTTTACCTTCAGGTTGAAATATGAATTGTT
TTTTTTTTGTCTGACAGGGAAGTAATGTATCAGGCAAAGATGGTCGCGTCCATTGCTCC
CTTAATATCAATACAGAAACTGGGCGCTTATCAGCTAGAAGGCCAAATTTGCAGGTATA
TATCAAAAAGCTTTGTGATATTGTTCAAAACAATATCGTTTGAACCTAAATTAAGTAATAA
ATTTGAGTAGTGAGGCCATTATGTAGCTAAGTTCCTCTTGTTATCTACTGGTAGCATATT
GTTAACAGAGACTCAATTTGATGTGAAATTATGTGATGTGTTACCTTGAGCTAGTACATA
ATTTCATTTCAAATTGGTTTTATTTTTTGGCAACTGTAAGTACACGATTTGTAATTGCATC
TACAATTTGTTACTTAACTTCCTCTTATCTGTCTAGAACCAACCTGCATTGGAGAAGGAT
CGGTACAAGATTCGTAAGGCCTTTGTAGCATCGCCTGGAAATACACTTGTTGTGGCTGA
TTATGGGCAGGTGAAAATATAAAACTCTGGTAGTTGTAGAGACTTTCATAATGGTATTAA
TCTTGTTCACTTGTTTACTGTGATTCTGTTCTATCTTCAGTTCGAACTTAGAATTCTGGCA
CATCTTACTGGTTGTAAAAGCATGATGGAAGCTTTTAAAGCAGGCGGAGATTTCCACTC
AAGGACAGCCATGAATATGTATCCACATGTTCGTGAAGCTGTAGAAAATGGGCAAGTGA
TCCTTGAATGGCATCCAGAACCTGGGGAAGACAAGCCACCTGTGCCGTTGTTAAAGGT
GCGTTCTTCGTTTAAAATTGTACCATAATTCTTGGATTTTGATTCTTATATCACAATGAGG
AAATGCTTTGAATAGCTATAAAAACAAGCGACATCTCTGTTCAATGTTCAAATCCCGTCG
TTGACTCAAAGAACTTATATCTGTGCCCAAATACTTCTAACAGTTTGCGAATATGTGTAA
TACTCAGGATGCCTTTGGTTCAGAGAGAAGAAAAGCCAAGATGCTTAACTTTTCAATTG
CGTATGGGAAAACCGCGGTCGGGCTTTCTAGAGATTGGAAGGTAGTGGAAAAATACTA
TTGAAACTATTAGGTTGATATAAACATGCGTTGATGTGATGAGTCTAAAACTGTTCAAAC
TTTTTTATCATCTAAAGGTCTCCACTAAAGAAGCTCAGGAAACAGTCGATCTCTGGTACA
ATGACAGGCAAGAAGTAAGGAAATGGCAAGAAATGCGTAAGAAAGAAGCTATAGAAGA
TGGGTATGTACTCACTTTACTAGGAAGGTCTCGTAGATTTCCGGCGTCAAAGTCACGTG
CTCAGAGAAATCATATCCAAAGAGCAGCAATCAACACTCCAGTCCAGGTTTCCATTCTA
TTTGTTTACTTTATCATAACACTAATATCCATTCTATTCTCCAATCCCAACTCTGAGCATC
GATGATCCACACTACAGGGAAGTGCAGCTGATGTTGCCATGTGTGCAATGTTGGAAATA
TCGATAAATCAACAGTTGAAGAAGCTTGGTTGGAGATTGCTTCTACAGGTAAACAACAA
CACCCCTCCAAAACAGGACGCAATCTATGAAAAATATAAGCTTTTAATTTTGTAACATAG
TGGTCTCTATTGGATTGGAAATTGAGTAACAAAGGGAACTTGTATTGGAAACAGATTCAT
GATGAAGTAATCTTGGAAGGACCAATTGAATCAGCGGAGATCGCAAAGGACATAGTTGT
GGACTGTATGTCTAAGCCCTTTAACGGAAGGAATATTCTCTCAGTCGATTTATCTGTTGA
TGCAAAATGTGCTCAAAACTGGTATGCTGCCAAATAG
Nucleotide substitutions which give rise to amino acid modifications D294A, E296A
and L803F have been highlighted in bold and underlined.
SEQ ID NO: 13 Arabidopsis modified POPB nucleotide sequence
ATGGGGGTTTCTCTTCGTCACTTGTCTCCTTCCTCTTTCTGGGTTTCGCGTCGTCCTCG
TGTTTCTTCTTCAATTCTTTCTTTTCTTGTCCCTCGCCGTCGAATCCTTTGCACCAGGTT
CACAGCTTTTCTCATCTTATCTGTTCCCTTTTCTCATTTTTGTTCATCGAGAATTAGATTT
ACTCATTCATTAGTCAATTGCTCTGTTCCGTAAAGTTGGTTGTTAGTTTTATATAGTGATG
ATGATTCACGTTTTGAGGTTGTTGAAGAAGCAGGTTAAAAATGTGTGTTTGCTCAATTGC
TCTGTTTCGTAAGTTTGTTGTTAGTTATATAGTGATGATTCGCGTTTTAAGTTTGTTGAAG
AAGCAGGTTAAAAATGTGTTTTGCTGCAATTTGGTTTCAGTTTCTCACTTAGTCTAAATT
GTTCCTGTGTTAAAGTTATTTAGGAGTCTGCTTTTGATTAAGCAGAAGAATGTATTTTAG
TTGAGGTTTGGTTATTATTCTGAGTTTGAAGGTGTTTTTCTTTGTGTTGATATCATTTTTT
GGTGGTTATGATTTGCAGGAAAGTAGCAATCATAAAGGGAAATGCGGGATATTCGACTG
CTACTGATTGTGGTGGTTCTCATGGGTTTCATCATTCAGGTCATCAAAGGTCATCCTCT
GTTGAGTTTAGTGGAGAGTGGAAACTTAATTTGGGATCTAAGACGGCGAGAATGGTTCC
GCCAACTGTGAAACAAGCTGGAGCTGTAAGTGCTTGGAGGGAGGAAGTCAATAATAAG
TTAAGAGGAAGAAATAGAGAATATGCTAACAACCAGGATGATGCTTTTGGTAATGGCAG
TTATATTTTAAAAGGCTTTGTCCCCAAAATAGATGATGTCCATTCTTATGGAAACGGCCA
GAACTTTGATTACAATCTGAAGCCTGGAACTGATATTACCACACTTGGTAGAGAATTGAA
TGGCTTTATGCAAACGAATAGTATAAGAGGATCGGTCGTAGCATTACCAAGCAAGGATA
TTGAGGTTGGGGAAACAACAGACGTGACTCTGAAACCTCTTAACAGTGATACGACTCTT
GACAATGCCAGCTATAAGAAAACAGCAACCATTTCCAAAGTGGAAAAATGCACCAACCT
ATCGCAAGTCCGTGCAAACCTCAAGAAAATATACAATAGAGTTCGTGTTGTTGATAATGT
GTCTAGTGCAAAGGAAACCGTGGCTCTGCTCATGAATCAATATAGGAATCTTGTCCATG
CTTGCGCTACAGCGGTATTGGGTCATGTGTTTGCTAATACATTGTTGTTCACATGGCCT
AGAAATGCTTATGTTCTTGACGTATAGACGCTTCGTTTAATTCTGCTTTTACTCTCTGATA
ATCTATTCTGTAGGTATCCAGGATTGATGTGAAGACTGAAACACCTGTAGACCATGGGG
AGATGATTTGTTTCAGTATCTATTGTGGATCAGAAGCAGATTTTGGAGATGGTAAATCAT
GTATCTGGGTAGATGTGCTTGGCGAAAATGGAAGGGATATCCTGGCTGAGTTTAAGCC
ATTTTTCGAAGACTCATCCATAAAAAAAGTGAGCTAAAACGAAAAACAACTTGCTGCGTT
TCTATTTATTCAGTGATAATTTGGTGTTACGAATCTTGTTGTGTGTTTTCCTGAAAAGATG
ATTGACTACTTCTTCACCTTGGCTCTCTTTTGTTTAGATGGAGGCACTAATGACTTCTGA
ATCATGTTTTAATTTTGTAAATCATTTGAAAATGTTTTCATGATCAAATAACGCTTATAAG
CTTGACCTTTCCTTTTACTTATAGGTATGGCATAACTACAGCTTTGATAACCACATCATC
AGAAATTATGGAATCAAGCTTTCTGGTTTTCATGGTGATACAATGCACATGGCACGATTG
TGGGATTCATCTAGACGGATATCGGGTGGTTATTCGCTTGAAGCACTTACAAGTGACCC
AAAAGTTCTTGGGGGAACTGAGACAAAGGAGGAAGCAGAATTATTTGGTAAAATATCAA
TGAAGAAGATTTTCGGCAAGGGAAAATTGAAAAAAGATGGATCAGAAGGAAAATTGGTG
ATCATTCCTCCTGTTAAAGAGCTACAAATGGAGGATCGAGAAGCTTGGATTTCGTACTC
AGCGTTGGATTCAATAAGCACTCTAAAGCTTTATGAGAGCATGAAGAAGCAACTGCAAG
CGAAGAAATGGTTTCTTGATGGAAAACTAATTTCAAAAAAGAACATGTTTGATTTCTACC
AAGAATATTGGCAACCTTTTGGTGAACTTCTTGCCAAAATGGAATCGGAAGGAATGCTT
GTAGATAGGGATTATTTGGCGCAGATTGAGATTGTAGCCAAAGCAGAACAAGAAATTGC
TGTTTCGAGGTTCCGTAATTGGGCTTCAAAGCATTGTCCAGATGCAAAGCATATGAATG
TTGGCAGTGACACGCAATTGCGACAACTCTTTTTTGGTGGCATTTCTAACAGGTATGCT
TCTTCCAAGTCTTGATTTGAACTGCCTTATTTTTCCATGTATCTTTAGAAGAAACCATGAC
ATTTTGGTATTTGACACGTACCATATCACTTTATTATAAAGAAAACCAAATGTATGAGACT
CTAGTTTAAGAATCTGGTGAAGATTGTAATCTTTTGCCTTGATAAAACACTTGCAGCTCT
CAGATTTAGTTTGTGAATCAAATTTTCTTCCCATTATGATGCAAAACAGGAGTTACATAAT
TTCGTTTCTTAGTGCTTATTTCTGATACTAAGTTTTGTTCATATCTGATACAGTTGTAATG
ATGAGGATCTTCCATATGAAAAACTTTTCAAAGTTCCCAATGTGGATAAGGTGATTGAAG
AAGGAAAAAAGAGAGCCACAAAATTCCGGAATATCAAACTGCATAGGATAAGCGACAGA
CCTTTGCCCACTGAAAAGTTCACTGCCTCAGGCTGGCCCTCTGTTAGTGGAGACACCTT
GAAAGCCTTAGCTGGGAAAGTCTCTGCAGAATATGACTATATGGAGGGCGTTTTAGATA
CTTGTCTAGAAGAAAACATTGGAGATGATGATTGTATTTCGCTACCAGATGAAGTTGTAG
AAACACAACACGTCAATACCTCTGTTGAATCAGACACATCTGCTTATGGAACAGCATTTG
ATGCATTTGGAGGGGGTGAAAGTGGAAAGGAAGCTTGCCATGCTATTGCTGCATTATGT
GAAGTTTGCTCCATTGATTCCTTAATATCAAATTTTATCCTTCCTTTACAGGTATGTTTTT
TCTTTTAAAACAATATTCCCTGGATTTACGAGCTACTTAATTATTTCCATTAGCATATCCT
TCAGGTTATTTTTTTATGGAATTTGTTTTCCTAAATGACAGGGAAGTAACGTGTCAGGAA
AAGATGGTCGTGTCCACTGCTCCCTGAATATCAACACTGAAACTGGACGCTTATCGGCT
AGAAGGCCAAATTTGCAGGTACATGGCTTGAACTTAATTTAAGAGTTAGCTTGAATGGA
GATGCAATCAAATAACTAAGCTTCTCTAATTTCTCTACTGATAACCTTGTAAATTTTTTTG
ATGTTGTGGTATGAAGAGCTAGTATATGCTAATTTTTGGATTGGTGTGCTCAAGGTCAAT
TTTTAGATAGAGGTATAGGACATCTAGAATTTGTTTGTAAAGTATTCCTTTTCTCTGTCTA
GAACCAACCTGCATTGGAGAAAGATCGGTACAAAATCCGTCAGGCCTTCATAGCATCAC
CTGGAAATTCACTTATTGTTGCTGATTATGGACAGGTGAAATGATATTGATATCTACTTTT
CTGATGACTAGTAAGCTAATTCACTTGTTGACTGTGAGGTGATTTTTCTATCTTCAGTTC
GAACTTAGGATTCTGGCACATCTTGCTAGTTGCGAAAGCATGAAGGAAGCTTTCATAGC
TGGTGGAGACTTCCACTCAAGAACAGCCATGAATATGTATCCTCATATTCGTGAAGCTG
TTGAAAATGGTGAAGTGCTCCTTGAATGGCATCCACAACCTGGACAAGAGAAGCCGCC
AGTGCCATTGTTGAAGGTGAATCCTGGTTAAATGTTTTTGAAACAGGGATTTTTAACTCT
TTTATTCATTTTGCTTGTAAGCTAAAAGAATAGTTCTATTAGTGCTGAACTGCAGAGCGG
GACTCTTTCATCCCTTTGGCACTTTAAGATTGCAACAGTCGACTTATACAGACTAGGGAA
ACTAAACTTTGTTCCTGAATTCGTCTAACAGCTGGCAAAATGTTTTCTTACTTATTCAGG
ATGCCTTTGCTTCTGAGAGAAGAAAAGCAAAGATGCTTAACTTCTCGATTGCGTATGGG
AAGACTGCTATTGGGCTGTCTAGAGATTGGAAGGTACTATCAACTAAGCCGTTAAGATT
GATATGAACAAGCTTGATATAAGATAATAAGCTCACAGAAGCCTGTTTTCCATCTTTCAG
GTATCAAGAGAAGAAGCTCAAGATACAGTTAATCTCTGGTATAATGACAGACAAGAAGT
CCGGAAATGGCAAGAACTACGCAAGAAAGAAGCTATACAAAAAGGGTATGTACTCACTT
TGTTGGGAAGGGCTCGTAAATTCCCTGAATACCGTTCACGTGCCCAAAAGAACCATATC
GAGCGAGCAGCAATCAACACTCCTGTTCAGGTTTGTTTGCACTATCATCAGATCTTAAC
CATCTTGAACTCTATCGACTTTAAACCTCAATCACCACTATTAATACAGGGAAGTGCGGC
TGATGTTGCTATGTGCGCTATGCTGGAGATATCAAATAATCAACGGCTAAAAGAGCTTG
GTTGGAAATTGCTTCTACAAGTAATACTTCTCACAGATTCTTTACATAACTACAGGTTAG
ATGATTGTTTATCTAAATGATTTCTTTTGGGCTACCTACAGGTTCATGATGAAGTAATCTT
GGAAGGACCAAGTGAGTCAGCGGAGAACGCTAAAGACATAGTTGTGAACTGCATGTCT
GAACCCTTCAACGGCAAGAATATTCTCTCAGTCGACTTATCTGTTGATGCTAAGTGTGC
TCAGAACTGGTATGCTGGCAAATAA
Nucleotide substitutions which give rise to amino acid modifications D287A, E289A
and L802F have been highlighted in bold and underlined.
SEQ ID NO: 14 Nicotiana tabacum modified POP expression construct
MASSVISSAA VATRINVAQA SMVAPENGLK SAVSFPVSSK QNLDITSIAS
NGGRVQCMSS LAVLGDSIKQ ISSHERKLES SGLQHKIEED STYGWIAETN
ALKASKAKSS YNSYKKISAA NCNVSASTNR RVKDEFFDVP TEVNTRMMRE
RITSSYSATT CISGGNLSSK SKPPYNPAGG EKKVVGNWRE YENHLPQVSV
GLTHSRVNGA RSVNKVDGSN VSHYKPLSKG SHLNGQLSSK IMEPKLEKVN
KLREGHASDQ LRHSVNGTET KVVTVKAKGV IQERAMNKME KNVIQAVTAD
VMNGAEANAK GVILERATNK MEKNAIESMA TDVVNGTKTR IVNDEGTGVS
QVSLRERLGA MYDKVHIVDN LSAAKEVVRK LTSQYRHLVH ACATAVAKID
VKQQTPVDHG EIICFSIYSG PEADFGDGKS CIWVDVLDGD GKNLLVEFAP
FFQDPSIRKV WHNYSEDNHV IENYGFKVSG FHADTMHMAR LWDSSRRTSG
GYSLEALTGD STVMRDARPV HAERLFHGEG LFGKISMKTI FGRKKLKKDG
TEGKVTVIPS VEELQKTERE LWICYSALDS ISTLMLYESL KNKLAKRIWT
FDGVRKGSMY EFYEKYWRPF GELLVQMETE GVLVDRAYLA EIEKVAKAEQ
QVAANRFRNW AAKYCHDAKY MNVGSDTQLR QLFFGGIQNR KNSDESLPYE
KEFKVPNIDK VTEEGKKAPT KFRKIRLHRI CDLIDTEMYT ASGWPSVSGD
ALKALSGKVS ADFDILDEAD DNAEEDPETS IDEALATNNE VPSQEPEVSI
YGSAYNAFGG GQKGIEACHA IAALCEMCSI GSLISNFILP LQGQDVSGEN
GRIHCSLNIN TETGRLSARR PNLQNQPALE KDRYKIRQAF VAAQGNSLIV
ADYGQFELRI LAHLANCKSM LDAFKAGGDF HSRTAMNMYT HIREAVENGQ
VLLEWHPQPG EEKPPVPLLK DAFGSERRKA KMLNFSIAYG KTTIGLARDW
KVSVKEAKET VDRWYRDRKE VSDWQEQRKF EAREFRRVHT LLGRARWFPS
VKNATGSVKG HIERAAINTP VQGSAADVAM CAMLEISKNA RLEELGWKLL
LQVHDEVILE GPEESENEAM AIVVDCMSKP FGGKNILRVD LSVDSKCAKN
WYSAKGSGSG SWSHPQFEK
Italics and underline indicates Petunia Rubisco Small subunit transit peptide. Bold
indicates modified positions. Double underline indicates linker. Bold and underline
indicates Strep tag II
SEQ ID NO: 15 Nicotiana tabacum POP native promoter
CGTTTAAGGAAGCATATCAAGGGTTGTCTTGTGCGTCCTCTAGGAATTCGTATTTAAGTT
GATTTAAGACAATTTGGGTTATTTTTAATTTGTTAAACTTATTTAAGTTTAATGTGTTAATT
TATAAGACAAGTTAATTTGAAGTATTATATGCAATGAAAATATGAAATGAAAGCCTTTGAA
GTTTGATCCTTACATATTGGTCTTATTAAATAATTTTATATGGCCACTTAATTTCAATTTTC
AAATTTAAAACTTTAAATTTTAAAGTTTAAAAGTTTAATTTTATACCTTAAAATACTTATTTT
ATTATTTAAAAATTTGCTAACACTTAAGTAACAATTACATTGAACAAGAAAACATAATACA
CTAAAAAAAAGAAAATTAACCCGTCCCGGTCCGGAACCGATAAGCCCGAACCCGGACG
GACCCACCAAAACCCGGAATATCCCAGCCCACTACCAGCACGGAATCCCAGCCCACTA
ACCAGCTCGCCCCACTAACCGGACGAGCTGTTTTTTCCCATGTCCAGCTCGGTCCAGC
CCGTCCGTTAAACACCCATATTCTAAATAGAGATTGAGTGTTCAATACGTCATGGGGTG
AGTTTAAGTGTCTAGGTGAAAATAGGGACAAGTTTATGAGTTTGTCTGTGTATTTATAAT
TTATAAATATTTATATGGTTATAAATTATTTCATTAAGAATTTTTTTTTTAAGATTAGTTATT
TTTTTGGGTAAGAGGAGAACCCGCATCTGCTACTACCTCTGTCACACCTCTGTCCTTCG
GGTGATCACTCTGCGGCGAATATTTTGTATGCACTGGATAAATTTATCTTATGTAATAAT
CTGCAAATCACACAGAAAATATAAATCGTACTAGGCAAGTCCTGTGCGACATATAACCG
GGAAGGCATTAAGGAGAATCCATCCGATGGATCATGGTTATTGAAAAGGAGTAACTAAC
AAAAAAGAAAAAACACGAGACGACAACATAGTCAAATAAATGTCCGTGCCAGCGAGCAA
AAGGGCTTTTCTTTTTCTTTTTGCTCAGAAGAGAGAGGAAAAAGGGTTTTAAGACGACT
CAGCTACTAATAATACAATAATTGTTCTTTGCTCTTCCGCCCCCTCTCTCTGTTTACAGT
GTAAGTATAGAAGACAGAAGAGTGTACTGTATACATATGGTCTCTGTTCTCTGCTTCTCC
ACTACCACTTCCAGACAAACAACAAATAAACCCTTGTAAAACCCTAAATTTTTCTTACTTT
TACAGTCTCACCCCCTTTAGTTGTTCATTTTATTTTTGTGGTTAAAATCAAAAAATACAAA
ATACACTACACTAATTTATTTATTATAAAGACGGCCGCTCTTTTGAGCAATAAGCTGCTA
GTAAGCAGAGTTGCC
SEQ ID NO: 16 Arabidopsis Heat Shock Protein 70 promoter (AtHSP70-P)
TAGAAGCGATAACAAAATAAAAAGGAAACAATAGTAATTAGATGGCGCAAAAATAAGATC
CAACGGCTGAGATCTTTACTCGTGAACGTTCTCGAAAGCTCTTTGCCGACCCACTCTTC
ATTCATATATAAACAAACACCTCTCTGCCTTCTCTTCCTCACACAATCATAAACACAACAA
CACTCACAAATTCTCTTAAAGCTCACAGACGAATTCTTTCTATTTTTAATCTTTCCGGCGA
ACAATTCTGATCTCTAATA
SEQ ID NO: 17 Arabidopsis Heat Shock Protein 18.2 3′UTR
ATATGAAGATGAAGATGAAATATTTGGTGTGTCAAATAAAAAGCTTGTGTGCTTAAGTTT
GTGTTTTTTTCTTGGCTTGTTGTGTTATGAATTTGTGGCTTTTTCTAATATTAAATGAATG
TAAGATCTCATTATAATGAATAAACAAATGTTTCTATAATCCATTGTGAATGTTTTGTTGG
ATCTCTTCTGCAGCATATAACTACTGTATGTGCTATGGTATGGACTATGGAATATGATTA
AAGATAAGATGGGCTCATAGAGTAAAACGAGGCGAGGGACCTATAAACCTCCCTTCATC
ATGCTATTTCATGATCTATTTTATAAAATAAAGATGTAGAAAAAAGTAAGCGTAATAACCG
CAAAACAAATGATTTAAAACATGGCACATAATGAGGAGATTAAGTTCGGTTTACGTTTAT
TTTAGTACTAATTGTAACGTGAGACTACGTATCGGGAATCGCCTAATTAAAGCATTAATG
CGAACCTGATTAGATTCACCGACCCTCCTATCGTGTCGA
SEQ ID NO: 18 GFP-GUS with chloroplast transit peptide amino acids sequence
MASSVISSAA VATRINVAQA SMVAPENGLK SAVSFPVSRK QNLDITSIAS
NGGRVQCMVS KGEELFTGVV PILVELDGDV NGHKESVSGE GEGDATYGKL
TLKFICTTGK LPVPWPTLVT TFGYGVQCFA RYPDHMKQHD FFKSAMPEGY
VQERTIFFKD DGNYKTRAEV KFEGDTLVNR IELKGIDFKE DGNILGHKLE
YNYNSHNVYI MADKQKNGIK VNFKIRHNIE DGSVQLADHY QQNTPIGDGP
VLLPDNHYLS TQSALSKDPN EKRDHMVLLE FVTAAGITHG MDELYKSNAA
DEVATQLLNF DLLKLAGDVE SNPGPMLRPV ETPTREIKKL DGLWAFSLDR
ENCGIDQRWW ESALQESRAI AVPGSENDQF ADADIRNYAG NVWYQREVFI
PKGWAGQRIV LRFDAVTHYG KVWVNNQEME HQGGYTPFEA DVTPYVIAGK
SVRITVCVNN ELNWQTIPPG MVITDENGKK KQSYFHDFEN YAGIHRSVML
YTTPNTWVDD ITVVTHVAQD CNHASVDWQV VANGDVSVEL RDADQQVVAT
GQGTSGTLQV VNPHLWQPGE GYLYELCVTA KSQTECDIYP LRVGIRSVAV
KGEQFLINHK PFYFTGFGRH EDADLRGKGF DNVLMVHDHA LMDWIGANSY
RTSHYPYAEE MLDWADEHGI VVIDETAAVG FNLSLGIGFE AGNKPKELYS
EEAVNGETQQ AHLQAIKELI ARDKNHPSVV MWSIANEPDT RPQGAREYFA
PLAEATRKLD PTRPITCVNV MFCDAHTDTI SDLEDVLCLN RYYGWYVQSG
DLETAEKVLE KELLAWQEKL HQPIIITEYG VDTLAGLHSM YTDMWSEEYQ
CAWLDMYHRV FDRVSAVVGE QVWNFADFAT SQGILRVGGN KKGIFTRDRK
PKSAAFLLQK RWTGMNFGEK PQQGGKQ
Note underlined sequence indicates Petunia Rubisco Small subunit transit peptide.

Amino Acid Sequences Alignment Between E. coli Poll and NtPOPtom in FIG. 2B:

| | | SEQ |
|---|---|---|
| Enzyme | Motif A sequence | ID NO |
| E. coli Poll | IVSADYSQIELRIMAHL | 69 |
| NtPOPtom | LIVADYGQLELRILAHL | 70 |
| NtPOPtom Exo-L903F | LIVADYGQFELRILAHL | 71 |
| | | SEQ |
| Enzyme | ExoI motif sequence | ID NO |
| E. coli Poll | VFAFDTETDSLDN | 72 |
| NtPOPtom | VHACDTEVAKIDV | 73 |
| NtPOPtom Exo-L903F | VHACATAVAKIDV | 74 |

### Amino Acid Sequences in FIG. 8C:

Upper sequence
(SEQ ID NO: 75)
MASSVISSAAVATRTNVAQASMVAPFNGLKSAVSFPVSSKQNLDITSIASNGGRVQCMSSL
AVL
Lower sequence
(SEQ ID NO: 76)
MAFLGFSVQSSPFKPTSYLWFSPHSFSSSRSFWASSGKALHRREDCKTQSVENASSSLAV
L
Homoplasmic mutations confirmed by Sanger sequencing (Ref. CPgenome NC_001879.2 is
the wild type plastid genome). Nucleotide substitutions are highlighted in bold and
underlined (data from FIG. 21).
A. PG2 rbcL
CPgenome sequence
(SEQ ID NO: 77)
GAAGGTAATGAAATTATTCGCGAGGCTTGCAAATGGAGCCCGGAACTAGCTGCTGC
TTGTGAAGTATGGAAAGAGATCGTATTTAATTTTGCAGCAGTGGA
PG2 rbcL sequences
(SEQ ID NO: 78)
GAAGGTAATGAAATTATTCGCGAGGCTTGCAAATGGAGCCCGGAACTAGCTGCTGC
TTATGAAGTATGGAAAGAGATCGTATTTAATTTTGCAGCAGTGGA
B. W1 ycf3 intron
(SEQ ID NO: 79)
CPgenome sequence
GGTTAATTACATGAGTTTCAAACTGAAATTTGGATGAATAATCCGTTTATTTAGTTTTA
TCTTTTTTCCCAC
W1 ycf3 sequences
(SEQ ID NO: 80)
GGTTAATTACATGAGTTTCAAACTGAAATTTGTATGAATAATCCGTTTATTTAGTTTTA
TCTTTTTTCCCAC
Heteroplasmic mutations confirmed by Sanger sequencing (Ref. CPgenome NC_001879.2
is the wild type plastid genome). Nucleotide substitutions are highlighted in bold and
underlined (data from FIG. 22).
A. W6 psaB
CPgenome sequence
(SEQ ID NO: 81)
GGAAGATTCATTAAACTGTGAAACGTTACCCTGCCATAATGTGATGTGCTTCCAATG
CCAATAAAAAGTAACCCATCCAATAG
W6_psaB sequence
(SEQ ID NO: 82)
GGAAGATTCATTAAACTGTGAAACGTTACCCTGCCATAATGAGATGTGCTTCCAATG
CCAATAAAAAGTAACCCATCCAATAG
B. W6 psbJ
CPgenome sequence
(SEQ ID NO: 83)
AAACCGATTACAAGAATACCAGCTACAGTACCTATTATCCAAAGAGGAATCCTTCCA
GTAGTATCGGCCATTTACCCCACTT
W6 psbJ sequence
(SEQ ID NO: 84)
AAACCGATTACAAGAATACCAGCTACAGTACCTATTATCCAAAGAGGAATCCTTCCA
GAAGTATCGGCCATTTACCCCACTT
C. W6 ndhF
CPgenome sequence
(SEQ ID NO: 85)
CCAATTCCCAAAAAATATAAATTTGTATCAAATTCGAACTAGTAACTAATCCCAACAT
GGAAGTACTGAAAAAACTCATATAA
W6_ndhF sequence
(SEQ ID NO: 86)
CCAATTCCCAAAAAATATAAATTTGTATCAAATTCGAACTGGTAACTAATCCCAACAT
GGAAGTACTGAAAAAACTCATATAA
Nucleotide sequences for c/ repressor alpha 1 and alpha 5 domain used for error rate
calculation in FIG. 26:
Alpha 1
(SEQ ID NO: 87)
ACACAAGAGCAGCTTGAGGACGCACGTCGCCTTAAAGCAATTTATGAA
Alpha 5
(SEQ ID NO: 88)
GAAGAATTCAGCCCTTCAATCGCCAGAGAAATCTACGAGATGTATGAAGCGGTT
Moss Physcomitrella.patens wild type Poll B, NCBI accession No. XP_024364015
SEQ ID NO: 89
MEGSVQRLVIQLYKSAVAAGFRTPCFTSPSFSAPSSRAFMPVHCFLLRIPFSHIRSPSKLVV
APIRPTRPHLLSSSRFTPCKDYSRRLERNYYVGSRILPANGRQSSFWRRNSDVKDDVTAAG
ARKSHEFPSAPESKNGTAEVSYKKAGERRTITVSRPLKWANTLAALSSTQLREAIRLASLDE
KVYDAVMLVKVLGLNDWSRKRKELNFIGGLLRDADPELMEQVLRACEDGDRLGSTSRRLS
LPYFRPHYFNSSSTGGNLSRLWNTSASKNFDSSSKSNLYTAMVGTVSAMQRKPSLSVQHT
REQVQLPNTVNTAPQFRFPQSIQHFKSSPSNFVPPVSRTVKVALQAVSKTKSHLTNGYALP
QLARRFGKLRAQNQNSHLLTSRKTPDVGVTSQAVDSGRGEAWERAAHQLGILVLAKPLSL
SALKTEVPLSEFEPEKSHSSTSIATDRNDYSKSFRLSSSCEKKSEIFKPVIVNERSCKSEIFKP
VIVKERSCKSEIFKPVIVKERSCKSEIFKPVIVNERSCKSEIFKPVIVKERPCKSLNFSDYIPEQ
KSGFSNSAEKIGEENQPSSHADQSESGQIKKTSAKRSITVKKAIVDNSPEESKLRTRSRKKH
VPVQLERKDLGENNVTGDSHVSALDSERSSNMDPYEERRPVSIKSVMIVDSVEKAEMVVE
QLMSEYKNVVHACDTEVAGIDVKKESPVGHGQITCFSIYCGPGADFGYGKNRLWVDVLDG
GDDVLRVFKRYFEDPSIQKVWHNYSFDKHILSRHGIHPQGFYADTMHLARLNDSARRGSK
GGYALEVLSADRKVMDYCSKNFTEEDGSVFVGKKSMKELFGKAKLKKDGTPGKIKVVPPV
DELQRDEELRDAWIHYSTLDAVCTWRLFVSLQHKLSNTPWSVAELRHKGSMYDFYEKYWR
PFGEVLVQMEAYGMLVDYDHLATVEKLARAQQKISVSRFRKWAARYCPNAARMNVGSDA
QIRQFLFGGTANRKDADQALPMERVFSTPNTDGFIEEGKKIAKKTKPMVITGLANHGIKIPVE
TYTSSGWPAVGGAAIRALAGKVSIDYSDIDDDAAEGVLEVDTEPEVSLTSAGVETDHEEDLS
VYGKAYKAFLGGQEGKEACMALAALCEVASINTLLSNFIEPLQGNDIKSVSDGRVHCSLNIN
TETGRLSARRPSLQNQPALEKDRYKIRQAFVAAPGKALVVADYGQLELRLLAHLADCKSMK
AAFIAGGDFHSRTAMNMYPHVREAVEKDRVLLEWEGLEKPPVPLLKDMFGSERRKAKMLN
FSIAYGKTAMGLAKDWNVKLDEAKATVDLWYSDRPEVLAWQKERKQEAHETLRVHTLLGR
ARHLPDINSSNSLLRSHMERAAINTPVQGSAADVAMCAMLEINQNARLRELGWKLLLQVHD
EVILEGPLESAEEAKELVIKSMMYPFNGENILDVELVVDGDYAENWYAAK
Note that positions in bold D691, E693, R1168, L1209, E1210 and N1368 are those which
may be modified.
Nicotiana tabacum modified POP nucleotide sequence for its use in mitochondria
SEQ ID NO: 90
ATGATGATGATGATGAGCCGCAGCGGCGCCAACCGGGTAGCGAACACCGCAATGTTTG
TGGCGAAGGGTTTAAGCGGCGAAGTTGGTGGTTTAAGAGCATTATATGGCGGTGGTGT
GAGGAGTGAGTCCAGCTTAGCAGTGCTTGGCGATTCCATTAAACAAATATCAAGTCACG
AAAGGAAGTTATTTTCTAGTGGACTACAACATAAAATTGAGGAAGACAGTACTTACGGAT
GGATCGCTGAAACTAATGCTCTTAAAGCTTCTAAAGCTAAAAGCTCTTATAATAGTTACA
AAAAGATTTCAGCAGCAAATTGCAATGTTAGTGCCTCTACAAATCGGAGGGTGAAAGAT
GAATTTTTTGATGTTCCAACTGAAGTCAATACGAGAATGATGCGTGAACGCATCACCTCT
AGCTATTCTGCCACAACCTGTATTTCAGGAGGCAACTTATCTTCAAAAAGCAAACCACCT
TATAACCCTGCTGGAGGAGAAAAGAAGGTAGTTGGAAACTGGAGAGAGTATGAAAACC
ATTTGCCACAAGTATCTGTAGGTCTTACCCATTCAAGAGTCAATGGAGCTAGGTCAGTA
AATAAGGTTGATGGTTCTAATGTATCTCATTACAAACCCTTATCAAAAGGTTCCCATCTT
AATGGGCAATTATCAAGCAAAATTATGGAACCAAAGTTGGAGAAGGTAAATAAACTAAG
GGAGGGTCATGCATCTGATCAACTAAGACACAGTGTGAATGGGACCGAGACCAAGGTA
GTTACTGTCAAAGCAAAAGGTGTTATCCAGGAGCGTGCTATGAATAAAATGGAGAAGAA
TGTAATTCAAGCCGTGACAGCTGATGTCATGAATGGAGCTGAGGCCAATGCAAAAGGG
GTTATCCTGGAGCGTGCAACTAATAAAATGGAAAAGAATGCAATTGAATCCATGGCAAC
TGATGTCGTGAATGGAACCAAGACTAGGATTGTCAATGATGAAGGAACAGGTGTAAGTC
AGGTTTCCCTCCGTGAGAGGCTGGGTGCAATGTATGACAAAGTTCATATAGTTGACAAT
TTATCAGCAGCTAAGGAAGTTGTCAGAAAGCTTACAAGTCAGTACAGGCATCTCGTCCA
TGCGTGTGCTACAGCGGTAGCAAAGATTGATGTTAAGCAGCAAACACCTGTTGATCATG
GAGAAATTATATGCTTCAGTATTTATTCTGGACCAGAGGCTGATTTTGGTGATGGGAAAT
CTTGTATCTGGGTAGATGTTCTTGATGGAGATGGCAAGAACCTCTTAGTTGAATTTGCT
CCATTTTTCCAAGACCCATCCATTAGAAAGGTCTGGCACAATTATAGCTTTGACAACCAT
GTCATCGAGAACTATGGGTTTAAAGTATCTGGCTTTCACGCTGACACAATGCACATGGC
ACGACTTTGGGATTCCTCTAGGCGAACTTCAGGTGGTTATTCACTAGAGGCACTAACAG
GTGATTCCACTGTCATGCGTGATGCTAGACCGGTCCATGCTGAAAGGCTGTTCCATGG
TGAAGGTCTGTTTGGTAAAATATCTATGAAAACGATCTTTGGTCGGAAAAAGCTGAAGA
AAGACGGAACTGAGGGTAAAGTTACTGTCATTCCTTCTGTTGAAGAGTTGCAAAAAACT
GAAAGAGAATTATGGATTTGTTATTCTGCATTAGACTCCATAAGCACATTGATGCTTTAC
GAGAGTTTGAAGAATAAACTGGCTAAACGGATCTGGACATTCGATGGAGTTCGTAAAGG
ATCGATGTATGAGTTTTATGAGAAATACTGGCGTCCATTTGGTGAGCTTCTGGTTCAAAT
GGAAACTGAGGGTGTGCTGGTTGACCGTGCCTATCTTGCTGAGATTGAGAAAGTGGCT
AAAGCTGAGCAGCAGGTTGCTGCTAATAGATTTCGTAACTGGGCAGCTAAGTACTGTCA
TGATGCAAAGTACATGAATGTTGGAAGTGATACACAGTTGCGTCAGCTGTTTTTTGGTG
GCATCCAGAATAGAAAGAATTCTGATGAGAGTCTACCATATGAGAAAGAATTCAAAGTT
CCAAATATCGATAAAGTAACTGAAGAAGGAAAGAAGGCTCCCACCAAATTTCGTAAAAT
CCGACTACATAGAATTTGTGATCTTATCGACACTGAGATGTACACTGCCAGTGGCTGGC
CTTCTGTTAGTGGGGATGCTTTGAAGGCTCTCTCTGGCAAAGTTTCTGCGGACTTTGAT
ATTCTTGATGAAGCGGATGACAATGCTGAGGAAGATCCTGAAACAAGCATTGATGAAGC
TTTAGCTACAAATAATGAAGTTCCCAGTCAAGAACCAGAAGTTTCTATTTATGGATCAGC
GTACAACGCCTTTGGAGGTGGGCAGAAAGGAATTGAGGCCTGCCATGCCATTGCAGCA
TTATGTGAAATGTGCTCCATAGGCTCTTTAATATCCAACTTTATACTCCCATTGCAAGGC
CAAGATGTATCGGGTGAGAATGGACGAATTCATTGTTCCCTGAATATTAACACTGAAAC
CGGGCGCCTGTCTGCGAGGAGACCAAATTTACAGAACCAGCCTGCTCTGGAGAAAGAT
AGGTACAAAATTCGTCAAGCTTTTGTAGCTGCACAAGGGAATTCCTTGATTGTGGCCGA
CTATGGGCAGTTCGAACTTAGGATTCTTGCACATCTTGCCAACTGTAAGAGCATGTTGG
ATGCTTTCAAAGCTGGTGGAGACTTTCATTCAAGGACTGCTATGAACATGTATACTCACA
TCCGTGAAGCCGTTGAAAATGGTCAGGTACTTCTTGAGTGGCATCCTCAACCAGGTGAA
GAAAAACCTCCGGTTCCTCTTCTAAAGGATGCTTTTGGCTCTGAAAGAAGGAAGGCAAA
GATGCTCAATTTTTCAATTGCATACGGGAAAACTACAATTGGACTTGCCCGCGATTGGA
AGGTATCCGTAAAGGAAGCTAAGGAAACAGTTGATCGGTGGTATAGAGATAGAAAAGAA
GTGTCAGATTGGCAGGAACAACGCAAATTTGAAGCACGCGAGTTCAGACGTGTTCACA
CACTTCTAGGACGGGCACGCTGGTTCCCATCAGTAAAAAATGCAACTGGATCTGTAAAA
GGTCACATAGAACGAGCTGCTATAAATACTCCAGTGCAGGGAAGTGCTGCAGATGTTG
CTATGTGCGCCATGTTAGAGATATCAAAGAATGCACGACTAGAGGAGCTTGGATGGAAA
TTGCTTTTACAGGTTCATGATGAAGTTATTTTGGAAGGGCCGGAAGAATCTGAAAACGA
AGCCATGGCAATAGTGGTTGACTGCATGTCCAAGCCTTTTGGTGGAAAGAATATTCTCA
GAGTTGACCTATCTGTTGATTCTAAATGTGCCAAAAACTGGTATTCTGCCAAGGGATCT
GGATCTGGATCTTGGTCTCATCCTCAGTTCGAGAAGTGA
Nucleotide substitutions have been highlighted in bold and underlined.
Nicotiana tabacum modified POP expression construct for its use in mitochondria
SEQ ID NO: 91
MMMMMSRSGANRVANTAMFVAKGLSGEVGGLRALYGGGVRSESSLAVLGDSIKQISSHE
RKLFSSGLQHKIEEDSTYGWIAETNALKASKAKSSYNSYKKISAANCNVSASTNRRVKDEFF
DVPTEVNTRMMRERITSSYSATTCISGGNLSSKSKPPYNPAGGEKKVVGNWREYENHLPQ
VSVGLTHSRVNGARSVNKVDGSNVSHYKPLSKGSHLNGQLSSKIMEPKLEKVNKLREGHA
SDQLRHSVNGTETKVVTVKAKGVIQERAMNKMEKNVIQAVTADVMNGAEANAKGVILERAT
NKMEKNAIESMATDVVNGTKTRIVNDEGTGVSQVSLRERLGAMYDKVHIVDNLSAAKEVVR
KLTSQYRHLVHACATAVAKIDVKQQTPVDHGEIICFSIYSGPEADFGDGKSCIWVDVLDGDG
KNLLVEFAPFFQDPSIRKVWHNYSFDNHVIENYGFKVSGFHADTMHMARLWDSSRRTSGG
YSLEALTGDSTVMRDARPVHAERLFHGEGLFGKISMKTIFGRKKLKKDGTEGKVTVIPSVEE
LQKTERELWICYSALDSISTLMLYESLKNKLAKRIWTFDGVRKGSMYEFYEKYWRPFGELLV
QMETEGVLVDRAYLAEIEKVAKAEQQVAANRFRNWAAKYCHDAKYMNVGSDTQLRQLFF
GGIQNRKNSDESLPYEKEFKVPNIDKVTEEGKKAPTKFRKIRLHRICDLIDTEMYTASGWPS
VSGDALKALSGKVSADFDILDEADDNAEEDPETSIDEALATNNEVPSQEPEVSIYGSAYNAF
GGGQKGIEACHAIAALCEMCSIGSLISNFILPLQGQDVSGENGRIHCSLNINTETGRLSARRP
NLQNQPALEKDRYKIRQAFVAAQGNSLIVADYGQFELRILAHLANCKSMLDAFKAGGDFHS
RTAMNMYTHIREAVENGQVLLEWHPQPGEEKPPVPLLKDAFGSERRKAKMLNFSIAYGKTT
IGLARDWKVSVKEAKETVDRWYRDRKEVSDWQEQRKFEAREFRRVHTLLGRARWFPSVK
NATGSVKGHIERAAINTPVQGSAADVAMCAMLEISKNARLEELGWKLLLQVHDEVILEGPEE
SENEAMAIVVDCMSKPFGGKNILRVDLSVDSKCAKNWYSAKGSGSGSWSHPQFEK
Italics and underline indicates soybean (Glycine max) Alternative Oxidase 1 (AOX1)
presequence. Bold indicates modified positions. Double underline indicates linker. Bold
and underline indicates Strep tag II.
mScarlet-I with mitochondrial presequence nucleotide sequence
SEQ ID NO: 92
ATGATGATGATGATGTCTAGGTCAGGAGGTAACAGGGTAGCTAACACAGCAATGTTTGT
GGCAAAGGGTTTGTCAGGTGAAGTTGGTGGTTTGAGAGCATTGTATGGTGGTGGTGTG
AGGAGTGAGTCCATGGTTTCTAAGGGAGAAGCTGTTATTAAGGAATTTATGAGATTTAA
GGTTCATATGGAAGGATCTATGAATGGACATGAATTTGAAATTGAAGGAGAAGGAGAAG
GAAGACCATATGAAGGAACTCAAACTGCTAAGCTTAAGGTTACTAAGGGAGGACCACTT
CCATTTTCTTGGGATATTCTTTCTCCACAATTTATGTATGGATCTAGAGCTTTTATTAAGC
ATCCAGCTGATATTCCAGATTATTATAAGCAATCTTTTCCAGAAGGATTTAAGTGGGAAA
GAGTTATGAATTTTGAAGATGGAGGAGCTGTTACTGTTACTCAAGATACTTCTCTTGAAG
ATGGAACTCTTATTTATAAGGTTAAGCTTAGAGGAACTAATTTTCCACCAGATGGACCAG
TTATGCAAAAGAAGACTATGGGATGGGAAGCTTCTACTGAAAGACTTTATCCAGAAGAT
GGAGTTCTTAAGGGAGATATTAAGATGGCTCTTAGACTTAAGGATGGAGGAAGATATCT
TGCTGATTTTAAGACTACTTATAAGGCTAAGAAGCCAGTTCAAATGCCAGGAGCTTATAA
TGTTGATAGAAAGCTTGATATTACTTCTCATAATGAAGATTATACTGTTGTTGAACAATAT
GAAAGATCTGAAGGAAGACATTCTACTGGAGGAATGGATGAACTTTATAAGTAA
mScarlet-I with mitochondrial presequence amino acid sequence
SEQ ID NO: 93
MMMMMSRSGGNRVANTAMFVAKGLSGEVGGLRALYGGGVRSESMVSKGEAVIKEFMRF
KVHMEGSMNGHEFEIEGEGEGRPYEGTQTAKLKVTKGGPLPFSWDILSPQFMYGSRAFIK
HPADIPDYYKQSFPEGFKWERVMNFEDGGAVTVTQDTSLEDGTLIYKVKLRGTNFPPDGPV
MQKKTMGWEASTERLYPEDGVLKGDIKMALRLKDGGRYLADFKTTYKAKKPVQMPGAYN
VDRKLDITSHNEDYTVVEQYERSEGRHSTGGMDELYK
Italics and underline indicates soybean (Glycine max) Alternative Oxidase 1 (AOX1)
presequence
B18SHetero-F primer
SEQ ID NO: 94
ACTACTATACCAAACGCACTCCAC
B18SHetero-R primer
SEQ ID NO: 95
CAACTACCTAATACTACGCAGGCT
Inter1Hetro-F primer
SEQ ID NO: 96
ATTAGTCGAATGAGCCCATCTCTC
Inter1Hetro-R primer
SEQ ID NO: 97
AGAAGTTTGACTAGTTGCTCCTCC
Nucleotide sequences in Figure 36
A. 18S rDNA
Reference mitochondrial genome from Nicotiana tabacum
(SEQ ID NO: 98)
GAACGCTAGCTATATGCTTAACACATGCAAGTCGAACG
B. Intergenic region
Reference mitochondrial genome from Nicotiana tabacum
(SEQ ID NO: 99)
GCTCTTCAATCGACGATCTACTGCTCCCTCTTAATAGC
cl repressor alpha 1 translation (FIG. 26)
(SEQ ID NO: 100)
TQEQLEDARRLKAIYE
cl repressor alpha 5 domain translation (FIG. 26)
(SEQ ID NO: 101)
EFSPSIAREIYEMYEAV

1. An organellar DNA Polymerase enzyme comprising an amino acid sequence according to SEQ ID NO: 1, or comprising an amino acid sequence having at least 35% identity thereto, or comprising a functional fragment thereof, wherein the amino acid sequence or functional fragment comprises a modification at or corresponding to position L903, and optionally one or more further modifications at the following positions: D390, E392, R862, E904, and N1065 of SEQ ID NO: 1, or positions corresponding thereto.

2. The organellar DNA polymerase according to claim 1, wherein the organellar DNA polymerase is error-prone.

3. The organellar DNA polymerase according to claim 1 or claim 2, having an error rate of between 1×10⁻⁵ and 1×10⁻² mutations per base, preferably between 4×10⁻⁵ and 8×10⁻³ mutations per base, preferably between 1×10⁻⁴ and 1×10⁻² mutations per base, preferably between 3×10⁻⁴ and 8×10⁻³ mutations per base, preferably between 1×10⁻³ and 1×10⁻² mutations per base, preferably between 1×10⁻³ and 8×10⁻³ mutations per base of DNA.

4. The organellar DNA polymerase according to any of claims 1-3, wherein the organellar DNA polymerase is a plant organellar DNA polymerase.

5. The organellar DNA polymerase according to any preceding claim, comprising an amino acid sequence having at least 35%, at least 40%, at least 45%, at least 50%, at least 55%, at least 60%, at least 65%, at least 70% identity, at least 75%, at least 80%, at least 85%, at least 90%, at least 95%, at least 96%, at least 97%, at least 98%, at least 99% identity to SEQ ID NO: 1, or a functional fragment thereof.

6. The organellar DNA polymerase according to any preceding claim, comprising a modification at or corresponding to position L903 and further modifications at the following positions: D390 and E392 of SEQ ID NO: 1, or positions corresponding thereto.

7. The organellar DNA polymerase according to any preceding claim, wherein each modification is an amino acid substitution, preferably a conservative amino acid substitution.

8. The organellar DNA polymerase according to any preceding claim, wherein position L903 or a position corresponding thereto, is substituted with an amino acid selected from Methionine (M), Asparagine, Phenylalanine (F) and Alanine (A).

9. The organellar DNA polymerase according to any preceding claim, wherein the modification at position L903, or a position corresponding thereto, is L903F.

10. The organellar DNA polymerase according to any preceding claim, wherein positions D390 or E392, or positions corresponding thereto, are each independently substituted with an amino acid selected from alanine (A), valine (V), Leucine (L), Isoleucine (I).

11. The organellar DNA polymerase according to any preceding claim, wherein the modification at position D390, or a position corresponding thereto, is D390A.

12. The organellar DNA polymerase according to any preceding claim, wherein the modification at position E392, or a position corresponding thereto, is E392A.

13. The organellar DNA polymerase according to any preceding claim, comprising an amino acid sequence according to SEQ ID NO: 1 wherein the amino acid sequence comprises one or more modifications, wherein the modifications comprise the substitution L903F, and optionally one or more further substitutions selected from the following: D390A and E392A.

14. The organellar DNA polymerase enzyme according to any preceding claim, comprising an amino acid sequence according to SEQ ID NO: 1 wherein the amino acid sequence comprises one or more modifications, wherein the modifications consist of the substitution L903F, and optionally one or more further substitutions selected from the following: D390A, and E392A.

15. The organellar DNA polymerase enzyme according to any preceding claim, comprising an amino acid sequence according to SEQ ID NO: 2, or an amino acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity to SEQ ID NO: 2, or a functional fragment thereof.

16. The organellar DNA polymerase enzyme according to any preceding claim, consisting of an amino acid sequence according to SEQ ID NO: 2

17. An isolated nucleic acid molecule comprising a sequence encoding the organellar DNA polymerase of any of claims 1-16.

18. The isolated nucleic acid molecule according to claim 17, which comprises a nucleic acid sequence according to SEQ ID NO: 4, or a nucleic acid sequence having at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity thereto.

19. The isolated nucleic acid molecule according to claims 17 or 18, which consists of a nucleic acid sequence according to SEQ ID NO: 4.

20. An expression vector comprising the isolated nucleic acid molecule of any of claims 17-19.

21. An organelle comprising the organellar DNA polymerase of any of claims 1-16, the isolated nucleic acid molecule of any of claims 17-19, or the expression vector of claim 20.

22. The organelle according to claim 21, wherein the organelle is a plastid or mitochondrion, preferably a plastid, preferably a chloroplast.

23. A cell comprising the organellar DNA polymerase of any of claims 1-16, the isolated nucleic acid molecule of any of claims 17-19, the expression vector of claim 20, or the organelle of any of claims 21-22.

24. A plant or part thereof comprising the organellar DNA polymerase of any of claims 1-16, the isolated nucleic acid molecule of any of claims 17-19, the expression vector of claim 20, the organelle of any of claims 21-22, or the cell of claim 23.

25. A seed capable of producing a plant or part thereof comprising the organellar DNA polymerase of any of claims 1-16, the isolated nucleic acid molecule of any of claims 17-19, the expression vector of claim 20, the organelle of any of claims 21-22 or the cell of claim 23.

26. A plant produced from the seed of claim 25.

27. A method of modifying a plant or part thereof, comprising:
(a) Introducing the organellar DNA polymerase any of claims 1-16, the isolated nucleic acid molecule of any of claims 17-19, or the expression vector of claim 20 into the plant or part thereof;
(b) Optionally inducing expression of the isolated nucleic acid molecule or expression vector in the plant or part thereof

28. The method according to claim 27, wherein introducing comprises transforming the organellar DNA polymerase any of claims 1-16, the isolated nucleic acid molecule of any of claims 17-19, or the expression vector of claim 20 into the plant or part thereof.

29. The method according to claim 28, wherein transforming the organellar DNA polymerase any of claims 1-16, the isolated nucleic acid molecule of any of claims 17-19, or the expression vector of claim 20 into the plant or part thereof comprises transforming the isolated nucleic acid molecule of any of claims 17-19, or the expression vector of claim 20 into one or more organelles of a plant or part thereof, preferably into one or more plastids of a plant or part thereof, preferably into one or more chloroplasts of a plant or part thereof.

30. A modified plant or part thereof produced by the method of any of claims 27 to 29.

31. A modified plant or part thereof according to claim 30, wherein the plant or part thereof is transgenic.

32. A method of modifying the organelle DNA of a plant or plant part, comprising, expressing in the plant or plant part, an organellar DNA polymerase of any of claims 1-16.

33. The method of claim 32, further comprising a step of introducing the organellar DNA polymerase any of claims 1-16, the isolated nucleic acid molecule of any of claims 17-19, or the expression vector of claim 20 into the plant or part thereof.

34. The method of claim 33, wherein introducing comprises transforming the organellar DNA polymerase any of claims 1-16, the isolated nucleic acid molecule of any of claims 17-19, or the expression vector of claim 20 into the plant or part thereof.

35. The method according to claim 34, wherein transforming the organellar DNA polymerase any of claims 1-16, the isolated nucleic acid molecule of any of claims 17-19, or the expression vector of claim 20 into the plant or part thereof comprises transforming the organellar DNA polymerase any of claims 1-16, the isolated nucleic acid molecule of any of claims 17-19, or the expression vector of claim 20 into one or more organelles of a plant or part thereof, preferably into one or more plastids of a plant or part thereof, preferably into one or more chloroplasts of a plant or part thereof.

36. A method of modifying organelle DNA in vitro or in vivo comprising:
(a) Contacting the organellar DNA polymerase according to claim 1 with organelle DNA under conditions suitable for the organellar DNA polymerase to modify the organelle DNA;
wherein optionally the contacting comprises introducing the organellar DNA polymerase any of claims 1-16, the isolated nucleic acid molecule of any of claims 17-19, or the expression vector of claim 20 into an organelle, and inducing expression thereof in the organelle.

37. The method according claim 36 wherein the method is a method of introducing transversion or transition mutations into organelle DNA

38. The method according to claim 37 wherein the transversion mutations are selected from A-T, A-C, G-T, and G-C, or vice versa.

39. The method according to claim 37 wherein the transition mutations are selected from A-G, and C-T or vice versa.

40. The method according claim 36 wherein the method is a method of introducing A-T transversion mutations, and A-G or C-T transition mutations into organelle DNA, preferably wherein the method is a method of introducing A-T transversion mutations into organelle DNA.

41. The method according to any of claims 36-40 wherein the method of modifying organelle DNA is in vivo.

42. The method according to any of claims 36-41 wherein the organelle is a plant organelle, preferably the organelle is a plastid, preferably a chloroplast.

43. The method according to any of claims 36-42, wherein the method is method of modifying organelle DNA in a plant.

44. A modified organelle comprising modified organelle DNA produced by the method of any of claims 36-43.

45. The modified organelle according to claim 44, wherein the organelle is a plastid or mitochondrion, preferably a plastid, preferably a chloroplast.

46. A plant or plant part comprising the modified organelle of claims 44 or 45.

47. A method of producing a plant having homoplasmic modified organelle DNA comprising;
(a) Introducing an error-prone organellar DNA polymerase, or a nucleic acid molecule encoding said polymerase, into one or more plants, and optionally inducing expression of the nucleic acid molecule in the or each plant, to modify the organelle DNA;
(b) Taking an explant from said plant(s) and culturing one or more shoots therefrom, or generating one or more F1 seedling(s) from said plant(s);
(c) Optionally exposing the or each shoot or seedling to a selection agent which selects for modified organelle DNA, and optionally a further a selection agent which selects for a trait of interest;
(d) Selecting those shoots or seedlings having modified organelle DNA, optionally by selecting those shoots or seedlings having resistance to the selection agent which selects for modified organelle DNA;
(e) Optionally selecting those shoots or seedlings having resistance to the further selection agent;
(f) Regenerating the or each selected shoot or seedling into a plant; and
(g) Optionally repeating steps (b) to (f) one or more times;
wherein the error prone-organellar DNA polymerase modifies the organelle DNA throughout the organelle genome, and is semi-dominant over endogenous organellar DNA polymerase present in the plant(s).

48. The method according to claim 47, wherein the error prone organellar DNA polymerase is the organellar DNA polymerase according to any of claims 1-16.

49. The method according to claims 47 or 48 wherein the organelle DNA is an organelle genome, preferably the method is a method of producing a plant having homoplasmic modified organelle genomes.

50. The method according to claims 47 to 49, wherein the organelle DNA is plastid DNA or mitochondrion DNA, preferably the organelle DNA is chloroplast DNA.

51. The method according to any of claims 47 to 50 wherein the organelle DNA is chloroplast DNA, step (c) is present, and the selection agent which selects for modified organelle DNA is spectinomycin.

52. The method according to any of claims 47-51 wherein the further a selection agent which selects for a trait of interest is a herbicide.

53. The method according to any of claims 47-52 wherein the error prone-organellar DNA polymerase is dominant over endogenous organellar DNA polymerase present in the plant(s).

54. A plant having homoplasmic modified organelle DNA produced by the method of any of claims 47-53.

55. The plant according to claim 54, having homoplasmic modified organelle genomes.


SEQUENCE LISTING (101 sequences)

SEQ ID NO: 1 - protein, 1152 aa, Nicotiana tabacum
MAFLGFSVQSSPFKPTSYLWFSPHSFSSSRSFWASSGKALHRREDCKTQSVENASSSLAVLGDSIKQISSHERKLFSSGLQHKIEEDSTYGWIAETNALKASKAKSSYNSYKKISAANCNVSASTNRRVKDEFFDVPTEVNTRMMRERITSSYSATTCISGGNLSSKSKPPYNPAGGEKKVVGNWREYENHLPQVSVGLTHSRVNGARSVNKVDGSNVSHYKPLSKGSHLNGQLSSKIMEPKLEKVNKLREGHASDQLRHSVNGTETKVVTVKAKGVIQERAMNKMEKNVIQAVTADVMNGAEANAKGVILERATNKMEKNAIESMATDVVNGTKTRIVNDEGTGVSQVSLRERLGAMYDKVHIVDNLSAAKEVVRKLTSQYRHLVHACDTEVAKIDVKQQTPVDHGEIICFSIYSGPEADFGDGKSCIWVDVLDGDGKNLLVEFAPFFQDPSIRKVWHNYSFDNHVIENYGFKVSGFHADTMHMARLWDSSRRTSGGYSLEALTGDSTVMRDARPVHAERLFHGEGLFGKISMKTIFGRKKLKKDGTEGKVTVIPSVEELQKTERELWICYSALDSISTLMLYESLKNKLAKRIWTFDGVRKGSMYEFYEKYWRPFGELLVQMETEGVLVDRAYLAEIEKVAKAEQQVAANRFRNWAAKYCHDAKYMNVGSDTQLRQLFFGGIQNRKNSDESLPYEKEFKVPNIDKVTEEGKKAPTKFRKIRLHRICDLIDTEMYTASGWPSVSGDALKALSGKVSADFDILDEADDNAEEDPETSIDEALATNNEVPSQEPEVSIYGSAYNAFGGGQKGIEACHAIAALCEMCSIGSLISNFILPLQGQDVSGENGRIHCSLNINTETGRLSARRPNLQNQPALEKDRYKIRQAFVAAQGNSLIVADYGQLELRILAHLANCKSMLDAFKAGGDFHSRTAMNMYTHIREAVENGQVLLEWHPQPGEEKPPVPLLKDAFGSERRKAKMLNFSIAYGKTTIGLARDWKVSVKEAKETVDRWYRDRKEVSDWQEQRKFEAREFRRVHTLLGRARWFPSVKNATGSVKGHIERAAINTPVQGSAADVAMCAMLEISKNARLEELGWKLLLQVHDEVILEGPEESENEAMAIVVDCMSKPFGGKNILRVDLSVDSKCAKNWYSAK

SEQ ID NO: 2 - protein, 1152 aa, synthetic construct
MAFLGFSVQSSPFKPTSYLWFSPHSFSSSRSFWASSGKALHRREDCKTQSVENASSSLAVLGDSIKQISSHERKLFSSGLQHKIEEDSTYGWIAETNALKASKAKSSYNSYKKISAANCNVSASTNRRVKDEFFDVPTEVNTRMMRERITSSYSATTCISGGNLSSKSKPPYNPAGGEKKVVGNWREYENHLPQVSVGLTHSRVNGARSVNKVDGSNVSHYKPLSKGSHLNGQLSSKIMEPKLEKVNKLREGHASDQLRHSVNGTETKVVTVKAKGVIQERAMNKMEKNVIQAVTADVMNGAEANAKGVILERATNKMEKNAIESMATDVVNGTKTRIVNDEGTGVSQVSLRERLGAMYDKVHIVDNLSAAKEVVRKLTSQYRHLVHACATAVAKIDVKQQTPVDHGEIICFSIYSGPEADFGDGKSCIWVDVLDGDGKNLLVEFAPFFQDPSIRKVWHNYSFDNHVIENYGFKVSGFHADTMHMARLWDSSRRTSGGYSLEALTGDSTVMRDARPVHAERLFHGEGLFGKISMKTIFGRKKLKKDGTEGKVTVIPSVEELQKTERELWICYSALDSISTLMLYESLKNKLAKRIWTFDGVRKGSMYEFYEKYWRPFGELLVQMETEGVLVDRAYLAEIEKVAKAEQQVAANRFRNWAAKYCHDAKYMNVGSDTQLRQLFFGGIQNRKNSDESLPYEKEFKVPNIDKVTEEGKKAPTKFRKIRLHRICDLIDTEMYTASGWPSVSGDALKALSGKVSADFDILDEADDNAEEDPETSIDEALATNNEVPSQEPEVSIYGSAYNAFGGGQKGIEACHAIAALCEMCSIGSLISNFILPLQGQDVSGENGRIHCSLNINTETGRLSARRPNLQNQPALEKDRYKIRQAFVAAQGNSLIVADYGQFELRILAHLANCKSMLDAFKAGGDFHSRTAMNMYTHIREAVENGQVLLEWHPQPGEEKPPVPLLKDAFGSERRKAKMLNFSIAYGKTTIGLARDWKVSVKEAKETVDRWYRDRKEVSDWQEQRKFEAREFRRVHTLLGRARWFPSVKNATGSVKGHIERAAINTPVQGSAADVAMCAMLEISKNARLEELGWKLLLQVHDEVILEGPEESENEAMAIVVDCMSKPFGGKNILRVDLSVDSKCAKNWYSAK

SEQ ID NO: 3 - DNA, 3461 bp, Nicotiana tabacum
  source 1..3461: mol_type = genomic DNA
atggcattcttggggttctcagttcagtcttcccctttcaaacccacctcttatctttggttttcccctcattccttctcctcttctcgctccttttgggcttcttctggtaaagccctccacaggagggaagattgcaaaactcagagtgtagaaaatgcatcgtccagcttagcagtgcttggcgattccattaaacaaatatcaagtcacgaaaggaagttattttctagtggactacaacataaaattgaggaagacagtacttacggatggatcgctgaaactaatgctcttaaagcttctaaagctaaaagctcttataatagttacaaaaagatttcagcagcaaattgcaatgttagtgcctctacaaatcggagggtgaaagatgaattttttgatgttccaactgaagtcaatacgagaatgatgcgtgaacgcatcacctctagctattctgccacaacctgtatttcaggaggcaacttatcttcaaaaagcaaaccaccttataaccctgctggaggagaaaagaaggtagttggaaactggagagagtatgaaaaccatttgccacaagtatctgtaggtcttacccattcaagagtcaatggagctaggtcagtaaataaggttgatggttctaatgtatctcattacaaacccttatcaaaaggttcccatcttaatgggcaattatcaagcaaaattatggaaccaaagttggagaaggtaaataaactaagggagggtcatgcatctgatcaactaagacacagtgtgaatgggaccgagaccaaggtagttactgtcaaagcaaaaggtgttatccaggagcgtgctatgaataaaatggagaagaatgtaattcaagccgtgacagctgatgtcatgaatggagctgaggccaatgcaaaaggggttatcctggagcgtgcaactaataaaatggaaaagaatgcaattgaatccatggcaactgatgtcgtgaatggaaccaagactaggattgtcaatgatgaaggaacaggtgtaagtcaggtttccctccgtgagaggctgggtgcaatgtatgacaaagttcatatagttgacaatttatcagcagctaaggaagttgtcagaaagcttacaagtcagtacaggcatctcgtccatgcgtgtgactacagacggtagcaaagattgatgttaagcagcaaacacctgttgatcatggagaaattatatgcttcagtatttattctggaccagaggctgattttggtgatgggaaatcttgtatctgggtagatgttcttgatggagatggcaagaacctcttagttgaatttgctccatttttccaagacccatccattagaaaggtctggcacaattatagctttgacaaccatgtcatcgagaactatgggtttaaagtatctggctttcacgctgacacaatgcacatggcacgactttgggattcctctaggcgaacttcaggtggttattcactagaggcactaacaggtgattccactgtcatgcgtgatgctagaccggtccatgctgaaaggctgttccatggtgaaggtctgtttggtaaaatatctatgaaaacgatctttggtcggaaaaagctgaagaaagacggaactgagggtaaagttactgtcattccttctgttgaagagttgcaaaaaactgaaagagaattatggatttgttattctgcattagactccataagcacattgatgctttacgagagtttgaagaataaactggctaaacggatctggacattcgatggagttcgtaaaggatcgatgtatgagttttatgagaaatactggcgtccatttggtgagcttctggttcaaatggaaactgagggtgtgctggttgaccgtgcctatcttgctgagattgagaaagtggctaaagctgagcagcaggttgctgctaatagatttcgtaactgggcagctaagtactgtcatgatgcaaagtacatgaatgttggaagtgatacacagttgcgtcagctgttttttggtggcatccagaatagaaagaattctgatgagagtctaccatatgagaaagaattcaaagttccaaatatcgataaagtaactgaagaaggaaagaaggctcccaccaaatttcgtaaaatccgactacatagaatttgtgatcttatcgacactgagatgtacactgccagtggctggccttctgttagtggggatgctttgaaggctctctctggcaaagtttctgcggactttgatattcttgatgaagcggatgacaatgctgaggaagatcctgaaacaagcattgatgaagctttagctacaaataatgaagttcccagtcaagaaccagaagtttctatttatggatcagcgtacaacgcctttggaggtgggcagaaaggaattgaggcctgccatgccattgcagcattatgtgaaatgtgctccataggctctttaatatccaactttatactcccattgcaaggccaagatgtatcgggtgagaatggacgaattcattgttccctgaatattaacactgaaaccgggcgcctgtctgcgaggagaccaaatttacagaaccagcctgctctggagaaagataggtacaaaattcgtcaagcttttgtagctgcacaagggaattccttgattgtggccgactatgggcagttggaacttaggattcttgcacatcttgccaactgtaagagcatgttggatgctttcaaagctggtggagactttcattcaaggactgctatgaacatgtatactcacatccgtgaagccgttgaaaatggtcaggtacttcttgagtggcatcctcaaccaggtgaagaaaaacctccggttcctcttctaaaggatgcttttggctctgaaagaaggaaggcaaagatgctcaatttttcaattgcatacgggaaaactacaattggacttgcccgcgattggaaggtatccgtaaaggaagctaaggaaacagttgatcggtggtatagagatagaaaagaagtgtcagattggcaggaacaacgcaaatttgaagcacgcgagttcagacgtgttcacacacttctaggacgggcacgctggttcccatcagtaaaaaatgcaactggatctgtaaaaggtcacatagaacgagctgctataaatactccagtgcagggaagtgctgcagatgttgctatgtgcgccatgttagagatatcaaagaatgcacgactagaggagcttggatggaaattgcttttacaggttcatgatgaagttattttggaagggccggaagaatctgaaaacgaagccatggcaatagtggttgactgcatgtccaagccttttggtggaaagaatattctcagagttgacctatctgttgattctaaatgtgccaaaaactggtattctgccaagtag

SEQ ID NO: 4 - DNA, 3510 bp, synthetic construct
  misc_feature 1..3510: note = Nicotiana tabacum modified POP sequence
  source 1..3510: mol_type = other DNA
atggcttcctctgtgatttcctctgcagctgttgctactcgcactaatgtggctcaagctagcatggttgcaccttttaatggtcttaagtctgctgtctccttcccagtttcaagcaagcaaaaccttgacatcacttccattgctagcaatggtggaagagtccaatgcatgtccagcttagcagtgcttggcgattccattaaacaaatatcaagtcacgaaaggaagttattttctagtggactacaacataaaattgaggaagacagtacttacggatggatcgctgaaactaatgctcttaaagcttctaaagctaaaagctcttataatagttacaaaaagatttcagcagcaaattgcaatgttagtgcctctacaaatcggagggtgaaagatgaattttttgatgttccaactgaagtcaatacgagaatgatgcgtgaacgcatcacctctagctattctgccacaacctgtatttcaggaggcaacttatcttcaaaaagcaaaccaccttataaccctgctggaggagaaaagaaggtagttggaaactggagagagtatgaaaaccatttgccacaagtatctgtaggtcttacccattcaagagtcaatggagctaggtcagtaaataaggttgatggttctaatgtatctcattacaaacccttatcaaaaggttcccatcttaatgggcaattatcaagcaaaattatggaaccaaagttggagaaggtaaataaactaagggagggtcatgcatctgatcaactaagacacagtgtgaatgggaccgagaccaaggtagttactgtcaaagcaaaaggtgttatccaggagcgtgctatgaataaaatggagaagaatgtaattcaagccgtgacagctgatgtcatgaatggagctgaggccaatgcaaaaggggttatcctggagcgtgcaactaataaaatggaaaagaatgcaattgaatccatggcaactgatgtcgtgaatggaaccaagactaggattgtcaatgatgaaggaacaggtgtaagtcaggtttccctccgtgagaggctgggtgcaatgtatgacaaagttcatatagttgacaatttatcagcagctaaggaagttgtcagaaagcttacaagtcagtacaggcatctcgtccatgcgtgtgctacagcggtagcaaagattgatgttaagcagcaaacacctgttgatcatggagaaattatatgcttcagtatttattctggaccagaggctgattttggtgatgggaaatcttgtatctgggtagatgttcttgatggagatggcaagaacctcttagttgaatttgctccatttttccaagacccatccattagaaaggtctggcacaattatagctttgacaaccatgtcatcgagaactatgggtttaaagtatctggctttcacgctgacacaatgcacatggcacgactttgggattcctctaggcgaacttcaggtggttattcactagaggcactaacaggtgattccactgtcatgcgtgatgctagaccggtccatgctgaaaggctgttccatggtgaaggtctgtttggtaaaatatctatgaaaacgatctttggtcggaaaaagctgaagaaagacggaactgagggtaaagttactgtcattccttctgttgaagagttgcaaaaaactgaaagagaattatggatttgttattctgcattagactccataagcacattgatgctttacgagagtttgaagaataaactggctaaacggatctggacattcgatggagttcgtaaaggatcgatgtatgagttttatgagaaatactggcgtccatttggtgagcttctggttcaaatggaaactgagggtgtgctggttgaccgtgcctatcttgctgagattgagaaagtggctaaagctgagcagcaggttgctgctaatagatttcgtaactgggcagctaagtactgtcatgatgcaaagtacatgaatgttggaagtgatacacagttgcgtcagctgttttttggtggcatccagaatagaaagaattctgatgagagtctaccatatgagaaagaattcaaagttccaaatatcgataaagtaactgaagaaggaaagaaggctcccaccaaatttcgtaaaatccgactacatagaatttgtgatcttatcgacactgagatgtacactgccagtggctggccttctgttagtggggatgctttgaaggctctctctggcaaagtttctgcggactttgatattcttgatgaagcggatgacaatgctgaggaagatcctgaaacaagcattgatgaagctttagctacaaataatgaagttcccagtcaagaaccagaagtttctatttatggatcagcgtacaacgcctttggaggtgggcagaaaggaattgaggcctgccatgccattgcagcattatgtgaaatgtgctccataggctctttaatatccaactttatactcccattgcaaggccaagatgtatcgggtgagaatggacgaattcattgttccctgaatattaacactgaaaccgggcgcctgtctgcgaggagaccaaatttacagaaccagcctgctctggagaaagataggtacaaaattcgtcaagcttttgtagctgcacaagggaattccttgattgtggccgactatgggcagttcgaacttaggattcttgcacatcttgccaactgtaagagcatgttggatgctttcaaagctggtggagactttcattcaaggactgctatgaacatgtatactcacatccgtgaagccgttgaaaatggtcaggtacttcttgagtggcatcctcaaccaggtgaagaaaaacctccggttcctcttctaaaggatgcttttggctctgaaagaaggaaggcaaagatgctcaatttttcaattgcatacgggaaaactacaattggacttgcccgcgattggaaggtatccgtaaaggaagctaaggaaacagttgatcggtggtatagagatagaaaagaagtgtcagattggcaggaacaacgcaaatttgaagcacgcgagttcagacgtgttcacacacttctaggacgggcacgctggttcccatcagtaaaaaatgcaactggatctgtaaaaggtcacatagaacgagctgctataaatactccagtgcagggaagtgctgcagatgttgctatgtgcgccatgttagagatatcaaagaatgcacgactagaggagcttggatggaaattgcttttacaggttcatgatgaagttattttggaagggccggaagaatctgaaaacgaagccatggcaatagtggttgactgcatgtccaagccttttggtggaaagaatattctcagagttgacctatctgttgattctaaatgtgccaaaaactggtattctgccaagggatctggatctggatcttggtctcatcctcagttcgagaagtga

SEQ ID NO: 5 - protein, 6 aa, synthetic construct
  REGION 1..6: note = linker peptide
GSGSGS

SEQ ID NO: 6 - protein, 8 aa, synthetic construct
  REGION 1..8: note = Strep-tag
WSHPQFEK

SEQ ID NO: 7 - protein, 1033 aa, Zea mays
MAVAGLAPSPAPALLRRFPCSAPPWAPSPFRPRRPRRGRSMMSPFTGARRQEYSHSSVLGIQDNRALKLPVCVNFNLQNGGAQEWADESRRLSLSKAGNSNGSTHLGSGIFHHEPLEDFKSSNQSLLHSVRQRMAPNSLANRHVNTELAKHHAIKHAAVAVSALTSVVNDDLKSVKRPKESEVEVHWSNGPKFHSHIPKISEVETSLRFDENATDGYAKDVNECSPEETVQPSRTRAPSSQESLDARKALSTIYDKVLVVDNVMSARSVVQLLTTKYRNYIHACDTEVANINVKQETPVGHGKVTCFSIYSGTKGAEADFGNGKTCIWVDVLDGGPDVLMEFVPFFEDSSIRKVWHNYSFDSHVIENYGIKVAGFHADTMHLARLWDSSRRTDGGYSLEGLTNDHRVMGVVPKELQKIGKRSMKTIFGRKKIKKDGSEGKITAIEPVEILQREDRELWICYSSLDSMSTLRLYESLKSKLERKPWTFDGYPRPGGSLYDFYEEYWCPFGAILVKMETAGMLVDRAYLSEIEKVAVAQRESAADKFRKWASKYCPDAKYMNVNSDTQIRQLFFGGIENIYKSGDFLPKSKAIKVPNNETAVSEGKKVPKYRTIDLFSIVEDLKTDIFTASGWPSVSGAALRNLAGKVPSDLVYSTDDVNDDECGIHSEISHCDLEDTSSYGTAYDAFGGGKEGKEACHAIAALCEICSINSLISNFILPLQGNHISCKEGRIHCSLNINTETGRLSARAPNLQNQPALEKDRYKIRQAFVAAPGNTLIVADYGQLELRILAHLADCKSMLDAFKAGGDFHSRTAMNMYQHIREAVEEEKVILEWHPQPGQEKPPVPLLKDAFGAERRKAKMLNFSIAYGKTAHGLARDWKVSVKEAKDTLKLWYSDRKEVLAWQMKQKQLAHEKREVYTLLGRSRRFPNMAHATSGQRGHIERAAINAPVQGSAADVAMCAMLEIDRNTRLKELGWTLLLQVHDEVILEGPSESAELAKSIVVECMSKPFYGTNILKVDLAVDAKCAQNWYAAK

SEQ ID NO: 8 - protein, 1049 aa, Arabidopsis thaliana
MGVSLRHLSPSSFWVSRRPRVSSSILSFLVPRRRILCTRSRLKMCVCSIALFRKKVAIIKGNAGYSTATDCGGSHGFHHSGHQRSSSVEFSGEWKLNLGSKTARMVPPTVKQAGAVSAWREEVNNKLRGRNREYANNQDDAFGNGSYILKGFVPKIDDVHSYGNGQNFDYNLKPGTDITTLGRELNGFMQTNSIRGSVVALPSKDIEVGETTDVTLKPLNSDTTLDNASYKKTATISKVEKCTNLSQVRANLKKIYNRVRVVDNVSSAKETVALLMNQYRNLVHACDTEVSRIDVKTETPVDHGEMICFSIYCGSEADFGDGKSCIWVDVLGENGRDILAEFKPFFEDSSIKKVWHNYSFDNHIIRNYGIKLSGFHGDTMHMARLWDSSRRISGGYSLEALTSDPKVLGGTETKEEAELFGKISMKKIFGKGKLKKDGSEGKLVIIPPVKELQMEDREAWISYSALDSISTLKLYESMKKQLQAKKWFLDGKLISKKNMFDFYQEYWQPFGELLAKMESEGMLVDRDYLAQIEIVAKAEQEIAVSRFRNWASKHCPDAKHMNVGSDTQLRQLFFGGISNSCNDEDLPYEKLFKVPNVDKVIEEGKKRATKFRNIKLHRISDRPLPTEKFTASGWPSVSGDTLKALAGKVSAEYDYMEGVLDTCLEENIGDDDCISLPDEVVETQHVNTSVESDTSAYGTAFDAFGGGESGKEACHAIAALCEVCSIDSLISNFILPLQGSNVSGKDGRVHCSLNINTETGRLSARRPNLQNQPALEKDRYKIRQAFIASPGNSLIVADYGQLELRILAHLASCESMKEAFIAGGDFHSRTAMNMYPHIREAVENGEVLLEWHPQPGQEKPPVPLLKDAFASERRKAKMLNFSIAYGKTAIGLSRDWKVSREEAQDTVNLWYNDRQEVRKWQELRKKEAIQKGYVLTLLGRARKFPEYRSRAQKNHIERAAINTPVQGSAADVAMCAMLEISNNQRLKELGWKLLLQVHDEVILEGPSESAENAKDIVVNCMSEPFNGKNILSVDLSVDAKCAQNWYAGK

SEQ ID NO: 9 - protein, 1050 aa, Arabidopsis thaliana
MAMGVSLTSHNNPLLRHLSPSSSWVSRSSSRLSSSPLPSFLFPCRRTLLQRKLASTDGNVGYCTTTVCQGFQHSVHQRSSSVVFNGEWELRSESNKVRMVPKIIKVGNQTEVAETHQVPGTVSAWREEANKLRERNGQIARNLDDNGYFNGSVPIISSAPSYETSQKIDYEFKPRGTTRSTTATLNKELIGITQSEPVVSLPRKGLDVGDNMDVNPKGEGIQRPLISDKSSGTANGNKNTVAISKVERSTEPSNVRENLGKIYDKVLIVDNVQAAKDTVAKLVNQFRNHVHSCDTEVSGIEVKEETPVDHGELICFSIYCGPEADFGNGKSCIWVDVLGENGREVLAEFKPYFEDSFIRKVWHNYSFDSHIIRNHGIEISGFHADTMHMARLWDSARRIKGGYSLEALTSDPKVLGGTQTKEEAEFLGKISMKTIFGKRKLKKDGSEGKIVVIPPVEELQREDREAWISYSALDAISTLKLYESMTKKLQLMDWHLDGKPVLGRTMLDFYHEFWRPFGELLVKMEAEGILVDREYLAEIEKVAKAEQQVAGSRFRNWASKYCPDAKYMNIGSDTQLRQLFFGGISNSHDEVLPVEKLFKVPNIDKVIEEGKKTPTKFRNIKLHRISDSPLSTENFTASGWPSVGGDVLKELAGKVSAEYDFMDDVSDISLEEVVEDDDVETSETQKSKTDDETDTSAYGTAYVAFGGGERGKEACHAIASLCEVCSIDSLISNFILPLQGSNVSGKDGRVHCSLNINTETGRLSARRPNLQNQPALEKDRYKIRKAFVASPGNTLVVADYGQLELRILAHLTGCKSMMEAFKAGGDFHSRTAMNMYPHVREAVENGQVILEWHPEPGEDKPPVPLLKDAFGSERRKAKMLNFSIAYGKTAVGLSRDWKVSTKEAQETVDLWYNDRQEVRKWQEMRKKEAIEDGYVLTLLGRSRRFPASKSRAQRNHIQRAAINTPVQGSAADVAMCAMLEISINQQLKKLGWRLLLQIHDEVILEGPIESAEIAKDIVVDCMSKPFNGRNILSVDLSVDAKCAQNWYAAK

SEQ ID NO: 10 - protein, 1050 aa, synthetic construct
  REGION 1..1050: note = Arabidopsis modified POPA sequence
MAMGVSLTSHNNPLLRHLSPSSSWVSRSSSRLSSSPLPSFLFPCRRTLLQRKLASTDGNVGYCTTTVCQGFQHSVHQRSSSVVFNGEWELRSESNKVRMVPKIIKVGNQTEVAETHQVPGTVSAWREEANKLRERNGQIARNLDDNGYFNGSVPIISSAPSYETSQKIDYEFKPRGTTRSTTATLNKELIGITQSEPVVSLPRKGLDVGDNMDVNPKGEGIQRPLISDKSSGTANGNKNTVAISKVERSTEPSNVRENLGKIYDKVLIVDNVQAAKDTVAKLVNQFRNHVHSCATAVSGIEVKEETPVDHGELICFSIYCGPEADFGNGKSCIWVDVLGENGREVLAEFKPYFEDSFIRKVWHNYSFDSHIIRNHGIEISGFHADTMHMARLWDSARRIKGGYSLEALTSDPKVLGGTQTKEEAEFLGKISMKTIFGKRKLKKDGSEGKIVVIPPVEELQREDREAWISYSALDAISTLKLYESMTKKLQLMDWHLDGKPVLGRTMLDFYHEFWRPFGELLVKMEAEGILVDREYLAEIEKVAKAEQQVAGSRFRNWASKYCPDAKYMNIGSDTQLRQLFFGGISNSHDEVLPVEKLFKVPNIDKVIEEGKKTPTKFRNIKLHRISDSPLSTENFTASGWPSVGGDVLKELAGKVSAEYDFMDDVSDISLEEVVEDDDVETSETQKSKTDDETDTSAYGTAYVAFGGGERGKEACHAIASLCEVCSIDSLISNFILPLQGSNVSGKDGRVHCSLNINTETGRLSARRPNLQNQPALEKDRYKIRKAFVASPGNTLVVADYGQFELRILAHLTGCKSMMEAFKAGGDFHSRTAMNMYPHVREAVENGQVILEWHPEPGEDKPPVPLLKDAFGSERRKAKMLNFSIAYGKTAVGLSRDWKVSTKEAQETVDLWYNDRQEVRKWQEMRKKEAIEDGYVLTLLGRSRRFPASKSRAQRNHIQRAAINTPVQGSAADVAMCAMLEISINQQLKKLGWRLLLQIHDEVILEGPIESAEIAKDIVVDCMSKPFNGRNILSVDLSVDAKCAQNWYAAK

SEQ ID NO: 11 - protein, 1049 aa, synthetic construct
  REGION 1..1049: note = Arabidopsis modified POPB sequence
MGVSLRHLSPSSFWVSRRPRVSSSILSFLVPRRRILCTRSRLKMCVCSIALFRKKVAIIKGNAGYSTATDCGGSHGFHHSGHQRSSSVEFSGEWKLNLGSKTARMVPPTVKQAGAVSAWREEVNNKLRGRNREYANNQDDAFGNGSYILKGFVPKIDDVHSYGNGQNFDYNLKPGTDITTLGRELNGFMQTNSIRGSVVALPSKDIEVGETTDVTLKPLNSDTTLDNASYKKTATISKVEKCTNLSQVRANLKKIYNRVRVVDNVSSAKETVALLMNQYRNLVHACATAVSRIDVKTETPVDHGEMICFSIYCGSEADFGDGKSCIWVDVLGENGRDILAEFKPFFEDSSIKKVWHNYSFDNHIIRNYGIKLSGFHGDTMHMARLWDSSRRISGGYSLEALTSDPKVLGGTETKEEAELFGKISMKKIFGKGKLKKDGSEGKLVIIPPVKELQMEDREAWISYSALDSISTLKLYESMKKQLQAKKWFLDGKLISKKNMFDFYQEYWQPFGELLAKMESEGMLVDRDYLAQIEIVAKAEQEIAVSRFRNWASKHCPDAKHMNVGSDTQLRQLFFGGISNSCNDEDLPYEKLFKVPNVDKVIEEGKKRATKFRNIKLHRISDRPLPTEKFTASGWPSVSGDTLKALAGKVSAEYDYMEGVLDTCLEENIGDDDCISLPDEVVETQHVNTSVESDTSAYGTAFDAFGGGESGKEACHAIAALCEVCSIDSLISNFILPLQGSNVSGKDGRVHCSLNINTETGRLSARRPNLQNQPALEKDRYKIRQAFIASPGNSLIVADYGQFELRILAHLASCESMKEAFIAGGDFHSRTAMNMYPHIREAVENGEVLLEWHPQPGQEKPPVPLLKDAFASERRKAKMLNFSIAYGKTAIGLSRDWKVSREEAQDTVNLWYNDRQEVRKWQELRKKEAIQKGYVLTLLGRARKFPEYRSRAQKNHIERAAINTPVQGSAADVAMCAMLEISNNQRLKELGWKLLLQVHDEVILEGPSESAENAKDIVVNCMSEPFNGKNILSVDLSVDAKCAQNWYAGK

SEQ ID NO: 12 - DNA, 5037 bp, synthetic construct
  misc_feature 1..5037: note = Arabidopsis modified POPA sequence
  source 1..5037: mol_type = other DNA
atggccatgggggtttctcttacttcccacaataaccctcttcttcgccacttatctccttcctcttcctgggtttctcgttcctcctctcgcctctcttcttctccactcccttccttcctctttccttgccgtcgaacccttcttcaaaggtttgcactttttttcacctttcgtgggttttagtaaattttgagactacacttgattatcgattactatgctctgttctatgctaaattttacttgattgtcgttactatgctctgttgtatgctcaattttaccttatttttgactaatttgctctgctcaagcctcagttttacttgattatcgattaagctactctgttataggctctgttttacttgattgtagattaatttgctctgttcgaagctcaatttgttcttgagtatgggataaagctgcttcgttctaggctcattttgttcttgtggggtttgttttgttttgctagctatgtttgtgactgcttttgaatcttttcaggaaactagcttccactgatggaaatgtcggttattgtactactacagtttgtcagggatttcagcattcggttcatcaacggtcgtcttctgttgtgtttaacggagagtgggaacttcgatcggaatctaacaaggtaaggatggttcctaagattatcaaggttggaaatcagacggaggtagccgaaactcatcaagttcctggaactgtaagtgcttggagggaagaagctaataagcttagagaaagaaatggtcaaattgctagaaaccttgatgacaatggttatttcaatggctctgtcccaattatatctagtgctccgtcttatgaaacctctcagaagattgattatgagttcaagcccagagggaccactaggtccactactgctacactcaataaggaattgattggcattacccaatctgaacctgttgtgtcattgccaaggaagggtttagatgttggggataatatggatgtaaatcctaaaggagagggaatacagagacctcttataagcgataagtcttctggtaccgcaaatggaaacaagaatactgttgccatttccaaagtggagagaagcactgaaccttcgaatgtccgtgaaaaccttgggaaaatatatgacaaggttctcattgttgataatgtgcaagctgcgaaggataccgtggctaagctcgtgaatcagtttaggaatcatgtccattcctgtgctacagcggtgtgctacatttttctatttaagttgtcatccctgcagctcagaaatatttatacaattgacaaactgattactctatgcttataggtgtccgggattgaggttaaggaagaaacgcctgtcgaccacggtgaattaatatgtttcagtatttactgtgggccagaagcagattttgggaatggaaaatcgtgcatctgggttgatgttcttggtgaaaatggcagggaggtgttggccgagtttaaaccatattttgaagactcgttcatcagaaaagtaagttataaactacaccttgattgtttctacatattctgtgatattttcattttctgagtccttctaaaacttattccactttcttgttgtcttttttactgtgaacatttttagctatccttaaacccttacaggttggctttcttcgtttcaggtctggcacaactacagtttcgatagccacatcattaggaaccatggaattgagatttctggttttcatgccgacactatgcacatggcacgattatgggattctgcacggcggataaaaggtggttattcgcttgaagcgcttacaagtgacccaaaagttcttgggggcacacagacaaaggaggaagctgagttccttggtaaaatttcgatgaagactatttttggcaaacggaaactgaaaaaagatggatctgaagggaaaatcgttgtcattccccctgttgaagagcttcagcgagaagatcgagaggcttggatttcatactctgccttggatgcgataagcacactaaagctttacgagagcatgacaaagaaactgcaattgatggattggcatcttgatggaaagccagttttaggaaggacaatgttggatttttaccatgagttctggcgaccctttggtgaacttcttgtaaaaatggaagcagaagggatacttgtagatagagagtatcttgctgagatagagaaagtagccaaagcggaacaacaagttgctggtagtaggtttcgtaattgggcatctaagtattgccccgatgcgaagtatatgaatattggcagtgacacacaattgcgtcagctcttttttggtggcatttccaacaggtatctttctcctacgtcatcgttaaatttctgctaagcataggtttaaattgaagttattatagggaaaatagtcatatcttggtatcttagaggttctttctttattatagagatttgagcttataatcctcttatctcaaaagttgttgaagactgtttttctttctcttggttcaaccagaactgcatacatagaaatctgagaagaaatgcctttcttatcaggtcattaacaagaattacatgattagtctctcagtgttacctctaataattaacttctgtttatttcttcctcagtagtcatgacgaggtgcttccagttgaaaagctcttcaaagtccccaatattgataaggttattgaagaaggcaaaaaaacaccgacgaagtttcgaaacatcaaactgcataggataagtgatagcccactgtcaacggaaaattttactgccagtggttggccctctgtaggtggggatgttttgaaagagttagctgggaaagtttctgctgaatacgactttatggatgatgtctcagatatttctctggaagaagttgttgaggatgatgatgtagaaacatcagaaacacaaaagtccaagacagatgatgaaaccgacacatctgcttatggaacagcatatgttgcatttggagggggtgaaaggggaaaggaggcatgtcatgctattgcctcattatgtgaagtttgctctatagactctttgatctcaaattttattcttccgttacaggtaattctgttttcttaacatcaccttctgaattttgagctgctttatctggtttaccttcaggttgaaatatgaattgttttttttttgtctgacagggaagtaatgtatcaggcaaagatggtcgcgtccattgctcccttaatatcaatacagaaactgggcgcttatcagctagaaggccaaatttgcaggtatatatcaaaaagctttgtgatattgttcaaaacaatatcgtttgaacctaaattaagtaataaatttgagtagtgaggccattatgtagctaagttcctcttgttatctactggtagcatattgttaacagagactcaatttgatgtgaaattatgtgatgtgttaccttgagctagtacataatttcatttcaaattggttttattttttggcaactgtaagtacacgatttgtaattgcatctacaatttgttacttaacttcctcttatctgtctagaaccaacctgcattggagaaggatcggtacaagattcgtaaggcctttgtagcatcgcctggaaatacacttgttgtggctgattatgggcaggtgaaaatataaaactctggtagttgtagagactttcataatggtattaatcttgttcacttgtttactgtgattctgttctatcttcagttcgaacttagaattctggcacatcttactggttgtaaaagcatgatggaagcttttaaagcaggcggagatttccactcaaggacagccatgaatatgtatccacatgttcgtgaagctgtagaaaatgggcaagtgatccttgaatggcatccagaacctggggaagacaagccacctgtgccgttgttaaaggtgcgttcttcgtttaaaattgtaccataattcttggattttgattcttatatcacaatgaggaaatgctttgaatagctataaaaacaagcgacatctctgttcaatgttcaaatcccgtcgttgactcaaagaacttatatctgtgcccaaatacttctaacagtttgcgaatatgtgtaatactcaggatgcctttggttcagagagaagaaaagccaagatgcttaacttttcaattgcgtatgggaaaaccgcggtcgggctttctagagattggaaggtagtggaaaaatactattgaaactattaggttgatataaacatgcgttgatgtgatgagtctaaaactgttcaaacttttttatcatctaaaggtctccactaaagaagctcaggaaacagtcgatctctggtacaatgacaggcaagaagtaaggaaatggcaagaaatgcgtaagaaagaagctatagaagatgggtatgtactcactttactaggaaggtctcgtagatttccggcgtcaaagtcacgtgctcagagaaatcatatccaaagagcagcaatcaacactccagtccaggtttccattctatttgtttactttatcataacactaatatccattctattctccaatcccaactctgagcatcgatgatccacactacagggaagtgcagctgatgttgccatgtgtgcaatgttggaaatatcgataaatcaacagttgaagaagcttggttggagattgcttctacaggtaaacaacaacacccctccaaaacaggacgcaatctatgaaaaatataagcttttaattttgtaacatagtggtctctattggattggaaattgagtaacaaagggaacttgtattggaaacagattcatgatgaagtaatcttggaaggaccaattgaatcagcggagatcgcaaaggacatagttgtggactgtatgtctaagccctttaacggaaggaatattctctcagtcgatttatctgttgatgcaaaatgtgctcaaaactggtatgctgccaaatag

SEQ ID NO: 13 - DNA, 5097 bp, synthetic construct
  misc_feature 1..5097: note = Arabidopsis modified POPB sequence
  source 1..5097: mol_type = other DNA
atgggggtttctcttcgtcacttgtctccttcctctttctgggtttcgcgtcgtcctcgtgtttcttcttcaattctttcttttcttgtccctcgccgtcgaatcctttgcaccaggttcacagcttttctcatcttatctgttcccttttctcatttttgttcatcgagaattagatttactcattcattagtcaattgctctgttccgtaaagttggttgttagttttatatagtgatgatgattcacgttttgaggttgttgaagaagcaggttaaaaatgtgtgtttgctcaattgctctgtttcgtaagtttgttgttagttatatagtgatgattcgcgttttaagtttgttgaagaagcaggttaaaaatgtgttttgctgcaatttggtttcagtttctcacttagtctaaattgttcctgtgttaaagttatttaggagtctgcttttgattaagcagaagaatgtattttagttgaggtttggttattattctgagtttgaaggtgtttttctttgtgttgatatcattttttggtggttatgatttgcaggaaagtagcaatcataaagggaaatgcgggatattcgactgctactgattgtggtggttctcatgggtttcatcattcaggtcatcaaaggtcatcctctgttgagtttagtggagagtggaaacttaatttgggatctaagacggcgagaatggttccgccaactgtgaaacaagctggagctgtaagtgcttggagggaggaagtcaataataagttaagaggaagaaatagagaatatgctaacaaccaggatgatgcttttggtaatggcagttatattttaaaaggctttgtccccaaaatagatgatgtccattcttatggaaacggccagaactttgattacaatctgaagcctggaactgatattaccacacttggtagagaattgaatggctttatgcaaacgaatagtataagaggatcggtcgtagcattaccaagcaaggatattgaggttggggaaacaacagacgtgactctgaaacctcttaacagtgatacgactcttgacaatgccagctataagaaaacagcaaccatttccaaagtggaaaaatgcaccaacctatcgcaagtccgtgcaaacctcaagaaaatatacaatagagttcgtgttgttgataatgtgtctagtgcaaaggaaaccgtggctctgctcatgaatcaatataggaatcttgtccatgcttgcgctacagcggtattgggtcatgtgtttgctaatacattgttgttcacatggcctagaaatgcttatgttcttgacgtatagacgcttcgtttaattctgcttttactctctgataatctattctgtaggtatccaggattgatgtgaagactgaaacacctgtagaccatggggagatgatttgtttcagtatctattgtggatcagaagcagattttggagatggtaaatcatgtatctgggtagatgtgcttggcgaaaatggaagggatatcctggctgagtttaagccatttttcgaagactcatccataaaaaaagtgagctaaaacgaaaaacaacttgctgcgtttctatttattcagtgataatttggtgttacgaatcttgttgtgtgttttcctgaaaagatgattgactacttcttcaccttggctctcttttgtttagatggaggcactaatgacttctgaatcatgttttaattttgtaaatcatttgaaaatgttttcatgatcaaataacgcttataagcttgacctttccttttacttataggtatggcataactacagctttgataaccacatcatcagaaattatggaatcaagctttctggttttcatggtgatacaatgcacatggcacgattgtgggattcatctagacggatatcgggtggttattcgcttgaagcacttacaagtgacccaaaagttcttgggggaactgagacaaaggaggaagcagaattatttggtaaaatatcaatgaagaagattttcggcaagggaaaattgaaaaaagatggatcagaaggaaaattggtgatcattcctcctgttaaagagctacaaatggaggatcgagaagcttggatttcgtactcagcgttggattcaataagcactctaaagctttatgagagcatgaagaagcaactgcaagcgaagaaatggtttcttgatggaaaactaatttcaaaaaagaacatgtttgatttctaccaagaatattggcaaccttttggtgaacttcttgccaaaatggaatcggaaggaatgcttgtagatagggattatttggcgcagattgagattgtagccaaagcagaacaagaaattgctgtttcgaggttccgtaattgggcttcaaagcattgtccagatgcaaagcatatgaatgttggcagtgacacgcaattgcgacaactcttttttggtggcatttctaacaggtatgcttcttccaagtcttgatttgaactgccttatttttccatgtatctttagaagaaaccatgacattttggtatttgacacgtaccatatcactttattataaagaaaaccaaatgtatgagactctagtttaagaatctggtgaagattgtaatcttttgccttgataaaacacttgcagctctcagatttagtttgtgaatcaaattttcttcccattatgatgcaaaacaggagttacataatttcgtttcttagtgcttatttctgatactaagttttgttcatatctgatacagttgtaatgatgaggatcttccatatgaaaaacttttcaaagttcccaatgtggataaggtgattgaagaaggaaaaaagagagccacaaaattccggaatatcaaactgcataggataagcgacagacctttgcccactgaaaagttcactgcctcaggctggccctctgttagtggagacaccttgaaagccttagctgggaaagtctctgcagaatatgactatatggagggcgttttagatacttgtctagaagaaaacattggagatgatgattgtatttcgctaccagatgaagttgtagaaacacaacacgtcaatacctctgttgaatcagacacatctgcttatggaacagcatttgatgcatttggagggggtgaaagtggaaaggaagcttgccatgctattgctgcattatgtgaagtttgctccattgattccttaatatcaaattttatccttcctttacaggtatgttttttcttttaaaacaatattccctggatttacgagctacttaattatttccattagcatatccttcaggttatttttttatggaatttgttttcctaaatgacagggaagtaacgtgtcaggaaaagatggtcgtgtccactgctccctgaatatcaacactgaaactggacgcttatcggctagaaggccaaatttgcaggtacatggcttgaacttaatttaagagttagcttgaatggagatgcaatcaaataactaagcttctctaatttctctactgataaccttgtaaatttttttgatgttgtggtatgaagagctagtatatgctaatttttggattggtgtgctcaaggtcaatttttagatagaggtataggacatctagaatttgtttgtaaagtattccttttctctgtctagaaccaacctgcattggagaaagatcggtacaaaatccgtcaggccttcatagcatcacctggaaattcacttattgttgctgattatggacaggtgaaatgatattgatatctacttttctgatgactagtaagctaattcacttgttgactgtgaggtgatttttctatcttcagttcgaacttaggattctggcacatcttgctagttgcgaaagcatgaaggaagctttcatagctggtggagacttccactcaagaacagccatgaatatgtatcctcatattcgtgaagctgttgaaaatggtgaagtgctccttgaatggcatccacaacctggacaagagaagccgccagtgccattgttgaaggtgaatcctggttaaatgtttttgaaacagggatttttaactcttttattcattttgcttgtaagctaaaagaatagttctattagtgctgaactgcagagcgggactctttcatccctttggcactttaagattgcaacagtcgacttatacagactagggaaactaaactttgttcctgaattcgtctaacagctggcaaaatgttttcttacttattcaggatgcctttgcttctgagagaagaaaagcaaagatgcttaacttctcgattgcgtatgggaagactgctattgggctgtctagagattggaaggtactatcaactaagccgttaagattgatatgaacaagcttgatataagataataagctcacagaagcctgttttccatctttcaggtatcaagagaagaagctcaagatacagttaatctctggtataatgacagacaagaagtccggaaatggcaagaactacgcaagaaagaagctatacaaaaagggtatgtactcactttgttgggaagggctcgtaaattccctgaataccgttcacgtgcccaaaagaaccatatcgagcgagcagcaatcaacactcctgttcaggtttgtttgcactatcatcagatcttaaccatcttgaactctatcgactttaaacctcaatcaccactattaatacagggaagtgcggctgatgttgctatgtgcgctatgctggagatatcaaataatcaacggctaaaagagcttggttggaaattgcttctacaagtaatacttctcacagattctttacataactacaggttagatgattgtttatctaaatgatttcttttgggctacctacaggttcatgatgaagtaatcttggaaggaccaagtgagtcagcggagaacgctaaagacatagttgtgaactgcatgtctgaacccttcaacggcaagaatattctctcagtcgacttatctgttgatgctaagtgtgctcagaactggtatgctggcaaataa

SEQ ID NO: 14 - protein, 1169 aa, synthetic construct
  REGION 1..1169: note = Nicotiana tabacum modified POP expression construct
  REGION 1..64: note = Petunia Rubisco Small subunit transit peptide
  REGION 1162..1169: note = Strep tag II
  REGION 1156..1161: note = Linker
MASSVISSAAVATRTNVAQASMVAPFNGLKSAVSFPVSSKQNLDITSIASNGGRVQCMSSLAVLGDSIKQISSHERKLFSSGLQHKIEEDSTYGWIAETNALKASKAKSSYNSYKKISAANCNVSASTNRRVKDEFFDVPTEVNTRMMRERITSSYSATTCISGGNLSSKSKPPYNPAGGEKKVVGNWREYENHLPQVSVGLTHSRVNGARSVNKVDGSNVSHYKPLSKGSHLNGQLSSKIMEPKLEKVNKLREGHASDQLRHSVNGTETKVVTVKAKGVIQERAMNKMEKNVIQAVTADVMNGAEANAKGVILERATNKMEKNAIESMATDVVNGTKTRIVNDEGTGVSQVSLRERLGAMYDKVHIVDNLSAAKEVVRKLTSQYRHLVHACATAVAKIDVKQQTPVDHGEIICFSIYSGPEADFGDGKSCIWVDVLDGDGKNLLVEFAPFFQDPSIRKVWHNYSFDNHVIENYGFKVSGFHADTMHMARLWDSSRRTSGGYSLEALTGDSTVMRDARPVHAERLFHGEGLFGKISMKTIFGRKKLKKDGTEGKVTVIPSVEELQKTERELWICYSALDSISTLMLYESLKNKLAKRIWTFDGVRKGSMYEFYEKYWRPFGELLVQMETEGVLVDRAYLAEIEKVAKAEQQVAANRFRNWAAKYCHDAKYMNVGSDTQLRQLFFGGIQNRKNSDESLPYEKEFKVPNIDKVTEEGKKAPTKFRKIRLHRICDLIDTEMYTASGWPSVSGDALKALSGKVSADFDILDEADDNAEEDPETSIDEALATNNEVPSQEPEVSIYGSAYNAFGGGQKGIEACHAIAALCEMCSIGSLISNFILPLQGQDVSGENGRIHCSLNINTETGRLSARRPNLQNQPALEKDRYKIRQAFVAAQGNSLIVADYGQFELRILAHLANCKSMLDAFKAGGDFHSRTAMNMYTHIREAVENGQVLLEWHPQPGEEKPPVPLLKDAFGSERRKAKMLNFSIAYGKTTIGLARDWKVSVKEAKETVDRWYRDRKEVSDWQEQRKFEAREFRRVHTLLGRARWFPSVKNATGSVKGHIERAAINTPVQGSAADVAMCAMLEISKNARLEELGWKLLLQVHDEVILEGPEESENEAMAIVVDCMSKPFGGKNILRVDLSVDSKCAKNWYSAKGSGSGSWSHPQFEK

SEQ ID NO: 15 - DNA, 1397 bp, Nicotiana tabacum
  source 1..1397: mol_type = genomic DNA
cgtttaaggaagcatatcaagggttgtcttgtgcgtcctctaggaattcgtatttaagttgatttaagacaatttgggttatttttaatttgttaaacttatttaagtttaatgtgttaatttataagacaagttaatttgaagtattatatgcaatgaaaatatgaaatgaaagcctttgaagtttgatccttacatattggtcttattaaataattttatatggccacttaatttcaattttcaaatttaaaactttaaattttaaagtttaaaagtttaattttataccttaaaatacttattttattatttaaaaatttgctaacacttaagtaacaattacattgaacaagaaaacataatacactaaaaaaaagaaaattaacccgtcccggtccggaaccgataagcccgaacccggacggacccaccaaaacccggaatatcccagcccactaccagcacggaatcccagcccactaaccagctcgccccactaaccggacgagctgttttttcccatgtccagctcggtccagcccgtccgttaaacacccatattctaaatagagattgagtgttcaatacgtcatggggtgagtttaagtgtctaggtgaaaatagggacaagtttatgagtttgtctgtgtatttataatttataaatatttatatggttataaattatttcattaagaattttttttttaagattagttatttttttgggtaagaggagaacccgcatctgctactacctctgtcacacctctgtccttcgggtgatcactctgcggcgaatattttgtatgcactggataaatttatcttatgtaataatctgcaaatcacacagaaaatataaatcgtactaggcaagtcctgtgcgacatataaccgggaaggcattaaggagaatccatccgatggatcatggttattgaaaaggagtaactaacaaaaaagaaaaaacacgagacgacaacatagtcaaataaatgtccgtgccagcgagcaaaagggcttttctttttctttttgctcagaagagagaggaaaaagggttttaagacgactcagctactaataatacaataattgttctttgctcttccgccccctctctctgtttacagtgtaagtatagaagacagaagagtgtactgtatacatatggtctctgttctctgcttctccactaccacttccagacaaacaacaaataaacccttgtaaaaccctaaatttttcttacttttacagtctcaccccctttagttgttcattttatttttgtggttaaaatcaaaaaatacaaaatacactacactaatttatttattataaagacggccgctcttttgagcaataagctgctagtaagcagagttgcc

SEQ ID NO: 16 - DNA, 260 bp, Arabidopsis thaliana
  source 1..260: mol_type = genomic DNA
tagaagcgataacaaaataaaaaggaaacaatagtaattagatggcgcaaaaataagatccaacggctgagatctttactcgtgaacgttctcgaaagctctttgccgacccactcttcattcatatataaacaaacacctctctgccttctcttcctcacacaatcataaacacaacaacactcacaaattctcttaaagctcacagacgaattctttctatttttaatctttccggcgaacaattctgatctctaata

SEQ ID NO: 17 - DNA, 521 bp, Arabidopsis thaliana
  source 1..521: mol_type = genomic DNA
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctgattagattcaccgaccctcctatcgtgtcga

SEQ ID NO: 18 - protein, 927 aa, synthetic construct
  REGION 1..927: note = GFP-GUS with chloroplast transit peptide
  REGION 1..57: note = Petunia Rubisco Small subunit transit peptide
MASSVISSAAVATRTNVAQASMVAPFNGLKSAVSFPVSRKQNLDITSIASNGGRVQCMVSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKSNAADEVATQLLNFDLLKLAGDVESNPGPMLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 19 - DNA, 24 bp, synthetic construct
  misc_feature 1..24: note = delNdeIPstI-F
  source 1..24: mol_type = other DNA
tacgagaaagaattcaaagttgca

SEQ ID NO: 20 - DNA, 18 bp, synthetic construct
  misc_feature 1..18: note = delNdeIPstI-R
  source 1..18: mol_type = other DNA
actttgaattctttctcg

SEQ ID NO: 21 - DNA, 35 bp, synthetic construct
  misc_feature 1..35: note = M13-F
  source 1..35: mol_type = other DNA
ttcccagtcacgacgttgtaaaacgacggccagtg

SEQ ID NO: 22 - DNA, 20 bp, synthetic construct
  misc_feature 1..20: note = pUN121-F
  source 1..20: mol_type = other DNA
cccccaagtctggctatgca

SEQ ID NO: 23 - DNA, 21 bp, synthetic construct
  misc_feature 1..21: note = pUN121-R
  source 1..21: mol_type = other DNA
caaccattatcaccgccagag

SEQ ID NO: 24 - DNA, 57 bp, synthetic construct
  misc_feature 1..57: note = CompetpUN-R1
  source 1..57: mol_type = other DNA
gcttagaacctttaccaaaggtgatgcggagagatgggtaagcacaaccaaaaaagc

SEQ ID NO: 25 - DNA, 53 bp, synthetic construct
  misc_feature 1..53: note = CompetPUN-R2
  source 1..53: mol_type = other DNA
cagtgattctgcattctggcttgaggttgaaggtaattccatgaccgcaccaa

SEQ ID NO: 26 - DNA, 52 bp, synthetic construct
  misc_feature 1..52: note = CompetPUN-R3
  source 1..52: mol_type = other DNA
caggctccaagccaagctttcctgacggaatgttaattctcgttgaccctga

SEQ ID NO: 27 - DNA, 50 bp, synthetic construct
  misc_feature 1..50: note = pUN121_mut
  source 1..50: mol_type = other DNA
tcagggtcaacgagaattaacattccgtcaggagcttggcttggagcctg

SEQ ID NO: 28 - DNA, 24 bp, synthetic construct
  misc_feature 1..24: note = N.tomNativeDPProm-F
  source 1..24: mol_type = other DNA
gattcggtaaggggtgagacgggt

SEQ ID NO: 29 - DNA, 24 bp, synthetic construct
  misc_feature 1..24: note = N.tomNativeDPProm-R
  source 1..24: mol_type = other DNA
ggagaaggaatgaggggaaaacca

SEQ ID NO: 30 - DNA, 27 bp, synthetic construct
  misc_feature 1..27: note = N.tomNativeDPProm-SacI-F
  source 1..27: mol_type = other DNA
gagctcgtttaaggaagcatatcaagg

SEQ ID NO: 31 - DNA, 19 bp, synthetic construct
  misc_feature 1..19: note = N.tomNativeDPProm-NcoI-R
  source 1..19: mol_type = other DNA
actgaactgagaaccccaa

SEQ ID NO: 32 - DNA, 28 bp, synthetic construct
  misc_feature 1..28: note = AtHSP70-F
  source 1..28: mol_type = other DNA
gacaatttaaaaaagacccatctcgaag

SEQ ID NO: 33 - DNA, 22 bp, synthetic construct
  misc_feature 1..22: note = AtHSP70-R
  source 1..22: mol_type = other DNA
ttcatggcgacttgattcttgg

SEQ ID NO: 34 - DNA, 24 bp, synthetic construct
  misc_feature 1..24: note = AtHSP70-SacI-F1
  source 1..24: mol_type = other DNA
ggagctcgaaggagctagaagcga

SEQ ID NO: 35 - DNA, 36 bp, synthetic construct
  misc_feature 1..36: note = AtHSP70-BsmB1-R1
  source 1..36: mol_type = other DNA
ccgtctccccattattagagatcagaattgttcgcc

SEQ ID NO: 36 - DNA, 28 bp, synthetic construct
  misc_feature 1..28: note = PetrbcS-JJ-F cloning
  source 1..28: mol_type = other DNA
accacataatccaatggttagctttact

SEQ ID NO: 37 - DNA, 28 bp, synthetic construct
  misc_feature 1..28: note = PetrbcS-JJ-R
  source 1..28: mol_type = other DNA
cgagtttcaagttccgagtaattacata

SEQ ID NO: 38 - DNA, 33 bp, synthetic construct
  misc_feature 1..33: note = PetrbcS-JJ-F_BsmBI
  source 1..33: mol_type = other DNA
ccgtctccatggcttcctctgtgatttcctctg

SEQ ID NO: 39 - DNA, 34 bp, synthetic construct
  misc_feature 1..34: note = PetrbcS-JJ-R_BsmBI
  source 1..34: mol_type = other DNA
ccgtctcctggacatgcattggactcttccacca

SEQ ID NO: 40 - DNA, 30 bp, synthetic construct
  misc_feature 1..30: note = GFP_BsmB1-F
  source 1..30: mol_type = other DNA
gacgtctctgcatggtgagcaagggcgagg

SEQ ID NO: 41 - DNA, 31 bp, synthetic construct
  misc_feature 1..31: note = GFP_XhoI-R
  source 1..31: mol_type = other DNA
gagactcgagttacttgtacagctcgtccat

SEQ ID NO: 42 - DNA, 35 bp, synthetic construct
  misc_feature 1..35: note = cpTargetingGFP_BsmB1-F
  source 1..35: mol_type = other DNA
cgtctctgcatggtgagcaagggcgaggagctgtt

SEQ ID NO: 43 - DNA, 35 bp, synthetic construct
  misc_feature 1..35: note = cpTargetingGFP_BsmB1-R
  source 1..35: mol_type = other DNA
cgtctcgcttgtacagctcgtccatgccgtgagtg

SEQ ID NO: 44 - DNA, 61 bp, synthetic construct
  misc_feature 1..61: note = LP4-2A linker-F
  source 1..61: mol_type = other DNA
cgtctcccaagtctaatgctgctgatgaagttgctactcaacttcttaattttgatcttct

SEQ ID NO: 45 - DNA, 78 bp, synthetic construct
  misc_feature 1..78: note = LP4-2A linker-R
  source 1..78: mol_type = other DNA
cgtctccacattggtcctggattagattcaacatctccagcaagcttaagaagatcaaaattaagaagttgagtagca

SEQ ID NO: 46 - DNA, 32 bp, synthetic construct
  misc_feature 1..32: note = GUStoLinker_BsmB1-F
  source 1..32: mol_type = other DNA
cgtctccatgttacgtcctgtagaaaccccaa

SEQ ID NO: 47 - DNA, 29 bp, synthetic construct
  misc_feature 1..29: note = GUStoLinker_BsmB1-R
  source 1..29: mol_type = other DNA
ctcgagctattgtttgcctccctgctgcg

SEQ ID NO: 48 - DNA, 26 bp, synthetic construct
  misc_feature 1..26: note = AtHSP18.2-F
  source 1..26: mol_type = other DNA
agaacgaagagaagaacgataagtgg

SEQ ID NO: 49 - DNA, 28 bp, synthetic construct
  misc_feature 1..28: note = AtHSP18.2-R
  source 1..28: mol_type = other DNA
tgttacatgaggagccttaaccaaaaca

SEQ ID NO: 50 - DNA, 24 bp, synthetic construct
  misc_feature 1..24: note = AtHSP18.2XhoI-F
  source 1..24: mol_type = other DNA
cctcgagatatgaagatgaagatg

SEQ ID NO: 51 - DNA, 31 bp, synthetic construct
  misc_feature 1..31: note = AtHSP18.2NotI-R
  source 1..31: mol_type = other DNA
ggcggccgctcgacacgataggagggtcggt

SEQ ID NO: 52 - DNA, 21 bp, synthetic construct
  misc_feature 1..21: note = Ntcp16S-F
  source 1..21: mol_type = other DNA
cgaactccgggcgaatatgaa

SEQ ID NO: 53 - DNA, 25 bp, synthetic construct
  misc_feature 1..25: note = Ntcp16S-R
  source 1..25: mol_type = other DNA
ggaaagaagacttccatctccaagt

SEQ ID NO: 54 - DNA, 21 bp, synthetic construct
  misc_feature 1..21: note = SeqNtcp16S-R
  source 1..21: mol_type = other DNA
ccgctttgtctacgaacaagg

SEQ ID NO: 55 - DNA, 24 bp, synthetic construct
  misc_feature 1..24: note = StrepTagRTPCR-F (MuPOP)
  source 1..24: mol_type = other DNA
tctggatctggatcttggtctcat

SEQ ID NO: 56 - DNA, 25 bp, synthetic construct
  misc_feature 1..25: note = AtUTRRTPCR-R (MuPOP)
  source 1..25: mol_type = other DNA
gccacaaattcataacacaacaagc

SEQ ID NO: 57 - DNA, 20 bp, synthetic construct
  misc_feature 1..20: note = EF1alpha-F (control)
  source 1..20: mol_type = other DNA
tgagatgcaccacgaagctc

SEQ ID NO: 58 - DNA, 22 bp, synthetic construct
  misc_feature 1..22: note = EF1alpha-R (control)
  source 1..22: mol_type = other DNA
ccaacattgtcaccaggaagtg

SEQ ID NO: 59 - DNA, 26 bp, synthetic construct
  misc_feature 1..26: note = PG2rbcl-F
  source 1..26: mol_type = other DNA
tcccctgttgggatgtactattaaac

SEQ ID NO: 60 - DNA, 23 bp, synthetic construct
  misc_feature 1..23: note = PG2rbcl-R
  source 1..23: mol_type = other DNA
cttatccaaaacgtccactgctg

SEQ ID NO: 61 - DNA, 24 bp, synthetic construct
  misc_feature 1..24: note = W1ycf3-F
  source 1..24: mol_type = other DNA
tcatacggctcagcagtcaattct

SEQ ID NO: 62 - DNA, 25 bp, synthetic construct
  misc_feature 1..25: note = W1ycf3-R
  source 1..25: mol_type = other DNA
cgatttttatcagctgtagcaaaga

SEQ ID NO: 63 - DNA, 23 bp, synthetic construct
  misc_feature 1..23: note = psaB_TA-F
  source 1..23: mol_type = other DNA
cagtagcccaaacaagatgtcca

SEQ ID NO: 64 - DNA, 24 bp, synthetic construct
  misc_feature 1..24: note = psaB_TA-R
  source 1..24: mol_type = other DNA
cgggttggttaaatgctgttaatg

SEQ ID NO: 65 - DNA, 24 bp, synthetic construct
  misc_feature 1..24: note = psbJ_TA-F
  source 1..24: mol_type = other DNA
ttacgctttcatgttgacgactca

SEQ ID NO: 66 - DNA, 24 bp, synthetic construct
  misc_feature 1..24: note = psbJ_TA-R
  source 1..24: mol_type = other DNA
tcattcggaaggatctcataattt

SEQ ID NO: 67 - DNA, 27 bp, synthetic construct
  misc_feature 1..27: note = ndhF_AG-F
  source 1..27: mol_type = other DNA
aaactccctgttatccaataaaaacct

SEQ ID NO: 68 - DNA, 27 bp, synthetic construct
  misc_feature 1..27: note = ndhF_AG-R
  source 1..27: mol_type = other DNA
agtcatggttttttcaatctacctgtc

SEQ ID NO: 69 - protein, 17 aa, Escherichia coli
IVSADYSQIELRIMAHL

SEQ ID NO: 70 - protein, 17 aa, synthetic construct
  REGION 1..17: note = NtPOPtom motif A
LIVADYGQLELRILAHL

SEQ ID NO: 71 - protein, 17 aa, synthetic construct
  REGION 1..17: note = NtPOPtom Exo- L903F motif A
LIVADYGQFELRILAHL

SEQ ID NO: 72 - protein, 13 aa, Escherichia coli
VFAFDTETDSLDN

SEQ ID NO: 73 - protein, 13 aa, synthetic construct
  REGION 1..13: note = NtPOPtom Exol motif
VHACDTEVAKIDV

SEQ ID NO: 74 - protein, 13 aa, synthetic construct
  REGION 1..13: note = NtPOPtom Exo- L903F Exol motif
VHACATAVAKIDV

SEQ ID NO: 75 - protein, 64 aa, synthetic construct
  REGION 1..64: note = MuPOP transit peptide upper sequence
MASSVISSAAVATRTNVAQASMVAPFNGLKSAVSFPVSSKQNLDITSIASNGGRVQCMSSLAVL

SEQ ID NO: 76 - protein, 61 aa, synthetic construct
  REGION 1..61: note = MuPOP transit peptide lower sequence
MAFLGFSVQSSPFKPTSYLWFSPHSFSSSRSFWASSGKALHRREDCKTQSVENASSSLAVL

SEQ ID NO: 77 - DNA, 101 bp, synthetic construct
  misc_feature 1..101: note = CPgenome sequence
  source 1..101: mol_type = other DNA
gaaggtaatgaaattattcgcgaggcttgcaaatggagcccggaactagctgctgcttgtgaagtatggaaagagatcgtatttaattttgcagcagtgga

SEQ ID NO: 78 - DNA, 101 bp, synthetic construct
  misc_feature 1..101: note = PG2 rbcL sequences
  source 1..101: mol_type = other DNA
gaaggtaatgaaattattcgcgaggcttgcaaatggagcccggaactagctgctgcttatgaagtatggaaagagatcgtatttaattttgcagcagtgga

SEQ ID NO: 79 - DNA, 72 bp, synthetic construct
  misc_feature 1..72: note = CPgenome sequence
  source 1..72: mol_type = other DNA
ggttaattacatgagtttcaaactgaaatttggatgaataatccgtttatttagttttatcttttttcccac

SEQ ID NO: 80 - DNA, 72 bp, synthetic construct
  misc_feature 1..72: note = W1 ycf3 sequences
  source 1..72: mol_type = other DNA
ggttaattacatgagtttcaaactgaaatttgtatgaataatccgtttatttagttttatcttttttcccac

SEQ ID NO: 81 - DNA, 83 bp, synthetic construct
  misc_feature 1..83: note = CPgenome sequence
  source 1..83: mol_type = other DNA
ggaagattcattaaactgtgaaacgttaccctgccataatgtgatgtgcttccaatgccaataaaaagtaacccatccaatag

SEQ ID NO: 82 - DNA, 83 bp, synthetic construct
  misc_feature 1..83: note = W6_psaB sequence
  source 1..83: mol_type = other DNA
ggaagattcattaaactgtgaaacgttaccctgccataatgagatgtgcttccaatgccaataaaaagtaacccatccaatag

SEQ ID NO: 83 - DNA, 82 bp, synthetic construct
  misc_feature 1..82: note = CPgenome sequence
  source 1..82: mol_type = other DNA
aaaccgattacaagaataccagctacagtacctattatccaaagaggaatccttccagtagtatcggccatttaccccactt

SEQ ID NO: 84 - DNA, 82 bp, synthetic construct
  misc_feature 1..82: note = W6 psbJ sequence
  source 1..82: mol_type = other DNA
aaaccgattacaagaataccagctacagtacctattatccaaagaggaatccttccagaagtatcggccatttaccccactt

SEQ ID NO: 85 - DNA, 83 bp, synthetic construct
  misc_feature 1..83: note = CPgenome sequence
  source 1..83: mol_type = other DNA
ccaattcccaaaaaatataaatttgtatcaaattcgaactagtaactaatcccaacatggaagtactgaaaaaactcatataa

SEQ ID NO: 86 - DNA, 83 bp, synthetic construct
  misc_feature 1..83: note = W6_ndhF sequence
  source 1..83: mol_type = other DNA
ccaattcccaaaaaatataaatttgtatcaaattcgaactggtaactaatcccaacatggaagtactgaaaaaactcatataa

SEQ ID NO: 87 - DNA, 48 bp, synthetic construct
  misc_feature 1..48: note = cl repressor alpha 1 domain
  source 1..48: mol_type = other DNA
acacaagagcagcttgaggacgcacgtcgccttaaagcaatttatgaa

SEQ ID NO: 88 - DNA, 54 bp, synthetic construct
  misc_feature 1..54: note = cI repressor alpha 5 domain
  source 1..54: mol_type = other DNA
gaagaattcagcccttcaatcgccagagaaatctacgagatgtatgaagcggtt

SEQ ID NO: 89 - protein, 1455 aa, Physcomitrella patens
MEGSVQRLVIQLYKSAVAAGFRTPCFTSPSFSAPSSRAFMPVHCFLLRIPFSHIRSPSKLVVAPIRPTRPHLLSSSRFTPCKDYSRRLERNYYVGSRILPANGRQSSFWRRNSDVKDDVTAAGARKSHEFPSAPESKNGTAEVSYKKAGERRTITVSRPLKWANTLAALSSTQLREAIRLASLDEKVYDAVMLVKVLGLNDWSRKRKELNFIGGLLRDADPELMEQVLRACEDGDRLGSTSRRLSLPYFRPHYFNSSSTGGNLSRLWNTSASKNFDSSSKSNLYTAMVGTVSAMQRKPSLSVQHTREQVQLPNTVNTAPQFRFPQSIQHFKSSPSNFVPPVSRTVKVALQAVSKTKSHLTNGYALPQLARRFGKLRAQNQNSHLLTSRKTPDVGVTSQAVDSGRGEAWERAAHQLGILVLAKPLSLSALKTEVPLSEFEPEKSHSSTSIATDRNDYSKSFRLSSSCEKKSEIFKPVIVNERSCKSEIFKPVIVKERSCKSEIFKPVIVKERSCKSEIFKPVIVNERSCKSEIFKPVIVKERPCKSLNFSDYIPEQKSGFSNSAEKIGEENQPSSHADQSESGQIKKTSAKRSITVKKAIVDNSPEESKLRTRSRKKHVPVQLERKDLGENNVTGDSHVSALDSERSSNMDPYEERRPVSIKSVMIVDSVEKAEMVVEQLMSEYKNVVHACDTEVAGIDVKKESPVGHGQITCFSIYCGPGADFGYGKNRLWVDVLDGGDDVLRVFKRYFEDPSIQKVWHNYSFDKHILSRHGIHPQGFYADTMHLARLNDSARRGSKGGYALEVLSADRKVMDYCSKNFTEEDGSVFVGKKSMKELFGKAKLKKDGTPGKIKVVPPVDELQRDEELRDAWIHYSTLDAVCTWRLFVSLQHKLSNTPWSVAELRHKGSMYDFYEKYWRPFGEVLVQMEAYGMLVDYDHLATVEKLARAQQKISVSRFRKWAARYCPNAARMNVGSDAQIRQFLFGGTANRKDADQALPMERVFSTPNTDGFIEEGKKIAKKTKPMVITGLANHGIKIPVETYTSSGWPAVGGAAIRALAGKVSIDYSDIDDDAAEGVLEVDTEPEVSLTSAGVETDHEEDLSVYGKAYKAFLGGQEGKEACMALAALCEVASINTLLSNFIEPLQGNDIKSVSDGRVHCSLNINTETGRLSARRPSLQNQPALEKDRYKIRQAFVAAPGKALVVADYGQLELRLLAHLADCKSMKAAFIAGGDFHSRTAMNMYPHVREAVEKDRVLLEWEGLEKPPVPLLKDMFGSERRKAKMLNFSIAYGKTAMGLAKDWNVKLDEAKATVDLWYSDRPEVLAWQKERKQEAHETLRVHTLLGRARHLPDINSSNSLLRSHMERAAINTPVQGSAADVAMCAMLEINQNARLRELGWKLLLQVHDEVILEGPLESAEEAKELVIKSMMYPFNGENILDVELVVDGDYAENWYAAK

SEQ ID NO: 90 - DNA, 3462 bp, synthetic construct
  source 1..3462: mol_type = other DNA
  misc_feature 1..3462: note = Nicotiana tabacum modified POP nucleotide sequence
atgatgatgatgatgagccgcagcggcgccaaccgggtagcgaacaccgcaatgtttgtggcgaagggtttaagcggcgaagttggtggtttaagagcattatatggcggtggtgtgaggagtgagtccagcttagcagtgcttggcgattccattaaacaaatatcaagtcacgaaaggaagttattttctagtggactacaacataaaattgaggaagacagtacttacggatggatcgctgaaactaatgctcttaaagcttctaaagctaaaagctcttataatagttacaaaaagatttcagcagcaaattgcaatgttagtgcctctacaaatcggagggtgaaagatgaattttttgatgttccaactgaagtcaatacgagaatgatgcgtgaacgcatcacctctagctattctgccacaacctgtatttcaggaggcaacttatcttcaaaaagcaaaccaccttataaccctgctggaggagaaaagaaggtagttggaaactggagagagtatgaaaaccatttgccacaagtatctgtaggtcttacccattcaagagtcaatggagctaggtcagtaaataaggttgatggttctaatgtatctcattacaaacccttatcaaaaggttcccatcttaatgggcaattatcaagcaaaattatggaaccaaagttggagaaggtaaataaactaagggagggtcatgcatctgatcaactaagacacagtgtgaatgggaccgagaccaaggtagttactgtcaaagcaaaaggtgttatccaggagcgtgctatgaataaaatggagaagaatgtaattcaagccgtgacagctgatgtcatgaatggagctgaggccaatgcaaaaggggttatcctggagcgtgcaactaataaaatggaaaagaatgcaattgaatccatggcaactgatgtcgtgaatggaaccaagactaggattgtcaatgatgaaggaacaggtgtaagtcaggtttccctccgtgagaggctgggtgcaatgtatgacaaagttcatatagttgacaatttatcagcagctaaggaagttgtcagaaagcttacaagtcagtacaggcatctcgtccatgcgtgtgctacagcggtagcaaagattgatgttaagcagcaaacacctgttgatcatggagaaattatatgcttcagtatttattctggaccagaggctgattttggtgatgggaaatcttgtatctgggtagatgttcttgatggagatggcaagaacctcttagttgaatttgctccatttttccaagacccatccattagaaaggtctggcacaattatagctttgacaaccatgtcatcgagaactatgggtttaaagtatctggctttcacgctgacacaatgcacatggcacgactttgggattcctctaggcgaacttcaggtggttattcactagaggcactaacaggtgattccactgtcatgcgtgatgctagaccggtccatgctgaaaggctgttccatggtgaaggtctgtttggtaaaatatctatgaaaacgatctttggtcggaaaaagctgaagaaagacggaactgagggtaaagttactgtcattccttctgttgaagagttgcaaaaaactgaaagagaattatggatttgttattctgcattagactccataagcacattgatgctttacgagagtttgaagaataaactggctaaacggatctggacattcgatggagttcgtaaaggatcgatgtatgagttttatgagaaatactggcgtccatttggtgagcttctggttcaaatggaaactgagggtgtgctggttgaccgtgcctatcttgctgagattgagaaagtggctaaagctgagcagcaggttgctgctaatagatttcgtaactgggcagctaagtactgtcatgatgcaaagtacatgaatgttggaagtgatacacagttgcgtcagctgttttttggtggcatccagaatagaaagaattctgatgagagtctaccatatgagaaagaattcaaagttccaaatatcgataaagtaactgaagaaggaaagaaggctcccaccaaatttcgtaaaatccgactacatagaatttgtgatcttatcgacactgagatgtacactgccagtggctggccttctgttagtggggatgctttgaaggctctctctggcaaagtttctgcggactttgatattcttgatgaagcggatgacaatgctgaggaagatcctgaaacaagcattgatgaagctttagctacaaataatgaagttcccagtcaagaaccagaagtttctatttatggatcagcgtacaacgcctttggaggtgggcagaaaggaattgaggcctgccatgccattgcagcattatgtgaaatgtgctccataggctctttaatatccaactttatactcccattgcaaggccaagatgtatcgggtgagaatggacgaattcattgttccctgaatattaacactgaaaccgggcgcctgtctgcgaggagaccaaatttacagaaccagcctgctctggagaaagataggtacaaaattcgtcaagcttttgtagctgcacaagggaattccttgattgtggccgactatgggcagttcgaacttaggattcttgcacatcttgccaactgtaagagcatgttggatgctttcaaagctggtggagactttcattcaaggactgctatgaacatgtatactcacatccgtgaagccgttgaaaatggtcaggtacttcttgagtggcatcctcaaccaggtgaagaaaaacctccggttcctcttctaaaggatgcttttggctctgaaagaaggaaggcaaagatgctcaatttttcaattgcatacgggaaaactacaattggacttgcccgcgattggaaggtatccgtaaaggaagctaaggaaacagttgatcggtggtatagagatagaaaagaagtgtcagattggcaggaacaacgcaaatttgaagcacgcgagttcagacgtgttcacacacttctaggacgggcacgctggttcccatcagtaaaaaatgcaactggatctgtaaaaggtcacatagaacgagctgctataaatactccagtgcagggaagtgctgcagatgttgctatgtgcgccatgttagagatatcaaagaatgcacgactagaggagcttggatggaaattgcttttacaggttcatgatgaagttattttggaagggccggaagaatctgaaaacgaagccatggcaatagtggttgactgcatgtccaagccttttggtggaaagaatattctcagagttgacctatctgttgattctaaatgtgccaaaaactggtattctgccaagggatctggatctggatcttggtctcatcctcagttcgagaagtga

SEQ ID NO: 91 - protein, 1153 aa, synthetic construct
  REGION 1..1153: note = Nicotiana tabacum modified POP expression construct
  REGION 1..48: note = soybean (Glycine max) Alternative Oxidase 1 (AOX1) presequence
  REGION 1146..1153: note = Strep tag II
  REGION 1140..1145: note = Linker
MMMMMSRSGANRVANTAMFVAKGLSGEVGGLRALYGGGVRSESSLAVLGDSIKQISSHERKLFSSGLQHKIEEDSTYGWIAETNALKASKAKSSYNSYKKISAANCNVSASTNRRVKDEFFDVPTEVNTRMMRERITSSYSATTCISGGNLSSKSKPPYNPAGGEKKVVGNWREYENHLPQVSVGLTHSRVNGARSVNKVDGSNVSHYKPLSKGSHLNGQLSSKIMEPKLEKVNKLREGHASDQLRHSVNGTETKVVTVKAKGVIQERAMNKMEKNVIQAVTADVMNGAEANAKGVILERATNKMEKNAIESMATDVVNGTKTRIVNDEGTGVSQVSLRERLGAMYDKVHIVDNLSAAKEVVRKLTSQYRHLVHACATAVAKIDVKQQTPVDHGEIICFSIYSGPEADFGDGKSCIWVDVLDGDGKNLLVEFAPFFQDPSIRKVWHNYSFDNHVIENYGFKVSGFHADTMHMARLWDSSRRTSGGYSLEALTGDSTVMRDARPVHAERLFHGEGLFGKISMKTIFGRKKLKKDGTEGKVTVIPSVEELQKTERELWICYSALDSISTLMLYESLKNKLAKRIWTFDGVRKGSMYEFYEKYWRPFGELLVQMETEGVLVDRAYLAEIEKVAKAEQQVAANRFRNWAAKYCHDAKYMNVGSDTQLRQLFFGGIQNRKNSDESLPYEKEFKVPNIDKVTEEGKKAPTKFRKIRLHRICDLIDTEMYTASGWPSVSGDALKALSGKVSADFDILDEADDNAEEDPETSIDEALATNNEVPSQEPEVSIYGSAYNAFGGGQKGIEACHAIAALCEMCSIGSLISNFILPLQGQDVSGENGRIHCSLNINTETGRLSARRPNLQNQPALEKDRYKIRQAFVAAQGNSLIVADYGQFELRILAHLANCKSMLDAFKAGGDFHSRTAMNMYTHIREAVENGQVLLEWHPQPGEEKPPVPLLKDAFGSERRKAKMLNFSIAYGKTTIGLARDWKVSVKEAKETVDRWYRDRKEVSDWQEQRKFEAREFRRVHTLLGRARWFPSVKNATGSVKGHIERAAINTPVQGSAADVAMCAMLEISKNARLEELGWKLLLQVHDEVILEGPEESENEAMAIVVDCMSKPFGGKNILRVDLSVDSKCAKNWYSAKGSGSGSWSHPQFEK

SEQ ID NO: 92 - DNA, 828 bp, synthetic construct
  source 1..828: mol_type = other DNA
  misc_feature 1..828: note = mScarlet-I with mitochondrial presequence nucleotide sequence
atgatgatgatgatgtctaggtcaggaggtaacagggtagctaacacagcaatgtttgtggcaaagggtttgtcaggtgaagttggtggtttgagagcattgtatggtggtggtgtgaggagtgagtccatggtttctaagggagaagctgttattaaggaatttatgagatttaaggttcatatggaaggatctatgaatggacatgaatttgaaattgaaggagaaggagaaggaagaccatatgaaggaactcaaactgctaagcttaaggttactaagggaggaccacttccattttcttgggatattctttctccacaatttatgtatggatctagagcttttattaagcatccagctgatattccagattattataagcaatcttttccagaaggatttaagtgggaaagagttatgaattttgaagatggaggagctgttactgttactcaagatacttctcttgaagatggaactcttatttataaggttaagcttagaggaactaattttccaccagatggaccagttatgcaaaagaagactatgggatgggaagcttctactgaaagactttatccagaagatggagttcttaagggagatattaagatggctcttagacttaaggatggaggaagatatcttgctgattttaagactacttataaggctaagaagccagttcaaatgccaggagcttataatgttgatagaaagcttgatattacttctcataatgaagattatactgttgttgaacaatatgaaagatctgaaggaagacattctactggaggaatggatgaactttataagtaa

SEQ ID NO: 93 - protein, 275 aa, synthetic construct
  REGION 1..275: note = mScarlet-I with mitochondrial presequence amino acid sequence
  REGION 1..43: note = soybean (Glycine max) Alternative Oxidase 1 (AOX1) presequence
MMMMMSRSGGNRVANTAMFVAKGLSGEVGGLRALYGGGVRSESMVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 94 - DNA, 24 bp, synthetic construct
  source 1..24: mol_type = other DNA
  misc_feature 1..24: note = B18SHetero-F primer
actactataccaaacgcactccac

SEQ ID NO: 95 - DNA, 24 bp, synthetic construct
  source 1..24: mol_type = other DNA
  misc_feature 1..24: note = B18SHetero-R primer
caactacctaatactacgcaggct

SEQ ID NO: 96 - DNA, 24 bp, synthetic construct
  source 1..24: mol_type = other DNA
  misc_feature 1..24: note = Inter1Hetro-F primer
attagtcgaatgagcccatctctc

SEQ ID NO: 97 - DNA, 24 bp, synthetic construct
  source 1..24: mol_type = other DNA
  misc_feature 1..24: note = Inter1Hetro-R primer
agaagtttgactagttgctcctcc

SEQ ID NO: 98 - DNA, 38 bp, Nicotiana tabacum
  source 1..38: mol_type = genomic DNA
gaacgctagctatatgcttaacacatgcaagtcgaacg

SEQ ID NO: 99 - DNA, 38 bp, Nicotiana tabacum
  source 1..38: mol_type = genomic DNA
gctcttcaatcgacgatctactgctccctcttaatagc

SEQ ID NO: 100 - protein, 16 aa, synthetic construct
  REGION 1..16: note = cI repressor alpha 1 translation (Fig. 26)
TQEQLEDARRLKAIYE

SEQ ID NO: 101 - protein, 17 aa, synthetic construct
  REGION 1..17: note = cI repressor alpha 5 domain translation (Fig. 26)
EFSPSIAREIYEMYEAV